(19) United States
(12) Patent Application Publication
(10) Pub. No.: US 2026/0234197 A1
(43) Pub. Date: Aug. 13, 2026

(54) ENGINEERED AAV POLYPEPTIDES

(71) Applicant: Embold Therapeutics, Inc., Redwood City, CA (US)

(72) Inventors: Hassibullah AKEEFE, San Ramon, CA (US); Chinping CHNG, Redwood City, CA (US); Elizabeth DEL GRECO, Windsor, CA (US); Sarah DESAUTEL, San Rafael, CA (US); Kahsay GEBRETSADIK, Fairfield, CA (US); Nikki D. KRUSE, Redwood City, CA (US); Ambuj KUMAR, Redwood City, CA (US); Mirella RIVERA-VELAZQUEZ, San Jose, CA (US); Marcus ROHOVIE, Redwood City, CA (US); Christopher Edward Schmitt, San Francisco, CA (US); Faezeh SEDIGHI, Redwood City, CA (US); Antoinette SERO, Redwood City, CA (US); Adam P. SILVERMAN, San Carlos, CA (US); Victoria WONG, San Francisco, CA (US)

(21) Appl. No.: 19/153,020

(22) PCT Filed: Feb. 2, 2024
(86) PCT No.: PCT/US2024/014362

§ 371 Date: Aug. 1, 2025

Related U.S. Application Data
(60) Provisional application No. 63/483,010, filed on Feb. 2, 2023.
(60) Provisional application No. 63/502,053, filed on May 12, 2023.

(51) Int. Cl. C07K 14/005; A61K 38/00; A61K 48/00; C07K 1/14; C12N 15/86

(52) U.S. Cl. CPC C07K 14/005; A61K 48/0033; C07K 1/14; C12N 15/86; A61K 38/00; C12N 2750/14122; C12N 2750/14143; C12N 2750/14151

(57) ABSTRACT

The present disclosure provides engineered AAV capsid polypeptides, engineered MAAP polypeptides, and engineered AAP polypeptides as well as recombinant polynucleotides encoding the engineered AAV polypeptides. The present disclosure further provides use of the engineered AAV polypeptides and recombinant polynucleotides for producing rAAV viruses or rAAV virions.

### CROSS-REFERENCE TO RELATED APPLICATIONS

This application claims the benefit of U.S. Provisional Application No. 63/483,010, filed Feb. 2, 2023, and U.S. Provisional Application No. 63/502,053, filed May 12, 2023, the contents of all of which are incorporated by reference herein.

### REFERENCE TO SEQUENCE LISTING, TABLE OR COMPUTER PROGRAM

The Sequence Listing concurrently submitted herewith as file name CX7-242WO2_ST26, created on Feb. 2, 2024, with a file size of 3,560,627 bytes, is part of the specification and is hereby incorporated by reference herein.

### TECHNICAL FIELD

The present disclosure relates to engineered or recombinant adeno-associated virus (rAAV) polypeptides, compositions thereof, polynucleotides encoding the engineered rAAV polypeptides, and uses of the engineered polypeptides and recombinant polynucleotides.

### BACKGROUND

Adeno-associated virus (AAV) is a single stranded DNA virus that has a genome with genes encoding rep and cap proteins. The rep gene encodes four rep proteins, Rep78, Rep68, Rep52, and Rep40, necessary for genome replication, and the cap gene encodes three structural proteins. VP1, VP2, and VP3, which assemble to form the viral capsid. The viral genome also encodes two additional proteins, MAAP and AAP, via alternative reading frames in the cap gene. AAP appears to function in capsid assembly. MAAP may be involved in capsid secretion. The AAV genome has inverted terminal repeats (ITR) at the 5′ and 3′ ends flanking the rep/cap genes. The ITRs contain the origin of replication, packaging signals, and affects persistence in cells following infection (see, e.g., Earley et al., Hum Gene Ther., 2020, 31 (3-4): 151-162).

AAV is dependent upon the presence of a helper virus, such as an adenovirus or herpesvirus, for active replication. In the absence of a helper, the AAV exists in a latent state in which its genome is maintained episomally or integrated into the host chromosome. Numerous human and non-human primate and other mammalian AAV serotypes have been identified and sequenced. These AAVs can be grouped into six genetic groups (clades A-F) and two clonal isolates (AAV4 and AAV5) based on antigenic reactivity and sequence analysis.

Because only the ITRs are necessary for virus propagation, the rep and cap genes can be replaced with heterologous DNA, allowing the engineering of AAV as gene delivery vehicles, such as for gene therapy. While other viral vectors have been developed for gene delivery (e.g., adenovirus, retrovirus, lentivirus, herpes simplex virus alphavirus, and flavivirus), recombinant adeno-associated viral (rAAV) vectors offer important advantages for use in gene therapy applications. First, AAV and AAV vectors have favorable safety profiles because, in part, the virus is not associated with any known human disease; the virus can replicate only under certain circumstances; and the vector rarely integrates into the host genome. Second, AAV has the ability to infect both dividing and non-dividing cells and displays differing ranges of tropism for cell types, including muscle, lung, and brain. Third, AAV can provide sustained, long-term expression of the transgene in various tissue cell types. These advantages of AAV vectors in gene therapy have resulted in approval of AAV based vectors for delivering genes encoding, among others, Factor IX for treating hemophilia B, human survival motor neuron protein (SMN) for treating spinal muscular dystrophy, and retinoid isomerohydrolase RPE65 for treating inherited retinal dystrophy.

Despite the advantages and success of AAV based gene therapy, vector design problems remain. Issues include limited tissue or cell tropism, poor infection of non-permissive cell types such as stem cells, challenges with high efficiency targeting of gene delivery to selected cell populations, and presence of neutralizing antibodies (NAbs) that block gene delivery to some patients. Accordingly, there is a need in the art for improved AAV vectors that address some of the drawbacks to current AAV based vectors.

### SUMMARY

The present disclosure provides engineered AAV polypeptides useful in the production of rAAV viruses and virions. In some embodiments, the engineered AAV polypeptide is an engineered AAV capsid polypeptide. In some embodiments, the engineered AAV capsid polypeptides arc characterized by, among others, improved expression, neutralizing antibody avoidance, and improved tropism for neuronal cells and CNS.

In some embodiments, the engineered adeno-associated virus (AAV) capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to an even-numbered SEQ ID NO. of SEQ ID NOs: 2-1134, wherein the amino acid sequence comprises one or more substitutions relative to the reference sequence corresponding to SEQ ID NO: 2, 278, or 522.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 2, 278, or 522, wherein the amino acid sequence comprises one or more substitutions relative to the reference sequence corresponding to SEQ ID NO: 2, 278, or 522.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 2, wherein the amino acid sequence comprises one or more substitutions relative to the reference sequence corresponding to SEQ ID NO: 2.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522, wherein the amino acid sequence comprises one or more substitutions relative to the reference sequence corresponding to SEQ ID NO: 2.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO. of SEQ ID NOs: 4-1134, wherein the amino acid sequence comprises one or more substitutions relative to the reference sequence corresponding to SEQ ID NO: 2.

In some embodiments, the amino acid sequence of the engineered AAV capsid polypeptide comprises at least a substitution at amino acid position 10, 14, 17, 22, 23, 24, 25, 31, 33, 45, 51, 53, 56, 60, 63, 66, 79, 81, 90, 100, 101, 119, 125, 126, 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to SEQ ID NO: 2.

In some embodiments, the amino acid sequence of the engineered AAV capsid polypeptide comprises at least two substitutions at amino acid positions 588, 665, 457, 495, 456, 505, 494, 497, 592, 586, 585, 706, 549, 552, 664, 661, 461, 668, 548, 510, 640, 419, 349, 504, 508, 453, 589, 56, and 416, wherein the amino acid positions are relative to the reference sequence corresponding to SEQ ID NO: 2.

In some embodiments, the amino acid sequence of the engineered AAV capsid polypeptide comprises at least two substitutions comprising a first and second substitution, wherein the substitutions are at amino acid positions (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, 56, and 416, wherein the first substitution is at a position selected from (a), and the second substitution is at a position selected from (a), (b) and (c), wherein the amino acid positions are relative to the reference sequence corresponding to SEQ ID NO: 2.

In some embodiments, the amino acid sequence of the engineered AAV capsid polypeptide comprises at least a substitution set at amino acid positions 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to the reference sequence corresponding to SEQ ID NO: 2.

In some embodiments, the amino acid sequence of the engineered AAV capsid polypeptide comprises at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 56/529, 331/449, 211/509/712, 331/416/478, 56/507, 56/331/584/597, 56/478, 349/465/479/504/508, 349/640, 331/584/597, 331/416/478/483/507/584/597, 56/416, 233/465/640, 125/508/510/589, 597, 331/597, 56/331/416/665, 125/465/504/508/510, 56/331/478/483/529/597, 384/479, 233/640, 125/419/504/508/530, 504/510/640, 419/504/508, 56/331/361, 416/665, 56/211/361/416/478/584, 56, 56/331/416, 175/211, 56/211/478/483/712, 349/504/508, 125/508/510, 331/416, 331, 479/504/510/589, 331/478, 56/331/478/483/597, 479, 125/504/508, 479/589, or 479/510/589, wherein the amino acid positions are relative to the reference sequence corresponding to SEQ ID NO: 2.

In some embodiments, the amino acid sequence of the engineered AAV capsid polypeptide comprises at least one substitution set forth in Tables 5.1, 5.2, 6.1, 6.2, 7.1, and 7.2, wherein the amino acid positions are relative to the reference sequence corresponding to SEQ ID NO: 2.

In some embodiments, the amino acid sequence of the engineered AAV capsid polypeptide comprises at least a substitution or substitution set of an engineered AAV capsid polypeptide set forth in Tables 5.1, 5.2, 6.1, 6.2, 7.1, and 7.2, wherein the amino acid positions are relative to the reference sequence corresponding to SEQ ID NO: 2.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to an even-numbered SEQ ID NO. of SEQ ID NOs: 4-1134.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to an even-numbered SEQ ID NO. of SEQ ID NOs: 4-1134, wherein the amino acid sequence comprises one or more substitutions relative to the reference sequence corresponding to SEQ ID NO: 278 or 522.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522, wherein the amino acid sequence comprises one or more substitutions relative to the reference sequence corresponding to SEQ ID NO: 278 or 522.

In some embodiments, the amino acid sequence of the AAV capsid polypeptide comprises at least a substitution at amino acid position 10, 14, 17, 22, 23, 24, 25, 31, 33, 45, 51, 53, 56, 60, 63, 66, 79, 81, 90, 100, 101, 119, 125, 126, 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to SEQ ID NO: 278 or 522.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278, wherein the amino acid sequence comprises one or more substitutions relative to the reference sequence corresponding to SEQ ID NO: 278.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to an even-numbered SEQ ID NO. of SEQ ID NOs: 332-722, wherein the amino acid sequence comprises one or more substitutions relative to the reference sequence corresponding to SEQ ID NO: 278.

In some embodiments, the amino acid sequence of the engineered AAV capsid polypeptide comprises at least a substitution or substitution set at amino acid position(s) 14, 173, 159, 157, 152, 485, 421, 391, 469, 628, 461, 165, 642, 595, 24, 414, 512, 519, 172, 168, 598, 449, 736, 156, 462, 162, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 155, 663, 401, 22, 175, 579, 444, 23, 459/508/663/667, 17, 412, 440, 387, 453/586, 419, 25, 472, 162/450, 585, 437, 10, 562, 169, 588, 565, 589, 495/497, 119/450, or 142/552, wherein the amino acid positions are relative to the reference sequence corresponding to SEQ ID NO: 278.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 552, wherein the amino acid sequence comprises one or more substitutions relative to the reference sequence corresponding to SEQ ID NO: 552.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to an even-numbered SEQ ID NO. of SEQ ID NOs: 724-1134, wherein the amino acid sequence comprises one or more substitutions relative to the reference sequence corresponding to SEQ ID NO: 552.

In some embodiments, the amino acid sequence of the engineered AAV capsid polypeptide comprises at least a substitution or substitution set at amino acid position(s) 725, 414/456/459/485/736, 193, 157/165/454, 157/165/668, 485/595, 165/454/485, 346, 421/456/459, 218, 205, 348, 421, 551, 165, 736, 529/552/585, 535, 529/585, 535/659, 594, 468, 101, 726, 468/592, 451, 559, 575, 465, 560, 273, 389/510, 470, 100, 724, 485/597, 510, 45, 256, 334, 63, 211, 264, 595, 391, 391/661, 200, 165/391/454/471, 157/165/391, 165/391, 157/391/421, 81, 401/414/485, 391/454/595, 529, 529/552/588/716, 493/585/667, 269, 552, 515, 565, 470/663, 414, 597, 564, 389, 60, 720, 451/452, 496, 214, or 320, wherein the amino acid positions are relative to the reference sequence corresponding to SEQ ID NO: 552.

In some embodiments, the amino acid sequence of the engineered AAV capsid polypeptide comprises at least a substitution or substitution set at amino acid position(s) 333, 190, 254, 100, 190/725, 31, 101, 258, 268, 214/451, 331, 328, 33, 274, 33/576, 560, 51/190, 60, 79/272, 263, 270, 267, 210/266, 259, 53, 259/466, 90, 264, 172/266, 90/535, 266, 66, 329, or 126/470, wherein the amino acid positions are relative to the reference sequence corresponding to SEQ ID NO: 552.

In some embodiments, the amino acid sequence of the engineered AAV capsid polypeptide comprises at least one substitution set forth in Tables 5.1, 5.2, 6.1, 6.2, 7.1, and 7.2, wherein the amino acid positions are relative to the reference sequence corresponding to SEQ ID NO: 2, 278, or 552.

In some embodiments, the amino acid sequence of the engineered AAV capsid polypeptide comprises at least a substitution or substitution set of an engineered AAV capsid polypeptide set forth in Tables 5.1, 5.2, 6.1, 6.2, 7.1, and 7.2, wherein the amino acid positions are relative to the reference sequence corresponding to SEQ ID NO: 2, 278, or 552.

In some embodiments, the engineered AAV capsid polypeptide comprises an amino acid sequence comprising an even-numbered SEQ ID NO. of SEQ ID NOs: 4-1134, optionally wherein the amino acid sequence has 1, 2, 3, 4, 5, 6, 7, 8, 9, or up to 10 substitutions.

In some embodiments, the engineered AAV capsid polypeptide is an engineered AAV VP2 capsid polypeptide based on the engineered AAV capsid polypeptide disclosed herein.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 138-736 of an even-numbered SEQ ID NO. of SEQ ID NOs: 2-1134, wherein the amino acid sequence comprises one or more substitutions relative to the reference sequence corresponding to residues 138-736 of SEQ ID NO: 2, 278, or 522.

In some embodiments, the engineered AAV VP2 capsid polypeptide comprises an amino acid scquence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 2, 278, or 522, wherein the amino acid sequence comprises one or more substitutions relative to the reference sequence corresponding to residues 138-736 of SEQ ID NO: 2, 278, or 522.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 2, wherein the amino acid sequence comprises one or more substitutions relative to the reference sequence corresponding to residues 138-736 of SEQ ID NO: 2.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522, wherein the amino acid sequence comprises one or more substitutions relative to the reference sequence corresponding to residues 138-736 of SEQ ID NO: 2.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO. of SEQ ID NOs: 4-1134, wherein the amino acid sequence comprises one or more substitutions relative to the reference sequence corresponding to residues 138-736 of SEQ ID NO: 2.

In some embodiments, the amino acid sequence of the engineered AAV VP2 capsid polypeptide comprises at least a substitution at amino acid position 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to SEQ ID NO: 2.

In some embodiments, the amino acid sequence of the engineered AAV VP2 capsid polypeptide comprises substitutions in at least two amino acid positions 588, 665, 457, 495, 456, 505, 494, 497, 592, 586, 585, 706, 549, 552, 664, 661, 461, 668, 548, 510, 640, 419, 349, 504, 508, 453, 589, and 416, wherein the amino acid positions are relative to the reference sequence corresponding to SEQ ID NO: 2.

In some embodiments, the amino acid sequence of the engineered AAV VP2 capsid polypeptide comprises at least two substitutions comprising a first and second substitution, wherein the substitutions are at amino acid positions (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, and 416, wherein the first substitution is at a position selected from (a), and the second substitution is at a position selected from (a), (b) and (c), wherein the amino acid positions are relative to the reference sequence corresponding to SEQ ID NO: 2.

In some embodiments, the amino acid sequence of the engineered AAV VP2 capsid polypeptide comprises at least a substitution set at amino acid positions 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to SEQ ID NO: 2.

In some embodiments, the amino acid sequence of the engineered AAV VP2 capsid polypeptide comprises at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 331/449, 211/509/712, 331/416/478, 507, 331/584/597, 478, 349/465/479/504/508, 349/640, 331/416/478/483/507/584/597, 416, 233/465/640, 508/510/589, 597, 331/597, 331/416/665, 465/504/508/510, 331/478/483/529/597, 384/479, 233/640, 419/504/508/530, 504/510/640, 419/504/508, 331/361, 416/665, 211/361/416/478/584, 331/416, 175/211, 211/478/483/712, 349/504/508, 508/510, 331/416, 331, 479/504/510/589, 331/478, 331/478/483/597, 479, 504/508, 479/589, or 479/510/589, wherein the amino acid positions are relative to the reference sequence corresponding to SEQ ID NO: 2.

In some embodiments, the amino acid sequence of the engineered AAV VP2 capsid polypeptide comprises at least one substitution between residues 138-736 set forth in Tables 5.1, 5.2, 6.1, 6.2, 7.1, and 7.2, wherein the amino acid positions are relative to the reference sequence corresponding to SEQ ID NO: 2.

In some embodiments, the amino acid sequence of the engineered AAV VP2 capsid polypeptide comprises at least a substitution or substitution set between residues 138-736 of an engineered AAV capsid polypeptide set forth in Tables 5.1, 5.2, 6.1, 6.2, 7.1, and 7.2, wherein the amino acid positions are relative to the reference sequence corresponding to SEQ ID NO: 2.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO. of SEQ ID NOs: 4-1134.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522, wherein the amino acid sequence comprises one or more substitutions relative to the reference sequence corresponding to residues 138-736 of SEQ ID NO: 278 or 522.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO. of SEQ ID NOs: 4-1134, wherein the amino acid sequence comprises one or more substitutions relative to the reference sequence corresponding to residues 138-736 of SEQ ID NO: 278 or 522.

In some embodiments, the amino acid sequence of the engineered AAV VP2 capsid polypeptide comprises at least a substitution at amino acid position 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 138-736 of SEQ ID NO: 278 or 522.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278, wherein the amino acid sequence comprises one or more substitutions relative to the reference sequence corresponding to residues 138-736 of SEQ ID NO: 278.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 138-736 of an even-numbered SEQ ID NO. of SEQ ID NOs: 332-722, wherein the amino acid sequence comprises one or more substitutions relative to the reference sequence corresponding to residues 138-736 of SEQ ID NO: 278.

In some embodiments, the amino acid sequence of the engineered AAV VP2 capsid polypeptide comprises at least a substitution or substitution set at amino acid position(s) 173, 159, 157, 152, 485, 421, 391, 469, 628, 461, 165, 642, 595, 414, 512, 519, 172, 168, 598, 449, 736, 156, 462, 162, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 155, 663, 401, 175, 579, 444, 459/508/663/667, 412, 440, 387, 453/586, 419, 472, 162/450, 585, 437, 562, 169, 588, 565, 589, 495/497, 119/450, or 142/552, wherein the amino acid positions are relative to the reference sequence corresponding to residues 138-736 of SEQ ID NO: 278.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 552, wherein the amino acid sequence comprises one or more substitutions relative to the reference sequence corresponding to residues 138-736 of SEQ ID NO: 552.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO. of SEQ ID NOs: 724-1134, wherein the amino acid sequence comprises one or more substitutions relative to the reference sequence corresponding to residues 138-736 of SEQ ID NO: 552.

In some embodiments, the amino acid sequence of the engineered AAV VP2 capsid polypeptide comprises at least a substitution or substitution set at amino acid position(s) 725, 414/456/459/485/736, 193, 157/165/454, 157/165/668, 485/595, 165/454/485, 346, 421/456/459, 218, 205, 348, 421, 551, 165, 736, 529/552/585, 535, 529/585, 535/659, 594, 468, 726, 468/592, 451, 559, 575, 465, 560, 273, 389/510, 470, 724, 485/597, 510, 256, 334, 63, 211, 264, 595, 391, 391/661, 200, 165/391/454/471, 157/165/391, 165/391, 157/391/421, 401/414/485, 391/454/595, 529, 529/552/588/716, 493/585/667, 269, 552, 515, 565, 470/663, 414, 597, 564, 389, 720, 451/452, 496, 214, or 320, wherein the amino acid positions are relative to the reference sequence corresponding to residues 138-736 of SEQ ID NO: 552.

In some embodiments, the amino acid sequence of the engineered AAV VP2 capsid polypeptide comprises at least a substitution or substitution set at amino acid position(s) 333, 190, 254, 190/725, 258, 268, 214/451, 331, 328, 274, 576, 560, 79/272, 263, 270, 267, 210/266, 259, 259/466, 264, 172/266, 535, 266, 329, or 470, wherein the amino acid positions are relative to the reference sequence corresponding to residues 138-736 of SEQ ID NO: 552.

In some embodiments, the amino acid sequence of the engineered AAV VP2 capsid polypeptide comprises at least one substitution between residues 138-736 set forth in Tables 5.1, 5.2, 6.1, 6.2, 7.1, and 7.2, wherein the amino acid positions are relative to the reference sequence corresponding to residues 138-736 of SEQ ID NO: 2, 278, or 552.

In some embodiments, the amino acid sequence of the engineered AAV VP2 capsid polypeptide comprises at least a substitution or substitution set between residues 138-736 of an engineered AAV capsid polypeptide set forth in Tables 5.1, 5.2, 6.1, 6.2, 7.1, and 7.2, wherein the amino acid positions are relative to the reference sequence corresponding to residues 138-736 of SEQ ID NO: 2, 278, or 552.

In some embodiments, the amino acid sequence of the engineered AAV VP2 capsid polypeptide comprises residues 138-736 of an even-numbered SEQ ID NO. of SEQ ID NOs: 4-1134, optionally wherein the amino acid sequence has 1, 2, 3, 4, 5, 6, 7, 8, 9, or up to 10 substitutions.

In some embodiments, the engineered AAV capsid polypeptide is an engineered AAV VP3 capsid polypeptide based on the engineered AAV capsid polypeptide disclosed herein.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of an even-numbered SEQ ID NO. of SEQ ID NOs: 2-1134, wherein the amino acid sequence comprises one or more substitutions relative to the reference sequence corresponding to residues 203-736 of SEQ ID NO: 2, 278, or 522.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of SEQ ID NO: 2, 278, or 522, wherein the amino acid sequence comprises one or more substitutions relative to the reference sequence corresponding to residues 203-736 of SEQ ID NO: 2, 278, or 522.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 2, wherein the amino acid sequence comprises one or more substitutions relative to the reference sequence corresponding to residues 203-736 of SEQ ID NO: 2.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278 or 522, wherein the amino acid sequence comprises one or more substitutions relative to the reference sequence corresponding to residues 203-736 of SEQ ID NO: 2.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO. of SEQ ID NOs: 4-1134, wherein the amino acid sequence comprises one or more substitutions relative to the reference sequence corresponding to residues 203-736 of SEQ ID NO: 2.

In some embodiments, the amino acid sequence of the AAV VP3 capsid polypeptide comprises at least a substitution at amino acid position 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 203-736 of SEQ ID NO: 2.

In some embodiments, the amino acid sequence of the engineered AAV VP3 capsid polypeptide comprises at least substitutions in at least two amino acid positions 588, 665, 457, 495, 456, 505, 494, 497, 592, 586, 585, 706, 549, 552, 664, 661, 461, 668, 548, 510, 640, 419, 349, 504, 508, 453, 589, and 416, wherein the amino acid positions are relative to the reference sequence corresponding to residues 203-736 of SEQ ID NO: 2.

In some embodiments, the amino acid sequence of the engineered AAV VP3 capsid polypeptide comprises at least two substitutions comprising a first and second substitutions, wherein the substitutions are at amino acid positions (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, and 416, wherein the first substitution is at a position selected from (a), and the second substitution is at a position selected from (a), (b) and (c), wherein the amino acid positions are relative to the reference sequence corresponding to residues 203-736 of SEQ ID NO: 2.

In some embodiments, the amino acid sequence of the AAV VP3 capsid polypeptide comprises at least a substitution set at amino acid positions 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 331/449, 211/509/712, 331/416/478, 507, 331/584/597, 478, 349/465/479/504/508, 349/640, 331/416/478/483/507/584/597, 416, 233/465/640, 508/510/589, 597, 331/597, 331/416/665, 465/504/508/510, 331/478/483/529/597, 384/479, 233/640, 419/504/508/530, 504/510/640, 419/504/508, 331/361, 416/665, 211/361/416/478/584, 331/416, 175/211, 211/478/483/712, 349/504/508, 508/510, 331/416, 331, 479/504/510/589, 331/478, 331/478/483/597, 479, 504/508, 479/589, or 479/510/589, wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution or substitution set between residues 203-736 set forth in Tables 5.1, 5.2, 6.1, 6.2, 7.1, and 7.2, wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution or substitution set between residues 203-736 of an engineered AAV capsid polypeptide set forth in Tables 5.1, 5.2, 6.1, 6.2, 7.1, and 7.2, wherein the amino acid positions are relative to the reference sequence corresponding to residues 203-736 of SEQ ID NO: 2.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278 or 522.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO. of SEQ ID NOs: 4-1134.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278 or 522, wherein the amino acid sequence comprises one or more substitutions relative to the reference sequence corresponding to residues 203-736 of SEQ ID NO: 278 or 522.

In some embodiments, the amino acid sequence of the engineered AAV VP3 capsid polypeptide comprises at least a substitution at amino acid position 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 203-736 of SEQ ID NO: 278, or 522.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278, wherein the amino acid sequence comprises one or more substitutions relative to the reference sequence corresponding to residues 203-736 of SEQ ID NO: 278.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of an even-numbered SEQ ID NO. of SEQ ID NOs: 332-722, wherein the amino acid sequence comprises one or more substitutions relative to the reference sequence corresponding to residues 203-736 of SEQ ID NO: 278.

In some embodiments, the amino acid sequence of the engineered AAV VP3 capsid polypeptide comprises at least a substitution or substitution set at amino acid position(s) 485, 421, 391, 469, 628, 461, 642, 595, 414, 512, 519, 598, 449, 736, 462, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 663, 401, 579, 444, 459/508/663/667, 412, 440, 387, 453/586, 419, 472, 450, 585, 437, 562, 169, 588, 565, 589, 495/497, or 142/552, wherein the amino acid positions are relative to the reference sequence corresponding to residues 203-736 of SEQ ID NO: 278.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 552, wherein the amino acid sequence comprises one or more substitutions relative to the reference sequence corresponding to residues 203-736 of SEQ ID NO: 552.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of an even-numbered SEQ ID NO. of SEQ ID NOs: 724-1134, wherein the amino acid sequence comprises one or more substitutions relative to the reference sequence corresponding to residues 203-736 of SEQ ID NO: 552.

In some embodiments, the amino acid sequence of the engineered AAV VP3 capsid polypeptide comprises at least a substitution or substitution set at amino acid position(s) 725, 414/456/459/485/736, 454, 668, 485/595, 454/485, 346, 421/456/459, 218, 205, 348, 421, 551, 736, 529/552/585, 535, 529/585, 535/659, 594, 468, 726, 468/592, 451, 559, 575, 465, 560, 273, 389/510, 470, 724, 485/597, 510, 256, 334, 211, 264, 595, 391, 391/661, 391/454/471, 391/421, 401/414/485, 391/454/595, 529, 529/552/588/716, 493/585/667, 269, 552, 515, 565, 470/663, 414, 597, 564, 389, 720, 451/452, 496, 214, or 320, wherein the amino acid positions are relative to the reference sequence corresponding to residues 203-736 of SEQ ID NO: 552.

In some embodiments, the amino acid sequence of the engineered AAV VP3 capsid polypeptide comprises at least a substitution or substitution set at amino acid position(s) 333, 254, 725, 258, 268, 214/451, 331, 328, 274, 576, 560, 272, 263, 270, 267, 210/266, 259, 259/466, 264, 266, 535, 329, or 470, wherein the amino acid positions are relative to the reference sequence corresponding to residues 203-736 of SEQ ID NO: 552.

In some embodiments, the amino acid sequence of the engineered AAV VP3 capsid polypeptide comprises at least one substitution between residues 203-736 set forth in Tables 5.1, 5.2, 6.1, 6.2, 7.1, and 7.2, wherein the amino acid positions are relative to the reference sequence corresponding to residues 203-736 of SEQ ID NO: 2, 278 or 522.

In some embodiments, the amino acid sequence of the engineered AAV VP3 capsid polypeptide comprises at least a substitution or substitution set between residues 203-736 of an engineered AAV capsid polypeptide set forth in Tables 5.1, 5.2, 6.1, 6.2, 7.1, and 7.2, wherein the amino acid positions are relative to the reference sequence corresponding to residues 203-736 of SEQ ID NO: 2, 278 or 522.

In some embodiments, the amino acid sequence of the engineered AAV VP3 capsid polypeptide comprises residues 203-736 of an even-numbered SEQ ID NO. of SEQ ID NOs: 4-1134, optionally wherein the amino acid sequence has 1, 2, 3, 4, 5, 6, 7, 8, 9, or up to 10 substitutions.

In some embodiments, the engineered AAV polypeptide is an engineered MAAP polypeptide disclosed herein.

In some embodiments, the engineered MAAP polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 1136, wherein the amino acid sequence comprises one or more substitutions relative to the reference sequence corresponding to SEQ ID NO: 1136.

In some embodiments, the amino acid sequence of the engineered MAAP polypeptide comprises at least a substitution at amino acid position 5, 7, 17, 27, 30, 34, 40, 64, 73, 74, 75, 93, 99, 100, or 116, or combinations thereof, wherein the amino acid positions are relative to SEQ ID NO: 1136.

In some embodiments, the amino acid sequence of engineered MAAP polypeptide comprises at least a substitution or substitution set at amino acid position(s) 99, 30, 17, 93, 116, 73, 5, 75, 34, 74, 27, 7, 73/74, 64, 40, or 100, wherein the amino acid positions are relative to SEQ ID NO: 1136.

In some embodiments, the engineered MAAP polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, or 1232.

In some embodiments, the amino acid sequence of the engineered MAAP polypeptide comprises SEQ ID NO: 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, or 1232, optionally wherein the amino acid sequence has 1, 2, 3, 4, 5, 6, 7, 8, 9, or up to 10 substitutions.

In some embodiments, the engineered AAV polypeptide is an engineered AAP polypeptide disclosed herein.

In some embodiments, the engineered AAP polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 1234, wherein the amino acid sequence comprises one or more substitutions relative to the reference sequence corresponding to SEQ ID NO: 1234.

In some embodiments, the amino acid sequence of the engineered AAP polypeptide comprises at least a substitution at amino acid position 14, 15, 31, 49, 58, 79, 83, 84, 85, 88, 89, 91, 92, 93, 94, 95, 97, 99, 100, 102, 104, 105, 120, 153, 154, 155, 156, 157, 158, 160, 173, 174, or 194, or combinations thereof, wherein the amino acid positions are relative to SEQ ID NO: 1234.

In some embodiments, the amino acid sequence of the engineered AAP polypeptide comprises at least a substitution or substitution set at amino acid position(s) 58, 156, 49/156, 174, 157/158, 160, 158, 15, 79, 104, 31/156, 83, 92/93, 102, 155/156, 14/15, 153, 194, 99, 85, 105, 97, 88, 94/95, 91/92, 100, 91, 92, 83/84, 89, 94, 94/95/173, 97/120, 95, or 154, wherein the amino acid positions are relative to SEQ ID NO: 1234.

In some embodiments, the engineered AAP polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, or 1410.

In some embodiments, the amino acid sequence of the engineered AAP polypeptide comprises SEQ ID NO: 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, or 1410, optionally wherein the amino acid sequence has 1, 2, 3, 4, 5, 6, 7, 8, 9, or up to 10 substitutions.

In another aspect, the present disclosure provides a recombinant polynucleotide encoding any of the engineered AAV polypeptide described herein.

In some embodiments, the recombinant polynucleotide comprises a polynucleotide sequence encoding an engineered AAV capsid polypeptide described herein.

In some embodiments, the recombinant polynucleotide comprises a polynucleotide sequence having at least 60%, 65%, 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to an SEQ ID NO: 277 or 521, wherein the recombinant polynucleotide encodes an AAV capsid polypeptide.

In some embodiments, the recombinant polynucleotide comprises a polynucleotide sequence having at least 60%, 65%, 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to an odd-numbered SEQ ID NO. of SEQ ID NOs: 3-1133, wherein the recombinant polynucleotide encodes an AAV capsid polypeptide.

In some embodiments, the recombinant polynucleotide comprises a polynucleotide sequence comprising a recombinant polynucleotide of an engineered AAV capsid polypeptide set forth in Tables 5.1, 5.2, 6.1, 6.2, 7.1, and 7.2.

In some embodiments, the recombinant polynucleotide comprises a polynucleotide sequence comprising an odd-numbered SEQ ID NO. of SEQ ID NOs: 3-1133.

In some embodiments, the recombinant polynucleotide comprises a polynucleotide sequence encoding an engineered AAV VP2 capsid polypeptide described herein.

In some embodiments, the recombinant polynucleotide comprises a polynucleotide sequence having at least 60%, 65%, 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 412-2208 of an odd-numbered SEQ ID NO. of SEQ ID NOs: 3-1133.

In some embodiments, the recombinant polynucleotide comprises a polynucleotide sequence comprising nucleotide residues 412-2208 of a recombinant polynucleotide of an engineered AAV capsid polypeptide set forth in Tables 5.1, 5.2, 6.1, 6.2, 7.1, and 7.2.

In some embodiments, the recombinant polynucleotide comprises a polynucleotide sequence comprising nucleotide residues 412-2208 of an odd-numbered SEQ ID NO. of SEQ ID NOs: 3-1133.

In some embodiments, the recombinant polynucleotide comprises a polynucleotide sequence encoding an engineered AAV VP3 capsid polypeptide described herein.

In some embodiments, the recombinant polynucleotide comprises a polynucleotide sequence comprising at least 60%, 65%, 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 607-2208 of an odd-numbered SEQ ID NO. of SEQ ID NOs: 3-1133.

In some embodiments, the recombinant polynucleotide comprises a polynucleotide sequence comprising nucleotide residues 607-2208 of a recombinant polynucleotide of an engineered AAV capsid polypeptide set forth in Tables 5.1, 5.2, 6.1, 6.2, 7.1, and 7.2.

In some embodiments, the recombinant polynucleotide comprises a polynucleotide sequence comprising nucleotide residues 607-2208 of an odd-numbered SEQ ID NO. of SEQ ID NOs: 3-1133.

In a further aspect, the present disclosure provides an expression vector comprising at least one recombinant polynucleotide encoding an engineered AAV capsid polypeptide, an engineered AAV VP2 capsid polypeptide, or an engineered AAV VP3 capsid polypeptide described herein.

In some embodiments, the recombinant polynucleotide encoding an AAV capsid polypeptide, an AAV VP2 capsid polypeptide, or an AAV VP3 capsid polypeptide in the expression vector is operably linked to a control sequence. In some embodiments, the control sequence comprises at least a promoter. In some embodiments, the promoter is a heterologous promoter.

In a further aspect, the present disclosure also provides a host cell comprising an expression vector comprising a recombinant polynucleotide encoding an AAV capsid polypeptide, an AAV VP2 capsid polypeptide, or an AAV VP3 capsid polypeptide. In some embodiments, the host cell is an insect cell or mammalian cell.

In some embodiments, the host cell is used in a method of producing an engineered AAV VP1, VP2, and/or VP3 capsid polypeptide, the method comprising culturing an appropriate host cell under suitable culture conditions such that the engineered AAV VP1, VP2, and/or VP3 capsid polypeptide is produced. In some embodiments, the method further comprises recovering the engineered AAV VP1, VP2 and/or VP3 capsid polypeptide. In some embodiments, the method further comprises purifying the engineered AAV VP1, VP2, and/or VP3 capsid polypeptide.

In another aspect, the present disclosure provides a recombinant polynucleotide comprising a polynucleotide sequence encoding an engineered MAAP polypeptide described herein.

In some embodiments, the recombinant polynucleotide comprises a polynucleotide sequence having at least 60%, 65%, 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to SEQ ID NO: 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, or 1231, wherein the polynucleotide sequence encodes an engineered MAAP polypeptide.

In some embodiments, the recombinant polynucleotide comprises a polynucleotide sequence comprising SEQ ID NO: 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, or 1231.

In a further aspect, the present disclosure provides an expression vector comprising a recombinant polynucleotide encoding an engineered MAAP polypeptide. In some embodiments, the recombinant polynucleotide encoding an engineered MAAP polypeptide in the expression vector is operably linked to a control sequence. In some embodiments, the control sequence comprises at least a promoter. In some embodiments, the promoter is a heterologous promoter.

In a further aspect, the present disclosure also provides a host cell comprising an expression vector comprising a recombinant polynucleotide encoding an engineered MAAP polypeptide. In some embodiments, the host cell is an insect cell or mammalian cell.

In some embodiments, the host cells is used in a method of producing an engineered MAAP polypeptide, the method comprising culturing an appropriate host cell under suitable culture conditions such that the engineered MAAP polypeptide is produced. In some embodiments, the method further comprises recovering the engineered MAAP polypeptide. In some embodiments, the method further comprises purifying the engineered MAAP polypeptide.

In another aspect, the present disclosure provides a recombinant polynucleotide comprising a polynucleotide sequence encoding an engineered AAP polypeptide described herein.

In some embodiments, the recombinant polynucleotide comprises a polynucleotide sequence having at least 60%, 65%, 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to SEQ ID NO: 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1367, 1371, 1373, 1375, 1377, 1379, 1381, 1383, 1385, 1387, 1389, 1391, 1393, 1395, 1397, 1399, 1401, 1403, 1405, 1407, or 1409, wherein the polynucleotide sequence encodes an engineered AAP polypeptide.

In some embodiments, the recombinant polynucleotide comprises a polynucleotide sequence comprising SEQ ID NO: 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1367, 1371, 1373, 1375, 1377, 1379, 1381, 1383, 1385, 1387, 1389, 1391, 1393, 1395, 1397, 1399, 1401, 1403, 1405, 1407, or 1409.

In a further aspect, the present disclosure provides an expression vector comprising a recombinant polynucleotide encoding an engineered AAP polypeptide. In some embodiments, the polynucleotide encoding an AAP polypeptide in the expression vector is operably linked to a control sequence. In some embodiments, the control sequence comprises at least a promoter. In some embodiments, the promoter is a heterologous promoter.

In a further aspect, the present disclosure also provides a host cell comprising an expression vector comprising a recombinant polynucleotide encoding an AAP polypeptide. In some embodiments, the host cell is an insect cell or mammalian cell.

In some embodiments, the host cell is used in a method of producing an engineered AAP polypeptide, the method comprising culturing an appropriate host cell under suitable culture conditions such that the engineered AAP polypeptide is produced. In some embodiments, the method further comprises recovering the engineered AAP polypeptide. In some embodiments, the method further comprises purifying the engineered AAP polypeptide.

In another aspect, the present disclosure provides a rAAV virus or rAAV virion comprising an engineered AAV capsid polypeptide described herein.

In some embodiments, the rAAV virus or rAAV virion comprises an engineered AAV VP1 capsid polypeptide, engineered AAV VP2 capsid polypeptide, and/or engineered AAV VP3 capsid polypeptide.

In some embodiments, the rAAV virus or rAAV virion comprises at least the engineered AAV VP1 capsid polypeptide. In some embodiments, the rAAV virus or rAAV virion comprises at least the engineered AAV VP2 capsid polypeptide. In some embodiments, the rAAV virus or rAAV virion comprises at least the engineered AAV VP3 capsid polypeptide.

In some embodiments, the rAAV virus or rAAV virion further comprises at least a rAAV vector. In some embodiments, the rAAV vector comprises an inverted terminal repeat and a transgene. In some embodiments, the transgene of the rAAV vector encodes a polypeptide. In some embodiments, the polypeptide encoded by the transgene comprises a reporter polypeptide, an enzyme, a signal transduction polypeptide, a ligand polypeptide, or a structural polypeptide.

In some embodiments, the transgene of the rAAV vector transcribes a non-coding RNA. In some embodiments, the non-coding RNA comprises an RNA effector molecule. In some embodiments, the non-coding RNA molecule comprises a shRNA, miRNA, or antisense-RNA.

In some embodiments, the present disclosure provides a host cell comprising a rAAV virus or rAAV virion described herein. In some embodiments, the host cell is an insect cell or mammalian cell.

In another aspect, the present disclosure further provides a method of preparing a rAAV virus or rAAV virion comprising an AAV capsid polypeptide described herein, the method comprising providing a competent cell comprising (i) an expression vector comprising a recombinant polynucleotide encoding an engineered AAV capsid polypeptide, and a suitable AAV helper virus or AAV helper plasmid, and (ii) culturing the cell under suitable conditions such that the AAV virus or virion is produced.

In some embodiments, the method of preparing a rAAV virus or rAAV virion further comprises providing an rAAV vector in the cell.

In another aspect, the present disclosure provides a method of transducing a cell with an rAAV virus or rAAV virion, comprising contacting a cell with a rAAV virus or rAAV virion described herein under conditions suitable for transduction of the cell by the rAAV virus or rAAV virion. In some embodiments, the cell is a neuronal cell or glial cell.

In another aspect, the present disclosure provides a method of transducing a rAAV virus or rAAV virion to the CNS of a subject, comprising administering a rAAV virus or rAAV virion described herein to a subject. In some embodiments, the rAAV virus or rAAV virion is administered intravenously, intrathecally, intranasally, or intracerebrally.

### BRIEF DESCRIPTION OF THE DRAWINGS

FIG. 1 provides a graph showing the transduction potency of purified AAV9 capsids, SEQ ID NO: 2, and AAV capsid variants, SEQ ID NO: 278 and SEQ ID NO: 1094, using human iPSC-derived neurons.

FIG. 2 provides a graph showing the total number of transduced cells in mouse brains, scaled to the mean of SEQ ID NO: 2, for SEQ ID NO: 2 and SEQ ID NO: 278.

### DETAILED DESCRIPTION

Adeno-associated virus (AAV) based vectors are effective vehicles for delivering heterologous genes into cells. Different AAV serotypes display different tropism, but AAVs of each serotype are capable of transducing cells of different tissue types. For example, tropism for CNS is seen for AAV1, AAV2, AAV4, AAV5, AAV8, and AAV9; tropism for heart is seen for AAV1, AAV8, and AAV9; tropism for liver is seen for AAV7, AAV8, and AAV9; tropism for lung is seen for AAV4, AAV5, AAV6, and AAV9; tropism for photoreceptors cells is seen for AAV2, AAV5, and AAV8; tropism for retinal pigment epithelium is seen for AAV1, AAV2, AAV4, AAV5, and AAV8; and tropism for skeletal muscle is seen for AAV1, AAV6, AAV7, AAV8, and AAV9. Modifications to the AAV capsid polypeptide can alter the tropism and/or transducing efficiency of an AAV.

Many AAV vectors are based on AAV9 serotype because of its ability to transduce to different cell types. For transduction to the CNS, AAV9 can bypass the blood-brain barrier (BBB), enabling intravascular administration as a non-invasive delivery route to achieve widespread CNS gene expression. However, effective transduction of AAV9 to CNS appears to require high doses of virus, which can arise from high biodistribution to peripheral tissues and presence of anti-AAV9-neutralizing antibodies (NAbs) in the human populations. The present disclosure provides engineered AAV capsid polypeptides based on AAV9 capsid, where the engineered AAV capsid polypeptide is efficiently expressed, effective in packaging transgenes, and exhibits high transduction of neuronal and glial cells.

### Abbreviations and Definitions

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well known and described in numerous texts and reference works well known to those of skill in the art. Standard techniques, or modifications thereof, are used for chemical syntheses and chemical analyses.

Although any suitable methods and materials similar or equivalent to those described herein find use in the practice of the present invention, exemplary methods and materials are described herein. It is to be understood that the present invention is not limited to the particular methodology, protocols, and reagents described, as these may vary, depending upon the context they are used by those of skill in the art. Accordingly, the terms defined immediately below are more fully described by reference to the application as a whole.

As used herein, the singular “a,” “an,” and “the” include the plural references, unless the context clearly indicates otherwise.

As used herein, the term “comprising” and its cognates are used in their inclusive sense (i.e., equivalent to the term “including” and its corresponding cognates).

It is also to be understood that where description of embodiments use the term “comprising” and its cognates, the embodiments can also be described using language “consisting essentially of” or “consisting of.”

Moreover, numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “about” means an acceptable error for a particular value. In some instances, “about” means within 0.05%, 0.5%, 1.0%, or 2.0%, of a given value range. In some instances, “about” means within 1, 2, 3, or 4 standard deviations of a given value.

Furthermore, the headings provided herein are not limitations of the various aspects or embodiments of the invention which can be had by reference to the application as a whole. Accordingly, the terms defined immediately below are more fully defined by reference to the application as a whole.

“ATCC” refers to the American Type Culture Collection whose biorepository collection includes genes and strains.

“NCBI” refers to National Center for Biological Information and the sequence databases provided therein.

“Adeno-associated virus,” or “AAV” refers to a replication defective, non-enveloped virus having a single stranded DNA genome and classified in the family of parvoviridae. As used herein, AAV includes an assembled native or recombinant AAV packaged with a DNA genome, and encompasses all naturally occurring subtypes and recombinant forms, except where provided otherwise. AAVs of different serotypes and organism origins, include, among others, AAV type I (AAV-I), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV10), AAV type 11 (AAV11), AAV type 12 (AAV12), AAV type 13 (AAV13), avian AAV, bovine AAV, canine AAV, equine AAV, primate AAV, non-primate AAV, and ovine AAV. While AAV has a single ssDNA genome, reference to AAV genome also encompasses a dsDNA genome used in the lifecycle of the AAV.

“rAAV” as used herein refers to an engineered or recombinant adeno-associated virus, where any component of the virus or virion is engineered or recombinant. In some embodiments, the rAAV has an engineered viral protein incorporated into the AAV, such as engineered or recombinant capsid protein. In some embodiments, the rAAV has a polynucleotide sequence not of AAV origin (e.g., a polynucleotide heterologous to AAV), such as a transgene of interest for the genetic transformation of a cell. In some embodiments, the rAAV has an engineered viral protein in the capsid and a polynucleotide heterologous to AAV.

“AAV virus,” “AAV virion,” or “AAV viral particle” refers to a virus, virion, or viral particle composed of at least one AAV capsid protein, and generally all of the capsid proteins of AAV. In some embodiments, the AAV virus, virion or viral particle comprises an encapsidated polynucleotide rAAV vector.

“rAAV vector” as used herein refers to an AAV vector comprising a polynucleotide sequence not of AAV origin (i.e., a polynucleotide heterologous to AAV), such as a heterologous sequence of interest or a transgene for the genetic transformation of a cell. In general, the heterologous polynucleotide is flanked by at least one, and generally by two inverted terminal repeat sequences (ITRs), as described herein. The term rAAV vector also encompasses rAAV vector plasmids.

“AAV capsid polypeptide” or AAV capsid protein” or “AAV capsid” refers to AAV viral proteins encoded by the cap gene and which comprise the major structural proteins of the AAV virion. In naturally occurring AAVs, the different capsid proteins VP1, VP2 and VP3 are generated through alternative splicing of the mRNA and use of an alternate translational start codon. Generally, the VP3 sequence is shared among all VP proteins and is sometimes referred to as the VP3 common region. VP2 is longer than VP3 and the VP2 N-terminal region is referred to as the VP1/VP2 common region. VP1 is longer than VP2 and this region is sometimes referred to as the VP1 unique (VP1u) region. The sequence and size of VP1, VP2 and VP3 proteins can vary, depending on the origin and serotype of the AAV. In some embodiments, “AAV capsid polypeptide” or “AAV capsid protein” refers to the longest polypeptide encoded by the cap gene.

“Assembly-activating protein,” or “AAP polypeptide” or “AAP protein” or “AAP” refers to AAV protein that promotes capsid assembly and is encoded by an alternative reading frame, ORF2, which results from a frame-shift in the VP2/3 reading frame of the cap gene. “rAAP” protein or polypeptide refers to a recombinant or engineered form of AAP.

“Membrane-associated accessory protein,” “MAAP polypeptide,” “MAAP protein” or “MAAP” refers to a protein or polypeptide encoded by a second ORF in the VP1/2 different from the capsid proteins. In AAV2 serotype, MAAP ORF uses a CTG (leucine) codon at the start codon for MAAP translation. MAAP may affect competitive exclusion of different serotype AAV genomes and production and intracellular distribution of AAV capsid proteins. “rMAAP” refers to recombinant or engineered forms of MAAP protein.

“Helper virus” refers to a virus that allows AAV to be replicated and packaged by an appropriate host cell. A variety of such helper viruses for AAV are known in the art, including adenoviruses, herpesviruses, and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper plasmid” as used herein refers to a recombinant vector that expresses proteins essential for production of AAV virus or viral particles. In some embodiments, the helper plasmid may contain genes for E1A, E1B, E2A, E4 ORF6, and VA RNA. In some embodiments, each of these genes can have its own promoter through which transcription can occur. Alternative promoters that may be used in the helper plasmid include, but are not limited to, CMV, RSV, MMTV, E1A, EF1a, actin, cytokeratin 14, cytokeratin 18, PGK as well as others known to those skilled in the art. In some embodiments, the helper genes are bounded by ITR sequences to allow replication of the plasmids.

“Packaging” refers to a series of intracellular events that result in the assembly and encapsidation of an AAV particle. AAV “rep” and “cap” genes refer to polynucleotide sequences encoding replication and encapsidation proteins of adeno-associated virus. AAV rep and cap arc referred to herein as AAV “packaging genes.”

“Tropism” or “cell tropism” as used herein refers to the ability of a virus or viral particle to transduce certain cell types. AAV virus or viral particle with varying tropism have the ability to transduce different cell types, e.g., neuronal or muscle cell types. The tropism of an AAV virus or viral particle may be altered by recombinant techniques, which can result in a retargeted AAV virus or viral particle, e.g., an AAV virus or viral particle is re-directed preferentially to specific cell types other than those normally infected by a naturally occurring AAV serotype.

“Inverted terminal repeat” (ITR) as used herein includes any viral terminal repeat or synthetic sequence that forms a hairpin structure and functions as an inverted terminal repeat (i.e., mediates the desired functions, such as replication, virus packaging, integration and/or provirus rescue). The ITR can be an AAV ITR or a non-AAV ITR sequence such as those of other parvoviruses (e.g., canine parvovirus (CPV), mouse parvovirus (MVM), human parvovirus B-19) or any other suitable virus sequence. For example, the SV40 hairpin that serves as the origin of SV40 replication can be used as an ITR. An ITR sequence can further be modified by truncation, substitution, deletion, insertion and/or addition. Further, the ITR can be partially or completely synthetic, such as the “double-D sequence” as described in U.S. Pat. No. 5,478,745. An “AAV terminal repeat” or “AAV ITR” may be from any AAV, including but not limited to serotypes 1, 2, 3, 3B, 4, 5, 6, 7, 8, 9, 10, 11, 12 or 13, or any other known AAV. An AAV terminal repeat need not have the native terminal repeat sequence (e.g., a native AAV ITR sequence may be altered by insertion, deletion, truncation and/or missense mutations), as long as the terminal repeat mediates the desired functions, e.g., replication, virus packaging, integration, and/or provirus rescue, and the like, to a region of the AAV genome comprising all elements involved in genome rescue, replication and packaging. Thus, ITR as used herein refers to the cis-elements needed for at least packaging of the genome.

“Transgene” refers to a gene which is artificially introduced into the genome of another organism. A transgene may also be referred to as a heterologous gene. In the case of AAV, the transgene is typically placed between two ITRs of the genome. A “transgene expression cassette” refers to a transgene operably linked to one or more control sequences appropriate for expression of the transgene of interest. In some embodiments, the transgene expression cassette comprises at least a promoter suitable for expression of the transgene.

“Protein,” “polypeptide,” and “peptide” are used interchangeably to denote a polymer of at least two amino acids covalently linked by an amide bond, regardless of length or post-translational modification (e.g., glycosylation or phosphorylation).

“Amino acids” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glycine (Gly or G), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about «-carbon (Co). For example, whereas “Ala” designates alanine without specifying the configuration about the xx-carbon, “D-Ala” and “L-Ala” designate D-alanine and L-alanine, respectively. When the one-letter abbreviations are used, upper case letters designate amino acids in the L-configuration about the x-carbon and lower case letters designate amino acids in the D-configuration about the xx-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Fusion protein,” and “chimeric protein” and “chimera” refer to hybrid proteins created through the joining of two or more polynucleotides that originally encode separate proteins. In some embodiments, fusion proteins are created by recombinant technology (e.g., molecular biology techniques known in the art).

“Polynucleotide,” “nucleic acid,” or “oligonucleotide” is used herein to denote a polymer comprising at least two nucleotides where the nucleotides are either deoxyribonucleotides or ribonucleotides or mixtures of deoxyribonucleotides and ribonucleotides. In some embodiments, the abbreviations used for genetically encoding nucleosides are conventional and are as follow: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When a polynucleotide, nucleic acid, or oligonucleotide sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid. The polynucleotide or nucleic acid may be single-stranded or double-stranded, or may include both single-stranded regions and double-stranded regions.

“Duplex” and “ds” refer to a double-stranded nucleic acid (e.g., DNA or RNA) molecule comprised of two single-stranded polynucleotides that are complementary in their sequence (A pairs to T or U, C pairs to G), arranged in an antiparallel 5′ to 3′ orientation, and held together by hydrogen bonds between the nucleobases (i.e., adenine [A], guanine [G], cytosine [C], thymine [T], uridine [U].

“Engineered,” “recombinant,” “non-naturally occurring,” and “variant,” when used with reference to a cell, a polynucleotide or a polypeptide refer to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence that can be isolated from a source in nature and which has not been intentionally modified by human manipulation.

“Coding sequence” refers to that part of a nucleic acid (e.g., a gene) that encodes an amino acid sequence of a protein.

“Gene” refers to a polynucleotide containing at least one open reading frame that encodes a particular polypeptide which is expressed after being transcribed and translated, or refers to a polynucleotide which when transcribed produces a non-coding RNA molecule.

“Percent (%) sequence identity” refers to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (see, e.g., Altschul et al., J. Mol. Biol., 1990, 215:403-410; and Altschul et al., Nucleic Acids Res., 1977, 3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (see Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W. T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see, e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, the phrase “a reference sequence corresponding to SEQ ID NO: 2, having an glycine at the residue corresponding to X582” (or “a reference sequence corresponding to SEQ ID NO: 2, having a glycine at the residue corresponding to position 582”) refers to a reference sequence in which the corresponding residue at position X582 in SEQ ID NO: 2 (e.g., a threonine), has been changed to glycine.

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to,” “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered AAV capsid polypeptide, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original polypeptide sequence. In some further embodiments, the mutation comprises a deletion, such that an amino acid is deleted from the original polypeptide sequence. Any number of mutations may be present in a given sequence. In some embodiments, the “substitution” comprises the deletion of an amino acid, and is denoted by “-” symbol. In some embodiments, the “substitution” comprises the replacement of an amino acid with a stop codon, and is denoted by “*” symbol.

“Amino acid difference” and “residue difference” refer to a difference in the amino acid residue at a position of a polypeptide sequence relative to the amino acid residue at a corresponding position in a reference sequence. The positions of amino acid differences generally are referred to herein as “Xn.” where n refers to the corresponding position in the reference sequence upon which the residue difference is based. For example, a “residue difference at position X582 as compared to SEQ ID NO: 2” (or a “residue difference at position 582 as compared to SEQ ID NO: 2”) refers to a difference of the amino acid residue at the polypeptide position corresponding to position 582 of SEQ ID NO: 2. Thus, if the reference polypeptide of SEQ ID NO: 2 has a threonine at position 582, then a “residue difference at position X582 as compared to SEQ ID NO: 2” refers to an amino acid substitution of any residue other than threonine at the position of the polypeptide corresponding to position 582 of SEQ ID NO: 2. In some instances herein, the specific amino acid residue difference at a position is indicated as “XnY” where “Xn” specified the corresponding residue and position of the reference polypeptide (as described above), and “Y” is the single letter identifier of the amino acid found in the engineered polypeptide (i.e., the different residue than in the reference polypeptide). In some instances (e.g., in the Tables in the Examples), the present disclosure also provides specific amino acid differences denoted by the conventional notation “AnB”, where A is the single letter identifier of the residue in the reference sequence. “n” is the number of the residue position in the reference sequence, and B is the single letter identifier of the residue substitution in the sequence of the engineered polypeptide. In some embodiments, the amino acid difference, e.g., a substitution, is denoted by the abbreviation “nB,” without the identifier for the residue in the reference sequence. In some embodiments, the phrase “an amino acid residue nB” denotes the presence of the amino residue in the engineered polypeptide, which may or may not be a substitution in context of a reference polypeptide.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X221M/X221R, X221M/R, or 22I/M/R). The present disclosure includes engineered polypeptide sequences comprising one or more amino acid differences that include either/or both conservative and non-conservative amino acid substitutions, as well as insertions and deletions of amino acids in the sequence.

“Amino acid substitution set” and “substitution set” refers to a group of amino acid substitutions within a polypeptide sequence. In some embodiments, substitution sets comprise 2, 3, 4, 5, 6, 7, 8, 9, 10, 11, 12, 13, 14, 15, or more amino acid substitutions. In some embodiments, a substitution set refers to the set of amino acid substitutions that is present in any of the engineered polypeptides listed in any of the Tables in the Examples. In these substitution sets, the individual substitutions are separated by a semicolon (“;”; e.g., K449R;H584L) or slash (“/”; e.g., K449R/H584L or 449R/584L).

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affect: (a) the structure of the peptide backbone in the area of the substitution (e.g., proline for glycine); (b) the charge or hydrophobicity; and/or (c) the bulk of the side chain. By way of example and not limitation, exemplary non-conservative substitutions include an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference polypeptide while retaining biological activity and/or retaining the improved properties of an engineered polypeptide. Deletions can be directed to the internal portions and/or terminal portions of the polypeptide. In various embodiments, the deletion can comprise a continuous segment or can be discontinuous. Deletions are indicated by “-”, and may be present in substitution sets.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally-occurring polypeptide.

“Functional fragment” and “biologically active fragment” are used interchangeably herein, to refer to a polypeptide that has an amino-terminal and/or carboxy-terminal deletion(s) and/or internal deletions, but where the remaining amino acid sequence is identical to the corresponding positions in the sequence to which it is being compared (e.g., a full length engineered polypeptide of the present disclosure) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The engineered polypeptides may be present within a cell, present in the cellular medium, or prepared in various forms, such as lysates or isolated preparations. As such, in some embodiments, the engineered polypeptides provided herein are isolated polypeptides.

“Substantially pure polypeptide” or “purified polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure engineered polypeptide composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated engineered polypeptides are substantially pure polypeptide compositions.

“Improved property” refers to an engineered polypeptide that exhibits an improvement in any property as compared to a reference polypeptide, such as a wild-type AAV capsid polypeptide or another engineered AAV capsid polypeptide. Improved properties include but are not limited to such properties as change in tropism, increased transduction, increased protein expression, increased packaging efficiency, increased thermostability, increased stability, increased pH stability, increased chemical stability, increased resistance to NAbs, and increased solubility.

“Codon optimized” refers to changes in the codons of the polynucleotide encoding a protein to those preferentially used in a particular organism such that the encoded protein is more efficiently expressed in that organism. Although the genetic code is degenerate, in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the engineered polypeptides are codon optimized for optimal production from the host organism selected for expression.

“Control sequence” or “control element” refers herein to include all components that are necessary or advantageous for the expression of a polynucleotide and/or polypeptide of the present disclosure. Each control sequence may be native or foreign to the nucleic acid sequence encoding the polypeptide. Such control sequences include, but are not limited to, leaders, polyadenylation sequences, promoter sequences, signal peptide sequences, initiation sequences, and transcription terminators. In some embodiments, the control sequences include a promoter, and transcriptional and translational stop signals.

“Operably linked” or “operatively linked” refers to a configuration in which a control sequence is appropriately placed (i.e., in a functional relationship) at a position relative to a polynucleotide of interest such that the control sequence directs or regulates the expression of the polynucleotide, and where relevant, expression of an encoded polypeptide of interest.

“Promoter” or “promoter sequence” refers to a nucleic acid sequence that is recognized by a host cell for expression of a polynucleotide of interest, such as a coding sequence. The promoter sequence contains transcriptional control sequences that mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Culturing” refers to the growing of a population of cells under suitable conditions using any suitable medium (e.g., liquid, gel, or solid).

“Vector” is a recombinant construct for introducing a polynucleotide of interest into a cell. In some embodiments, the vector is an expression vector that is operably linked to a suitable control sequence or element capable of effecting the expression in a suitable host of the polynucleotide or a polypeptide encoded in the polynucleotide. In some embodiments, an “expression vector” has a promoter sequence operably linked to the polynucleotide (e.g., transgene) to drive expression in a host cell, and in some embodiments, also comprises a transcription terminator sequence.

“Expression” includes any step involved in the production of the polypeptide including, but not limited to, transcription, post-transcriptional modification, translation, and post-translational modification. In some embodiments, the term also encompasses secretion of the polypeptide from a cell.

“Produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Heterologous” or “recombinant” refers to the relationship between two or more nucleic acid or polypeptide sequences (e.g., a promoter sequence, signal peptide, terminator sequence, etc.) that are derived from different sources and are not associated in nature.

“Host cell” and “host strain” refer to suitable hosts for expression vectors comprising a polynucleotide provided herein (e.g., a polynucleotide sequences encoding at least one polypeptide of interest). In some embodiments, the host cells are prokaryotic or eukaryotic cells that have been transformed or transfected with vectors constructed using recombinant DNA techniques as known in the art.

“Subject” as used herein refer to a mammal, including, but not limited to, human and non-human primates, including simians and humans; domesticated mammals (e.g., horses, sheep, dogs, cats, etc.) and rodents (e.g., mice, rats, etc.). In some embodiments, a “subject” selected for treatment is referred to as a patient, particularly a human patient.

“Pharmaceutically acceptable” refers to a material or substance that can be administered to a subject without causing any undesirable biological effects or interacting in a deleterious manner with any of the components in which it is contained and that possesses the desired biological activity.

“Carrier” when used in reference to a pharmaceutical composition means any of the standard pharmaceutical carrier, buffers, and excipients, such as stabilizers, preservatives, and adjuvants.

“Excipient” refers to any pharmaceutically acceptable additive, carrier, diluent, adjuvant, or other ingredient, other than the active pharmaceutical ingredient (API; e.g., the recombinant factor VIII polypeptides of the present disclosure).

“Gene therapy” refers to the delivery of a gene, polydeoxyribonucleotide, or polynucleotide sequence(s) with a gene therapy vector to cells or tissues for the modification of those cells or tissues for the treatment or prevention of a disease. Gene therapy may include replacing a mutated gene that causes disease with a healthy copy of the gene; inactivating, or “knocking out,” a mutated gene that is functioning improperly; or providing a functional copy of a gene sufficient to treat and/or prevent the disease or condition. In some embodiments, gene therapy is used in the treatment of disease in patients.

“Treating” or “treatment” of a disease, disorder, or syndrome, as used herein, includes (i) preventing the disease, disorder, or syndrome from occurring in a subject, i.e., causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome. i.e., arresting its development; and (iii) relieving the disease, disorder, or syndrome, i.e., causing regression of the disease, disorder, or syndrome. As such, the terms “treating.” “treat” and “treatment” encompass preventative (e.g., prophylactic), as well as palliative treatment. As is known in the art, adjustments for systemic versus localized delivery, age, body weight, general health, sex, diet, time of administration, drug interaction and the severity of the condition may be necessary, and will be ascertainable by one of ordinary skill in the art.

### Engineered AAV Polypeptides

The present disclosure relates to engineered AAV polypeptides, in particular engineered AAV capsid polypeptides, engineered MAAP polypeptides, and AAP polypeptides. In some embodiments, the engineered AAV capsid polypeptides include engineered AAV VP1, VP2, and/or VP3 capsid polypeptides. In some embodiments, rAAV virus or rAAV virion comprising the engineered AAV capsid polypeptides is used to deliver transgenes to cells, for example for gene therapy.

Engineered rAAV Capsid Polypeptides

In one aspect, the present disclosure provides engineered adeno-associated (AAV) capsid polypeptides and rAAV viruses comprising the AAV capsid polypeptides. In particular, the rAAV viruses or rAAV virions comprising the engineered AAV capsid polypeptides are characterized by, among others, efficient transduction to neural and glial cells and CNS.

In some embodiments, the engineered AAV capsid polypeptides provided herein include engineered VP1, VP2, and VP3 capsid polypeptides. In some embodiments, the engineered AAV capsid polypeptide comprises an engineered VP1 capsid polypeptide. In some embodiments, the engineered AAV capsid polypeptide comprises an engineered AAV VP2 capsid polypeptide. In some embodiments, the engineered AAV capsid polypeptide comprises an engineered AAV VP3 capsid polypeptide.

It is to be understood that while the engineered AAV capsid polypeptides described herein are based on the amino acid sequence of the corresponding polypeptides from AAV9 virus, equivalent amino acid positions in capsid polypeptides based on other AAV serotypes (e.g., AAV1, AAV2, AAV3, AAV4, AAV5, AAV6, AAV7, AAV8, AAV9, AAV10, AAV11, AAV12, AAV13, and variants and recombinants thereof) can be identified and modified according to the present disclosure.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to an even-numbered SEQ ID NO. of SEQ ID NOs: 2-1134, wherein the amino acid sequence comprises one or more substitutions relative to the reference sequence corresponding to SEQ ID NO: 2, 278, or 522.

In some embodiments, an engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 2, 278, or 522, wherein the amino acid sequence comprises one or more substitutions relative to the reference sequence corresponding to SEQ ID NO: 2, 278, or 522.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 2, wherein the amino acid sequence comprises one or more substitutions relative to the reference sequence corresponding to SEQ ID NO: 2.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522, wherein the amino acid sequence comprises one or more substitutions relative to the reference sequence corresponding to SEQ ID NO: 2.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO. of SEQ ID NOs: 4-1134, wherein the amino acid sequence comprises one or more substitutions relative to the reference sequence corresponding to SEQ ID NO: 2.

In some embodiments, the amino acid sequence of the engineered AAV capsid polypeptide comprises at least a substitution at amino acid position 10, 14, 17, 22, 23, 24, 25, 31, 33, 45, 51, 53, 56, 60, 63, 66, 79, 81, 90, 100, 101, 119, 125, 126, 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to SEQ ID NO: 2.

In some embodiments, the amino acid sequence of the engineered AAV capsid polypeptide comprises at least a substitution or amino acid residue 101, 14L/R/V, 17T, 22I/M/R, 23R, 24E/R/T/V/W, 25G/Q/S, 31L, 33E/F/G/L/R, 45F, 51E, 53C/P/R/S/W, 56F, 60K/S/Y, 63Q, 66R, 79H, 81E, 90A/D/G/L/R/T, 100A/L/P/R/S/T/V, 101E/R/S/V, 119S, 125V, 126R, 142N, 152Q, 155G/R, 156G/W, 157C/L/Q/S/T/W, 159C/R, 162G/V, 165G/V, 168R, 169V, 172D/E/W, 173S/T/W, 175D/E/G, 190A/C/D/H/L/S/V, 193S, 200P, 205P, 210S, 211M, 214D, 218S, 233T, 250S, 254C, 2561, 258T, 259K/S, 263A/T/W, 264E/G/N/S, 266F/H/L/M/Q/S/T/V, 267A/L/V, 2681, 269T, 270A/H/L/M/P/Q/V, 272G, 273T, 274F, 320V, 328T, 329G, 331R/S/T, 333A/I/N/S, 334V, 346S, 348T, 349E, 361Q, 384N, 387G, 389F/M/P, 391L/Q, 401A/G, 412R, 414D/Q/R/T, 416S/T/Y, 419D/G, 421R/T, 437V, 440A, 444V, 449E/R/T, 450G/N, 451G/H/L/N/P/S/T/V/Y, 452D/G, 453F/L/Q/R/W, 454P/Q/R, 455R, 456F/I/K/L/M/N/P/V, 457F/G/L/R/T, 458S, 459A/C/M/R, 460A/C/G/L, 461G/W, 462A/E/M, 465I/K/L/N/P/Q/T, 466T, 468A/Q/V, 468W/Y, 469D/W, 470G/L/Q, 4711, 472Q/W, 478W, 479L, 483C, 485G/L/P/Q/S/W, 489C, 490P/V, 492Q/R/V/W, 493T, 4941, 495F/N, 496D, 497C/L/R/S, 499G, 500A/D/G/H/L/R/W, 501M/N, 504T, 505T/Y, 507T, 508K, 509Y, 510C/G, 510E/H/I/Q/T, 512G/L, 515L/T/V, 519S, 528I, 529A/D/V, 530D, 532C/Q/R/T/V, 535A/G/L, 537R, 538G, 539A, 545P, 547D/E/H/Q/R/W, 548A/L/M/R/V, 549M/W, 550A, 551F/M, 552I/K/R/S/V/W, 553K, 554G/I/L/Q/R/S/V, 555R/S/W, 556G/R/T, 559C, 560H/L, 562L/R, 564A/D, 565G/K/T, 575D, 576Y, 579L, 582G/L/R, 584C/L/V, 585I/M/T, 586A/G/I/I/K/N/R/V/W, 587I/Q/V, 588L/P/R/S/T/V, 589R/T/Y, 592A/G/H/L/Y, 594M/N/Q/S/T/V/W, 595F/G, 597C/I/I/N, 598G/T/W, 628Q, 640L, 642E/T/W, 659N/Q/R, 660G/M, 661H/K/S/V/W, 662P, 663D/R, 664G, 665A/G/M/R/W, 666I/P, 667N/T, 668A/C/R, 705W, 706W, 709G, 710G/V, 712D, 716D, 720F, 724I/V, 725A/G/H/M/W, 726H/I, or 736M, or combinations thereof, wherein the amino acid positions are relative to the reference sequence corresponding to SEQ ID NO: 2.

In some embodiments, the amino acid sequence of the engineered AAV capsid polypeptide comprises at least a substitution W10I, N14L/R/V, E17T, W22I/M/R, W23R, A24E/R/T/V/W, L25G/Q/S, Q31L, K33E/F/G/L/R, L45F, K51E, L53C/P/R/S/W, G56F, D60K/S/Y, E63Q, N66R, Y79H, Q81E, Y90A/D/G/L/R/T, F100A/L/P/R/S/T/V, Q101E/R/S/V, F119S, L125V, L126R, K142N, E152Q, S155G/R, S156G/W, A157C/L/Q/S/T/W, 1159C/R, S162G/V, Q165G/V, K168R, K169V, N172D/E/W, F173S/T/W, Q175D/E/G, E190A/C/D/H/L/S/V, A193S, S200P, S205P, P210S, V211M, N214D, A218S, Q233T, P250S, N254C, L256I, K258T, Q259K/S, S263A/T/W, T264E/G/N/S, G266F/H/L/M/Q/S/T/V, G267A/L/V, S268I, S269T, N270A/H/L/M/P/Q/V, N272G, A273T, Y274F, I320V, N328T, N329G, V331R/S/T, T333A/I/N/S, L334V, T346S, S348T, D349E, E361Q, D384N, Q387G, V389F/M/P, R391L/Q, P401A/G, Q412R, S414D/Q/R/T, E416S/T/Y, N419D/G, P421R/T, N437V, 1440A, L444V, K449E/R/T, T450G/N, 1451G/H/L/N/P/S/T/V/Y, N452D/G, G453F/L/Q/R/W, S454P/Q/R, G455R, Q456F/I/K/L/M/N/P/V, N457F/G/L/R/T, Q458S, Q459A/C/M/R, T460A/C/G/L, L461G/W, K462A/E/M, V465I/K/L/N/P/Q/T, A466T, P468A/Q/V, P468W/Y, S469D/W, N470G/L/Q, M471I, A472Q/W, Y478W, I479L, S483C, R485G/L/P/Q/S/W, V489C, S490P/V, T492Q/R/V/W, V493T, T494I, Q495F/N, N496D. N497C/L/R/S, S499G, F500A/D/G/H/L/R/W, F501M/N, P504T, G505T/Y, S507T, S508K, W509Y, A510C/G, A510E/H/I/Q/T, N512G/L, N515L/T/V, N519S, K528I, E529A/D/V. G530D, D532C/Q/R/T/V, F535A/G/L, L537R, S538G, G539A, K545P, G547D/E/H/Q/R/W, T548A/L/M/R/V, G549M/W, R550A, D551F/M, N552I/K/R/S/V/W, V553K, D554G/I/L/Q/R/S/V, A555R/S/W, D556G/R/T, M559C, I560H/L, N562L/R, E564A/D, E565G/K/T, E575D, S576Y, Q579L, T582G/L/R, H584C/L/V, Q585I/M/T, S586A/G/I/K/N/R/V/W, A5871/Q/V, Q588L/P/R/S/T/V, A589R/T/Y, Q592A/G/H/L/Y, G594M/N/Q/S/T/V/W, W595F/G, Q597C/I/I/N, N598G/T/W, N628Q, M640L, H642E/T/W, P659N/Q/R, T660G/M, A661H/K/S/V/W, F662P, N663D/R, K664G, D665A/G/M/R/W, K666I/P, L667N/T, N668A/C/R, Y705W, Y706W, N709G, N710G/V, E712D, N716D, V720F, P724I/V, R725A/G/H/M/W, P726H/I, or L736M, or combinations thereof, wherein the amino acid positions are relative to the reference sequence corresponding to SEQ ID NO: 2.

In some embodiments, the amino acid sequence of the engineered AAV capsid polypeptide comprises substitutions in at least two of amino acid positions 588, 665, 457, 495, 456, 505, 494, 497, 592, 586, 585, 706, 549, 552, 664, 661, 461, 668, 548, 510, 640, 419, 349, 504, 508, 453, 589, 56, and 416, wherein the amino acid positions are relative to the reference sequence corresponding to SEQ ID NO: 2.

In some embodiments, the amino acid sequence of the engineered AAV capsid polypeptide comprises at least two substitutions comprising at least a first and second substitution, wherein the substitutions are at amino acid positions selected from (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, 56, and 416, wherein the first substitution is at a position selected from (a), and the second substitution is at a position selected from (a), (b) and (c), wherein the amino acid positions are relative to the reference sequence corresponding to SEQ ID NO: 2. In some embodiments, the first substitution is at a position selected from (a) and the second substitution is at a position selected from (b) and (c). In some embodiments, the first substitution is at a position selected from (a) and the second substitution is at a position selected from (b). In some embodiments, the first substitution is at a position selected from (a) and the second substitution is selected from (c).

In some embodiments, the at least two substitutions comprising the first and second substitutions at the foregoing amino acid positions are selected from 56F, 349E, 416S/T/Y, 419D/G, 453F/L/Q/R/W, 456F/I/K/L/M/N/P/V, 457F/G/L/R/T, 461G/W, 4941, 495F/N, 497C/L/R/S, 501M/N, 504T, 505T/Y, 508K, 510E/H/I/Q/T, 548A/L/M/R/V, 549M/W, 552I/K/R/S/V/W, 585I/M/T, 586A/G/I/K/N/R/V/W, 588L/P/R/Q/S/T/V, 589R/T/Y, 592A/G/H/L/Y, 640L, 661H/K/S/V/W. 664G, 665A/G/M/R/W, 668A/C/R, and 706W, wherein the amino acid positions are relative to the reference sequence corresponding to SEQ ID NO: 2. In some embodiments, the at least two substitutions comprising the first and second substitutions at the foregoing amino acid positions are selected from 56F, 349E, 416T, 419D, 453R, 456F, 457G, 461W, 4941, 495F, 497S, 501M/N, 504T, 505Y, 508K, 510H, 548V, 549M, 552I/R, 585, 586I, 588R, 589R, 592A, 640L, 661H, 664G, 665G. 668R, and 706W, wherein the amino acid positions are relative to the reference sequence corresponding to SEQ ID NO: 2.

In some embodiments, the amino acid sequence of the engineered AAV capsid polypeptide comprises at least a substitution set at amino acid position(s) 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to SEQ ID NO: 2.

In some embodiments, the amino acid sequence of the engineered AAV capsid polypeptide comprises at least a substitution set 588R/665G, 457G/495F/588R/665G, 461W/588R/665G, 588R/665G/668R, 495F/588R/665G, 494L/495F/505Y/588R/664G/665G, 494L/588R/665G, 588R/592A/664G/665G. 456F/497S/588R/592A/665G, 494L/497S/588R/665G, 456F/495F/505Y/588R/665G, 456F/457G/588R/665G, 497S/505Y/586I/588R/665G, 456F/457G/494L/497S/505Y/588R/661H/665G/706W, 457G/497S/549M/5521/588R/664G/665G, 585M/588R/665G/706W, 495F/588R/661H/665G, 497S/548V/5521/588R/664G/665G, 456F/457G/494L/497S/505Y/588R/661H/665G, 456F/494L/497S/588R/661H/665G, or 457G/494L/497S/588R/665G, wherein the amino acid positions are relative to SEQ ID NO: 2.

In some embodiments, the amino acid sequence of the engineered AAV capsid polypeptide comprises at least a substitution set Q588R/D665G, N457G/Q495F/Q588R/D665G, L461W/Q588R/D665G, Q588R/D665G/N668R, Q495F/Q588R/D665G, T494L/Q495F/G505Y/Q588R/K664G/D665G, T494L/Q588R/D665G, Q588R/Q592A/K664G/D665G, Q456F/N497S/Q588R/Q592A/D665G, T494L/N497S/Q588R/D665G, Q456F/Q495F/G505Y/Q588R/D665G, Q456F/N457G/Q588R/D665G, N497S/G505Y/S586I/Q588R/D665G, Q456F/N457G/T494L/N497S/G505Y/Q588R/A661H/D665G/Y706W, N457G/N497S/G549M/N552I/Q588R/K664G/D665G, Q585M/Q588R/D665G/Y706W, Q495F/Q588R/A661H/D665G, N497S/T548V/N552I/Q588R/K664G/D665G, Q456F/N457G/T494L/N497S/G505Y/Q588R/A661H/D665G, Q456F/T494L/N497S/Q588R/A661H/D665G, or N457G/T494L/N497S/Q588R/D665G, wherein the amino acid positions are relative to SEQ ID NO: 2.

In some embodiments, the amino acid sequence of the engineered AAV capsid polypeptide comprises at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 56/529, 331/449, 211/509/712, 331/416/478, 56/507, 56/331/584/597, 56/478, 349/465/479/504/508, 349/640, 331/584/597, 331/416/478/483/507/584/597, 56/416, 233/465/640, 125/508/510/589, 597, 331/597, 56/331/416/665, 125/465/504/508/510, 56/331/478/483/529/597, 384/479, 233/640, 125/419/504/508/530, 504/510/640, 419/504/508, 56/331/361, 416/665, 56/211/361/416/478/584, 56, 56/331/416, 175/211, 56/211/478/483/712, 349/504/508, 125/508/510, 331/416, 331, 479/504/510/589, 331/478, 56/331/478/483/597, 479, 125/504/508, 479/589, or 479/510/589, wherein the amino acid positions are relative to the reference sequence corresponding to SEQ ID NO: 2.

In some embodiments, the amino acid sequence of the engineered AAV capsid polypeptide comprises at least a substitution or substitution set 547Q, 453R, 547E, 555W, 535A, 501M, 555R, 456L, 550A, 497L, 529Y, 705W, 554V, 500D, 548L, 582G, 586W, 453L, 584C, 535G, 585M, 592A, 459A, 492Q, 459R, 661W, 592L, 6661, 535L, 587V, 547R, 500A, 552S, 706W, 552W, 661V, 661H, 500L, 586A, 499G, 529V, 586R, 665M, 666P, 497R, 548R, 552R, 554R, 538G, 548M, 552V, 556T, 492R, 532V, 495N, 588R, 460G, 456M, 456V, 663R, 453F, 665R. 5541, 454R, 554S, 660G, 501N, 554G, 489C, 460C, 457G, 500G, 554L, 453W, 5521, 500R, 709G, 494L, 495F, 659N, 584V, 493T, 505Y, 668C, 547H, 589R, 584L, 662P, 456K, 500W, 547D, 556R, 456P, 588P, 659R, 456F, 585T, 532Q, 548A, 667N, 537R, 586V, 457L, 547W, 492V, 452G, 549M, 710V, 582R, 556G, 548V, 459M, 453Q, 460A, 586G, 588L, 532T, 592Y, 457T, 582L, 552K, 532C, 500H, 555S, 640L, 529A, 25S/492W, 665W, 554Q, 5851, 457R, 545P. 211M/416T, 539A, 529D, 589T/640L, 592G, 588R/665G, 551F, 504T/510H, 586K, 710G, 455R, 551M, 508K, 457F, 465Q/589T/640L, 497S, 419D/504T/508K/510H, 667T, 549W, 5281, 497C, 532R, 661S, 490P, 664G, 553K, 505T, 592H, 5861, 660M, 458S, 494I, 361Q/483C, 449R/584L, 384N/479L/504T/508K/510H, 507T/509Y/529D, 490V, 510H/640L, 233T/508K, 56F/529D, 331T/449R, 211M/509Y/712D, 331T/416T/478W, 668A, 56F/507T, 56F/331T/584L/597N, 56F/478W, 349E/465Q/479L/504T/508K, 349E/640L, 331T/584L/597N, 331T/416T/478W/483C/507T/584L/597N, 56F/416T, 233T/465Q/640L, 125V/508K/510H/589T, 597N, 331T/597N, 665A, 56F/331T/416T/665A, 125V/465Q/504T/508K/510H, 56F/331T/478W/483C/529D/597N, 384N/479L, 233T/640L, 125V/419D/504T/508K/530D, 504T/510H/640L, 419D/504T/508K, 56F/331T/361Q, 416T/665A, 56F/211M/361Q/416T/478W/584L, 56F, 56F/331T/416T, 175E/211M, 56F/211M/478W/483C/712D, 349E/504T/508K, 125V/508K/510H, 331T/416T, 331T, 479L/504T/510H/589T, 331T/478W, 56F/331T/478W/483C/597N, 479L, 125V/504T/508K, 479L/589T, or 479L/510H/589T, wherein the amino acid positions are relative to the reference sequence corresponding to SEQ ID NO: 2.

In some embodiments, the amino acid sequence of the engineered AAV capsid polypeptide comprises at least a substitution or substitution set G547Q, G453R, G547E, A555W, F535A, F501M, A555R, Q456L, R550A, N497L, E529Y, Y705W, D554V, E500D, T548L, T582G, S586W, G453L, H584C, F535G, Q585M, Q592A, Q459A, T492Q, Q459R, A661W, Q592L, K666I, F535L, A587V, G547R, E500A, N552S, Y706W, N552W, A661V, A661H, E500L, S586A, S499G, E529V, S586R, D665M, K666P, N497R, T548R, N552R, D554R, S538G, T548M, N552V, D556T, T492R, D532V, Q495N, Q588R, T460G, Q456M, Q456V, N663R, G453F, D665R, D554I, S454R, D554S, T660G, F501N, D554G, V489C, T460C, N457G, E500G, D554L, G453W, N5521, E500R, N709G, T494L, Q495F, P659N, H584V, V493T, G505Y, N668C, G547H, A589R, H584L, F662P, Q456K, E500W, G547D, D556R, Q456P, Q588P, P659R, Q456F, Q585T, D532Q, T548A, L667N, L537R, S586V, N457L, G547W, T492V, N452G, G549M, N710V, T582R, D556G, T548V, Q459M, G453Q, T460A, S586G, Q588L, D532T, Q592Y, N457T, T582L, N552K, D532C, E500H, A555S, M640L, E529A, L25S/T492W, D665W, D554Q, Q585I, N457R, K545P, V211M/E416T, G539A, E529D, A589T/M640L, Q592G, Q588R/D665G, D551F, P504T/A510H, S586K, N710G, G455R, D551M, S508K, N457F, V465Q/A589T/M640L, N497S, N419D/P504T/S508K/A510H, L667T, G549W, K528I, N497C, D532R, A661S, S490P, K664G, V553K, G505T, Q592H, S586I, T660M, Q458S, T494I, E361Q/S483C, K449R/H584L, D384N/1479L/P504T/S508K/A510H, S507T/W509Y/E529D, S490V, A510H/M640L, Q233T/S508K, G56F/E529D, V331T/K449R, V211M/W509Y/E712D, V331T/E416T/Y478W, N668A, G56F/S507T, G56F/V331T/H584L/Q597N, G56F/Y478W, D349E/V465Q/1479L/P504T/S508K, D349E/M640L, V331T/H584L/Q597N, V331T/E416T/Y478W/S483C/S507T/H584L/Q597N, G56F/E416T, Q233T/V465Q/M640L, L125V/S508K/A510H/A589T, Q597N, V331T/Q597N, D665A, G56F/V331T/E416T/D665A, L125V/V465Q/P504T/S508K/A510H, G56F/V331T/Y478W/S483C/E529D/Q597N, D384N/1479L, Q233T/M640L, L125V/N419D/P504T/S508K/G530D, P504T/A510H/M640L, N419D/P504T/S508K, G56F/V331T/E361Q, E416T/D665A, G56F/V211M/E361Q/E416T/Y478W/H584L, G56F, G56F/V331T/E416T, Q175E/V211M, G56F/V211M/Y478W/S483C/E712D, D349E/P504T/S508K, L125V/S508K/A510H, V331T/E416T, V331T, I479L/P504T/A510H/A589T, V331T/Y478W, G56F/V331T/Y478W/S483C/Q597N, 1479L, L125V/P504T/S508K, I479L/A589T, or 1479L/A510H/A589T, wherein the amino acid positions are relative to the reference sequence corresponding to SEQ ID NO: 2.

In some embodiments, the amino acid sequence of the engineered AAV capsid polypeptide comprises at least a substitution or substitution set at amino acid position(s) 14/588/665, 173/588/665, 159/588/665, 157/588/665, 152/588/665, 485/588/665, 421/588/665, 391/588/665, 469/588/665, 588/665/628, 461/588/665, 165/588/665, 588/642/665, 588/595/665, 24/588/665, 414/588/665, 512/588/665, 519/588/665, 168/588/665, 588/598/665, 449/588/665, 588/665/736, 156/588/665, 462/588/665, 162/588/665, 416/588/665, 456/588/665, 587/588/665, 586/588/665, 588/665/668, 465/588/665, 454/588/665, 495/505/532/588/665, 494/497/588/665, 497/548/552/588/664/665, 588/661/665, 497/505/586/588/665, 495/588/665, 585/588/665/706, 588/665/706, 416/588/640/665, 665/456/457/494/497/505/588/661/706, 457/495/588/665, 505/588/665, 456/495/505/588/665, 457/497/549/552/588/664/665, 494/505/588/592/665, 456/457/588/665, 548/552/592/588/661/665/706, 456/457/494/497/505/588/661/665, 494/588/665, 494/495/505/588/664/665, 505/588/664/665, 456/494/497/588/661/665, 493/588/640/665, 457/548/555/588/592/661/665, 588/661/664/665, 457/588/592/661/665, 416/588/661/665, 588/664/665, 588/592/664/665, 457/494/497/588/665, 457/588/661/664/665, 456/497/588/592/665, 497/505/588/665, 155/588/665, 588/663/665, 401/588/665, 22/588/665, 175/588/665, 579/588/665, 172/588/665, 444/588/665, 23/588/665, 459/508/588/663/665/667, 17/588/665, 412/588/665, 440/588/665, 387/588/665, 453/586/588/665, 419/588/665, 25/588/665, 472/588/665, 162/450/588/665, 588/665, 437/588/665, 10/588/665, 562/588/665, 169/588/665, 565/588/665, 588/589/665, 495/497/588/665, 119/450/588/665, or 142/552/588/665, wherein the amino acid positions are relative to the reference sequence corresponding to SEQ ID NO: 2.

In some embodiments, the amino acid sequence of the engineered AAV capsid polypeptide comprises at least a substitution or substitution set 14R/588R/665G, 173S/588R/665G, 159R/588R/665G, 157T/588R/665G, 152Q/588R/665G, 485G/588R/665G, 421G/588R/665G, 391Q/588R/665G, 469W/588R/665G, 588R/665G/628Q, 461W/588R/665G, Q165G/Q588R/D665G, 14L/588R/665G, 469D/588R/665G, 588R/642E/665G, 588R/595F/665G, 24R/588R/665G, 414R/588R/665G, 512G/588R/665G, 519S/588R/665G, 157S/588R/665G, 14V/588R/665G, 157S/588R/665G, 168R/588R/665G, 588R/598W/665G, 449E/588R/665G, 449R/588R/665G, 588R/665G/736M, 588R/598T/665G, 156G/588R/665G, 24E/588R/665G, 462A/588R/665G, 24T/588R/665G, 588R/595G/665G, 414Q/588R/665G, 485S/588R/665G, 162V/588R/665G, 485L/588R/665G, 588R/642W/665G, 416Y/588R/665G, 416S/588R/665G, 4561/588R/665G, 5871/588R/665G, 586N/588R/665G, 588R/665G/668R, 465K/588R/665G, 454P/588R/665G, 495F/505Y/532T/588R/665G, 494L/497S/588R/665G, 497S/548V/5521/588R/664G/665G, 588R/661H/665G, 497S/505Y/586I/588R/665G, 495F/588R/665G, 495F/588R/665G, 585M/588R/665G/706W, 456F/588R/665G, 588R/665G/706W, 416T/588R/640L/665G, 665G/456F/457G/494L/497S/505Y/588R/661H/706W, 457G/495F/588R/665G, 505Y/588R/665G, 456F/495F/505Y/588R/665G, 457G/497S/549M/5521/588R/664G/665G, 494L/505Y/588R/592A/665G, 456F/457G/588R/665G, 548V/5521/592A/588R/661H/665G/706W, 456F/457G/494L/497S/505Y/588R/661H/665G, 494L/588R/665G, 494L/495F/505Y/588R/664G/665G, 505Y/588R/664G/665G, 456F/494L/497S/588R/661H/665G, 493T/588R/640L/665G, 457G/548V/555W/588R/592A/661H/665G, 588R/661H/664G/665G, 457G/588R/592A/661H/665G, 416T/588R/661W/665G, 588R/664G/665G, 588R/592A/664G/665G, 457G/494L/497S/588R/665G, 457G/588R/661H/664G/665G, 456F/497S/588R/592A/665G, 497S/505Y/588R/665G, 155G/588R/665G, 588R/663R/665G, 401A/588R/665G, 157L/588R/665G, 22R/588R/665G, 175G/588R/665G, 579L/588R/665G, 172E/588R/665G, 24W/588R/665G, 444V/588R/665G, 23R/588R/665G, 421R/588R/665G, 459R/508K/588R/663R/665G/667T, 414T/588R/665G, 17T/588R/665G, 462M/588R/665G, 412R/588R/665G, 221/588R/665G, 461G/588R/665G, 157Q/588R/665G, 391L/588R/665G, 485W/588R/665G, 173T/588R/665G, 440A/588R/665G, 387G/588R/665G, 159C/588R/665G, 453R/586K/588R/665G, 419G/588R/665G, 25Q/588R/665G, 472W/588R/665G, 462E/588R/665G, 162G/450N/588R/665G, 22M/588R/665G, 414D/588R/665G, 588R/665G, 175D/588R/665G, 156W/588R/665G, 437V/588R/665G, 157C/588R/665G, 512L/588R/665G, 588R/598G/665G, 449T/588R/665G, 173W/588R/665G, 472Q/588R/665G, 25G/588R/665G, 10I/588R/665G, 162G/588R/665G, 562L/588R/665G, 169V/588R/665G, 155R/588R/665G, 588R/642T/665G, 24V/588R/665G, 588V/665G, 588S/665G, 587Q/588R/665G, 588T/665G, 562R/588R/665G, 565K/588R/665G, 588R/589Y/665G, 495F/497S/588R/665G, 485P/588R/665G, 119S/450G/588R/665G, or 142N/552S/588R/665G, wherein the amino acid positions are relative to the reference sequence corresponding to SEQ ID NO: 2.

In some embodiments, the amino acid sequence of the engineered AAV capsid polypeptide comprises at least a substitution or substitution set at amino acid position(s) 457/495/588/665/725, 414/456/457/459/485/495/588/665/736, 193/457/495/588/665, 157/165/454/457/495/588/665, 157/165/457/495/588/665/668, 457/485/495/588/595/665, 165/454/457/485/495/588/665, 346/457/495/588/665, 421/456/457/459/495/588/665, 218/457/495/588/665, 205/457/495/588/665, 348/457/495/588/665, 421/457/495/588/665, 457/495/551/588/665, 165/457/495/588/665, 457/495/588/665/736, 457/495/529/552/585/588/665, 457/495/535/588/665, 457/495/529/585/588/665, 457/495/535/588/659/665, 457/495/588/594/665, 457/468/495/588/665, 101/457/495/588/665, 457/495/588/665/726, 457/468/495/588/592/665, 451/457/495/588/665, 457/495/559/588/665, 457/495/575/588/665, 457/465/495/588/665, 457/495/560/588/665, 273/457/495/588/665, 389/457/495/510/588/665, 457/470/495/588/665, 100/457/495/588/665, 457/495/588/665/724, 457/485/495/588/597/665, 457/495/510/588/665, 45/457/495/588/665, 256/457/495/588/665, 334/457/495/588/665, 63/457/495/588/665, 211/457/495/588/665, 264/457/495/588/665, 457/495/588/595/665, 391/457/495/588/665, 391/457/495/588/661/665, 200/457/495/588/665, 165/391/454/457/471/495/588/665, 157/165/391/457/495/588/665, 165/391/457/495/588/665, 157/391/421/457/495/588/665, 81/457/495/588/665, 401/414/457/485/495/588/665, 391/454/457/495/588/595/665, 457/495/529/588/665, 457/495/529/552/665/716, 457/493/495/585/588/665/667, 269/457/495/588/665, 457/495/552/588/665, 457/495/515/588/665, 457/495/565/588/665, 457/470/495/588/663/665, 414/457/495/588/665, 457/495/588/597/665, 457/495/597/588/665, 457/495/564/588/665, 389/457/495/588/665, 60/457/495/588/665, 457/495/588/665/720, 451/452/457/495/588/665, 457/495/496/588/665, 214/457/495/588/665, or 320/457/495/588/665, wherein the amino acid positions are relative to the reference sequence corresponding to SEQ ID NO: 2.

In some embodiments, the amino acid sequence of the engineered AAV capsid polypeptide comprises at least a substitution or substitution set 457G/495F/588R/665G/725G, 414R/456N/457G/459C/485P/495F/588R/665G/736M, 193S/457G/495F/588R/665G, 157W/165V/454Q/457G/495F/588R/665G, 157W/165V/457G/495F/588R/665G/668R, 457G/485S/495F/588R/595F/665G, 165V/454Q/457G/485S/495F/588R/665G, 346S/457G/495F/588R/665G, 421G/456N/457G/459C/495F/588R/665G, 218S/457G/495F/588R/665G, 205P/457G/495F/588R/665G, 348T/457G/495F/588R/665G, 421G/457G/495F/588R/665G, 457G/495F/551F/588R/665G, 165V/457G/495F/588R/665G, 457G/495F/588R/665G/736M, 457G/495F/529A/552S/585T/588R/665G, 457G/495F/535G/588R/665G, 457G/495F/529A/585T/588R/665G, 457G/495F/535G/588R/659Q/665G, 457G/495F/588R/594W/665G, 457G/468V/495F/588R/665G, 101E/457G/495F/588R/665G, 457G/495F/588R/665G/726H, 457G/495F/588R/594V/665G, 457G/468W/495F/588R/592L/665G, 451P/457G/495F/588R/665G, 457G/495F/588R/594T/665G, 457G/495F/559C/588R/665G, 457G/495F/575D/588R/665G, 457G/465P/495F/588R/665G, 457G/4651/495F/588R/665G, 457G/495F/560L/588R/665G, 457G/468A/495F/588R/665G, 451L/457G/495F/588R/665G, 457G/495F/588R/594N/665G, 273T/457G/495F/588R/665G, 389M/457G/495F/510C/588R/665G, 451Y/457G/495F/588R/665G, 457G/470Q/495F/588R/665G, 100L/457G/495F/588R/665G, 457G/465T/495F/588R/665G, 457G/468Q/495F/588R/665G, 457G/495F/588R/594Q/665G, 457G/495F/588R/594S/665G, 457G/495F/588R/665G/724V, 457G/485Q/495F/588R/597I/665G, 457G/495F/510T/588R/665G, 45F/457G/495F/588R/665G, 2561/457G/495F/588R/665G, 334V/457G/495F/588R/665G, 63Q/457G/495F/588R/665G, 211M/457G/495F/588R/665G, 264S/457G/495F/588R/665G, 457G/495F/588R/594M/665G, 457G/495F/588R/665G/725A, 457G/495F/588R/595F/665G, 391Q/457G/495F/588R/665G, 391Q/457G/495F/588R/661K/665G, 200P/457G/495F/588R/665G, 165V/391Q/454Q/457G/4711/495F/588R/665G, 157W/165V/391Q/457G/495F/588R/665G, 165V/391Q/457G/495F/588R/665G, 157W/391Q/421T/457G/495F/588R/665G, 81E/457G/495F/588R/665G, 401G/414R/457G/485P/495F/588R/665G, 391Q/454Q/457G/495F/588R/595F/665G, 457G/495F/529A/588R/665G, 457G/495F/529A/552S/665G/716D, 457G/493T/495F/585T/588R/665G/667T, 269T/457G/495F/588R/665G, 457G/495F/552S/588R/665G, 457G/495F/515V/588R/665G, 457G/495F/588R/665G/724I, 457G/465N/495F/588R/665G, 451G/457G/495F/588R/665G, 451H/457G/495F/588R/665G, 457G/495F/565T/588R/665G, 457G/495F/510G/588R/665G, 451S/457G/495F/588R/665G, 457G/470G/495F/588R/663D/665G, 414R/N457G/495F/588R/665G, 457G/495F/588R/597C/665G, 457G/465L/495F/588R/665G, 101S/457G/495F/588R/665G, 457G/495F/5971/588R/665G, 457G/495F/565G/588R/665G, 457G/495F/564D/588R/665G, 457G/495F/588R/665G/725W, 389P/457G/495F/588R/665G, 451T/457G/495F/588R/665G, 457G/495F/510E/588R/665G, 457G/468Y/495F/588R/665G, 457G/495F/515T/588R/665G, 457G/495F/564A/588R/665G, 457G/495F/510C/588R/665G, 457G/495F/515L/588R/665G, 457G/468W/495F/588R/665G, 60Y/457G/495F/588R/665G, 457G/495F/588R/665G/726I, 457G/495F/588R/665G/720F, 451V/452D/457G/495F/588R/665G, 451N/457G/495F/588R/665G, 457G/495F/5101/588R/665G, 457G/495F/496D/588R/665G, 389F/457G/495F/588R/665G, 457G/495F/510Q/588R/665G, 457G/495F/588R/665G/725M, 214D/457G/495F/588R/665G, or 320V/457G/495F/588R/665G, wherein the amino acid positions are relative to the reference sequence corresponding to SEQ ID NO: 2.

In some embodiments, the amino acid sequence of the engineered AAV capsid polypeptide comprises at least a substitution or substitution set at amino acid position(s) 333/457/495/588/665, 190/457/495/588/665, 254/457/495/588/665, 100/457/495/588/665, 190/457/495/588/665/725, 31/457/495/588/665, 101/457/495/588/665, 258/457/495/588/665, 268/457/495/588/665, 214/451/457/495/588/665, 331/457/495/588/665, 328/457/495/588/665, 33/457/495/588/665, 274/457/495/588/665, 33/457/495/576/588/665, 457/495/560/588/665, 51/190/457/495/588/665, 60/457/495/588/665, 79/272/457/495/588/665, 263/457/495/588/665, 270/457/495/588/665, 267/457/495/588/665, 210/266/457/495/588/665, 259/457/495/588/665, 53/457/495/588/665, 259/457/466/495/588/665, 90/457/495/588/665, 264/457/495/588/665, 172/266/457/495/588/665, 90/457/495/535/588/665, 266/457/495/588/665, 66/457/495/588/665, 329/457/495/588/665, or 126/457/470/495/588/665, wherein the amino acid positions are relative to the reference sequence corresponding to SEQ ID NO: 2.

In some embodiments, the amino acid sequence of the engineered AAV capsid polypeptide comprises at least a substitution or substitution set 333S/457G/495F/588R/665G, 190A/457G/495F/588R/665G, 254C/457G/495F/588R/665G, 100T/457G/495F/588R/665G, 190D/457G/495F/588R/665G/725H, 31L/457G/495F/588R/665G, 3331/457G/495F/588R/665G, 101R/457G/495F/588R/665G, 258T/457G/495F/588R/665G, 2681/457G/495F/588R/665G, 214D/451L/457G/495F/588R/665G, 101V/457G/495F/588R/665G, 331R/457G/495F/588R/665G, 190L/457G/495F/588R/665G, 328T/457G/495F/588R/665G, 33L/457G/495F/588R/665G, 274F/457G/495F/588R/665G, 33R/457G/495F/576Y/588R/665G, 333N/457G/495F/588R/665G, 331T/457G/495F/588R/665G, 457G/495F/560H/588R/665G, 33G/457G/495F/588R/665G, 190C/457G/495F/588R/665G, 51E/190V/457G/495F/588R/665G, 60S/457G/495F/588R/665G, 79H/272G/457G/495F/588R/665G, 263A/457G/495F/588R/665G, 270L/457G/495F/588R/665G, 100P/457G/495F/588R/665G, 263T/457G/495F/588R/665G, 267L/457G/495F/588R/665G, 210S/266F/457G/495F/588R/665G, 60K/457G/495F/588R/665G, 259K/457G/495F/588R/665G, 267L/457G/495F/588R/665G, 53R/457G/495F/588R/665G, 263T/457G/495F/588R/665G, 33F/457G/495F/588R/665G, 259S/457G/466T/495F/588R/665G, 100A/457G/495F/588R/665G, 331T/457G/495F/588R/665G, 90L/457G/495F/588R/665G, 190H/457G/495F/588R/665G, 333A/457G/495F/588R/665G, 333A/457G/495F/588R/665G, 264E/457G/495F/588R/665G, 270H/457G/495F/588R/665G, 172D/266V/457G/495F/588R/665G, 90R/457G/495F/588R/665G, 90A/457G/495F/535G/588R/665G, 267V/457G/495F/588R/665G, 270P/457G/495F/588R/665G, 264N/457G/495F/588R/665G, 100S/457G/495F/588R/665G, 266M/457G/495F/588R/665G, 100R/457G/495F/588R/665G, 190S/457G/495F/588R/665G, 270Q/457G/495F/588R/665G, 66R/457G/495F/588R/665G, 100V/457G/495F/588R/665G, 266Q/457G/495F/588R/665G, 33E/457G/495F/588R/665G, 53W/457G/495F/588R/665G, 266L/457G/495F/588R/665G, 264G/457G/495F/588R/665G, 53C/457G/495F/588R/665G, 53P/457G/495F/588R/665G, 90D/457G/495F/588R/665G, 100V/457G/495F/588R/665G, 90T/457G/495F/588R/665G, 270M/457G/495F/588R/665G, 267A/457G/495F/588R/665G, 270A/457G/495F/588R/665G, 190V/457G/495F/588R/665G, 329G/457G/495F/588R/665G, 53S/457G/495F/588R/665G, 266S/457G/495F/588R/665G, 270V/457G/495F/588R/665G, 263W/457G/495F/588R/665G, 266H/457G/495F/588R/665G, 126R/457G/470L/495F/588R/665G, 266T/457G/495F/588R/665G, 331S/457G/495F/588R/665G, or 100S/457G/495F/588R/665G, wherein the amino acid positions are relative to the reference sequence corresponding to SEQ ID NO: 2.

In some embodiments, the amino acid sequence of the engineered AAV capsid polypeptide comprises at least a substitution at an amino acid position set forth in Tables 5.1, 5.2, 6.1, 6.2, 7.1, and 7.2, wherein the amino acid positions are relative to the reference sequence corresponding to SEQ ID NO: 2.

In some embodiments, the amino acid sequence of the engineered AAV capsid polypeptide comprises at least one substitution set forth in Tables 5.1, 5.2, 6.1, 6.2, 7.1, and 7.2, wherein the amino acid positions are relative to the reference sequence corresponding to SEQ ID NO: 2.

In some embodiments, the amino acid sequence of the engineered AAV capsid polypeptide comprises at least a substitution or substitution set at amino acid position(s) set forth in Tables 5.1, 5.2, 6.1, 6.2, 7.1, and 7.2, wherein the amino acid positions are relative to the reference sequence corresponding to SEQ ID NO: 2.

In some embodiments, the amino acid sequence of the engineered AAV capsid polypeptide comprises at least a substitution or substitution set of an engineered AAV capsid polypeptide set forth in Tables 5.1, 5.2, 6.1, 6.2, 7.1, and 7.2, wherein the amino acid positions are relative to the reference sequence corresponding to SEQ ID NO: 2.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to an even-numbered SEQ ID NO. of SEQ ID NOs: 4-1134.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to an even-numbered SEQ ID NO. of SEQ ID NOs: 4-1134, wherein the amino acid sequence comprises one or more substitutions relative to the reference sequence corresponding to SEQ ID NO: 278 or 522.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522, wherein the amino acid sequence comprises one or more substitutions relative to the reference sequence corresponding to SEQ ID NO: 278 or 522.

In some embodiments, the amino acid sequence of the engineered AAV capsid polypeptide comprises at least a substitution at amino acid position 10, 14, 17, 22, 23, 24, 25, 31, 33, 45, 51, 53, 56, 60, 63, 66, 79, 81, 90, 100, 101, 119, 125, 126, 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to SEQ ID NO: 278 or 522.

In some embodiments, the amino acid sequence of the engineered AAV capsid polypeptide comprises at least a substitution or amino acid residue 101, 14L/R/V, 17T, 22I/M/R, 23R, 24E/R/T/V/W, 25G/Q/S, 31L, 33E/F/G/L/R, 45F, 51E, 53C/P/R/S/W, 56F, 60K/S/Y, 63Q, 66R, 79H, 81E, 90A/D/G/L/R/T, 100A/L/P/R/S/T/V, 101E/R/S/V, 119S, 125V, 126R, 142N, 152Q, 155G/R, 156G/W, 157C/L/Q/S/T/W, 159C/R, 162G/V, 165G/V, 168R, 169V, 172D/E/W, 173S/T/W, 175D/E/G, 190A/C/D/H/L/S/V, 193S, 200P, 205P, 210S, 211M, 214D, 218S, 233T, 254C, 250S, 2561, 258T, 259K/S, 263A/T/W, 264E/G/N/S, 266F/H/L/M/Q/S/T/V, 267A/L/V, 2681, 269T, 270A/H/L/M/P/Q/V, 272G, 273T, 274F, 320V, 328T, 329G, 331R/S/T, 333A/I/N/S, 334V, 346S, 348T, 349E, 361Q, 384N, 387G, 389F/M/P, 391L/Q, 401A/G, 412R, 414D/Q/R/T, 416S/T/Y, 419D/G, 421R/T, 437V, 440A, 444V, 449E/R/T, 450G/N, 451G/H/L/N/P/S/T/V/Y, 452D/G, 453F/L/Q/R/W, 454P/Q/R, 455R, 456F/I/K/L/M/N/P/V, 457F/G/L/R/T, 458S, 459A/C/M/R, 460A/C/G/L, 461G/W, 462A/E/M, 465I/K/L/N/P/Q/T, 466T, 468A/Q/V, 468W/Y, 469D/W, 470G/L/Q, 4711, 472Q/W, 478W, 479L, 483C, 485G/L/P/Q/S/W, 489C, 490P/V, 492Q/R/V/W, 493T, 4941, 495F/N, 496D, 497C/L/R/S, 499G, 500A/D/G/H/L/R/W, 501M/N, 504T, 505T/Y, 507T, 508K, 509Y, 510C/G, 510E/H/I/Q/T, 512G/L, 515L/T/V, 519S, 5281, 529A/D/V, 530D, 532C/Q/R/T/V, 535A/G/L, 537R, 538G, 539A, 545P, 547D/E/H/Q/R/W, 548A/L/M/R/V, 549M/W, 550A, 551F/M, 5521/K/R/S/V/W, 553K, 554G/I/L/Q/R/S/V, 555R/S/W, 556G/R/T, 559C, 560H/L, 562L/R, 564A/D, 565G/K/T, 575D, 576Y, 579L, 582G/L/R, 584C/L/V, 585I/M/T, 586A/G/I/K/N/R/V/W, 5871/Q/V, 588L/P/R/Q/S/T/V, 589R/T/Y, 592A/G/H/L/Y, 594M/N/Q/S/T/V/W, 595F/G, 597C/I/I/N, 598G/T/W, 628Q, 640L, 642E/T/W, 659N/Q/R, 660G/M, 661H/K/S/V/W, 662P, 663D/R, 664G, 665A/G/M/R/W, 666I/P, 667N/T, 668A/C/R, 705W, 706W, 709G, 710G/V, 712D, 716D, 720F, 724I/V, 725A/G/H/M/W, 726H/I, or 736M, or combinations thereof, wherein the amino acid positions are relative to the reference sequence corresponding to SEQ ID NO: 278 or 522.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278, wherein the amino acid sequence comprises one or more substitutions relative to the reference sequence corresponding to SEQ ID NO: 278.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to an even-numbered SEQ ID NO. of SEQ ID NOs: 332-722, wherein the amino acid sequence comprises one or more substitutions relative to the reference sequence corresponding to SEQ ID NO: 278.

In some embodiments, the amino acid sequence of the engineered AAV capsid polypeptide comprises at least a substitution or substitution set at amino acid position(s) 14, 173, 159, 157, 152, 485, 421, 391, 469, 628, 461, 165, 642, 595, 24, 414, 512, 519, 172, 168, 598, 449, 736, 156, 462, 162, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 155, 663, 401, 22, 175, 579, 444, 23, 459/508/663/667, 17, 412, 440, 387, 453/586, 419, 25, 472, 162/450, 585, 437, 10, 562, 169, 588, 565, 589, 495/497, 119/450, or 142/552, wherein the amino acid positions are relative to the reference sequence corresponding to SEQ ID NO: 278.

In some embodiments, the amino acid sequence of the engineered AAV capsid polypeptide comprises at least a substitution or substitution set 14R, 173S, 159R, 157T, 152Q, 485G, 421G, 391Q, 469W, 628Q, 461W, 165G, 14L, 469D, 642E, 595F, 24R, 414R, 512G, 519S, 157S, 14V, 172W, 168R, 598W, 449E, 449R, 736M, 598T, 156G, 24E, 462A, 24T, 595G, 414Q, 485S, 162V, 485L, 642W, 416Y, 416S, 4561, 5871, 586N, 668R, 465K, 454P, 495F/505Y/532T, 494L/497S, 497S/548V/5521/664G, 661H, 497S/505Y/5861, 495F, 555W, 585M/706W, 456F, 706W, 416T/640L, 456F/457G/494L/497S/505Y/661H/706W, 457G/495F, 505Y, 456F/495F/505Y, 457G/497S/549M/5521/664G, 494L/505Y/592A, 456F/457G, 548V/5521/592A/661H/706W, 456F/457G/494L/497S/505Y/661H, 494L, 494L/495F/505Y/664G, 505Y/664G, 456F/494L/497S/661H, 493T/640L, 457G/548V/555W/592A/661H, 661H/664G, 457G/592A/661H, 416T/661W, 664G, 592A/664G, 457G/494L/497S, 457G/661H/664G, 456F/497S/592A, 497S/505Y, 155G, 663R, 401A, 157L, 22R, 175G, 579L, 172E, 24W, 444V, 23R, 421R, 459R/508K/663R/667T, 414T, 17T, 462M, 412R, 22I, 461G, 157Q, 391L, 485W, 173T, 440A, 387G, 159C, 453R/586K, 419G, 25Q, 472W, 462E, 162G/450N, 22M, 414D, 585T, 175D, 156W, 437V, 157C, 512L, 598G, 449T, 173W, 472Q, 25G, 10I, 162G, 562L, 169V, 155R, 642T, 24V, 588V, 588S, 587Q, 588T, 562R, 565K, 589Y, 495F/497S, 485P, 119S/450G, or 142N/552S, wherein the amino acid positions are relative to the reference sequence corresponding to SEQ ID NO: 278.

In some embodiments, the amino acid sequence of the engineered AAV capsid polypeptide comprises at least a substitution or substitution set at amino acid position(s) N14R, F173S, I159R, A157T, E152Q, R485G, P421G, R391Q, S469W, N628Q, L461W, Q165G, N14L, S469D, H642E, W595F, A24R, S414R, N512G, N519S, A157S, N14V, N172W, K168R, N598W, K449E, K449R, L736M, N598T, S156G, A24E, K462A, A24T, W595G, S414Q, R485S, S162V, R485L, H642W, E416Y, E416S, Q456I, A587I, S586N, N668R, V465K, S454P, Q495F/G505Y/D532T, T494L/N497S, N497S/T548V/N552I/K664G, A661H, N497S/G505Y/S586I, Q495F, A555W, Q585M/Y706W, Q456F, Y706W, E416T/M640L, Q456F/N457G/T494L/N497S/G505Y/A661H/Y706W, N457G/Q495F, G505Y, Q456F/Q495F/G505Y, N457G/N497S/G549M/N552I/K664G, T494L/G505Y/Q592A, Q456F/N457G, T548V/N552I/Q592A/A661H/Y706W, Q456F/N457G/T494L/N497S/G505Y/A661H, T494L, T494L/Q495F/G505Y/K664G, G505Y/K664G, Q456F/T494L/N497S/A661H, V493T/M640L, N457G/T548V/A555W/Q592A/A661H, A661H/K664G, N457G/Q592A/A661H, E416T/A661W, K664G, Q592A/K664G, N457G/T494L/N497S, N457G/A661H/K664G, Q456F/N497S/Q592A, N497S/G505Y, S155G, N663R, P401A, A157L, W22R, Q175G, Q579L, N172E, A24W, L444V, W23R, P421R, Q459R/S508K/N663R/L667T, S414T, E17T, K462M, Q412R, W22I, L461G, A157Q, R391L, R485W, F173T, 1440A, Q387G, 1159C, G453R/S586K, N419G, L25Q, A472W, K462E, S162G/T450N, W22M, S414D, Q585T, Q175D, S156W, N437V, A157C, N512L, N598G, K449T, F173W, A472Q, L25G, W10I, S162G, N562L, K169V, S155R, H642T, A24V, R588V, R588S, A587Q, R588T, N562R, E565K, A589Y, Q495F/N497S, R485P, F119S/T450G, or K142N/N552S, wherein the amino acid positions are relative to the reference sequence corresponding to SEQ ID NO: 278.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 552, wherein the amino acid sequence comprises one or more substitutions relative to the reference sequence corresponding to SEQ ID NO: 552.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to an even-numbered SEQ ID NO. of SEQ ID NOs: 724-1134, wherein the amino acid sequence comprises one or more substitutions relative to the reference sequence corresponding to SEQ ID NO: 552.

In some embodiments, the amino acid sequence of the engineered AAV capsid polypeptide comprises at least a substitution or substitution set at amino acid position(s) 725, 414/456/459/485/736, 193, 157/165/454, 157/165/668, 485/595, 165/454/485, 346, 421/456/459, 218, 205, 348, 421, 551, 165, 736, 529/552/585, 535, 529/585, 535/659, 594, 468, 101, 726, 468/592, 451, 559, 575, 465, 560, 273, 389/510, 470, 100, 724, 485/597, 510, 45, 256, 334, 63, 211, 264, 595, 391, 391/661, 200, 165/391/454/471, 157/165/391, 165/391, 157/391/421, 81, 401/414/485, 391/454/595, 529, 529/552/588/716, 493/585/667, 269, 552, 515, 565, 470/663, 414, 597, 564, 389, 60, 720, 451/452, 496, 214, or 320, wherein the amino acid positions are relative to the reference sequence corresponding to SEQ ID NO: 552.

In some embodiments, the amino acid sequence of the engineered AAV capsid polypeptide comprises at least a substitution or substitution set 725G, 414R/456N/459C/485P/736M, 193S, 157W/165V/454Q, 157W/165V/668R, 485S/595F, 165V/454Q/485S, 346S, 421G/456N/459C, 218S, 205P, 348T, 421G, 551F, 165V, 736M, 529A/552S/585T, 535G, 529A/585T, 535G/659Q, 594W, 468V, 101E, 726H, 594V, 468W/592L, 451P, 594T, 559C, 575D, 465P, 4651, 560L, 468A, 451L, 594N, 273T, 389M/510C, 451Y, 470Q, 100L, 465T, 468Q, 594Q, 594S, 724V, 485Q/5971, 510T, 45F, 2561, 334V, 63Q, 211M, 264S, 594M, 725A, 595F, 391Q, 391Q/661K, 200P, 165V/391Q/454Q/471I, A157W/Q165V/R391Q, 165V/391Q, 157W/391Q/421T, 81E, 401G/414R/485P, 391Q/454Q/595F, 529A, 529A/552S/588Q/716D, 493T/585T/667T, 269T, 552S, 515V, 7241, 465N, 451G, 451H, 565T, 510G, 451S, 470G/663D, 414R, 597C, 465L, 101S, 597I, 565G, 564D, 725W, 389P, 451T, 510E, 468Y, 515T, 564A, 510C, 515L, 468W, 60Y, 7261, 720F, 451V/452D, 45 IN, 510I, 496D, 389F, 510Q, 725M, 214D, or 320V, wherein the amino acid positions are relative to the reference sequence corresponding to SEQ ID NO: 552.

In some embodiments, the amino acid sequence of the engineered AAV capsid polypeptide comprises at least a substitution or substitution set R725G, S414R/Q456N/Q459C/R485P/L736M, A193S, A157W/Q165V/S454Q, A157W/Q165V/N668R, R485S/W595F, Q165V/S454Q/R485S, T346S, P421G/Q456N/Q459C, A218S, S205P, S348T, P421G, D551F, Q165V, L736M, E529A/N552S/Q585T, F535G, E529A/Q585T, F535G/P659Q, G594W, P468V, Q101E, P726H, G594V, P468W/Q592L, 1451P, G594T, M559C, E575D, V465P, V4651, 1560L, P468A, 1451L, G594N, A273T, V389M/A510C, 1451Y, N470Q, F100L, V465T, P468Q, G594Q, G594S, P724V, R485Q/Q597I, A510T, L45F, L256I, 1334V, E63Q, V211M, T264S, G594M, R725A, W595F, R391Q, R391Q/A661K, S200P, Q165V/R391Q/S454Q/M471I, A157W/Q165V/R391Q, Q165V/R391Q, A157W/R391Q/P421T, Q81E, P401G/S414R/R485P, R391Q/S454Q/W595F, E529A, E529A/N552S/R588Q/N716D, V493T/Q585T/L667T, S269T, N552S, N515V, P724I, V465N, 1451G, I451H, E565T, A510G, 1451S, N470G/N663D, S414R, Q597C, V465L, Q101S, Q597I, E565G, E564D, R725W, V389P, I451T, A510E, P468Y, N515T, E564A, A510C, N515L, P468W, D60Y, P726I, V720F, I451V/N452D, 1451N, A510I, N496D, V389F, A510Q, R725M, N214D, or I320V, wherein the amino acid positions are relative to the reference sequence corresponding to SEQ ID NO: 552.

In some embodiments, the amino acid sequence of the engineered AAV capsid polypeptide comprises at least a substitution or substitution set at amino acid position(s) 333, 190, 254, 100, 190/725, 31, 101, 258, 268, 214/451, 331, 328, 33, 274, 33/576, 560, 51/190, 60, 79/272, 263, 270, 267, 210/266, 259, 53, 259/466, 90, 264, 172/266, 90/535, 266, 66, 329, or 126/470, wherein the amino acid positions are relative to the reference sequence corresponding to SEQ ID NO: 552.

In some embodiments, the amino acid sequence of the engineered AAV capsid polypeptide comprises at least a substitution or substitution set 333S, 190A, 254C, 100T, 190D/725H, 31L, 333I, 101R, 258T, 2681, 214D/451L, 101V, 331R, 190L, 328T, 33L, 274F, 33R/576Y, 333N, 331T, 560H, 33G, 190C, 51E/190V, 60S, 79H/272G, 263A, 270L, 100P, 263T, 267L, 210S/266F, 60K, 259K, 267L, 53R, 263T, 33F, 259S/466T, 100A, 331T, 90L, 190H, 333A, 90G, 264E, 270H, 172D/266V, 90R, 90A/535G, 267V, 270P, 264N, 100S, 266M, 100R, 190S, 270Q, 66R, 100V, 266Q, 33E, 53W, 266L, 264G, 53C, 53P, 90D, 100V, 90T, 270M, 267A, 270A, 190V, 329G, 53S, 266S, 270V, 263W, 266H, 126R/470L, 266T, 331S, or 100S, wherein the amino acid positions are relative to the reference sequence corresponding to SEQ ID NO: 552.

In some embodiments, the amino acid sequence of the engineered AAV capsid polypeptide comprises at least a substitution or substitution set T333S, E190A, N254C, F100T, E190D/R725H, Q31L, T333I, Q101R, K258T, S2681, N214D/1451L, Q101V, V331R, E190L, N328T, K33L, Y274F, K33R/S576Y, T333N, V331T, 1560H, K33G, E190C, K51E/E190V, D60S, Y79H/N272G, S263A, N270L, F100P, S263T, G267L, P210S/G266F, D60K, Q259K, G267L, L53R, S263T, K33F, Q259S/A466T, F100A, V331T, Y90L, E190H, T333A, Y90G, T264E, N270H, N172D/G266V, Y90R, Y90A/F535G, G267V, N270P, T264N, F100S, G266M, F100R, E190S, N270Q, N66R, F100V, G266Q, K33E, L53W, G266L, T264G, L53C, L53P, Y90D, F100V, Y90T, N270M, G267A, N270A, E190V, N329G, L53S, G266S, N270V, S263W, G266H, L126R/N470L, G266T, V331S, or F100S, wherein the amino acid positions are relative to the reference sequence corresponding to SEQ ID NO: 552.

In some embodiments, the amino acid sequence of the engineered AAV capsid polypeptide comprises at least a substitution at an amino acid position set forth in Tables 5.1, 5.2, 6.1, 6.2, 7.1, and 7.2, wherein the amino acid positions are relative to the reference sequence corresponding to SEQ ID NO: 2, 278, or 552.

In some embodiments, the amino acid sequence of the engineered AAV capsid polypeptide comprises at least one substitution set forth in Tables 5.1, 5.2, 6.1, 6.2, 7.1, and 7.2, wherein the amino acid positions are relative to the reference sequence corresponding to SEQ ID NO: 2, 278, or 552.

In some embodiments, the amino acid sequence of the engineered AAV capsid polypeptide comprises at least a substitution or substitution set at amino acid position(s) set forth in Tables 5.1, 5.2, 6.1, 6.2, 7.1, and 7.2, wherein the amino acid positions are relative to the reference sequence corresponding to SEQ ID NO: 2, 278, or 552.

In some embodiments, the amino acid sequence of the engineered AAV capsid polypeptide comprises at least a substitution or substitution set of an engineered AAV capsid polypeptide set forth in Tables 5.1, 5.2, 6.1, 6.2, 7.1, and 7.2, wherein the amino acid positions are relative to the reference sequence corresponding to SEQ ID NO: 2, 278, or 552.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a sequence corresponding to an engineered AAV capsid polypeptide set forth in Tables 5.1, 5.2, 6.1, 6.2, 7.1, and 7.2.

In some embodiments, the engineered AAV capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to SEQ ID NO, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, or 1134.

In some embodiments, the engineered AAV capsid polypeptide comprises an amino acid sequence comprising an even-numbered SEQ ID NO, of SEQ ID NOs: 4-1134, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions, as further discussed below.

In some embodiments, the engineered AAV capsid polypeptide comprises an amino acid sequence comprising SEQ ID NO, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, or 1134, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions.

As noted in some embodiments, the amino acid sequence optionally has 1, 2, 3, 4, 5, 6, 7, 8, 9, or up to 10 substitutions. In some embodiments, the amino acid sequence of the engineered AAV capsid polypeptide has 1, 2, 3, 4, up to 5 substitutions. In some embodiments, the substitutions comprises conservative and/or non-conservative substitutions. In some embodiments, the substitutions comprises conservative substitutions.

In another aspect, an engineered AAV capsid polypeptide comprises an engineered AAV VP2 capsid polypeptide. In some embodiments, the engineered AAV VP2 capsid polypeptide comprises a substitution or substitution set at amino acid positions encompassed in amino acid residues 138-736 of the engineered AAV capsid polypeptide described above. In some embodiments, the engineered AAV VP2 capsid polypeptide comprises amino acid residues 138-736 of an engineered AAV capsid polypeptide described herein.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 138-736 of an even-numbered SEQ ID NO, of SEQ ID NOs: 2-1134, wherein the amino acid sequence comprises one or more substitutions relative to the reference sequence corresponding to residues 138-736 of SEQ ID NO: 2, 278, or 522.

In some embodiments, an engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 2, 278, or 522, wherein the amino acid sequence comprises one or more substitutions relative to the reference sequence corresponding to residues 138-736 of SEQ ID NO: 2, 278, or 522, In some embodiments, the amino acid positions of the substitution(s) are relative to the amino acid sequence corresponding to SEQ ID NO: 2, 278, or 522.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 138-736 of SEQ ID NO: 2, wherein the amino acid sequence comprises one or more substitutions relative to the reference sequence corresponding to residues 138-736 of SEQ ID NO: 2.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522, wherein the amino acid sequence comprises one or more substitutions relative to the reference sequence corresponding to residues 138-736 of SEQ ID NO: 2.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 138-736 of an even-numbered SEQ ID NO, of SEQ ID NOs: 4-1134, wherein the amino acid sequence comprises one or more substitutions relative to the reference sequence corresponding to residues 138-736 of SEQ ID NO: 2.

In some embodiments, the amino acid sequence of the engineered AAV VP2 capsid polypeptide comprises at least a substitution at amino acid position 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution 142N, 152Q, 155G/R, 156G/W, 157C/L/Q/S/T/W, 159C/R, 162G/V, 165G/V, 168R, 169V, 172D/E/W, 173S/T/W, 175D/E/G, 190A/C/D/H/L/S/V, 193S, 200P, 205P, 210S, 211M, 214D, 218S, 233T, 254C, 250S, 2561, 258T, 259K/S, 263A/T/W, 264E/G/N/S, 266F/H/L/M/Q/S/T/V, 267A/L/V, 2681, 269T, 270A/H/L/M/P/Q/V, 272G, 273T, 274F, 320V, 328T, 329G, 331R/S/T, 333A/I/N/S, 334V, 346S, 348T, 349E, 361Q, 384N, 387G, 389F/M/P, 391L/Q, 401A/G, 412R, 414D/Q/R/T, 416S/T/Y, 419D/G, 421R/T, 437V, 440A, 444V, 449E/R/T, 450G/N, 451G/H/L/N/P/S/T/V/Y, 452D/G, 453F/L/Q/R/W, 454P/Q/R, 455R, 456F/I/K/L/M/N/P/V, 457F/G/L/R/T, 458S, 459A/C/M/R, 460A/C/G/L, 461G/W, 462A/E/M, 465I/K/L/N/P/Q/T, 466T, 468A/Q/V, 468W/Y, 469D/W, 470G/L/Q, 4711, 472Q/W, 478W, 479L, 483C, 485G/L/P/Q/S/W, 489C, 490P/V, 492Q/R/V/W, 493T, 4941, 495F/N, 496D, 497C/L/R/S, 499G, 500A/D/G/H/L/R/W, 501M/N, 504T, 505T/Y, 507T, 508K, 509Y, 510C/G, 510E/H/I/Q/T, 512G/L, 515L/T/V, 519S, 528I, 529A/D/V, 530D, 532C/Q/R/T/V, 535A/G/L, 537R, 538G, 539A, 545P, 547D/E/H/Q/R/W, 548A/L/M/R/V, 549M/W, 550A, 551F/M, 552I/K/R/S/V/W, 553K, 554G/I/L/Q/R/S/V, 555R/S/W, 556G/R/T, 559C, 560H/L, 562L/R, 564A/D, 565G/K/T, 575D, 576Y, 579L, 582G/L/R, 584C/L/V, 585I/M/T, 586A/G/I/I/K/N/R/V/W, 587I/Q/V, 588L/P/R/S/T/V, 589R/T/Y, 592A/G/H/L/Y, 594M/N/Q/S/T/V/W, 595F/G, 597C/I/I/N, 598G/T/W, 628Q, 640L, 642E/T/W, 659N/Q/R, 660G/M, 661H/K/S/V/W, 662P, 663D/R, 664G, 665A/G/M/R/W, 666I/P, 667N/T, 668A/C/R, 705W, 706W, 709G, 710G/V, 712D, 716D, 720F, 724I/V, 725A/G/H/M/W, 726H/I, or 736M, or combinations thereof, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution K142N, E152Q, S155G/R, S156G/W, A157C/L/Q/S/T/W, 1159C/R, S162G/V, Q165G/V, K168R, K169V, N172D/E/W, F173S/T/W, Q175D/E/G, E190A/C/D/H/L/S/V, A193S, S200P, S205P, P210S, V211M, N214D, A218S, Q233T, P250S, N254C, L256I, K258T, Q259K/S, S263A/T/W, T264E/G/N/S, G266F/H/L/M/Q/S/T/V, G267A/L/V, S268I, S269T, N270A/H/L/M/P/Q/V, N272G, A273T, Y274F, 1320V, N328T, N329G, V331R/S/T, T333A/I/N/S, L334V, T346S, S348T, D349E, E361Q, D384N, Q387G, V389F/M/P, R391L/Q, P401A/G, Q412R, S414D/Q/R/T, E416S/T/Y, N419D/G, P421R/T, N437V, 1440A, L444V, K449E/R/T, T450G/N, I451G/H/L/N/P/S/T/V/Y, N452D/G, G453F/L/Q/R/W, S454P/Q/R, G455R, Q456F/I/K/L/M/N/P/V, N457F/G/L/R/T, Q458S, Q459A/C/M/R, T460A/C/G/L, L461G/W, K462A/E/M, V465I/K/L/N/P/Q/T, A466T, P468A/Q/V, P468W/Y, S469D/W, N470G/L/Q, M471I, A472Q/W, Y478W, 1479L, S483C, R485G/L/P/Q/S/W, V489C, S490P/V, T492Q/R/V/W, V493T, T494I, Q495F/N, N496D, N497C/L/R/S, S499G, F500A/D/G/H/L/R/W, F501M/N, P504T, G505T/Y, S507T, S508K, W509Y, A510C/G, A510E/H/I/Q/T, N512G/L, N515L/T/V, N519S, K528I, E529A/D/V, G530D, D532C/Q/R/T/V, F535A/G/L, L537R, S538G, G539A, K545P, G547D/E/H/Q/R/W, T548A/L/M/R/V, G549M/W, R550A, D551F/M, N552I/K/R/S/V/W, V553K, D554G/I/L/Q/R/S/V, A555R/S/W, D556G/R/T, M559C, I560H/L, N562L/R, E564A/D, E565G/K/T, E575D, S576Y, Q579L, T582G/L/R, H584C/L/V, Q585I/M/T, S586A/G/I/I/K/N/R/V/W, A587I/Q/V, Q588L/P/R/S/T/V, A589R/T/Y, Q592A/G/H/L/Y, G594M/N/Q/S/T/V/W, W595F/G, Q597C/I/I/N, N598G/T/W, N628Q, M640L, H642E/T/W, P659N/Q/R, T660G/M, A661H/K/S/V/W, F662P, N663D/R, K664G, D665A/G/M/R/W, K666I/P, L667N/T, N668A/C/R, Y705W, Y706W, N709G, N710G/V, E712D, N716D, V720F, P724I/V, R725A/G/H/M/W, P726H/I, or L736M, or combinations thereof, wherein the amino acid positions are relative to the reference sequence corresponding to residues 138-736 of SEQ ID NO: 2.

In some embodiments, the amino acid sequence of the engineered AAV VP2 capsid polypeptide comprises substitutions in at least two amino acid positions 588, 665, 457, 495, 456, 505, 494, 497, 592, 586, 585, 706, 549, 552, 664, 661, 461, 668, 548, 510, 640, 419, 349, 504, 508, 453, 589, and 416, wherein the amino acid positions are relative to the reference sequence corresponding to residues 138-736 of SEQ ID NO: 2.

In some embodiments, the amino acid sequence of the engineered AAV VP2 capsid polypeptide comprises at least two substitutions comprising at least a first and second substitution, wherein the substitutions are at amino acid positions (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, 56, and 416, wherein the first substitution is at a position selected from (a), and the second substitution is at a position selected from (a), (b) and (c), wherein the amino acid positions are relative to the reference sequence corresponding to SEQ ID NO: 2, In some embodiments, the first substitution is at a position selected from (a) and the second substitution is at a position selected from (b) and (c), In some embodiments, the first substitution is at a position selected from (a) and the second substitution is at a position selected from (b), In some embodiments, the first substitution is at a position selected from (a) and the second substitution is selected from (c).

In some embodiments of the engineered AAV VP2 capsid polypeptide, the at least two substitutions comprising the first and second substitutions at the foregoing amino acid positions are selected from 349E, 416S/T/Y, 419D/G, 453F/L/Q/R/W, 456F/I/K/L/M/N/P/V, 457F/G/L/R/T, 461G/W, 4941, 495F/N, 497C/L/R/S, 501M/N, 504T, 505T/Y, 508K, 510E/H/I/Q/T, 548A/L/M/R/V, 549M/W, 552I/K/R/S/V/W, 585I/M/T, 586A/G/I/K/N/R/V/W, 588L/P/R/Q/S/T/V, 589R/T/Y, 592A/G/H/L/Y, 640L, 661H/K/S/V/W, 664G, 665A/G/M/R/W, 668A/C/R, and 706W, wherein the amino acid positions are relative to the reference sequence corresponding to residues 138-736 of SEQ ID NO: 2, In some embodiments, the at least two substitutions comprising the first and second substitutions at the foregoing amino acid positions are selected from 349E, 416T, 419D, 453R, 456F, 457G, 461W, 4941, 495F, 497S, 501M/N, 504T, 505Y, 508K, 510H, 548V, 549M, 552I/R, 585, 586I, 588R, 589R, 592A, 640L, 661H, 664G, 665G, 668R, and 706W, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution set at amino acid positions 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution set 588R/665G, 457G/495F/588R/665G, 461W/588R/665G, 588R/665G/668R, 495F/588R/665G, 494L/495F/505Y/588R/664G/665G, 494L/588R/665G, 588R/592A/664G/665G, 456F/497S/588R/592A/665G, 494L/497S/588R/665G, 456F/495F/505Y/588R/665G, 456F/457G/588R/665G, 497S/505Y/586I/588R/665G, 456F/457G/494L/497S/505Y/588R/661H/665G/706W, 457G/497S/549M/5521/588R/664G/665G, 585M/588R/665G/706W, 495F/588R/661H/665G, 497S/548V/5521/588R/664G/665G, 456F/457G/494L/497S/505Y/588R/661H/665G, 456F/494L/497S/588R/661H/665G, or 457G/494L/497S/588R/665G, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution set Q588R/D665G, N457G/Q495F/Q588R/D665G, L461W/Q588R/D665G, Q588R/D665G/N668R, Q495F/Q588R/D665G, T494L/Q495F/G505Y/Q588R/K664G/D665G, T494L/Q588R/D665G, Q588R/Q592A/K664G/D665G, Q456F/N497S/Q588R/Q592A/D665G, T494L/N497S/Q588R/D665G, Q456F/Q495F/G505Y/Q588R/D665G, Q456F/N457G/Q588R/D665G, N497S/G505Y/S586I/Q588R/D665G, Q456F/N457G/T494L/N497S/G505Y/Q588R/A661H/D665G/Y706W, N457G/N497S/G549M/N552I/Q588R/K664G/D665G, Q585M/Q588R/D665G/Y706W, Q495F/Q588R/A661H/D665G, N497S/T548V/N552I/Q588R/K664G/D665G, Q456F/N457G/T494L/N497S/G505Y/Q588R/A661H/D665G, or Q456F/T494L/N497S/Q588R/A661H/D665G, or N457G/T494L/N497S/Q588R/D665G, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 331/449, 211/509/712, 331/416/478, 507, 331/584/597, 478, 349/465/479/504/508, 349/640, 331/416/478/483/507/584/597, 416, 233/465/640, 508/510/589, 597, 331/597, 331/416/665, 465/504/508/510, 331/478/483/529/597, 384/479, 233/640, 419/504/508/530, 504/510/640, 419/504/508, 331/361, 416/665, 211/361/416/478/584, 331/416, 175/211, 211/478/483/712, 349/504/508, 508/510, 331/416, 331, 479/504/510/589, 331/478, 331/478/483/597, 479, 504/508, 479/589, or 479/510/589, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution or substitution set 547Q, 453R, 547E, 555W, 535A, 501M, 555R, 456L, 550A, 497L, 529Y, 705W, 554V, 500D, 548L, 582G, 586W, 453L, 584C, 535G, 585M, 592A, 459A, 492Q, 459R, 661W, 592L, 666I, 535L, 587V, 547R, 500A, 552S, 706W, 552W, 661V, 661H, 500L, 586A, 499G, 529V, 586R, 665M, 666P, 497R, 548R, 552R, 554R, 538G, 548M, 552V, 556T, 492R, 532V, 495N, 588R, 460G, 456M, 456V, 663R, 453F, 665R, 5541, 454R, 554S, 660G, 501N, 554G, 489C, 460C, 457G, 500G, 554L, 453W, 5521, 500R, 709G, 494L, 495F, 659N, 584V, 493T, 505Y, 668C, 547H, 589R, 584L, 662P, 456K, 500W, 547D, 556R, 456P, 588P, 659R, 456F, 585T, 532Q, 548A, 667N, 537R, 586V, 457L, 547W, 492V, 452G, 549M, 710V, 582R, 556G, 548V, 459M, 453Q, 460A, 586G, 588L, 532T, 592Y, 457T, 582L, 552K, 532C, 500H, 555S, 640L, 529A, 25S/492W, 665W, 554Q, 5851, 457R, 545P, 211M/416T, 539A, 529D, 589T/640L, 592G, 588R/665G, 551F, 504T/510H, 586K, 710G, 455R, 551M, 508K, 457F, 465Q/589T/640L, 497S, 419D/504T/508K/510H, 667T, 549W, 528I, 497C, 532R, 661S, 490P, 664G, 553K, 505T, 592H, 5861, 660M, 458S, 494I, 361Q/483C, 449R/584L, 384N/479L/504T/508K/510H, 507T/509Y/529D, 490V, 510H/640L, 233T/508K, 331T/449R, 211M/509Y/712D, 331T/416T/478W, 668A, 507T, 331T/584L/597N, 478W, 349E/465Q/479L/504T/508K, 349E/640L, 331T/584L/597N, 331T/416T/478W/483C/507T/584L/597N, 416T, 233T/465Q/640L, 508K/510H/589T, 597N, 331T/597N, 665A, 331T/416T/665A, 465Q/504T/508K/510H, 331T/478W/483C/529D/597N, 384N/479L, 233T/640L, 419D/504T/508K/530D, 504T/510H/640L, 419D/504T/508K, 331T/361Q, 416T/665A, 211M/361Q/416T/478W/584L, 331T/416T, 175E/211M, 211M/478W/483C/712D, 349E/504T/508K, 508K/510H, 331T/416T, 331T, 479L/504T/510H/589T, 331T/478W, 331T/478W/483C/597N, 479L, 504T/508K, 479L/589T, or 479L/510H/589T, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution or substitution set G547Q, G453R, G547E, A555W, F535A, F501M, A555R, Q456L, R550A, N497L, E529Y, Y705W, D554V, E500D, T548L, T582G, S586W, G453L, H584C, F535G, Q585M, Q592A, Q459A, T492Q, Q459R, A661W, Q592L, K666I, F535L, A587V, G547R, E500A, N552S, Y706W, N552W, A661V, A661H, E500L, S586A, S499G, E529V, S586R, D665M, K666P, N497R, T548R, N552R, D554R, S538G, T548M, N552V, D556T, T492R, D532V, Q495N, Q588R, T460G, Q456M, Q456V, N663R, G453F, D665R, D554I, S454R, D554S, T660G, F501N, D554G, V489C, T460C, N457G, E500G, D554L, G453W, N5521, E500R, N709G, T494L, Q495F, P659N, H584V, V493T, G505Y, N668C, G547H, A589R, H584L, F662P, Q456K, E500W, G547D, D556R, Q456P, Q588P, P659R, Q456F, Q585T, D532Q, T548A, L667N, L537R, S586V, N457L, G547W, T492V, N452G, G549M, N710V, T582R, D556G, T548V, Q459M, G453Q, T460A, S586G, Q588L, D532T, Q592Y, N457T, T582L, N552K, D532C, E500H, A555S, M640L, E529A, L25S/T492W, D665W, D554Q, Q585I, N457R, K545P, V211M/E416T, G539A, E529D, A589T/M640L, Q592G, Q588R/D665G, D551F, P504T/A510H, S586K, N710G, G455R, D551M, S508K, N457F, V465Q/A589T/M640L, N497S, N419D/P504T/S508K/A510H, L667T, G549W, K528I, N497C, D532R, A661S, S490P, K664G, V553K, G505T, Q592H, S586I, T660M, Q458S, T494I, E361Q/S483C, K449R/H584L, D384N/1479L/P504T/S508K/A510H, S507T/W509Y/E529D, S490V, A510H/M640L, Q233T/S508K, V331T/K449R, V211M/W509Y/E712D, V331T/E416T/Y478W, N668A, S507T, V331T/H584L/Q597N, Y478W, D349E/V465Q/1479L/P504T/S508K, D349E/M640L, V331T/H584L/Q597N, V331T/E416T/Y478W/S483C/S507T/H584L/Q597N, E416T, Q233T/V465Q/M640L, S508K/A510H/A589T, Q597N, V331T/Q597N, D665A, V331T/E416T/D665A, L125V/V465Q/P504T/S508K/A510H, V331T/Y478W/S483C/E529D/Q597N, D384N/1479L, Q233T/M640L, L125V/N419D/P504T/S508K/G530D, P504T/A510H/M640L, N419D/P504T/S508K, G56F/V331T/E361Q, E416T/D665A, V211M/E361Q/E416T/Y478W/H584L, V331T/E416T, Q175E/V211M, V211M/Y478W/S483C/E712D, D349E/P504T/S508K, S508K/A510H, V331T/E416T, V331T, I479L/P504T/A510H/A589T, V331T/Y478W, V331T/Y478W/S483C/Q597N, 1479L, P504T/S508K, 1479L/A589T, or I479L/A510H/A589T, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution or substitution set at amino acid position(s) 173/588/665, 159/588/665, 157/588/665, 152/588/665, 485/588/665, 421/588/665, 391/588/665, 469/588/665, 588/665/628, 461/588/665, 165/588/665, 588/642/665, 588/595/665, 414/588/665, 512/588/665, 519/588/665, 168/588/665, 588/598/665, 449/588/665, 588/665/736, 156/588/665, 462/588/665, 162/588/665, 416/588/665, 456/588/665, 587/588/665, 586/588/665, 588/665/668, 465/588/665, 454/588/665, 495/505/532/588/665, 494/497/588/665, 497/548/552/588/664/665, 588/661/665, 497/505/586/588/665, 495/588/665, 585/588/665/706, 588/665/706, 416/588/640/665, 665/456/457/494/497/505/588/661/706, 457/495/588/665, 505/588/665, 456/495/505/588/665, 457/497/549/552/588/664/665, 494/505/588/592/665, 456/457/588/665, 548/552/592/588/661/665/706, 456/457/494/497/505/588/661/665, 494/588/665, 494/495/505/588/664/665, 505/588/664/665, 456/494/497/588/661/665, 493/588/640/665, 457/548/555/588/592/661/665, 588/661/664/665, 457/588/592/661/665, 416/588/661/665, 588/664/665, 588/592/664/665, 457/494/497/588/665, 457/588/661/664/665, 456/497/588/592/665, 497/505/588/665, 155/588/665, 588/663/665, 401/588/665, 175/588/665, 579/588/665, 172/588/665, 444/588/665, 459/508/588/663/665/667, 412/588/665, 440/588/665, 387/588/665, 453/586/588/665, 419/588/665, 472/588/665, 162/450/588/665, 588/665, 437/588/665, 562/588/665, 169/588/665, 565/588/665, 588/589/665, 495/497/588/665, 450/588/665, or 142/552/588/665, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution or substitution set 173S/588R/665G, 159R/588R/665G, 157T/588R/665G, 152Q/588R/665G, 485G/588R/665G, 421G/588R/665G, 391Q/588R/665G, 469W/588R/665G, 588R/665G/628Q, 461W/588R/665G, Q165G/Q588R/D665G, 469D/588R/665G, 588R/642E/665G, 588R/595F/665G, 414R/588R/665G, 512G/588R/665G, 519S/588R/665G, 157S/588R/665G, 157S/588R/665G, 168R/588R/665G, 588R/598W/665G, 449E/588R/665G, 449R/588R/665G, 588R/665G/736M, 588R/598T/665G, 156G/588R/665G, 462A/588R/665G, 588R/595G/665G, 414Q/588R/665G, 485S/588R/665G, 162V/588R/665G, 485L/588R/665G, 588R/642W/665G, 416Y/588R/665G, 416S/588R/665G, 4561/588R/665G, 5871/588R/665G, 586N/588R/665G, 588R/665G/668R, 465K/588R/665G, 454P/588R/665G, 495F/505Y/532T/588R/665G, 494L/497S/588R/665G, 497S/548V/5521/588R/664G/665G, 588R/661H/665G, 497S/505Y/586I/588R/665G, 495F/588R/665G, 495F/588R/665G, 585M/588R/665G/706W, 456F/588R/665G, 588R/665G/706W, 416T/588R/640L/665G, 665G/456F/457G/494L/497S/505Y/588R/661H/706W, 457G/495F/588R/665G, 505Y/588R/665G, 456F/495F/505Y/588R/665G, 457G/497S/549M/5521/588R/664G/665G, 494L/505Y/588R/592A/665G, 456F/457G/588R/665G, 548V/5521/592A/588R/661H/665G/706W, 456F/457G/494L/497S/505Y/588R/661H/665G, 494L/588R/665G, 494L/495F/505Y/588R/664G/665G, 505Y/588R/664G/665G, 456F/494L/497S/588R/661H/665G, 493T/588R/640L/665G, 457G/548V/555W/588R/592A/661H/665G, 588R/661H/664G/665G, 457G/588R/592A/661H/665G, 416T/588R/661W/665G, 588R/664G/665G, 588R/592A/664G/665G, 457G/494L/497S/588R/665G, 457G/588R/661H/664G/665G, 456F/497S/588R/592A/665G, 497S/505Y/588R/665G, 155G/588R/665G, 588R/663R/665G, 401A/588R/665G, 157L/588R/665G, 175G/588R/665G, 579L/588R/665G, 172E/588R/665G, 444V/588R/665G, 421R/588R/665G, 459R/508K/588R/663R/665G/667T, 414T/588R/665G, 462M/588R/665G, 412R/588R/665G, 221/588R/665G, 461G/588R/665G, 157Q/588R/665G, 391L/588R/665G, 485W/588R/665G, 173T/588R/665G, 440A/588R/665G, 387G/588R/665G, 159C/588R/665G, 453R/586K/588R/665G, 419G/588R/665G, 472W/588R/665G, 462E/588R/665G, 162G/450N/588R/665G, 414D/588R/665G, 588R/665G, 175D/588R/665G, 156W/588R/665G, 437V/588R/665G, 157C/588R/665G, 512L/588R/665G, 588R/598G/665G, 449T/588R/665G, 173W/588R/665G, 472Q/588R/665G, 162G/588R/665G, 562L/588R/665G, 169V/588R/665G, 155R/588R/665G, 588R/642T/665G, 588V/665G, 588S/665G, 587Q/588R/665G, 588T/665G, 562R/588R/665G, 565K/588R/665G, 588R/589Y/665G, 495F/497S/588R/665G, 485P/588R/665G, 450G/588R/665G, or 142N/552S/588R/665G, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution or substitution set at amino acid position(s) 457/495/588/665/725, 414/456/457/459/485/495/588/665/736, 193/457/495/588/665, 157/165/454/457/495/588/665, 157/165/457/495/588/665/668, 457/485/495/588/595/665, 165/454/457/485/495/588/665, 346/457/495/588/665, 421/456/457/459/495/588/665, 218/457/495/588/665, 205/457/495/588/665, 348/457/495/588/665, 421/457/495/588/665, 457/495/551/588/665, 165/457/495/588/665, 457/495/588/665/736, 457/495/529/552/585/588/665, 457/495/535/588/665, 457/495/529/585/588/665, 457/495/535/588/659/665, 457/495/588/594/665, 457/468/495/588/665, 457/495/588/665/726, 457/468/495/588/592/665, 451/457/495/588/665, 457/495/559/588/665, 457/495/575/588/665, 457/465/495/588/665, 457/495/560/588/665, 273/457/495/588/665, 389/457/495/510/588/665, 457/470/495/588/665, 457/495/588/665/724, 457/485/495/588/597/665, 457/495/510/588/665, 256/457/495/588/665, 334/457/495/588/665, 211/457/495/588/665, 264/457/495/588/665, 457/495/588/595/665, 391/457/495/588/665, 391/457/495/588/661/665, 200/457/495/588/665, 165/391/454/457/471/495/588/665, 157/165/391/457/495/588/665, 165/391/457/495/588/665, 157/391/421/457/495/588/665, 401/414/457/485/495/588/665, 391/454/457/495/588/595/665, 457/495/529/588/665, 457/495/529/552/665/716, 457/493/495/585/588/665/667, 269/457/495/588/665, 457/495/552/588/665, 457/495/515/588/665, 457/495/565/588/665, 457/470/495/588/663/665, 414/457/495/588/665, 457/495/588/597/665, 457/495/597/588/665, 457/495/564/588/665, 389/457/495/588/665, 457/495/588/665/720, 451/452/457/495/588/665, 457/495/496/588/665, 214/457/495/588/665, or 320/457/495/588/665, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution or substitution set 457G/495F/588R/665G/725G, 414R/456N/457G/459C/485P/495F/588R/665G/736M, 193S/457G/495F/588R/665G, 157W/165V/454Q/457G/495F/588R/665G, 157W/165V/457G/495F/588R/665G/668R, 457G/485S/495F/588R/595F/665G, 165V/454Q/457G/485S/495F/588R/665G, 346S/457G/495F/588R/665G, 421G/456N/457G/459C/495F/588R/665G, 218S/457G/495F/588R/665G, 205P/457G/495F/588R/665G, 348T/457G/495F/588R/665G, 421G/457G/495F/588R/665G, 457G/495F/551F/588R/665G, 165V/457G/495F/588R/665G, 457G/495F/588R/665G/736M, 457G/495F/529A/552S/585T/588R/665G, 457G/495F/535G/588R/665G, 457G/495F/529A/585T/588R/665G, 457G/495F/535G/588R/659Q/665G, 457G/495F/588R/594W/665G, 457G/468V/495F/588R/665G, 457G/495F/588R/665G/726H, 457G/495F/588R/594V/665G, 457G/468W/495F/588R/592L/665G, 451P/457G/495F/588R/665G, 457G/495F/588R/594T/665G, 457G/495F/559C/588R/665G, 457G/495F/575D/588R/665G, 457G/465P/495F/588R/665G, 457G/4651/495F/588R/665G, 457G/495F/560L/588R/665G, 457G/468A/495F/588R/665G, 451L/457G/495F/588R/665G, 457G/495F/588R/594N/665G, 273T/457G/495F/588R/665G, 389M/457G/495F/510C/588R/665G, 451Y/457G/495F/588R/665G, 457G/470Q/495F/588R/665G, 457G/465T/495F/588R/665G, 457G/468Q/495F/588R/665G, 457G/495F/588R/594Q/665G, 457G/495F/588R/594S/665G, 457G/495F/588R/665G/724V, 457G/485Q/495F/588R/5971/665G, 457G/495F/510T/588R/665G, 45F/457G/495F/588R/665G, 2561/457G/495F/588R/665G, 334V/457G/495F/588R/665G, 211M/457G/495F/588R/665G, 264S/457G/495F/588R/665G, 457G/495F/588R/594M/665G, 457G/495F/588R/665G/725A, 457G/495F/588R/595F/665G, 391Q/457G/495F/588R/665G, 391Q/457G/495F/588R/661K/665G, 200P/457G/495F/588R/665G, 165V/391Q/454Q/457G/4711/495F/588R/665G, 157W/165V/391Q/457G/495F/588R/665G, 165V/391Q/457G/495F/588R/665G, 157W/391Q/421T/457G/495F/588R/665G, 401G/414R/457G/485P/495F/588R/665G, 391Q/454Q/457G/495F/588R/595F/665G, 457G/495F/529A/588R/665G, 457G/495F/529A/552S/665G/716D, 457G/493T/495F/585T/588R/665G/667T, 269T/457G/495F/588R/665G, 457G/495F/552S/588R/665G, 457G/495F/515V/588R/665G, 457G/495F/588R/665G/724I, 457G/465N/495F/588R/665G, 451G/457G/495F/588R/665G, 451H/457G/495F/588R/665G, 457G/495F/565T/588R/665G, 457G/495F/510G/588R/665G, 451S/457G/495F/588R/665G, 457G/470G/495F/588R/663D/665G, 414R/N457G/495F/588R/665G, 457G/495F/588R/597C/665G, 457G/465L/495F/588R/665G, 457G/495F/5971/588R/665G, 457G/495F/565G/588R/665G, 457G/495F/564D/588R/665G, 457G/495F/588R/665G/725W, 389P/457G/495F/588R/665G, 451T/457G/495F/588R/665G, 457G/495F/510E/588R/665G, 457G/468Y/495F/588R/665G, 457G/495F/515T/588R/665G, 457G/495F/564A/588R/665G, 457G/495F/510C/588R/665G, 457G/495F/515L/588R/665G, 457G/468W/495F/588R/665G, 457G/495F/588R/665G/726I, 457G/495F/588R/665G/720F, 451V/452D/457G/495F/588R/665G, 451N/457G/495F/588R/665G, 457G/495F/5101/588R/665G, 457G/495F/496D/588R/665G, 389F/457G/495F/588R/665G, 457G/495F/510Q/588R/665G, 457G/495F/588R/665G/725M, 214D/457G/495F/588R/665G, or 320V/457G/495F/588R/665G, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution or substitution set at amino acid position(s) 333/457/495/588/665, 190/457/495/588/665, 254/457/495/588/665, 190/457/495/588/665/725, 258/457/495/588/665, 268/457/495/588/665, 214/451/457/495/588/665, 331/457/495/588/665, 328/457/495/588/665, 274/457/495/588/665, 457/495/576/588/665, 457/495/560/588/665, 60/457/495/588/665, 79/272/457/495/588/665, 263/457/495/588/665, 270/457/495/588/665, 267/457/495/588/665, 210/266/457/495/588/665, 259/457/495/588/665, 259/457/466/495/588/665, 90/457/495/588/665, 264/457/495/588/665, 172/266/457/495/588/665, 266/457/495/588/665, 66/457/495/588/665, 329/457/495/588/665, or 457/470/495/588/665, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution or substitution set 333S/457G/495F/588R/665G, 190A/457G/495F/588R/665G, 254C/457G/495F/588R/665G, 190D/457G/495F/588R/665G/725H, 31L/457G/495F/588R/665G, 3331/457G/495F/588R/665G, 258T/457G/495F/588R/665G, 2681/457G/495F/588R/665G, 214D/451L/457G/495F/588R/665G, 331R/457G/495F/588R/665G, 190L/457G/495F/588R/665G, 328T/457G/495F/588R/665G, 274F/457G/495F/588R/665G, 457G/495F/576Y/588R/665G, 333N/457G/495F/588R/665G, 331T/457G/495F/588R/665G, 457G/495F/560H/588R/665G, 190C/457G/495F/588R/665G, 272G/457G/495F/588R/665G, 263A/457G/495F/588R/665G, 270L/457G/495F/588R/665G, 263T/457G/495F/588R/665G, 267L/457G/495F/588R/665G, 210S/266F/457G/495F/588R/665G, 259K/457G/495F/588R/665G, 267L/457G/495F/588R/665G, 263T/457G/495F/588R/665G, 259S/457G/466T/495F/588R/665G, 331T/457G/495F/588R/665G, 190H/457G/495F/588R/665G, 333A/457G/495F/588R/665G, 333A/457G/495F/588R/665G, 264E/457G/495F/588R/665G, 270H/457G/495F/588R/665G, 172D/266V/457G/495F/588R/665G, 267V/457G/495F/588R/665G, 270P/457G/495F/588R/665G, 264N/457G/495F/588R/665G, 266M/457G/495F/588R/665G, 190S/457G/495F/588R/665G, 270Q/457G/495F/588R/665G, 266Q/457G/495F/588R/665G, 266L/457G/495F/588R/665G, 264G/457G/495F/588R/665G, 270M/457G/495F/588R/665G, 267A/457G/495F/588R/665G, 270A/457G/495F/588R/665G, 190V/457G/495F/588R/665G, 329G/457G/495F/588R/665G, 266S/457G/495F/588R/665G, 270V/457G/495F/588R/665G, 263W/457G/495F/588R/665G, 266H/457G/495F/588R/665G, 457G/470L/495F/588R/665G, 266T/457G/495F/588R/665G, or 331S/457G/495F/588R/665G, wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution at an amino acid position between amino acid residues 138-736 of AAV capsid polypeptide, wherein the amino acid positions are set forth in Tables 5.1, 5.2, 6.1, 6.2, 7.1, and 7.2, and wherein the amino acid positions are relative to the reference sequence corresponding to residues 138-736 of SEQ ID NO: 2.

In some embodiments, the amino acid sequence of the engineered AAV VP2 capsid polypeptide comprises at least one substitution between amino acid residues 138-736 of AAV capsid polypeptide, wherein the substitutions are set forth in Tables 5.1, 5.2, 6.1, 6.2, 7.1, and 7.2, and wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution or substitution set at amino acid position(s) between amino acid residues 138-736 of AAV capsid polypeptide, wherein the substitution or substitution set are set forth in Tables 5.1, 5.2, 6.1, 6.2, 7.1, and 7.2, and wherein the amino acid positions are relative to the reference sequence corresponding to residues 138-736 of SEQ ID NO: 2.

In some embodiments, the amino acid sequence of the engineered AAV VP2 capsid polypeptide comprises at least a substitution or substitution set between amino acid residues 138-736 of an engineered AAV capsid polypeptide set forth in Tables 5.1, 5.2, 6.1, 6.2, 7.1, and 7.2, and wherein the amino acid positions are relative to the reference sequence corresponding to residues 138-736 of SEQ ID NO: 2.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO, of SEQ ID NOs: 4-1134.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO, of SEQ ID NOs: 4-1134, wherein the amino acid sequence comprises one or more substitutions relative to the reference sequence corresponding to residues 138-736 of SEQ ID NO: 278 or 522.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522, wherein the amino acid sequence comprises one or more substitutions relative to the reference sequence corresponding to residues 138-736 of SEQ ID NO: 278 or 522.

In some embodiments, the amino acid sequence of the engineered AAV VP2 capsid polypeptide comprises at least a substitution at amino acid position 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 138-736 of SEQ ID NO: 278 or 522.

In some embodiments, the amino acid sequence of the engineered AAV VP2 capsid polypeptide comprises at least a substitution or amino acid residue 142N, 152Q, 155G/R, 156G/W, 157C/L/Q/S/T/W, 159C/R, 162G/V, 165G/V, 168R, 169V, 172D/E/W, 173S/T/W, 175D/E/G, 190A/C/D/H/L/S/V, 193S, 200P, 205P, 210S, 211M, 214D, 218S, 233T, 250S, 254C, 256I, 258T, 259K/S, 263A/T/W, 264E/G/N/S, 266F/H/L/M/Q/S/T/V, 267A/L/V, 268I, 269T, 270A/H/L/M/P/Q/V, 272G, 273T, 274F, 320V, 328T, 329G, 331R/S/T, 333A/I/N/S, 334V, 346S, 348T, 349E, 361Q, 384N, 387G, 389F/M/P, 391L/Q, 401A/G, 412R, 414D/Q/R/T, 416S/T/Y, 419D/G, 421R/T, 437V, 440A, 444V, 449E/R/T, 450G/N, 451G/H/L/N/P/S/T/V/Y, 452D/G, 453F/L/Q/R/W, 454P/Q/R, 455R, 456F/I/K/L/M/N/P/V, 457F/G/L/R/T, 458S, 459A/C/M/R, 460A/C/G/L, 461G/W, 462A/E/M, 465I/K/L/N/P/Q/T, 466T, 468A/Q/V, 468W/Y, 469D/W, 470G/L/Q, 4711, 472Q/W, 478W, 479L, 483C, 485G/L/P/Q/S/W, 489C, 490P/V, 492Q/R/V/W, 493T, 4941, 495F/N, 496D, 497C/L/R/S, 499G, 500A/D/G/H/L/R/W, 501M/N, 504T, 505T/Y, 507T, 508K, 509Y, 510C/G, 510E/H/I/Q/T, 512G/L, 515L/T/V, 519S, 5281, 529A/D/V, 530D, 532C/Q/R/T/V, 535A/G/L, 537R, 538G, 539A, 545P, 547D/E/H/Q/R/W, 548A/L/M/R/V, 549M/W, 550A, 551F/M, 552I/K/R/S/V/W, 553K, 554G/I/L/Q/R/S/V, 555R/S/W, 556G/R/T, 559C, 560H/L, 562L/R, 564A/D, 565G/K/T, 575D, 576Y, 579L, 582G/L/R, 584C/L/V, 585I/M/T, 586A/G/I/I/K/N/R/V/W, 587I/Q/V, 588L/P/R/Q/S/T/V, 589R/T/Y, 592A/G/H/L/Y, 594M/N/Q/S/T/V/W, 595F/G, 597C/I/I/N, 598G/T/W, 628Q, 640L, 642E/T/W, 659N/Q/R, 660G/M, 661H/K/S/V/W, 662P, 663D/R, 664G, 665A/G/M/R/W, 666I/P, 667N/T, 668A/C/R, 705W, 706W, 709G, 710G/V, 712D, 716D, 720F, 724I/V, 725A/G/H/M/W, 726H/I, or 736M, or combinations thereof, wherein the amino acid positions are relative to the reference sequence corresponding to residues 138-736 of SEQ ID NO: 278 or 522.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278, wherein the amino acid sequence comprises one or more substitutions relative to the reference sequence corresponding to residues 138-736 of SEQ ID NO: 278.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 138-736 of an even-numbered SEQ ID NO, of SEQ ID NOs: 332-722, wherein the amino acid sequence comprises one or more substitutions relative to the reference sequence corresponding to residues 138-736 of SEQ ID NO: 278.

In some embodiments, the amino acid sequence of the engineered AAV VP2 capsid polypeptide comprises at least a substitution or substitution set at amino acid position(s) 173, 159, 157, 152, 485, 421, 391, 469, 628, 461, 165, 642, 595, 414, 512, 519, 172, 168, 598, 449, 736, 156, 462, 162, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 155, 663, 401, 175, 579, 444, 459/508/663/667, 412, 440, 387, 453/586, 419, 472, 162/450, 585, 437, 562, 169, 588, 565, 589, 495/497, 119/450, or 142/552, wherein the amino acid positions are relative to the reference sequence corresponding to residues 138-736 of SEQ ID NO: 278.

In some embodiments, the amino acid sequence of the engineered AAV VP2 capsid polypeptide comprises at least a substitution or substitution set 173S, 159R, 157T, 152Q, 485G, 421G, 391Q, 469W, 628Q, 461W, 165G, 469D, 642E, 595F, 414R, 512G, 519S, 157S, 172W, 168R, 598W, 449E, 449R, 736M, 598T, 156G, 462A, 595G, 414Q, 485S, 162V, 485L, 642W, 416Y, 416S, 4561, 5871, 586N, 668R, 465K, 454P, 495F/505Y/532T, 494L/497S, 497S/548V/5521/664G, 661H, 497S/505Y/586I, 495F, 555W, 585M/706W, 456F, 706W, 416T/640L, 456F/457G/494L/497S/505Y/661H/706W, 457G/495F, 505Y, 456F/495F/505Y, 457G/497S/549M/5521/664G, 494L/505Y/592A, 456F/457G, 548V/5521/592A/661H/706W, 456F/457G/494L/497S/505Y/661H, 494L, 494L/495F/505Y/664G, 505Y/664G, 456F/494L/497S/661H, 493T/640L, 457G/548V/555W/592A/661H, 661H/664G, 457G/592A/661H, 416T/661W, 664G, 592A/664G, 457G/494L/497S, 457G/661H/664G, 456F/497S/592A, 497S/505Y, 155G, 663R, 401A, 157L, 175G, 579L, 172E, 444V, 421R, 459R/508K/663R/667T, 414T, 17T, 462M, 412R, 221, 461G, 157Q, 391L, 485W, 173T, 440A, 387G, 159C, 453R/586K, 419G, 472W, 462E, 162G/450N, 414D, 585T, 175D, 156W, 437V, 157C, 512L, 598G, 449T, 173W, 472Q, 25G, 162G, 562L, 169V, 155R, 642T, 588V, 588S, 587Q, 588T, 562R, 565K, 589Y, 495F/497S, 485P, 450G, or 142N/552S, wherein the amino acid positions are relative to the reference sequence corresponding to residues 138-736 of SEQ ID NO: 278.

In some embodiments, the amino acid sequence of the engineered AAV VP2 capsid polypeptide comprises at least a substitution or substitution set F173S, 1159R, A157T, E152Q, R485G, P421G, R391Q, S469W, N628Q, L461W, Q165G, S469D, H642E, W595F, S414R, N512G, N519S, A157S, N172W, K168R, N598W, K449E, K449R, L736M, N598T, S156G, K462A, W595G, S414Q, R485S, S162V, R485L, H642W, E416Y, E416S, Q456I, A587I, S586N, N668R, V465K, S454P, Q495F/G505Y/D532T, T494L/N497S, N497S/T548V/N552I/K664G, A661H, N497S/G505Y/S586I, Q495F, A555W, Q585M/Y706W, Q456F, Y706W, E416T/M640L, Q456F/N457G/T494L/N497S/G505Y/A661H/Y706W, N457G/Q495F, G505Y, Q456F/Q495F/G505Y, N457G/N497S/G549M/N552I/K664G, T494L/G505Y/Q592A, Q456F/N457G, T548V/N552I/Q592A/A661H/Y706W, Q456F/N457G/T494L/N497S/G505Y/A661H, T494L, T494L/Q495F/G505Y/K664G, G505Y/K664G, Q456F/T494L/N497S/A661H, V493T/M640L, N457G/T548V/A555W/Q592A/A661H, A661H/K664G, N457G/Q592A/A661H, E416T/A661W, K664G, Q592A/K664G, N457G/T494L/N497S, N457G/A661H/K664G, Q456F/N497S/Q592A, N497S/G505Y, S155G, N663R, P401A, A157L, W22R, Q175G, Q579L, N172E, L444V, P421R, Q459R/S508K/N663R/L667T, S414T, K462M, Q412R, L461G, A157Q, R391L, R485W, F173T, I440A, Q387G, I159C, G453R/S586K, N419G, A472W, K462E, S162G/T450N, W22M, S414D, Q585T, Q175D, S156W, N437V, A157C, N512L, N598G, K449T, F173W, A472Q, S162G, N562L, K169V, S155R, H642T, R588V, R588S, A587Q, R588T, N562R, E565K, A589Y, Q495F/N497S, R485P, F119S/T450G, or K142N/N552S, wherein the amino acid positions are relative to the reference sequence corresponding to residues 138-736 of SEQ ID NO: 278.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 138-736 of SEQ ID NO: 552, wherein the amino acid sequence comprises one or more substitutions relative to the reference sequence corresponding to residues 138-736 of SEQ ID NO: 552.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO, of SEQ ID NOs: 724-1134, wherein the amino acid sequence comprises one or more substitutions relative to the reference sequence corresponding to residues 138-736 of SEQ ID NO: 552.

In some embodiments, the amino acid sequence of the engineered AAV VP2 capsid polypeptide comprises at least a substitution or substitution set at amino acid position(s) 725, 414/456/459/485/736, 193, 157/165/454, 157/165/668, 485/595, 165/454/485, 346, 421/456/459, 218, 205, 348, 421, 551, 165, 736, 529/552/585, 535, 529/585, 535/659, 594, 468, 726, 468/592, 451, 559, 575, 465, 560, 273, 389/510, 470, 724, 485/597, 510, 256, 334, 63, 211, 264, 595, 391, 391/661, 200, 165/391/454/471, 157/165/391, 165/391, 157/391/421, 401/414/485, 391/454/595, 529, 529/552/588/716, 493/585/667, 269, 552, 515, 565, 470/663, 414, 597, 564, 389, 720, 451/452, 496, 214, or 320, wherein the amino acid positions are relative to the reference sequence corresponding to residues 138-736 of SEQ ID NO: 552.

In some embodiments, the amino acid sequence of the engineered AAV VP2 capsid polypeptide comprises at least a substitution or substitution set 725G, 414R/456N/459C/485P/736M, 193S, 157W/165V/454Q, 157W/165V/668R, 485S/595F, 165V/454Q/485S, 346S, 421G/456N/459C, 218S, 205P, 348T, 421G, 551F, 165V, 736M, 529A/552S/585T, 535G, 529A/585T, 535G/659Q, 594W, 468V, 726H, 594V, 468W/592L, 451P, 594T, 559C, 575D, 465P, 4651, 560L, 468A, 451L, 594N, 273T, 389M/510C, 451Y, 470Q, 465T, 468Q, 594Q, 594S, 724V, 485Q/5971, 510T, 256I, 334V, 211M, 264S, 594M, 725A, 595F, 391Q, 391Q/661K, 200P, 165V/391Q/454Q/471I, A157W/Q165V/R391Q, 165V/391Q, 157W/391Q/421T, 401G/414R/485P, 391Q/454Q/595F, 529A, 529A/552S/588Q/716D, 493T/585T/667T, 269T, 552S, 515V, 724I, 465N, 451G, 451H, 565T, 510G, 451S, 470G/663D, 414R, 597C, 465L, 5971, 565G, 564D, 725W, 389P, 451T, 510E, 468Y, 515T, 564A, 510C, 515L, 468W, 7261, 720F, 451V/452D, 451N, 5101, 496D, 389F, 510Q, 725M, 214D, or 320V, wherein the amino acid positions are relative to the reference sequence corresponding to residues 138-736 of SEQ ID NO: 552.

In some embodiments, the amino acid sequence of the engineered AAV VP2 capsid polypeptide comprises at least a substitution or substitution set R725G, S414R/Q456N/Q459C/R485P/L736M, A193S, A157W/Q165V/S454Q, A157W/Q165V/N668R, R485S/W595F, Q165V/S454Q/R485S, T346S, P421G/Q456N/Q459C, A218S, S205P, S348T, P421G, D551F, Q165V, L736M, E529A/N552S/Q585T, F535G, E529A/Q585T, F535G/P659Q, G594W, P468V, P726H, G594V, P468W/Q592L, I451P, G594T, M559C, E575D, V465P, V465I, I560L, P468A, I451L, G594N, A273T, V389M/A510C, 1451Y, N470Q, V465T, P468Q, G594Q, G594S, P724V, R485Q/Q597I, A510T, L256I, I334V, V211M, T264S, G594M, R725A, W595F, R391Q, R391Q/A661K, S200P, Q165V/R391Q/S454Q/M471I, A157W/Q165V/R391Q, Q165V/R391Q, A157W/R391Q/P421T, Q81E, P401G/S414R/R485P, R391Q/S454Q/W595F, E529A, E529A/N552S/R588Q/N716D, V493T/Q585T/L667T, S269T, N552S, N515V, P724I, V465N, 1451G, 1451H, E565T, A510G, 1451S, N470G/N663D, S414R, Q597C, V465L, Q597I, E565G, E564D, R725W, V389P, 1451T, A510E, P468Y, N515T, E564A, A510C, N515L, P468W, P726I, V720F, 1451V/N452D, 1451N, A5101, N496D, V389F, A510Q, R725M, N214D, or 1320V, wherein the amino acid positions are relative to the reference sequence corresponding to residues 138-736 of SEQ ID NO: 552.

In some embodiments, the amino acid sequence of the engineered AAV VP2 capsid polypeptide comprises at least a substitution or substitution set at amino acid position(s) 333, 190, 254, 190/725, 258, 268, 214/451, 331, 328, 274, 576, 560, 79/272, 263, 270, 267, 210/266, 259, 259/466, 264, 172/266, 535, 266, 329, or 470, wherein the amino acid positions are relative to the reference sequence corresponding to residues 138-736 of SEQ ID NO: 552.

In some embodiments, the amino acid sequence of the engineered AAV VP2 capsid polypeptide comprises at least a substitution or substitution set 333S, 190A, 254C, 190D/725H, 3331, 258T, 2681, 214D/451L, 331R, 190L, 328T, 274F, 576Y, 333N, 331T, 560H, 190C, 272G, 263A, 270L, 263T, 267L, 210S/266F, 259K, 267L, 263T, 259S/466T, 331T, 190H, 333A, 264E, 270H, 172D/266V, 535G, 267V, 270P, 264N, 266M, 190S, 270Q, 266Q, 266L, 264G, 270M, 267A, 270A, 190V, 329G, 266S, 270V, 263W, 266H, 470L, 266T, or 331S, wherein the amino acid positions are relative to the reference sequence corresponding to residues 138-736 of SEQ ID NO: 552.

In some embodiments, the amino acid sequence of the engineered AAV VP2 capsid polypeptide comprises at least a substitution or substitution set T333S, E190A, N254C, E190D/R725H, T333I, K258T, S2681, N214D/1451L, 331R, E190L, N328T, Y274F, S576Y, T333N, V331T, I560H, E190C, E190V, N272G, S263A, N270L, S263T, G267L, P210S/G266F, Q259K, G267L, S263T, Q259S/A466T, V331T, E190H, T333A, T264E, N270H, N172D/G266V, F535G, G267V, N270P, T264N, G266M, E190S, N270Q, G266Q, G266L, T264G, N270M, G267A, N270A, E190V, N329G, G266S, N270V, S263W, G266H, N470L, G266T, or V331S, wherein the amino acid positions are relative to the reference sequence corresponding to residues 138-736 of SEQ ID NO: 552.

In some embodiments, the amino acid sequence of the engineered AAV VP2 capsid polypeptide comprises at least a substitution at an amino acid position between residues 138-736 of AAV capsid polypeptide, wherein the amino acid positions are set forth in Tables 5.1, 5.2, 6.1, 6.2, 7.1, and 7.2, and wherein the amino acid positions are relative to the reference sequence corresponding to residues 138-736 of SEQ ID NO: 2, 278, or 552.

In some embodiments, the amino acid sequence of the engineered AAV VP2 capsid polypeptide comprises at least one substitution set between residues 138-736 of AAV capsid polypeptide, wherein the substitutions are forth in Tables 5.1, 5.2, 6.1, 6.2, 7.1, and 7.2, and wherein the amino acid positions are relative to the reference sequence corresponding to residues 138-736 of SEQ ID NO: 2, 278, or 552.

In some embodiments, the amino acid sequence of the engineered AAV VP2 capsid polypeptide comprises at least a substitution or substitution set at amino acid position(s) between residues 138-736 of AAV capsid polypeptide, wherein the substitution or substitution set are set forth in Tables 5.1, 5.2, 6.1, 6.2, 7.1, and 7.2, and wherein the amino acid positions are relative to the reference sequence corresponding to residues 138-736 of SEQ ID NO: 2, 278, or 552.

In some embodiments, the amino acid sequence of the engineered AAV VP2 capsid polypeptide comprises at least a substitution or substitution set between residues 138-736 of an engineered AAV capsid polypeptide set forth in Tables 5.1, 5.2, 6.1, 6.2, 7.1, and 7.2, wherein the amino acid positions are relative to the reference sequence corresponding to residues 138-736 of SEQ ID NO: 2, 278, or 552.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 138-736 an engineered AAV capsid polypeptide set forth in Tables 5.1, 5.2, 6.1, 6.2, 7.1, and 7.2.

In some embodiments, the engineered AAV VP2 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a sequence corresponding to residues 138-736 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 338, 340, 342, 344, 346, 350, 352, 354, 356, 358, 360, 362, 364, 366, 368, 370, 372, 374, 376, 378, 380, 382, 384, 386, 388, 390, 392, 396, 398, 400, 402, 404, 406, 408, 410, 412, 414, 416, 418, 420, 422, 424, 426, 428, 430, 432, 436, 438, 440, 442, 444, 446, 448, 450, 452, 454, 456, 460, 462, 464, 468, 470, 472, 474, 478, 480, 482, 484, 486, 488, 490, 492, 496, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 608, 610, 612, 614, 618, 622, 624, 626, 630, 632, 634, 638, 640, 642, 644, 646, 648, 650, 652, 654, 656, 658, 662, 664, 666, 670, 672, 674, 676, 678, 680, 682, 684, 686, 688, 690, 696, 698, 700, 702, 704, 708, 710, 712, 714, 716, 718, 720, 722, 724, 726, 728, 730, 732, 734, 736, 738, 740, 742, 744, 746, 748, 750, 752, 754, 756, 758, 760, 762, 764, 768, 770, 776, 778, 782, 784, 786, 788, 796, 798, 800, 804, 808, 810, 812, 814, 816, 818, 820, 824, 826, 828, 830, 832, 834, 836, 838, 840, 844, 848, 852, 854, 856, 858, 860, 862, 866, 868, 872, 876, 878, 880, 884, 886, 888, 890, 892, 896, 898, 900, 902, 904, 906, 908, 910, 912, 914, 916, 920, 922, 924, 926, 928, 930, 932, 934, 936, 938, 942, 946, 948, 950, 954, 956, 962, 964, 966, 968, 970, 974, 976, 980, 982, 986, 988, 992, 994, 996, 1000, 1004, 1006, 1008, 1012, 1016, 1018, 1020, 1022, 1026, 1028, 1032, 1034, 1036, 1038, 1044, 1050, 1052, 1054, 1056, 1062, 1064, 1070, 1074, 1076, 1078, 1080, 1082, 1084, 1086, 1090, 1094, 1096, 1098, 1100, 1102, 1104, 1106, 1108, 1110, 1112, 1114, 1116, 1118, 1120, 1122, 1124, 1126, 1128, 1132, or 1134.

In some embodiments, the engineered AAV VP2 capsid polypeptide comprises an amino acid sequence comprising residues 138-736 of an even-numbered SEQ ID NO, of SEQ ID NOs: 4-1134, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions.

In some embodiments, the engineered AAV VP2 capsid polypeptide comprises an amino acid sequence comprising residues 138-736 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 338, 340, 342, 344, 346, 350, 352, 354, 356, 358, 360, 362, 364, 366, 368, 370, 372, 374, 376, 378, 380, 382, 384, 386, 388, 390, 392, 396, 398, 400, 402, 404, 406, 408, 410, 412, 414, 416, 418, 420, 422, 424, 426, 428, 430, 432, 436, 438, 440, 442, 444, 446, 448, 450, 452, 454, 456, 460, 462, 464, 468, 470, 472, 474, 478, 480, 482, 484, 486, 488, 490, 492, 496, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 608, 610, 612, 614, 618, 622, 624, 626, 630, 632, 634, 638, 640, 642, 644, 646, 648, 650, 652, 654, 656, 658, 662, 664, 666, 670, 672, 674, 676, 678, 680, 682, 684, 686, 688, 690, 696, 698, 700, 702, 704, 708, 710, 712, 714, 716, 718, 720, 722, 724, 726, 728, 730, 732, 734, 736, 738, 740, 742, 744, 746, 748, 750, 752, 754, 756, 758, 760, 762, 764, 768, 770, 776, 778, 782, 784, 786, 788, 796, 798, 800, 804, 808, 810, 812, 814, 816, 818, 820, 824, 826, 828, 830, 832, 834, 836, 838, 840, 844, 848, 852, 854, 856, 858, 860, 862, 866, 868, 872, 876, 878, 880, 884, 886, 888, 890, 892, 896, 898, 900, 902, 904, 906, 908, 910, 912, 914, 916, 920, 922, 924, 926, 928, 930, 932, 934, 936, 938, 942, 946, 948, 950, 954, 956, 962, 964, 966, 968, 970, 974, 976, 980, 982, 986, 988, 992, 994, 996, 1000, 1004, 1006, 1008, 1012, 1016, 1018, 1020, 1022, 1026, 1028, 1032, 1034, 1036, 1038, 1044, 1050, 1052, 1054, 1056, 1062, 1064, 1070, 1074, 1076, 1078, 1080, 1082, 1084, 1086, 1090, 1094, 1096, 1098, 1100, 1102, 1104, 1106, 1108, 1110, 1112, 1114, 1116, 1118, 1120, 1122, 1124, 1126, 1128, 1132, or 1134, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions.

In some embodiments, the amino acid sequence of the engineered AAV VP2 capsid polypeptide has 1, 2, 3, 4, up to 5 substitutions. In some embodiments, the substitutions comprises conservative and/or non-conservative substitutions. In some embodiments, the substitutions comprises conservative substitutions.

In another aspect, an engineered AAV capsid polypeptide comprises an engineered AAV VP3 capsid polypeptide. In some embodiments, the engineered AAV VP3 capsid polypeptide comprises a substitution or substitution set at amino acid positions encompassed in amino acid residues 203-736 of the engineered AAV capsid polypeptide described above. In some embodiments, the engineered AAV VP3 capsid polypeptide comprises amino acid residues 203-736 of an engineered AAV capsid polypeptide described herein.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of an even-numbered SEQ ID NO, of SEQ ID NOs: 2-1134, wherein the amino acid sequence comprises one or more substitutions relative to the reference sequence corresponding to residues 203-736 of SEQ ID NO: 2, 278, or 522.

In some embodiments, an engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of SEQ ID NO: 2, 278, or 522, wherein the amino acid sequence comprises one or more substitutions relative to the reference sequence corresponding to residues 203-736 of SEQ ID NO: 2, 278, or 522.

In some embodiments, the engineered AAV VP3 capsid polypeptides comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of SEQ ID NO: 2, wherein the amino acid sequence comprises one or more substitutions relative to the reference sequence corresponding to residues 203-736 of SEQ ID NO: 2.

In some embodiments, the engineered AAV VP3 capsid polypeptides comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of SEQ ID NO: 278 or 522, wherein the amino acid sequence comprises one or more substitutions relative to the reference sequence corresponding to residues 203-736 of SEQ ID NO: 2.

In some embodiments, the engineered AAV VP3 capsid polypeptides comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of an even-numbered SEQ ID NO, of SEQ ID NOs: 4-1134, wherein the amino acid sequence comprises one or more substitutions relative to the reference sequence corresponding to residues 203-736 of SEQ ID NO: 2.

In some embodiments, the amino acid sequence of the engineered AAV VP3 capsid polypeptide comprises at least a substitution at amino acid position 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution 205P, 210S, 211M, 214D, 218S, 233T, 254C, 250S, 2561, 258T, 259K/S, 263A/T/W, 264E/G/N/S, 266F/H/L/M/Q/S/T/V, 267A/L/V, 2681, 269T, 270A/H/L/M/P/Q/V, 272G, 273T, 274F, 320V, 328T, 329G, 331R/S/T, 333A/I/N/S, 334V, 346S, 348T, 349E, 361Q, 384N, 387G, 389F/M/P, 391L/Q, 401A/G, 412R, 414D/Q/R/T, 416S/T/Y, 419D/G, 421R/T, 437V, 440A, 444V, 449E/R/T, 450G/N, 451G/H/L/N/P/S/T/V/Y, 452D/G, 453F/L/Q/R/W, 454P/Q/R, 455R, 456F/I/K/L/M/N/P/V, 457F/G/L/R/T, 458S, 459A/C/M/R, 460A/C/G/L, 461G/W, 462A/E/M, 465I/K/L/N/P/Q/T, 466T, 468A/Q/V, 468W/Y, 469D/W, 470G/L/Q, 4711, 472Q/W, 478W, 479L, 483C, 485G/L/P/Q/S/W, 489C, 490P/V, 492Q/R/V/W, 493T, 4941, 495F/N, 496D, 497C/L/R/S, 499G, 500A/D/G/H/L/R/W, 501M/N, 504T, 505T/Y, 507T, 508K, 509Y, 510C/G, 510E/H/I/Q/T, 512G/L, 515L/T/V, 519S, 528I, 529A/D/V, 530D, 532C/Q/R/T/V, 535A/G/L, 537R, 538G, 539A, 545P, 547D/E/H/Q/R/W, 548A/L/M/R/V, 549M/W, 550A, 551F/M, 5521/K/R/S/V/W, 553K, 554G/I/L/Q/R/S/V, 555R/S/W, 556G/R/T, 559C, 560H/L, 562L/R, 564A/D, 565G/K/T, 575D, 576Y, 579L, 582G/L/R, 584C/L/V, 585I/M/T, 586A/G/I/I/K/N/R/V/W, 5871/Q/V, 588L/P/R/S/T/V, 589R/T/Y, 592A/G/H/L/Y, 594M/N/Q/S/T/V/W, 595F/G, 597C/I/I/N, 598G/T/W, 628Q, 640L, 642E/T/W, 659N/Q/R, 660G/M, 661H/K/S/V/W, 662P, 663D/R, 664G, 665A/G/M/R/W, 666I/P, 667N/T, 668A/C/R, 705W, 706W, 709G, 710G/V, 712D, 716D, 720F, 724I/V, 725A/G/H/M/W, 726H/I, or 736M, or combinations thereof, wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution S205P, P210S, V211M, N214D, A218S, Q233T, P250S, N254C, L256I, K258T, Q259K/S, S263A/T/W, T264E/G/N/S, G266F/H/L/M/Q/S/T/V, G267A/L/V, S2681, S269T, N270A/H/L/M/P/Q/V, N272G, A273T, Y274F, I320V, N328T, N329G, V331R/S/T, T333A/I/N/S, L334V, T346S, S348T, D349E, E361Q, D384N, Q387G, V389F/M/P, R391L/Q, P401A/G, Q412R, S414D/Q/R/T, E416S/T/Y, N419D/G, P421R/T, N437V, 1440A, L444V, K449E/R/T, T450G/N, 1451G/H/L/N/P/S/T/V/Y, N452D/G, G453F/L/Q/R/W, S454P/Q/R, G455R, Q456F/I/K/L/M/N/P/V, N457F/G/L/R/T, Q458S, Q459A/C/M/R, T460A/C/G/L, L461G/W, K462A/E/M, V465I/K/L/N/P/Q/T, A466T, P468A/Q/V, P468W/Y, S469D/W, N470G/L/Q, M471I, A472Q/W, Y478W, 1479L, S483C, R485G/L/P/Q/S/W, V489C, S490P/V, T492Q/R/V/W, V493T, T494I, Q495F/N, N496D, N497C/L/R/S, S499G, F500A/D/G/H/L/R/W, F501M/N, P504T, G505T/Y, S507T, S508K, W509Y, A510C/G, A510E/H/I/Q/T, N512G/L, N515L/T/V, N519S, K528I, E529A/D/V, G530D, D532C/Q/R/T/V, F535A/G/L, L537R, S538G, G539A, K545P, G547D/E/H/Q/R/W, T548A/L/M/R/V, G549M/W, R550A, D551F/M, N552I/K/R/S/V/W, V553K, D554G/I/L/Q/R/S/V, A555R/S/W, D556G/R/T, M559C, 1560H/L, N562L/R, E564A/D, E565G/K/T, E575D, S576Y, Q579L, T582G/L/R, H584C/L/V, Q585I/M/T, S586A/G/I/I/K/N/R/V/W, A5871/Q/V, Q588L/P/R/S/T/V, A589R/T/Y, Q592A/G/H/L/Y, G594M/N/Q/S/T/V/W, W595F/G, Q597C/I/I/N, N598G/T/W, N628Q, M640L, H642E/T/W, P659N/Q/R, T660G/M, A661H/K/S/V/W, F662P, N663D/R, K664G, D665A/G/M/R/W, K666I/P, L667N/T, N668A/C/R, Y705W, Y706W, N709G, N710G/V, E712D, N716D, V720F, P724I/V, R725A/G/H/M/W, P726H/I, or L736M, or combinations thereof, wherein the amino acid positions are relative to the reference sequence corresponding to residues 203-736 of SEQ ID NO: 2.

In some embodiments, the amino acid sequence of the engineered AAV VP3 capsid polypeptide comprises substitutions in at least two amino acid positions 588, 665, 457, 495, 456, 505, 494, 497, 592, 586, 585, 706, 549, 552, 664, 661, 461, 668, 548, 510, 640, 419, 349, 504, 508, 453, 589, and 416, wherein the amino acid positions are relative to the reference sequence corresponding to residues 203-736 of SEQ ID NO: 2.

In some embodiments, the amino acid sequence of the engineered AAV VP3 capsid polypeptide comprises at least two substitutions comprising a first and second substitution, wherein the substitutions are at amino acid positions selected from (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, 56, and 416, wherein the first substitution is at a position selected from (a), and the second substitution is at a position selected from (a), (b) and (c), wherein the amino acid positions are relative to the reference sequence corresponding to residues 203-736 of SEQ ID NO: 2, In some embodiments, the first substitution is at a position selected from (a) and the second substitution is at a position selected from (b) and (c), In some embodiments, the first substitution is at a position selected from (a) and the second substitution is at a position selected from (b), In some embodiments, the first substitution is at a position selected from (a) and the second substitution is selected from (c).

In some embodiments of the engineered AAV VP3 capsid polypeptide, the at least two substitutions comprising the first and second substitutions at the foregoing amino acid positions are selected from 349E, 416S/T/Y, 419D/G, 453F/L/Q/R/W, 456F/I/K/L/M/N/P/V, 457F/G/L/R/T, 461G/W, 4941, 495F/N, 497C/L/R/S, 501M/N, 504T, 505T/Y, 508K, 510E/H/I/Q/T, 548A/L/M/R/V, 549M/W, 552I/K/R/S/V/W, 585I/M/T, 586A/G/I/K/N/R/V/W, 588L/P/R/Q/S/T/V, 589R/T/Y, 592A/G/H/L/Y, 640L, 661H/K/S/V/W, 664G, 665A/G/M/R/W, 668A/C/R, and 706W, wherein the amino acid positions are relative to the reference sequence corresponding to residues 203-736 of SEQ ID NO: 2, In some embodiments, the at least two substitutions comprising the first and second substitutions at the foregoing amino acid positions are selected from 349E, 416T, 419D, 453R, 456F, 457G, 461W, 4941, 495F, 497S, 501M/N, 504T, 505Y, 508K, 510H, 548V, 549M, 552I/R, 585, 586I, 588R, 589R, 592A, 640L, 661H, 664G, 665G, 668R, and 706W, wherein the amino acid positions are relative to the reference sequence corresponding to residues 203-736 of SEQ ID NO: 2.

In some embodiments, the amino acid sequence of the engineered AAV VP3 capsid comprises at least a substitution set at amino acid positions 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution set 588R/665G, 457G/495F/588R/665G, 461W/588R/665G, 588R/665G/668R, 495F/588R/665G, 494L/495F/505Y/588R/664G/665G, 494L/588R/665G, 588R/592A/664G/665G, 456F/497S/588R/592A/665G, 494L/497S/588R/665G, 456F/495F/505Y/588R/665G, 456F/457G/588R/665G, 497S/505Y/586I/588R/665G, 456F/457G/494L/497S/505Y/588R/661H/665G/706W, 457G/497S/549M/5521/588R/664G/665G, 585M/588R/665G/706W, 495F/588R/661H/665G, 497S/548V/5521/588R/664G/665G, 456F/457G/494L/497S/505Y/588R/661H/665G, 456F/494L/497S/588R/661H/665G, or 457G/494L/497S/588R/665G, wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution set Q588R/D665G, N457G/Q495F/Q588R/D665G, L461W/Q588R/D665G, Q588R/D665G/N668R, Q495F/Q588R/D665G, T494L/Q495F/G505Y/Q588R/K664G/D665G, T494L/Q588R/D665G, Q588R/Q592A/K664G/D665G, Q456F/N497S/Q588R/Q592A/D665G, T494L/N497S/Q588R/D665G, Q456F/Q495F/G505Y/Q588R/D665G, Q456F/N457G/Q588R/D665G, N497S/G505Y/S586I/Q588R/D665G, Q456F/N457G/T494L/N497S/G505Y/Q588R/A661H/D665G/Y706W, N457G/N497S/G549M/N552I/Q588R/K664G/D665G, Q585M/Q588R/D665G/Y706W, Q495F/Q588R/A661H/D665G, N497S/T548V/N552I/Q588R/K664G/D665G, Q456F/N457G/T494L/N497S/G505Y/Q588R/A661H/D665G, Q456F/T494L/N497S/Q588R/A661H/D665G, or N457G/T494L/N497S/Q588R/D665G, wherein the amino acid positions are relative to residues 203-736 of SEQ ID NO: 2.

In some embodiments, the amino acid sequence of the engineered AAV VP3 capsid polypeptide comprises at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 331/449, 211/509/712, 331/416/478, 507, 331/584/597, 478, 349/465/479/504/508, 349/640, 331/416/478/483/507/584/597, 416, 233/465/640, 508/510/589, 597, 331/597, 331/416/665, 465/504/508/510, 331/478/483/529/597, 384/479, 233/640, 419/504/508/530, 504/510/640, 419/504/508, 331/361, 416/665, 211/361/416/478/584, 331/416, 175/211, 211/478/483/712, 349/504/508, 508/510, 331/416, 331, 479/504/510/589, 331/478, 331/478/483/597, 479, 504/508, 479/589, or 479/510/589, wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution or substitution set 547Q, 453R, 547E, 555W, 535A, 501M, 555R, 456L, 550A, 497L, 529Y, 705W, 554V, 500D, 548L, 582G, 586W, 453L, 584C, 535G, 585M, 592A, 459A, 492Q, 459R, 661W, 592L, 666I, 535L, 587V, 547R, 500A, 552S, 706W, 552W, 661V, 661H, 500L, 586A, 499G, 529V, 586R, 665M, 666P, 497R, 548R, 552R, 554R, 538G, 548M, 552V, 556T, 492R, 532V, 495N, 588R, 460G, 456M, 456V, 663R, 453F, 665R, 5541, 454R, 554S, 660G, 501N, 554G, 489C, 460C, 457G, 500G, 554L, 453W, 5521, 500R, 709G, 494L, 495F, 659N, 584V, 493T, 505Y, 668C, 547H, 589R, 584L, 662P, 456K, 500W, 547D, 556R, 456P, 588P, 659R, 456F, 585T, 532Q, 548A, 667N, 537R, 586V, 457L, 547W, 492V, 452G, 549M, 710V, 582R, 556G, 548V, 459M, 453Q, 460A, 586G, 588L, 532T, 592Y, 457T, 582L, 552K, 532C, 500H, 555S, 640L, 529A, 25S/492W, 665W, 554Q, 5851, 457R, 545P, 211M/416T, 539A, 529D, 589T/640L, 592G, 588R/665G, 551F, 504T/510H, 586K, 710G, 455R, 551M, 508K, 457F, 465Q/589T/640L, 497S, 419D/504T/508K/510H, 667T, 549W, 528I, 497C, 532R, 661S, 490P, 664G, 553K, 505T, 592H, 5861, 660M, 458S, 4941, 361Q/483C, 449R/584L, 384N/479L/504T/508K/510H, 507T/509Y/529D, 490V, 510H/640L, 233T/508K, 331T/449R, 211M/509Y/712D, 331T/416T/478W, 668A, 507T, 331T/584L/597N, 478W, 349E/465Q/479L/504T/508K, 349E/640L, 331T/584L/597N, 331T/416T/478W/483C/507T/584L/597N, 416T, 233T/465Q/640L, 508K/510H/589T, 597N, 331T/597N, 665A, 331T/416T/665A, 465Q/504T/508K/510H, 331T/478W/483C/529D/597N, 384N/479L, 233T/640L, 419D/504T/508K/530D, 504T/510H/640L, 419D/504T/508K, 331T/361Q, 416T/665A, 211M/361Q/416T/478W/584L, 331T/416T, 175E/211M, 211M/478W/483C/712D, 349E/504T/508K, 508K/510H, 331T/416T, 331T, 479L/504T/510H/589T, 331T/478W, 331T/478W/483C/597N, 479L, 504T/508K, 479L/589T, or 479L/510H/589T, wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution or substitution set G547Q, G453R, G547E, A555W, F535A, F501M, A555R, Q456L, R550A, N497L, E529Y, Y705W, D554V, E500D, T548L, T582G, S586W, G453L, H584C, F535G, Q585M, Q592A, Q459A, T492Q, Q459R, A661W, Q592L, K666I, F535L, A587V, G547R, E500A, N552S, Y706W, N552W, A661V, A661H, E500L, S586A, S499G, E529V, S586R, D665M, K666P, N497R, T548R, N552R, D554R, S538G, T548M, N552V, D556T, T492R, D532V, Q495N, Q588R, T460G, Q456M, Q456V, N663R, G453F, D665R, D554I, S454R, D554S, T660G, F501N, D554G, V489C, T460C, N457G, E500G, D554L, G453W, N552I, E500R, N709G, T494L, Q495F, P659N, H584V, V493T, G505Y, N668C, G547H, A589R, H584L, F662P, Q456K, E500W, G547D, D556R, Q456P, Q588P, P659R, Q456F, Q585T, D532Q, T548A, L667N, L537R, S586V, N457L, G547W, T492V, N452G, G549M, N710V, T582R, D556G, T548V, Q459M, G453Q, T460A, S586G, Q588L, D532T, Q592Y, N457T, T582L, N552K, D532C, E500H, A555S, M640L, E529A, L25S/T492W, D665W, D554Q, Q5851, N457R, K545P, V211M/E416T, G539A, E529D, A589T/M640L, Q592G, Q588R/D665G, D551F, P504T/A510H, S586K, N710G, G455R, D551M, S508K, N457F, V465Q/A589T/M640L, N497S, N419D/P504T/S508K/A510H, L667T, G549W, K528I, N497C, D532R, A661S, S490P, K664G, V553K, G505T, Q592H, S586I, T660M, Q458S, T494I, E361Q/S483C, K449R/H584L, D384N/479L/P504T/S508K/A510H, S507T/W509Y/E529D, S490V, A510H/M640L, Q233T/S508K, V331T/K449R, V211M/W509Y/E712D, V331T/E416T/Y478W, N668A, S507T, V331T/H584L/Q597N, Y478W, D349E/V465Q/1479L/P504T/S508K, D349E/M640L, V331T/H584L/Q597N, V331T/E416T/Y478W/S483C/S507T/H584L/Q597N, E416T, Q233T/V465Q/M640L, S508K/A510H/A589T, Q597N, V331T/Q597N, D665A, V331T/E416T/D665A, L125V/V465Q/P504T/S508K/A510H, V331T/Y478W/S483C/E529D/Q597N, D384N/1479L, Q233T/M640L, L125V/N419D/P504T/S508K/G530D, P504T/A510H/M640L, N419D/P504T/S508K, G56F/V331T/E361Q, E416T/D665A, V211M/E361Q/E416T/Y478W/H584L, V331T/E416T, Q175E/V211M, V211M/Y478W/S483C/E712D, D349E/P504T/S508K, S508K/A510H, V331T/E416T, V331T, I479L/P504T/A510H/A589T, V331T/Y478W, V331T/Y478W/S483C/Q597N, I479L, P504T/S508K, I479L/A589T, or I479L/A510H/A589T, wherein the amino acid positions are relative to the reference sequence corresponding to residues 203-736 of SEQ ID NO: 2, In some embodiments, the amino acid sequence of the engineered AAV VP3 capsid polypeptide comprises at least a substitution or substitution set at amino acid position(s) 485/588/665, 421/588/665, 391/588/665, 469/588/665, 588/665/628, 461/588/665, 588/642/665, 588/595/665, 414/588/665, 512/588/665, 519/588/665, 588/598/665, 449/588/665, 588/665/736, 462/588/665, 416/588/665, 456/588/665, 587/588/665, 586/588/665, 588/665/668, 465/588/665, 454/588/665, 495/505/532/588/665, 494/497/588/665, 497/548/552/588/664/665, 588/661/665, 497/505/586/588/665, 495/588/665, 585/588/665/706, 588/665/706, 416/588/640/665, 665/456/457/494/497/505/588/661/706, 457/495/588/665, 505/588/665, 456/495/505/588/665, 457/497/549/552/588/664/665, 494/505/588/592/665, 456/457/588/665, 548/552/592/588/661/665/706, 456/457/494/497/505/588/661/665, 494/588/665, 494/495/505/588/664/665, 505/588/664/665, 456/494/497/588/661/665, 493/588/640/665, 457/548/555/588/592/661/665, 588/661/664/665, 457/588/592/661/665, 416/588/661/665, 588/664/665, 588/592/664/665, 457/494/497/588/665, 457/588/661/664/665, 456/497/588/592/665, 497/505/588/665, 155/588/665, 588/663/665, 401/588/665, 579/588/665, 444/588/665, 459/508/588/663/665/667, 412/588/665, 440/588/665, 387/588/665, 453/586/588/665, 419/588/665, 472/588/665, 450/588/665, 588/665, 437/588/665, 562/588/665, 565/588/665, 588/589/665, 495/497/588/665, or 552/588/665, wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution or substitution set 485G/588R/665G, 421G/588R/665G, 391Q/588R/665G, 469W/588R/665G, 588R/665G/628Q, 461W/588R/665G, 469D/588R/665G, 588R/642E/665G, 588R/595F/665G, 414R/588R/665G, 512G/588R/665G, 519S/588R/665G, 588R/598W/665G, 449E/588R/665G, 449R/588R/665G, 588R/665G/736M, 588R/598T/665G, 462A/588R/665G, 588R/595G/665G, 414Q/588R/665G, 485S/588R/665G, 485L/588R/665G, 588R/642W/665G, 416Y/588R/665G, 416S/588R/665G, 4561/588R/665G, 5871/588R/665G, 586N/588R/665G, 588R/665G/668R, 465K/588R/665G, 454P/588R/665G, 495F/505Y/532T/588R/665G, 494L/497S/588R/665G, 497S/548V/5521/588R/664G/665G, 588R/661H/665G, 497S/505Y/586I/588R/665G, 495F/588R/665G, 495F/588R/665G, 585M/588R/665G/706W, 456F/588R/665G, 588R/665G/706W, 416T/588R/640L/665G, 665G/456F/457G/494L/497S/505Y/588R/661H/706W, 457G/495F/588R/665G, 505Y/588R/665G, 456F/495F/505Y/588R/665G, 457G/497S/549M/5521/588R/664G/665G, 494L/505Y/588R/592A/665G, 456F/457G/588R/665G, 548V/5521/592A/588R/661H/665G/706W, 456F/457G/494L/497S/505Y/588R/661H/665G, 494L/588R/665G, 494L/495F/505Y/588R/664G/665G, 505Y/588R/664G/665G, 456F/494L/497S/588R/661H/665G, 493T/588R/640L/665G, 457G/548V/555W/588R/592A/661H/665G, 588R/661H/664G/665G, 457G/588R/592A/661H/665G, 416T/588R/661W/665G, 588R/664G/665G, 588R/592A/664G/665G, 457G/494L/497S/588R/665G, 457G/588R/661H/664G/665G, 456F/497S/588R/592A/665G, 497S/505Y/588R/665G, 588R/663R/665G, 401A/588R/665G, 579L/588R/665G, 444V/588R/665G, 421R/588R/665G, 459R/508K/588R/663R/665G/667T, 414T/588R/665G, 462M/588R/665G, 412R/588R/665G, 221/588R/665G, 461G/588R/665G, 391L/588R/665G, 485W/588R/665G, 440A/588R/665G, 387G/588R/665G, 453R/586K/588R/665G, 419G/588R/665G, 472W/588R/665G, 462E/588R/665G, 450N/588R/665G, 414D/588R/665G, 588R/665G, 437V/588R/665G, 512L/588R/665G, 588R/598G/665G, 449T/588R/665G, 472Q/588R/665G, 562L/588R/665G, 588R/642T/665G, 588V/665G, 588S/665G, 587Q/588R/665G, 588T/665G, 562R/588R/665G, 565K/588R/665G, 588R/589Y/665G, 495F/497S/588R/665G, 485P/588R/665G, or 552S/588R/665G, wherein the amino acid positions are relative to the reference sequence corresponding to residues 203-736 of SEQ ID NO: 2.

In some embodiments, the engineered AAV VP3 capsid polypeptide comprises at least a substitution or substitution set at amino acid position(s) 457/495/588/665/725, 414/456/457/459/485/495/588/665/736, 457/485/495/588/595/665, 454/457/485/495/588/665, 346/457/495/588/665, 421/456/457/459/495/588/665, 218/457/495/588/665, 205/457/495/588/665, 348/457/495/588/665, 421/457/495/588/665, 457/495/551/588/665, 457/495/588/665/736, 457/495/529/552/585/588/665, 457/495/535/588/665, 457/495/529/585/588/665, 457/495/535/588/659/665, 457/495/588/594/665, 457/468/495/588/665, 457/495/588/665/726, 457/468/495/588/592/665, 451/457/495/588/665, 457/495/559/588/665, 457/495/575/588/665, 457/465/495/588/665, 457/495/560/588/665, 273/457/495/588/665, 389/457/495/510/588/665, 457/470/495/588/665, 457/495/588/665/724, 457/485/495/588/597/665, 457/495/510/588/665, 256/457/495/588/665, 334/457/495/588/665, 211/457/495/588/665, 264/457/495/588/665, 457/495/588/595/665, 391/457/495/588/665, 391/457/495/588/661/665, 391/454/457/471/495/588/665, 391/421/457/495/588/665, 401/414/457/485/495/588/665, 391/454/457/495/588/595/665, 457/495/529/588/665, 457/495/529/552/665/716, 457/493/495/585/588/665/667, 269/457/495/588/665, 457/495/552/588/665, 457/495/515/588/665, 457/495/565/588/665, 457/470/495/588/663/665, 414/457/495/588/665, 457/495/588/597/665, 457/495/597/588/665, 457/495/564/588/665, 389/457/495/588/665, 457/495/588/665/720, 451/452/457/495/588/665, 457/495/496/588/665, 214/457/495/588/665, or 320/457/495/588/665, wherein the amino acid positions are relative to the reference sequence corresponding to residues 203-736 of SEQ ID NO: 2.

In some embodiments, the engineered AAV VP3 capsid polypeptide comprises at least a substitution or substitution set 457G/495F/588R/665G/725G, 414R/456N/457G/459C/485P/495F/588R/665G/736M, 454Q/457G/495F/588R/665G, 457G/495F/588R/665G/668R, 457G/485S/495F/588R/595F/665G, 454Q/457G/485S/495F/588R/665G, 346S/457G/495F/588R/665G, 421G/456N/457G/459C/495F/588R/665G, 218S/457G/495F/588R/665G, 205P/457G/495F/588R/665G, 348T/457G/495F/588R/665G, 421G/457G/495F/588R/665G, 457G/495F/551F/588R/665G, 457G/495F/588R/665G/736M, 457G/495F/529A/552S/585T/588R/665G, 457G/495F/535G/588R/665G, 457G/495F/529A/585T/588R/665G, 457G/495F/535G/588R/659Q/665G, 457G/495F/588R/594W/665G, 457G/468V/495F/588R/665G, 457G/495F/588R/665G/726H, 457G/495F/588R/594V/665G, 457G/468W/495F/588R/592L/665G, 451P/457G/495F/588R/665G, 457G/495F/588R/594T/665G, 457G/495F/559C/588R/665G, 457G/495F/575D/588R/665G, 457G/465P/495F/588R/665G, 457G/4651/495F/588R/665G, 457G/495F/560L/588R/665G, 457G/468A/495F/588R/665G, 451L/457G/495F/588R/665G, 457G/495F/588R/594N/665G, 273T/457G/495F/588R/665G, 389M/457G/495F/510C/588R/665G, 451Y/457G/495F/588R/665G, 457G/470Q/495F/588R/665G, 457G/465T/495F/588R/665G, 457G/468Q/495F/588R/665G, 457G/495F/588R/594Q/665G, 457G/495F/588R/594S/665G, 457G/495F/588R/665G/724V, 457G/485Q/495F/588R/5971/665G, 457G/495F/510T/588R/665G, 45F/457G/495F/588R/665G, 2561/457G/495F/588R/665G, 334V/457G/495F/588R/665G, 211M/457G/495F/588R/665G, 264S/457G/495F/588R/665G, 457G/495F/588R/594M/665G, 457G/495F/588R/665G/725A, 457G/495F/588R/595F/665G, 391Q/457G/495F/588R/665G, 391Q/457G/495F/588R/661K/665G, 391Q/454Q/457G/4711/495F/588R/665G, 391Q/421T/457G/495F/588R/665G, 401G/414R/457G/485P/495F/588R/665G, 391Q/454Q/457G/495F/588R/595F/665G, 457G/495F/529A/588R/665G, 457G/495F/529A/552S/665G/716D, 457G/493T/495F/585T/588R/665G/667T, 269T/457G/495F/588R/665G, 457G/495F/552S/588R/665G, 457G/495F/515V/588R/665G, 457G/495F/588R/665G/724I, 457G/465N/495F/588R/665G, 451G/457G/495F/588R/665G, 451H/457G/495F/588R/665G, 457G/495F/565T/588R/665G, 457G/495F/510G/588R/665G, 451S/457G/495F/588R/665G, 457G/470G/495F/588R/663D/665G, 414R/N457G/495F/588R/665G, 457G/495F/588R/597C/665G, 457G/465L/495F/588R/665G, 457G/495F/5971/588R/665G, 457G/495F/565G/588R/665G, 457G/495F/564D/588R/665G, 457G/495F/588R/665G/725W, 389P/457G/495F/588R/665G, 451T/457G/495F/588R/665G, 457G/495F/510E/588R/665G, 457G/468Y/495F/588R/665G, 457G/495F/515T/588R/665G, 457G/495F/564A/588R/665G, 457G/495F/510C/588R/665G, 457G/495F/515L/588R/665G, 457G/468W/495F/588R/665G, 457G/495F/588R/665G/726I, 457G/495F/588R/665G/720F, 451V/452D/457G/495F/588R/665G, 451N/457G/495F/588R/665G, 457G/495F/5101/588R/665G, 457G/495F/496D/588R/665G, 389F/457G/495F/588R/665G, 457G/495F/510Q/588R/665G, 457G/495F/588R/665G/725M, 214D/457G/495F/588R/665G, or 320V/457G/495F/588R/665G, wherein the amino acid positions are relative to the reference sequence corresponding to residues 203-736 of SEQ ID NO: 2.

In some embodiments, the engineered AAV VP3 capsid polypeptide comprises at least a substitution or substitution set at amino acid position(s) 333/457/495/588/665, 254/457/495/588/665, 258/457/495/588/665, 268/457/495/588/665, 214/451/457/495/588/665, 331/457/495/588/665, 328/457/495/588/665, 274/457/495/588/665, 457/495/576/588/665, 457/495/560/588/665, 272/457/495/588/665, 263/457/495/588/665, 270/457/495/588/665, 267/457/495/588/665, 210/266/457/495/588/665, 259/457/495/588/665, 259/457/466/495/588/665, 264/457/495/588/665, 266/457/495/588/665, 329/457/495/588/665, or 457/470/495/588/665, wherein the amino acid positions are relative to the reference sequence corresponding to residues 203-736 of SEQ ID NO: 2.

In some embodiments, the engineered AAV VP3 capsid polypeptide comprises at least a substitution or substitution set 333S/457G/495F/588R/665G, 254C/457G/495F/588R/665G, 457G/495F/588R/665G/725H, 3331/457G/495F/588R/665G, 258T/457G/495F/588R/665G, 2681/457G/495F/588R/665G, 214D/451L/457G/495F/588R/665G, 331R/457G/495F/588R/665G, 328T/457G/495F/588R/665G, 274F/457G/495F/588R/665G, 457G/495F/576Y/588R/665G, 333N/457G/495F/588R/665G, 331T/457G/495F/588R/665G, 457G/495F/560H/588R/665G, 272G/457G/495F/588R/665G, 263A/457G/495F/588R/665G, 270L/457G/495F/588R/665G, 263T/457G/495F/588R/665G, 267L/457G/495F/588R/665G, 210S/266F/457G/495F/588R/665G, 259K/457G/495F/588R/665G, 267L/457G/495F/588R/665G, 263T/457G/495F/588R/665G, 259S/457G/466T/495F/588R/665G, 331T/457G/495F/588R/665G, 333A/457G/495F/588R/665G, 333A/457G/495F/588R/665G, 264E/457G/495F/588R/665G, 270H/457G/495F/588R/665G, 266V/457G/495F/588R/665G, 267V/457G/495F/588R/665G, 270P/457G/495F/588R/665G, 264N/457G/495F/588R/665G, 266M/457G/495F/588R/665G, 270Q/457G/495F/588R/665G, 266Q/457G/495F/588R/665G, 266L/457G/495F/588R/665G, 264G/457G/495F/588R/665G, 270M/457G/495F/588R/665G, 267A/457G/495F/588R/665G, 270A/457G/495F/588R/665G, 329G/457G/495F/588R/665G, 266S/457G/495F/588R/665G, 270V/457G/495F/588R/665G, 263W/457G/495F/588R/665G, 266H/457G/495F/588R/665G, 457G/470L/495F/588R/665G, 266T/457G/495F/588R/665G, or 331S/457G/495F/588R/665G, wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution at an amino acid position between residues 203-736 of AAV capsid polypeptide, wherein the amino acid positions are set forth in Tables 5.1, 5.2, 6.1, 6.2, 7.1, and 7.2, and wherein the amino acid positions are relative to the reference sequence corresponding to residues 203-736 of SEQ ID NO: 2.

In some embodiments, the amino acid sequence of the engineered AAV VP3 capsid polypeptide comprises at least one substitution between residues 203-736 of AAV capsid polypeptide, wherein the substitutions are set forth in Tables 5.1, 5.2, 6.1, 6.2, 7.1, and 7.2, and wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution or substitution set at amino acid position(s) between residues 203-736 of AAV capsid polypeptide, wherein the substitution or substitution set is set forth in Tables 5.1, 5.2, 6.1, 6.2, 7.1, and 7.2, and wherein the amino acid positions are relative to the reference sequence corresponding to residues 203-736 of SEQ ID NO: 2.

In some embodiments, the amino acid sequence of the engineered AAV VP3 capsid polypeptide comprises at least a substitution or substitution set between residues 203-736 of an engineered AAV capsid polypeptide set forth in Tables 5.1, 5.2, 6.1, 6.2, 7.1, and 7.2, and wherein the amino acid positions are relative to the reference sequence corresponding to residues 203-736 of SEQ ID NO: 2.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278 or 522.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO, of SEQ ID NOs: 4-1134.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO, of SEQ ID NOs: 4-1134, wherein the amino acid sequence comprises one or more substitutions relative to the reference sequence corresponding to residues 203-736 of SEQ ID NO: 278 or 522.

In some embodiments, an engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278 or 522, wherein the amino acid sequence comprises one or more substitutions relative to the reference sequence corresponding to residues 203-736 of SEQ ID NO: 278 or 522.

In some embodiments, the amino acid sequence of the engineered AAV VP3 capsid polypeptide comprises at least a substitution at amino acid position 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 203-736 of SEQ ID NO: 278 or 522.

In some embodiments, the amino acid sequence of the engineered AAV VP3 capsid polypeptide comprises at least a substitution or amino acid residue 205P, 210S, 211M, 214D, 218S, 233T, 250S, 254C, 2561, 258T, 259K/S, 263A/T/W, 264E/G/N/S, 266F/H/L/M/Q/S/T/V, 267A/L/V, 2681, 269T, 270A/H/L/M/P/Q/V, 272G, 273T, 274F, 320V, 328T, 329G, 331R/S/T, 333A/I/N/S, 334V, 346S, 348T, 349E, 361Q, 384N, 387G, 389F/M/P, 391L/Q, 401A/G, 412R, 414D/Q/R/T, 416S/T/Y, 419D/G, 421R/T, 437V, 440A, 444V, 449E/R/T, 450G/N, 451G/H/L/N/P/S/T/V/Y, 452D/G, 453F/L/Q/R/W, 454P/Q/R, 455R, 456F/I/K/L/M/N/P/V, 457F/G/L/R/T, 458S, 459A/C/M/R, 460A/C/G, 461G/W, 462A/E/M, 465I/K/L/N/P/Q/T, 466T, 468A/Q/V, 468W/Y, 469D/W, 470G/L/Q, 471I, 472Q/W, 478W, 479L, 483C, 485G/L/P/Q/S/W, 489C, 490P/V, 492Q/R/V/W, 493T, 494I, 495F/N, 496D, 497C/L/R/S, 499G, 500A/D/G/H/L/R/W, 501M/N, 504T, 505T/Y, 507T, 508K, 509Y, 510C/G, 510E/H/I/Q/T, 512G/L, 515L/T/V, 519S, 5281, 529A/D/V, 530D, 532C/Q/R/T/V, 535A/G/L, 537R, 538G, 539A, 545P, 547D/E/H/Q/R/W, 548A/L/M/R/V, 549M/W, 550A, 551F/M, 552I/K/R/S/V/W, 553K, 554G/I/L/Q/R/S/V, 555R/S/W, 556G/R/T, 559C, 560H/L, 562L/R, 564A/D, 565G/K/T, 575D, 576Y, 579L, 582G/L/R, 584C/L/V, 585I/M/T, 586A/G/I/I/K/N/R/V/W, 587I/Q/V, 588L/P/R/Q/S/T/V, 589R/T/Y, 592A/G/H/L/Y, 594M/N/Q/S/T/V/W, 595F/G, 597C/I/I/N, 598G/T/W, 628Q, 640L, 642E/T/W, 659N/Q/R, 660G/M, 661H/K/S/V/W, 662P, 663D/R, 664G, 665A/G/M/R/W, 666I/P, 667N/T, 668A/C/R, 705W, 706W, 709G, 710G/V, 712D, 716D, 720F, 724I/V, 725A/G/H/M/W, 726H/I, or 736M, or combinations thereof, wherein the amino acid positions are relative to the reference sequence corresponding to residues 203-736 of SEQ ID NO: 278 or 522.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278, wherein the amino acid sequence comprises one or more substitutions relative to the reference sequence corresponding to residues 203-736 of SEQ ID NO: 278.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO, of SEQ ID NOs: 332-722, wherein the amino acid sequence comprises one or more substitutions relative to the reference sequence corresponding to residues 203-736 of SEQ ID NO: 278.

In some embodiments, the amino acid sequence of the engineered AAV VP3 capsid polypeptide comprises at least a substitution or substitution set at amino acid position(s) 485, 421, 391, 469, 628, 461, 642, 595, 414, 512, 519, 598, 449, 736, 462, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 663, 401, 579, 444, 459/508/663/667, 412, 440, 387, 453/586, 419, 472, 450, 585, 437, 562, 169, 588, 565, 589, 495/497, or 142/552, wherein the amino acid positions are relative to the reference sequence corresponding to residues 203-736 of SEQ ID NO: 278.

In some embodiments, the amino acid sequence of the engineered AAV VP3 capsid polypeptide comprises at least a substitution or substitution set 485G, 421G, 391Q, 469W, 628Q, 461W, 469D, 642E, 595F, 414R, 512G, 519S, 598W, 449E, 449R, 736M, 598T, 462A, 595G, 414Q, 485S, 485L, 642W, 416Y, 416S, 4561, 5871, 586N, 668R, 465K, 454P, 495F/505Y/532T, 494L/497S, 497S/548V/5521/664G, 661H, 497S/505Y/586I, 495F, 555W, 585M/706W, 456F, 706W, 416T/640L, 456F/457G/494L/497S/505Y/661H/706W, 457G/495F, 505Y, 456F/495F/505Y, 457G/497S/549M/5521/664G, 494L/505Y/592A, 456F/457G, 548V/5521/592A/661H/706W, 456F/457G/494L/497S/505Y/661H, 494L, 494L/495F/505Y/664G, 505Y/664G, 456F/494L/497S/661H, 493T/640L, 457G/548V/555W/592A/661H, 661H/664G, 457G/592A/661H, 416T/661W, 664G, 592A/664G, 457G/494L/497S, 457G/661H/664G, 456F/497S/592A, 497S/505Y, 663R, 401A, 579L, 444V, 421R, 459R/508K/663R/667T, 414T, 462M, 412R, 461G, 391L, 485W, 440A, 387G, 453R/586K, 419G, 472W, 462E, 450N, 414D, 585T, 437V, 512L, 598G, 449T, 472Q, 562L, 642T, 588V, 588S, 587Q, 588T, 562R, 565K, 589Y, 495F/497S, 485P, 450G, or 552S, wherein the amino acid positions are relative to the reference sequence corresponding to residues 203-736 of SEQ ID NO: 278.

In some embodiments, the amino acid sequence of the engineered AAV VP3 capsid polypeptide comprises at least a substitution or substitution set R485G, P421G, R391Q, S469W, N628Q, L461W, S469D, H642E, W595F, S414R, N512G, N519S, N598W, K449E, K449R, L736M, N598T, K462A, W595G, S414Q, R485S, R485L, H642W, E416Y, E416S, Q456I, A587I, S586N, N668R, V465K, S454P, Q495F/G505Y/D532T, T494L/N497S, N497S/T548V/N552I/K664G, A661H, N497S/G505Y/S586I, Q495F, A555W, Q585M/Y706W, Q456F, Y706W, E416T/M640L, Q456F/N457G/T494L/N497S/G505Y/A661H/Y706W, N457G/Q495F, G505Y, Q456F/Q495F/G505Y, N457G/N497S/G549M/N5521/K664G, T494L/G505Y/Q592A, Q456F/N457G, T548V/N552I/Q592A/A661H/Y706W, Q456F/N457G/T494L/N497S/G505Y/A661H, T494L, T494L/Q495F/G505Y/K664G, G505Y/K664G, Q456F/T494L/N497S/A661H, V493T/M640L, N457G/T548V/A555W/Q592A/A661H, A661H/K664G, N457G/Q592A/A661H, E416T/A661W, K664G, Q592A/K664G, N457G/T494L/N497S, N457G/A661H/K664G, Q456F/N497S/Q592A, N497S/G505Y, N663R, P401A, Q579L, L444V, P421R, Q459R/S508K/N663R/L667T, S414T, K462M, Q412R, L461G, R391L, R485W, 1440A, Q387G, G453R/S586K, N419G, A472W, K462E, T450N, S414D, Q585T, N437V, N512L, N598G, K449T, A472Q, N562L, H642T, R588V, R588S, A587Q, R588T, N562R, E565K, A589Y, Q495F/N497S, R485P, T450G, or N552S, wherein the amino acid positions are relative to the reference sequence corresponding to residues 203-736 of SEQ ID NO: 278.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 552, wherein the amino acid sequence comprises one or more substitutions relative to the reference sequence corresponding to residues 203-736 of SEQ ID NO: 552.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of an even-numbered SEQ ID NO, of SEQ ID NOs: 724-1134, wherein the amino acid sequence comprises one or more substitutions relative to the reference sequence corresponding to residues 203-736 of SEQ ID NO: 552.

In some embodiments, the amino acid sequence of the engineered AAV VP3 capsid polypeptide comprises at least a substitution or substitution set at amino acid position(s) 725, 414/456/459/485/736, 454, 668, 485/595, 454/485, 346, 421/456/459, 218, 205, 348, 421, 551, 736, 529/552/585, 535, 529/585, 535/659, 594, 468, 726, 468/592, 451, 559, 575, 465, 560, 273, 389/510, 470, 724, 485/597, 510, 256, 334, 211, 264, 595, 391, 391/661, 391/454/471, 391/421, 401/414/485, 391/454/595, 529, 529/552/588/716, 493/585/667, 269, 552, 515, 565, 470/663, 414, 597, 564, 389, 720, 451/452, 496, 214, or 320, wherein the amino acid positions are relative to the reference sequence corresponding to residues 203-736 of SEQ ID NO: 552.

In some embodiments, the amino acid sequence of the engineered AAV VP3 capsid polypeptide capsid comprises at least a substitution or substitution set 725G, 414R/456N/459C/485P/736M, 454Q, 668R, 485S/595F, 454Q/485S, 346S, 421G/456N/459C, 218S, 205P, 348T, 421G, 551F, 736M, 529A/552S/585T, 535G, 529A/585T, 535G/659Q, 594W, 468V, 726H, 594V, 468W/592L, 451P, 594T, 559C, 575D, 465P, 4651, 560L, 468A, 451L, 594N, 273T, 389M/510C, 451Y, 470Q, 465T, 468Q, 594Q, 594S, 724V, 485Q/597I, 510T, 256I, 334V, 211M, 264S, 594M, 725A, 595F, 391Q, 391Q/661K, 391Q/454Q/4711, 391Q/421T, 401G/414R/485P, 391Q/454Q/595F, 529A, 529A/552S/588Q/716D, 493T/585T/667T, 269T, 552S, 515V, 724I, 465N, 451G, 451H, 565T, 510G, 451S, 470G/663D, 414R, 597C, 465L, 5971, 565G, 564D, 725W, 389P, 451T, 510E, 468Y, 515T, 564A, 510C, 515L, 468W, 7261, 720F, 451V/452D, 451N, 5101, 496D, 389F, 510Q, 725M, 214D, or 320V, wherein the amino acid positions are relative to the reference sequence corresponding to residues 203-736 of SEQ ID NO: 552.

In some embodiments, the amino acid sequence of the engineered AAV VP3 capsid polypeptide comprises at least a substitution or substitution set R725G, S414R/Q456N/Q459C/R485P/L736M, Q165V/S454Q, Q165V/N668R, R485S/W595F, S454Q/R485S, T346S, P421G/Q456N/Q459C, A218S, S205P, S348T, P421G, D551F, L736M, E529A/N552S/Q585T, F535G, E529A/Q585T, F535G/P659Q, G594W, P468V, P726H, G594V, P468W/Q592L, I451P, G594T, M559C, E575D, V465P, V465I, I560L, P468A, I451L, G594N, A273T, V389M/A510C, 1451Y, N470Q, V465T, P468Q, G594Q, G594S, P724V, R485Q/Q597I, A510T, L2561, 1334V, V211M, T264S, G594M, R725A, W595F, R391Q, R391Q/A661K, S200P, R391Q/S454Q/M471I, Q165V/R391Q, R391Q/P42IT, P401G/S414R/R485P, R391Q/S454Q/W595F, E529A, E529A/N552S/R588Q/N716D, V493T/Q585T/L667T, S269T, N552S, N515V, P724I, V465N, 1451G, 1451H, E565T, A510G, 1451S, N470G/N663D, S414R, Q597C, V465L, Q597I, E565G, E564D, R725W, V389P, I451T, A510E, P468Y, N515T, E564A, A510C, N515L, P468W, P726I, V720F, 1451V/N452D, 1451N, A510I, N496D, V389F, A510Q, R725M, N214D, or I320V, wherein the amino acid positions are relative to the reference sequence corresponding to residues 203-736 of SEQ ID NO: 552.

In some embodiments, the amino acid sequence of the engineered AAV VP3 capsid polypeptide comprises at least a substitution or substitution set at amino acid position(s) 333, 254, 725, 258, 268, 214/451, 331, 328, 274, 576, 560, 272, 263, 270, 267, 210/266, 259, 259/466, 264, 266, 535, 329, or 470, wherein the amino acid positions are relative to the reference sequence corresponding to residues 203-736 of SEQ ID NO: 552.

In some embodiments, the amino acid sequence of the engineered AAV VP3 capsid polypeptide comprises at least a substitution or substitution set 333S, 254C, 725H, 3331, 258T, 268I, 214D/451L, 331R, 328T, 274F, 576Y, 333N, 331T, 560H, 272G, 263A, 270L, 263T, 267L, 210S/266F, 259K, 267L, 263T, 259S/466T, 331T, 190H, 333A, 90G, 264E, 270H, 266V, 535G, 267V, 270P, 264N, 266M, 270Q, 266Q, 266L, 264G, 270M, 267A, 270A, 329G, 266S, 270V, 263W, 266H, 470L, 266T, or 331S, wherein the amino acid positions are relative to the reference sequence corresponding to residues 203-736 of SEQ ID NO: 552.

In some embodiments, the amino acid sequence of the engineered AAV VP3 capsid polypeptide comprises at least a substitution or substitution set T333S, N254C, R725H, T333I, K258T, S2681, N214D/1451L, 331R, E190L, N328T, Y274F, S576Y, T333N, V331T, I560H, N272G, S263A, N270L, S263T, G267L, P210S/G266F, Q259K, G267L, S263T, Q259S/A466T, V331T, T333A, T264E, N270H, G266V, F535G, G267V, N270P, T264N, G266M, N270Q, G266Q, G266L, T264G, N270M, G267A, N270A, E190V, N329G, G266S, N270V, S263W, G266H, N470L, G266T, or V331S, wherein the amino acid positions are relative to the reference sequence corresponding to residues 203-736 of SEQ ID NO: 552.

In some embodiments, the amino acid sequence of the engineered AAV VP3 capsid polypeptide comprises at least a substitution at an amino acid position between residues 203-736 of AAV capsid polypeptide, wherein the amino acid positions are set forth in Tables 5.1, 5.2, 6.1, 6.2, 7.1, and 7.2, and wherein the amino acid positions are relative to the reference sequence corresponding to residues 203-736 of SEQ ID NO: 2, 278, or 552.

In some embodiments, the amino acid sequence of the engineered AAV VP3 capsid polypeptide comprises at least one substitution set between residues 203-736 of AAV capsid polypeptide, wherein the substitutions are forth in Tables 5.1, 5.2, 6.1, 6.2, 7.1, and 7.2, and wherein the amino acid positions are relative to the reference sequence corresponding to residues 203-736 of SEQ ID NO: 2, 278, or 552.

In some embodiments, the amino acid sequence of the engineered AAV VP3 capsid polypeptide comprises at least a substitution or substitution set at amino acid position(s) between residues 203-736 of AAV capsid polypeptide, wherein the substitution or substitution set are set forth in Tables 5.1, 5.2, 6.1, 6.2, 7.1, and 7.2, wherein the amino acid positions are relative to the reference sequence corresponding to residues 203-736 of SEQ ID NO: 2, 278, or 552.

In some embodiments, the amino acid sequence of the engineered AAV VP3 capsid polypeptide comprises at least a substitution or substitution set between residues 203-736 of an engineered AAV capsid polypeptide set forth in Tables 5.1, 5.2, 6.1, 6.2, 7.1, and 7.2, wherein the amino acid positions are relative to the reference sequence corresponding to residues 203-736 of SEQ ID NO: 2, 278, or 552.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a sequence corresponding to residues 203-736 an engineered AAV capsid polypeptide set forth in Tables 5.1, 5.2, 6.1, 6.2, 7.1, and 7.2.

In some embodiments, the engineered AAV VP3 capsid polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 203-736 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 338, 340, 342, 344, 346, 350, 352, 354, 356, 358, 360, 362, 364, 366, 368, 370, 372, 374, 376, 378, 380, 382, 384, 386, 388, 390, 392, 396, 398, 400, 402, 404, 406, 408, 410, 412, 414, 416, 418, 420, 422, 424, 426, 428, 430, 432, 444, 446, 448, 450, 452, 454, 460, 462, 464, 468, 470, 472, 482, 484, 486, 488, 490, 496, 500, 502, 504, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 600, 602, 610, 612, 618, 622, 624, 626, 630, 632, 634, 638, 642, 644, 646, 650, 652, 656, 658, 662, 664, 666, 670, 672, 678, 682, 684, 686, 690, 698, 704, 708, 710, 712, 714, 716, 718, 720, 722, 724, 726, 730, 732, 734, 736, 738, 740, 742, 744, 746, 748, 750, 754, 756, 758, 760, 762, 764, 768, 770, 776, 782, 784, 786, 788, 796, 798, 800, 804, 808, 810, 812, 814, 816, 818, 820, 824, 826, 828, 830, 832, 834, 836, 840, 844, 848, 852, 854, 856, 858, 860, 862, 866, 868, 876, 878, 880, 884, 886, 888, 890, 892, 896, 898, 900, 902, 904, 906, 910, 916, 920, 922, 924, 926, 928, 930, 932, 934, 936, 938, 942, 946, 948, 950, 954, 956, 962, 964, 966, 968, 970, 974, 976, 980, 982, 986, 988, 994, 996, 1000, 1004, 1006, 1008, 1012, 1016, 1018, 1020, 1022, 1026, 1028, 1034, 1036, 1038, 1044, 1050, 1052, 1054, 1056, 1062, 1064, 1070, 1074, 1076, 1078, 1080, 1082, 1084, 1090, 1094, 1096, 1098, 1100, 1102, 1104, 1106, 1108, 1110, 1112, 1114, 1116, 1118, 1120, 1122, 1124, 1126, 1128, 1132, or 1134.

In some embodiments, the engineered AAV VP3 capsid polypeptide comprises an amino acid sequence comprising residues 203-736 of an even-numbered SEQ ID NO, of SEQ ID NOs: 4-1134, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions.

In some embodiments, the engineered AAV VP3 capsid polypeptide comprises an amino acid sequence comprising residues 203-736 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 338, 340, 342, 344, 346, 350, 352, 354, 356, 358, 360, 362, 364, 366, 368, 370, 372, 374, 376, 378, 380, 382, 384, 386, 388, 390, 392, 396, 398, 400, 402, 404, 406, 408, 410, 412, 414, 416, 418, 420, 422, 424, 426, 428, 430, 432, 444, 446, 448, 450, 452, 454, 460, 462, 464, 468, 470, 472, 482, 484, 486, 488, 490, 496, 500, 502, 504, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 600, 602, 610, 612, 618, 622, 624, 626, 630, 632, 634, 638, 642, 644, 646, 650, 652, 656, 658, 662, 664, 666, 670, 672, 678, 682, 684, 686, 690, 698, 704, 708, 710, 712, 714, 716, 718, 720, 722, 724, 726, 730, 732, 734, 736, 738, 740, 742, 744, 746, 748, 750, 754, 756, 758, 760, 762, 764, 768, 770, 776, 782, 784, 786, 788, 796, 798, 800, 804, 808, 810, 812, 814, 816, 818, 820, 824, 826, 828, 830, 832, 834, 836, 840, 844, 848, 852, 854, 856, 858, 860, 862, 866, 868, 876, 878, 880, 884, 886, 888, 890, 892, 896, 898, 900, 902, 904, 906, 910, 916, 920, 922, 924, 926, 928, 930, 932, 934, 936, 938, 942, 946, 948, 950, 954, 956, 962, 964, 966, 968, 970, 974, 976, 980, 982, 986, 988, 994, 996, 1000, 1004, 1006, 1008, 1012, 1016, 1018, 1020, 1022, 1026, 1028, 1034, 1036, 1038, 1044, 1050, 1052, 1054, 1056, 1062, 1064, 1070, 1074, 1076, 1078, 1080, 1082, 1084, 1090, 1094, 1096, 1098, 1100, 1102, 1104, 1106, 1108, 1110, 1112, 1114, 1116, 1118, 1120, 1122, 1124, 1126, 1128, 1132, or 1134, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions.

In some embodiments, the amino acid sequence of the engineered AAV capsid polypeptide has 1, 2, 3, 4, up to 5 substitutions. In some embodiments, the substitutions comprises conservative and/or non-conservative substitutions. In some embodiments, the substitutions comprises conservative substitutions.

### Engineered MAAP Polypeptides

As discussed above, the polynucleotide sequence encoding the AAV capsid polypeptide also encodes the MAAP polypeptide in a second open-reading frame (ORF) in the AAV VP1/2 cap gene sequence but different in sequence from the capsid proteins. The function of MAAP may affect competitive exclusion of different serotype AAV genomes and production and intracellular distribution of AAV capsid proteins. Accordingly, the present disclosure further provides engineered MAAP polypeptides.

In some embodiments, an engineered MAAP polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 1136, wherein the amino acid sequence comprises one or more substitutions relative to the reference sequence corresponding to SEQ ID NO: 1136.

In some embodiments, the amino acid sequence of the engineered MAAP polypeptide comprises at least a substitution at amino acid position 5, 7, 17, 27, 30, 34, 40, 64, 73, 74, 75, 93, 99, 100, or 116, or combinations thereof, wherein the amino acid positions are relative to SEQ ID NO: 1136.

In some embodiments, the amino acid sequence of the engineered MAAP polypeptide comprises at least a substitution 5Y, 7L/K, 17G, 27G/Q/R/V/*, 30L/V, 34R/V, 40E, 64F/G/I/P/R/V, 73R, 74F/G/L/P/R/Y, 75A/E/G/W, 93P, 99C, 100V, or 116T, or combinations thereof, wherein the amino acid positions are relative to SEQ ID NO: 1136.

In some embodiments, the amino acid sequence of the engineered MAAP polypeptide comprises at least a substitution or substitution set at amino acid position(s) 99, 30, 17, 93, 116, 73, 5, 75, 34, 74, 27, 7, 73/74, 64, 40, or 100, wherein the amino acid positions are relative to SEQ ID NO: 1136.

In some embodiments, the amino acid sequence of the engineered MAAP polypeptide comprises at least a substitution or substitution set 99C, 30L/V, 17G, 93P, 116T, 73R/74L, 5Y, 75E/W/A/G, 34V/R, 74L/P/G/R, 27G/V/Q/R/V/*, 7L/K, 73R/74R, 64F/V/G/P/R/I, 40E, 73R/74F, 73R/74Y, or 100V, wherein the amino acid positions are relative to SEQ ID NO: 1136.

In some embodiments, the amino acid sequence of the engineered MAAP polypeptide comprises at least a substitution or substitution set F99C, A30L/V, E17G, S93P, R116T, S73R/S74L, N5Y, R75E/W/A/G, T34V/R, S74L/P/G/R, L27G/V/Q/R/V/*, R7L/K, S73R/S74R, T64F/V/G/P/R/I, T40E, S73R/S74F, T64I, S73R/S74Y, T64R, or L100V, wherein the amino acid positions are relative to SEQ ID NO: 1136.

In some embodiments, the engineered MAAP polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to SEQ ID NO: 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, or 1232.

In some embodiments, the engineered MAAP polypeptide comprises an amino acid sequence comprising SEQ ID NO: 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, or 1232, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions.

In some embodiments, the amino acid sequence of the engineered MAAP capsid polypeptide has 1, 2, 3, 4, up to 5 substitutions. In some embodiments, the substitutions comprises conservative and/or non-conservative substitutions. In some embodiments, the substitutions comprises conservative substitutions.

### Engineered AAP Polypeptides

In a further aspect, the present disclosure also provides engineered AAP polypeptides encoded by an alternative reading frame, ORF2 resulting from a frame-shift in the VP2/3 reading frame of the AAV cap gene.

In some embodiments, an engineered AAP polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 1234, wherein the amino acid sequence comprises one or more substitutions relative to the reference sequence corresponding to SEQ ID NO: 1234.

In some embodiments, the amino acid sequence of the engineered AAP polypeptide comprises at least a substitution at amino acid position 14, 15, 31, 49, 58, 79, 83, 84, 85, 88, 89, 91, 92, 93, 94, 95, 97, 99, 100, 102, 104, 105, 120, 153, 154, 155, 156, 157, 158, 160, 173, 174, or 194, or combinations thereof, wherein the amino acid positions are relative to SEQ ID NO: 1234.

In some embodiments, the amino acid sequence of the engineered AAP comprises at least a substitution 14D, 15A/I/R/S/T/V/Y, 31A, 49H, 58R, 79V, 83L/R, 84R/V, 85Y, 88G/L/R, 89S/T/V, 91D/K/L/P/R/S/T/W, 92C/E/H/S/V, 93S, 94H, 95R/S/W, 97G/M, 99S, 100L, 102I, 104H, 105L, 120R, 153I/R, 154G, 155D, 156L/P/Q/R/V/*, 157S, 158I/L/R/S, 160R, 173R, 174I/S, or 194P, or combinations thereof, wherein the amino acid positions are relative to SEQ ID NO: 1234.

In some embodiments, the amino acid sequence of the engineered AAP polypeptide comprises at least a substitution or substitution set at amino acid position(s) 58, 156, 49/156, 174, 157/158, 160, 158, 15, 79, 104, 31/156, 83, 92/93, 102, 155/156, 14/15, 153, 194, 99, 85, 105, 97, 88, 94/95, 91/92, 100, 91, 92, 83/84, 89, 94, 94/95/173, 97/120, 95, or 154, wherein the amino acid positions are relative to SEQ ID NO: 1234.

In some embodiments, the amino acid sequence of the engineered AAP polypeptide comprises at least a substitution or substitution set 58R, 156R, 49H/156*, 156*, 174S, 174I, 157S/158L, 160R, 158L, 15R, 79V, 104H, 31A/156L, 15I, 158S, 83L, 92E/93S, 1021, 155D/156V, 14D/15Y, 153R, 194P, 99S, 1581, 85Y, 156Q, 105L, 14D/15A, 15S, 97G, 88L, 94H/95W, 91D/92C, 100L, 91S, 92V, 83R/84R, 91D/92S, 88R, 83R/84V, 156P, 14D/15T, 158R, 89S, 94H, 91L, 92S, 94H/95R/173R, 89T, 91W, 15V, 94H/95R, 91K, 89V, 97M/120R, 95W, 92H, 95R, 1531, 154G, 91P, 95S, 88G, 91T, 91R, or 155D/156R, wherein the amino acid positions are relative to SEQ ID NO: 1234.

In some embodiments, the amino acid sequence of the AAP engineered polypeptide comprises at least a substitution or substitution set N58R, S156R, P49H/S156*, S156*, T174S, T174I, R157S/P158L, P160R, P158L, N15R, 179V, P104H, V31A/S156L, N15I, P158S, S83L, D92E/L93S, T102I, E155D/S156V, E14D/N15Y, T153R, R194P, T99S, P158I, S85Y, S156Q, P105L, E14D/N15A, N15S, T97G, A88L, Q94H/M95W, E91D/D92C, S100L, E91S, D92V, S83R/K84R, E91D/D92S, A88R, S83R/K84V, S156P, E14D/N15T, P158R, H89S, Q94H, E91L, D92S, Q94H/M95R/Q173R, H89T, E91W, N15V, Q94H/M95R, E91K, H89V, T97M/H120R, M95W, D92H, M95R, T153I, M154G, E91P, M95S, A88G, E91T, E91R, or E155D/S156R, wherein the amino acid positions are relative to SEQ ID NO: 1234.

In some embodiments, the engineered AAP polypeptide comprises an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to SEQ ID NO: 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, or 1410.

In some embodiments, the engineered AAP polypeptide comprises an amino acid sequence comprising SEQ ID NO: 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, or 1410, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions.

In some embodiments, the amino acid sequence of the engineered AAP capsid polypeptide has 1, 2, 3, 4, up to 5 substitutions. In some embodiments, the substitutions comprises conservative and/or non-conservative substitutions. In some embodiments, the substitutions comprises conservative substitutions.

### Polynucleotides Encoding Engineered Polypeptides, Expression Vectors and Host Cells

In another aspect, the present disclosure provides recombinant polynucleotides encoding the engineered AAV capsid polypeptides, e.g., polynucleotides encoding AAV VP1 capsid polypeptides, AAV VP2 capsid polypeptides, and/or AAV VP3 capsid polypeptides. In some embodiments, the present disclosure further provides recombinant polynucleotides encoding engineered MAAP polypeptides or engineered AAP polypeptides.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the engineered polypeptides disclosed herein, Thus, having knowledge of a particular amino acid sequence, those skilled in the art could make any number of different nucleic acids by simply modifying the sequence of one or more codons in a way which does not change the amino acid sequence of the protein. In this regard, the present disclosure specifically contemplates each and every possible variation of polynucleotides that could be made encoding the polypeptides described herein, including the engineered AAV capsid polypeptides (e.g., engineered AAV VP1 capsid polypeptides, engineered AAV VP2 capsid polypeptides, and engineered AAV VP3 capsid polypeptides), the engineered MAAP polypeptides, and the engineered AAP polypeptides, by selecting combinations based on the possible codon choices, and all such variations are to be considered specifically disclosed for any polypeptide described herein, including the polypeptide variants provided in Tables 5.1, 5.2, 6.1, 6.2, 7.1, and 7.2.

In some embodiments, the recombinant polynucleotide comprises a polynucleotide sequence encoding the subject polypeptides, wherein the polynucleotide sequence comprise codons preferably selected to fit the host cell in which the protein is being produced. In some embodiments, preferred codons used in bacteria are used for expression in bacteria, preferred codons used in fungi are typically used for expression in fungi, preferred codons used in insect cells are used for expression in insect cells, and preferred codons used in mammals are used for expression in mammals and mammalian cells. In some embodiments, codon optimized polynucleotides encoding the engineered polypeptides contain preferred codons at about 40%, 50%, 60%, 70%, 80%, or greater than 90% of codon positions of the full length coding region. In some embodiments, the present disclosure provides recombinant polynucleotide sequences in which the codons are optimized for expression in mammalian cells or tissues.

### Polynucleotides Encoding Engineered AAV Capsid Polypeptides

In some embodiments, the recombinant polynucleotide comprises a polynucleotide sequence encoding the engineered AAV capsid polypeptide of the present disclosure. Accordingly, the present disclosure provides recombinant polynucleotides encoding each any every engineered AAV capsid polypeptide, including AAV VP1 capsid polypeptide, AAV VP2 capsid polypeptide, and AAV VP3 capsid polypeptide described herein. Accordingly, by way of example and not limitation, the recombinant polynucleotide comprises a polynucleotide sequence encoding an engineered AAV VP1 capsid polypeptide described herein.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO, of SEQ ID NOs: 2-1134, wherein the amino acid sequence comprises one or more substitutions relative to the reference sequence corresponding to SEQ ID NO: 2, 278, or 522.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 2, 278, or 522, wherein the amino acid sequence comprises one or more substitutions relative to the reference sequence corresponding to SEQ ID NO: 2, 278, or 522.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 2, wherein the amino acid sequence comprises one or more substitutions relative to the reference sequence corresponding to SEQ ID NO: 2.

In some embodiments, a recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522, wherein the amino acid sequence comprises one or more substitutions relative to the reference sequence corresponding to SEQ ID NO: 2.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO, of SEQ ID NOs: 4-1134, wherein the amino acid sequence comprises one or more substitutions relative to the reference sequence corresponding to SEQ ID NO: 2.

In some embodiments, the recombinant polynucleotide comprises a polynucleotide sequence encoding the engineered AAV capsid polypeptide comprising an amino acid sequence comprising at least a substitution at amino acid position 10, 14, 17, 22, 23, 24, 25, 31, 33, 45, 51, 53, 56, 60, 63, 66, 79, 81, 90, 100, 101, 119, 125, 126, 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to SEQ ID NO: 2.

In some embodiments, the recombinant polynucleotide comprises a polynucleotide sequence encoding the engineered AAV capsid polypeptide comprising an amino acid sequence comprising substitutions in at least two amino acid positions 588, 665, 457, 495, 456, 505, 494, 497, 592, 586, 585, 706, 549, 552, 664, 661, 461, 668, 548, 510, 640, 419, 349, 504, 508, 453, 589, 56, and 416, wherein the amino acid positions are relative to the reference sequence corresponding to SEQ ID NO: 2.

In some embodiments, the recombinant polynucleotide comprises a polynucleotide sequence encoding the engineered AAV capsid polypeptide comprising an amino acid sequence comprising at least a first and second substitutions at amino acid positions selected from (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, 56, and 416, wherein the first substitution is at a position selected from (a), and the second substitution is at a position selected from (a), (b) and (c), wherein the amino acid positions are relative to the reference sequence corresponding to SEQ ID NO: 2.

In some embodiments, the recombinant polynucleotide comprises a polynucleotide sequence encoding the engineered AAV capsid polypeptide comprising an amino acid sequence comprising at least a substitution set at amino acid positions 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to SEQ ID NO: 2.

In some embodiments, the recombinant polynucleotide comprises a polynucleotide sequence encoding the engineered AAV capsid polypeptide comprising an amino acid sequence comprising at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 56/529, 331/449, 211/509/712, 331/416/478, 56/507, 56/331/584/597, 56/478, 349/465/479/504/508, 349/640, 331/584/597, 331/416/478/483/507/584/597, 56/416, 233/465/640, 125/508/510/589, 597, 331/597, 56/331/416/665, 125/465/504/508/510, 56/331/478/483/529/597, 384/479, 233/640, 125/419/504/508/530, 504/510/640, 419/504/508, 56/331/361, 416/665, 56/211/361/416/478/584, 56, 56/331/416, 175/211, 56/211/478/483/712, 349/504/508, 125/508/510, 331/416, 331, 479/504/510/589, 331/478, 56/331/478/483/597, 479, 125/504/508, 479/589, or 479/510/589, wherein the amino acid positions are relative to the reference sequence corresponding to SEQ ID NO: 2.

In some embodiments, the recombinant polynucleotide comprises a polynucleotide sequence encoding the engineered AAV capsid polypeptide comprising at least a substitution at an amino acid position set forth in Tables 5.1, 5.2, 6.1, 6.2, 7.1, and 7.2, wherein the amino acid positions are relative to the reference sequence corresponding to SEQ ID NO: 2.

In some embodiments, the recombinant polynucleotide comprises a polynucleotide sequence encoding the engineered AAV capsid polypeptide comprising at least one substitution set forth in Tables 5.1, 5.2, 6.1, 6.2, 7.1, and 7.2, wherein the amino acid positions are relative to the reference sequence corresponding to SEQ ID NO: 2.

In some embodiments, the recombinant polynucleotide comprises a polynucleotide sequence encoding the engineered AAV capsid polypeptide comprising at least a substitution or substitution set at amino acid position(s) set forth in Tables 5.1, 5.2, 6.1, 6.2, 7.1, and 7.2, wherein the amino acid positions are relative to the reference sequence corresponding to SEQ ID NO: 2.

In some embodiments, the recombinant polynucleotide comprises a polynucleotide sequence encoding the engineered AAV capsid polypeptide comprising at least a substitution or substitution set of an engineered AAV capsid polypeptide set forth in Tables 5.1, 5.2, 6.1, 6.2, 7.1, and 7.2, wherein the amino acid positions are relative to the reference sequence corresponding to SEQ ID NO: 2.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to an even-numbered SEQ ID NO, of SEQ ID NOs: 4-1134.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to an even-numbered SEQ ID NO, of SEQ ID NOs: 4-1134, wherein the amino acid sequence comprises one or more substitutions relative to the reference sequence corresponding to SEQ ID NO: 278 or 522.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522, wherein the amino acid sequence comprises one or more substitutions relative to the reference sequence corresponding to SEQ ID NO: 278 or 522.

In some embodiments, the recombinant polynucleotide comprises a polynucleotide sequence encoding the engineered AAV capsid polypeptide comprising an amino acid sequence comprising at least a substitution at amino acid position 10, 14, 17, 22, 23, 24, 25, 31, 33, 45, 51, 53, 56, 60, 63, 66, 79, 81, 90, 100, 101, 119, 125, 126, 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to SEQ ID NO: 278 or 522.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278, wherein the amino acid sequence comprises one or more substitutions relative to the reference sequence corresponding to SEQ ID NO: 278.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO, of SEQ ID NOs: 332-722, wherein the amino acid sequence comprises one or more substitutions relative to the reference sequence corresponding to SEQ ID NO: 278.

In some embodiments, the recombinant polynucleotide comprises a polynucleotide sequence encoding the engineered AAV capsid polypeptide comprising an amino acid sequence comprising at least a substitution or substitution set at amino acid position(s) 14, 173, 159, 157, 152, 485, 421, 391, 469, 628, 461, 165, 642, 595, 24, 414, 512, 519, 172, 168, 598, 449, 736, 156, 462, 162, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 155, 663, 401, 22, 175, 579, 444, 23, 459/508/663/667, 17, 412, 440, 387, 453/586, 419, 25, 472, 162/450, 585, 437, 10, 562, 169, 588, 565, 589, 495/497, 119/450, or 142/552, wherein the amino acid positions are relative to the reference sequence corresponding to SEQ ID NO: 278.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 552, wherein the amino acid sequence comprises one or more substitutions relative to the reference sequence corresponding to SEQ ID NO: 552.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to an even-numbered SEQ ID NO, of SEQ ID NOs: 724-1134, wherein the amino acid sequence comprises one or more substitutions relative to the reference sequence corresponding to SEQ ID NO: 552.

In some embodiments, the recombinant polynucleotide comprises a polynucleotide sequence encoding the engineered AAV capsid polypeptide comprising an amino acid sequence comprising at least a substitution or substitution set at amino acid position(s) 725, 414/456/459/485/736, 193, 157/165/454, 157/165/668, 485/595, 165/454/485, 346, 421/456/459, 218, 205, 348, 421, 551, 165, 736, 529/552/585, 535, 529/585, 535/659, 594, 468, 101, 726, 468/592, 451, 559, 575, 465, 560, 273, 389/510, 470, 100, 724, 485/597, 510, 45, 256, 334, 63, 211, 264, 595, 391, 391/661, 200, 165/391/454/471, 157/165/391, 165/391, 157/391/421, 81, 401/414/485, 391/454/595, 529, 529/552/588/716, 493/585/667, 269, 552, 515, 565, 470/663, 414, 597, 564, 389, 60, 720, 451/452, 496, 214, or 320, wherein the amino acid positions are relative to the reference sequence corresponding to SEQ ID NO: 552.

In some embodiments, the recombinant polynucleotide comprises a polynucleotide sequence encoding the engineered AAV capsid polypeptide comprising an amino acid sequence comprising at least a substitution or substitution set at amino acid position(s) 333, 190, 254, 100, 190/725, 31, 101, 258, 268, 214/451, 331, 328, 33, 274, 33/576, 560, 51/190, 60, 79/272, 263, 270, 267, 210/266, 259, 53, 259/466, 90, 264, 172/266, 90/535, 266, 66, 329, or 126/470, wherein the amino acid positions are relative to the reference sequence corresponding to SEQ ID NO: 552.

In some embodiments, the recombinant polynucleotide comprises a polynucleotide sequence encoding the engineered AAV capsid polypeptide comprising an amino acid sequence comprising at least a substitution at an amino acid position set forth in Tables 5.1, 5.2, 6.1, 6.2, 7.1, and 7.2, wherein the amino acid positions are relative to the reference sequence corresponding to SEQ ID NO: 2, 278, or 552.

In some embodiments, the recombinant polynucleotide comprises a polynucleotide sequence encoding the engineered AAV capsid polypeptide comprising an amino acid sequence comprising at least one substitution set forth in Tables 5.1, 5.2, 6.1, 6.2, 7.1, and 7.2, wherein the amino acid positions are relative to the reference sequence corresponding to SEQ ID NO: 2, 278, or 552.

In some embodiments, the recombinant polynucleotide comprises a polynucleotide sequence encoding the engineered AAV capsid polypeptide comprising an amino acid sequence comprising at least a substitution or substitution set at amino acid position(s) set forth in Tables 5.1, 5.2, 6.1, 6.2, 7.1, and 7.2, wherein the amino acid positions are relative to the reference sequence corresponding to SEQ ID NO: 2, 278, or 552.

In some embodiments, the recombinant polynucleotide comprises a polynucleotide sequence encoding the engineered AAV capsid polypeptide comprising an amino acid sequence comprising at least a substitution or substitution set of an engineered AAV capsid polypeptide set forth in Tables 5.1, 5.2, 6.1, 6.2, 7.1, and 7.2, wherein the amino acid positions are relative to the reference sequence corresponding to SEQ ID NO: 2, 278, or 552.

In some embodiments, the recombinant polynucleotide comprises a polynucleotide sequence encoding the engineered AAV capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a sequence corresponding to an engineered AAV capsid polypeptide set forth in Tables 5.1, 5.2, 6.1, 6.2, 7.1, and 7.2.

In some embodiments, the recombinant polynucleotide comprises a polynucleotide sequence encoding the engineered AAV capsid polypeptide comprising an amino acid sequence comprising an even-numbered SEQ ID NO, of SEQ ID NOs: 4-1134, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions.

In some embodiments, the recombinant polynucleotide comprises a polynucleotide sequence having at least 60%, 65%, 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to an odd-numbered SEQ ID NO, of SEQ ID NOs: 3-1133, wherein the recombinant polynucleotide encodes an AAV capsid polypeptide.

In some embodiments, the recombinant polynucleotide comprises a polynucleotide sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to SEQ ID NO, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 222, 224, 226, 228, 230, 232, 234, 236, 238, 240, 242, 244, 246, 248, 250, 252, 254, 256, 258, 260, 262, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 756, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 894, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, or 1133.

In some embodiments, the recombinant polynucleotide comprises a polynucleotide sequence comprising SEQ ID NO, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 222, 224, 226, 228, 230, 232, 234, 236, 238, 240, 242, 244, 246, 248, 250, 252, 254, 256, 258, 260, 262, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 756, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 894, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, or 1133.

In some embodiments, the recombinant polynucleotide comprises a polynucleotide sequence encoding an engineered AAV VP2 capsid polypeptide of an AAV capsid polypeptide described herein.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 138-736 of an even-numbered SEQ ID NO, of SEQ ID NOs: 2-1134, wherein the amino acid sequence comprises one or more substitutions relative to the reference sequence corresponding to residues 138-736 of SEQ ID NO: 2, 278, or 522.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 2, 278, or 522, wherein the amino acid sequence comprises one or more substitutions relative to the reference sequence corresponding to residues 138-736 of SEQ ID NO: 2, 278, or 522.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 2, wherein the amino acid sequence comprises one or more substitutions relative to the reference sequence corresponding to residues 138-736 of SEQ ID NO: 2.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522, wherein the amino acid sequence comprises one or more substitutions relative to the reference sequence corresponding to residues 138-736 of SEQ ID NO: 2.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO, of SEQ ID NOs: 4-1134, wherein the amino acid sequence comprises one or more substitutions relative to the reference sequence corresponding to residues 138-736 of SEQ ID NO: 2.

In some embodiments, the recombinant polynucleotide comprises a polynucleotide sequence encoding the engineered AAV VP2 capsid polypeptide comprising an amino acid sequence comprising at least a substitution at amino acid position 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 138-736 of SEQ ID NO: 2.

In some embodiments, the recombinant polynucleotide comprises a polynucleotide sequence encoding the engineered AAV VP2 polypeptide comprising an amino acid sequence comprising substitutions in at least two amino acid positions 588, 665, 457, 495, 456, 505, 494, 497, 592, 586, 585, 706, 549, 552, 664, 661, 461, 668, 548, 510, 640, 419, 349, 504, 508, 453, 589, and 416, wherein the amino acid positions are relative to the reference sequence corresponding to residues 138-736 of SEQ ID NO: 2.

In some embodiments, the recombinant polynucleotide comprises a polynucleotide sequence encoding the engineered AAV VP2 capsid polypeptide comprising an amino acid sequence comprising at least a first and second substitutions at amino acid positions selected from (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, and 416, wherein the first substitution is at a position selected from (a), and the second substitution is at a position selected from (a), (b) and (c), wherein the amino acid positions are relative to the reference sequence corresponding to residues 138-736 of SEQ ID NO: 2.

In some embodiments, the recombinant polynucleotide comprises a polynucleotide sequence encoding the engineered AAV VP2 polypeptide comprising an amino acid sequence comprising at least a substitution set at amino acid positions 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to the reference sequence corresponding to residues 138-736 of SEQ ID NO: 2.

In some embodiments, the recombinant polynucleotide comprises a polynucleotide sequence encoding the engineered AAV VP2 polypeptide comprising an amino acid sequence comprising at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 331/449, 211/509/712, 331/416/478, 507, 331/584/597, 478, 349/465/479/504/508, 349/640, 331/416/478/483/507/584/597, 416, 233/465/640, 508/510/589, 597, 331/597, 331/416/665, 465/504/508/510, 331/478/483/529/597, 384/479, 233/640, 419/504/508/530, 504/510/640, 419/504/508, 331/361, 416/665, 211/361/416/478/584, 331/416, 175/211, 211/478/483/712, 349/504/508, 508/510, 331/416, 331, 479/504/510/589, 331/478, 331/478/483/597, 479, 504/508, 479/589, or 479/510/589, wherein the amino acid positions are relative to the reference sequence corresponding to residues 138-736 of SEQ ID NO: 2.

In some embodiments, the recombinant polynucleotide comprises a polynucleotide sequence encoding the engineered AAV VP2 capsid polypeptide comprising an amino acid sequence comprising at least a substitution at an amino acid position between amino acid residues 138-736 of AAV capsid polypeptide, wherein the amino acid positions are set forth in Tables 5.1, 5.2, 6.1, 6.2, 7.1, and 7.2, and wherein the amino acid positions are relative to the reference sequence corresponding to residues 138-736 of SEQ ID NO: 2.

In some embodiments, the recombinant polynucleotide comprises a polynucleotide sequence encoding the engineered AAV VP2 capsid polypeptide comprising an amino acid sequence comprising at least one substitution between amino acid residues 138-736, wherein the substitutions are set forth in Tables 5.1, 5.2, 6.1, 6.2, 7.1, and 7.2, and wherein the amino acid positions are relative to the reference sequence corresponding to residues 138-736 of SEQ ID NO: 2.

In some embodiments, the recombinant polynucleotide comprises a polynucleotide sequence encoding the engineered AAV VP2 capsid polypeptide comprising an amino acid sequence comprising at least a substitution or substitution set at amino acid position(s) between amino acid residues 138-736 of AAV capsid polypeptide, wherein the substitution or substitution set are set forth in Tables 5.1, 5.2, 6.1, 6.2, 7.1, and 7.2, and wherein the amino acid positions are relative to the reference sequence corresponding to residues 138-736 of SEQ ID NO: 2.

In some embodiments, the recombinant polynucleotide comprises a polynucleotide sequence encoding the engineered AAV VP2 capsid polypeptide comprising an amino acid sequence comprising at least a substitution or substitution set between amino acid residues 138-736 of an engineered AAV capsid polypeptide set forth in Tables 5.1, 5.2, 6.1, 6.2, 7.1, and 7.2, and wherein the amino acid positions are relative to the reference sequence corresponding to residues 138-736 of SEQ ID NO: 2.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO, of SEQ ID NOs: 4-1134.

the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO, of SEQ ID NOs: 4-1134, wherein the amino acid sequence comprises one or more substitutions relative to the reference sequence corresponding to residues 138-736 of SEQ ID NO: 278 or 522.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522, wherein the amino acid sequence comprises one or more substitutions relative to the reference sequence corresponding to residues 138-736 of SEQ ID NO: 278 or 522.

In some embodiments, the recombinant polynucleotide comprises a polynucleotide sequence encoding the engineered AAV VP2 capsid polypeptide comprising an amino acid sequence comprising at least a substitution at amino acid position 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 138-736 of SEQ ID NO: 278 or 522.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278, wherein the amino acid sequence comprises one or more substitutions relative to the reference sequence corresponding to residues 138-736 of SEQ ID NO: 278.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO, of SEQ ID NOs: 332-722, wherein the amino acid sequence comprises one or more substitutions relative to the reference sequence corresponding to residues 138-736 of SEQ ID NO: 278.

In some embodiments, the recombinant polynucleotide comprises a polynucleotide sequence encoding the engineered AAV VP2 capsid polypeptide comprising an amino acid sequence comprising at least a substitution or substitution set at amino acid position(s) 173, 159, 157, 152, 485, 421, 391, 469, 628, 461, 165, 642, 595, 414, 512, 519, 172, 168, 598, 449, 736, 156, 462, 162, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 155, 663, 401, 175, 579, 444, 459/508/663/667, 412, 440, 387, 453/586, 419, 472, 162/450, 585, 437, 562, 169, 588, 565, 589, 495/497, 119/450, or 142/552, wherein the amino acid positions are relative to the reference sequence corresponding to residues 138-736 of SEQ ID NO: 278.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 552, wherein the amino acid sequence comprises one or more substitutions relative to the reference sequence corresponding to residues 138-736 of SEQ ID NO: 552.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO, of SEQ ID NOs: 724-1134, wherein the amino acid sequence comprises one or more substitutions relative to the reference sequence corresponding to residues 138-736 of SEQ ID NO: 552.

In some embodiments, the recombinant polynucleotide comprises a polynucleotide sequence encoding the engineered AAV VP2 capsid polypeptide comprising an amino acid sequence comprising at least a substitution or substitution set at amino acid position(s) 725, 414/456/459/485/736, 193, 157/165/454, 157/165/668, 485/595, 165/454/485, 346, 421/456/459, 218, 205, 348, 421, 551, 165, 736, 529/552/585, 535, 529/585, 535/659, 594, 468, 726, 468/592, 451, 559, 575, 465, 560, 273, 389/510, 470, 724, 485/597, 510, 256, 334, 63, 211, 264, 595, 391, 391/661, 200, 165/391/454/471, 157/165/391, 165/391, 157/391/421, 401/414/485, 391/454/595, 529, 529/552/588/716, 493/585/667, 269, 552, 515, 565, 470/663, 414, 597, 564, 389, 720, 451/452, 496, 214, or 320, wherein the amino acid positions are relative to the reference sequence corresponding to residues 138-736 of SEQ ID NO: 552.

In some embodiments, the recombinant polynucleotide comprises a polynucleotide sequence encoding the AAV VP2 capsid polypeptide comprising an amino acid sequence comprising at least a substitution or substitution set at amino acid position(s) 333, 190, 254, 190/725, 258, 268, 214/451, 331, 328, 274, 576, 560, 79/272, 263, 270, 267, 210/266, 259, 259/466, 264, 172/266, 535, 266, 329, or 470, wherein the amino acid positions are relative to the reference sequence corresponding to residues 138-736 of SEQ ID NO: 552.

In some embodiments, the recombinant polynucleotide comprises a polynucleotide sequence encoding the engineered AAV VP2 capsid polypeptide comprising an amino acid sequence comprising at least a substitution at an amino acid position between residues 138-736 of AAV capsid polypeptide, wherein the amino acid positions are set forth in Tables 5.1, 5.2, 6.1, 6.2, 7.1, and 7.2, and wherein the amino acid positions are relative to the reference sequence corresponding to residues 138-736 of SEQ ID NO: 2, 278, or 552.

In some embodiments, the recombinant polynucleotide comprises a polynucleotide sequence encoding the engineered AAV VP2 capsid polypeptide comprising an amino acid sequence comprising at least one substitution between residues 138-736 of AAV capsid polypeptide, wherein the substitutions are set forth in Tables 5.1, 5.2, 6.1, 6.2, 7.1, and 7.2, and wherein the amino acid positions are relative to the reference sequence corresponding to residues 138-736 of SEQ ID NO: 2, 278, or 552.

In some embodiments, the recombinant polynucleotide comprises a polynucleotide sequence encoding the engineered AAV VP2 capsid polypeptide comprising an amino acid sequence comprising at least a substitution or substitution set at amino acid position(s) between residues 138-736 of AAV capsid polypeptide, wherein the substitution or substitution set are set forth in Tables 5.1, 5.2, 6.1, 6.2, 7.1, and 7.2, and wherein the amino acid positions are relative to the reference sequence corresponding to residues 138-736 of SEQ ID NO: 2, 278, or 552.

In some embodiments, the recombinant polynucleotide comprises a polynucleotide sequence encoding the engineered AAV VP2 capsid polypeptide comprising an amino acid sequence comprising at least a substitution or substitution set between residues 138-736 of an engineered AAV capsid polypeptide set forth in Tables 5.1, 5.2, 6.1, 6.2, 7.1, and 7.2, wherein the amino acid positions are relative to the reference sequence corresponding to residues 138-736 of SEQ ID NO: 2, 278, or 552.

In some embodiments, the recombinant polynucleotide comprises a polynucleotide sequence encoding the engineered AAV VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 138-736 of an engineered AAV capsid polypeptide set forth in Tables 5.1, 5.2, 6.1, 6.2, 7.1, and 7.2.

In some embodiments, the recombinant polynucleotide comprises a polynucleotide sequence encoding the engineered AAV VP2 capsid polypeptide comprising an amino acid sequence comprising residues 138-736 of an even-numbered SEQ ID NO, of SEQ ID NOs: 4-1134, optionally wherein the amino acid sequence has 1, 2, 3, 4, 5, 6, 7, 8, 9, or up to 10 substitutions.

In some embodiments, the recombinant polynucleotide comprises a polynucleotide sequence having at least 60%, 65%, 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 412-2208 of an odd-numbered SEQ ID NO, of SEQ ID NOs: 3-1133, wherein the recombinant polynucleotide encodes an AAV VP2 capsid polypeptide.

In some embodiments, the recombinant polynucleotide comprises a polynucleotide sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 412-2208 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 337, 339, 341, 343, 345, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 435, 437, 439, 441, 443, 445, 447, 449, 451, 453, 455, 459, 461, 463, 467, 469, 471, 473, 477, 479, 481, 483, 485, 487, 489, 491, 495, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 607, 609, 611, 613, 617, 621, 623, 625, 629, 631, 633, 637, 639, 641, 643, 645, 647, 649, 651, 653, 655, 657, 661, 663, 665, 669, 671, 673, 675, 677, 679, 681, 683, 685, 687, 689, 695, 697, 699, 701, 703, 707, 709, 711, 713, 715, 717, 719, 721, 723, 725, 727, 729, 731, 733, 735, 737, 739, 741, 743, 745, 747, 749, 751, 753, 755, 757, 759, 761, 763, 767, 769, 775, 777, 781, 783, 785, 787, 795, 797, 799, 801, 807, 809, 811, 813, 815, 817, 819, 823, 825, 827, 829, 831, 833, 835, 837, 839, 843, 847, 851, 853, 855, 857, 859, 861, 865, 867, 871, 875, 877, 879, 883, 885, 887, 889, 891, 895, 897, 899, 901, 903, 905, 907, 909, 911, 913, 915, 919, 921, 923, 925, 927, 929, 931, 933, 935, 937, 941, 945, 947, 949, 953, 955, 961, 963, 965, 967, 969, 973, 975, 979, 981, 985, 987, 991, 993, 995, 999, 1003, 1005, 1007, 1011, 1015, 1017, 1019, 1021, 1025, 1027, 1031, 1033, 1035, 1037, 1043, 1049, 1051, 1053, 1055, 1061, 1063, 1069, 1073, 1075, 1077, 1079, 1081, 1083, 1085, 1089, 1093, 1095, 1097, 1099, 1101, 1103, 1105, 1107, 1109, 1111, 1113, 1115, 1117, 1119, 1121, 1123, 1125, 1127, 1131, or 1133, wherein the recombinant polynucleotide encodes an engineered AAV VP2 capsid polypeptide.

In some embodiments, the recombinant polynucleotide comprises a polynucleotide sequence comprising nucleotide residues 412-2208 of an odd-numbered SEQ ID NO, of SEQ ID NOs: 39-1133.

In some embodiments, the recombinant polynucleotide comprises a polynucleotide sequence comprising nucleotide residues 412-2208 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 337, 339, 341, 343, 345, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 435, 437, 439, 441, 443, 445, 447, 449, 451, 453, 455, 459, 461, 463, 467, 469, 471, 473, 477, 479, 481, 483, 485, 487, 489, 491, 495, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 607, 609, 611, 613, 617, 621, 623, 625, 629, 631, 633, 637, 639, 641, 643, 645, 647, 649, 651, 653, 655, 657, 661, 663, 665, 669, 671, 673, 675, 677, 679, 681, 683, 685, 687, 689, 695, 697, 699, 701, 703, 707, 709, 711, 713, 715, 717, 719, 721, 723, 725, 727, 729, 731, 733, 735, 737, 739, 741, 743, 745, 747, 749, 751, 753, 755, 757, 759, 761, 763, 767, 769, 775, 777, 781, 783, 785, 787, 795, 797, 799, 801, 807, 809, 811, 813, 815, 817, 819, 823, 825, 827, 829, 831, 833, 835, 837, 839, 843, 847, 851, 853, 855, 857, 859, 861, 865, 867, 871, 875, 877, 879, 883, 885, 887, 889, 891, 895, 897, 899, 901, 903, 905, 907, 909, 911, 913, 915, 919, 921, 923, 925, 927, 929, 931, 933, 935, 937, 941, 945, 947, 949, 953, 955, 961, 963, 965, 967, 969, 973, 975, 979, 981, 985, 987, 991, 993, 995, 999, 1003, 1005, 1007, 1011, 1015, 1017, 1019, 1021, 1025, 1027, 1031, 1033, 1035, 1037, 1043, 1049, 1051, 1053, 1055, 1061, 1063, 1069, 1073, 1075, 1077, 1079, 1081, 1083, 1085, 1089, 1093, 1095, 1097, 1099, 1101, 1103, 1105, 1107, 1109, 1111, 1113, 1115, 1117, 1119, 1121, 1123, 1125, 1127, 1131, or 1133.

In some embodiments, the recombinant polynucleotide comprises a polynucleotide sequence encoding an engineered AAV VP3 capsid polypeptide of an AAV capsid polypeptide described herein.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO, of SEQ ID NOs: 2-1134, wherein the amino acid sequence comprises one or more substitutions relative to the reference sequence corresponding to residues 203-736 of SEQ ID NO: 2, 278, or 522.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 2, 278, or 522, wherein the amino acid sequence comprises one or more substitutions relative to the reference sequence corresponding to residues 203-736 of SEQ ID NO: 2, 278, or 522.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 2, wherein the amino acid sequence comprises one or more substitutions relative to the reference sequence corresponding to residues 203-736 of SEQ ID NO: 2.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278 or 522, wherein the amino acid sequence comprises one or more substitutions relative to the reference sequence corresponding to residues 203-736 of SEQ ID NO: 2.

In some embodiments, the recombinant polynucleotide comprises a polynucleotide sequence encoding the engineered AAV VP3 capsid polypeptides comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO, of SEQ ID NOs: 4-1134, wherein the amino acid sequence comprises one or more substitutions relative to the reference sequence corresponding to residues 203-736 of SEQ ID NO: 2.

In some embodiments, the recombinant polynucleotide comprises a polynucleotide sequence encoding the engineered AAV VP3 capsid polypeptide comprising an amino acid sequence comprising at least a substitution at amino acid position 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 203-736 of SEQ ID NO: 2.

In some embodiments, the recombinant polynucleotide comprises a polynucleotide sequence encoding the engineered AAV VP3 capsid polypeptide comprising an amino acid sequence comprising substitutions in at least two amino acid positions 588, 665, 457, 495, 456, 505, 494, 497, 592, 586, 585, 706, 549, 552, 664, 661, 461, 668, 548, 510, 640, 419, 349, 504, 508, 453, 589, and 416, wherein the amino acid positions are relative to the reference sequence corresponding to residues 203-736 of SEQ ID NO: 2.

In some embodiments, the recombinant polynucleotide comprises a polynucleotide sequence encoding the engineered AAV VP3 capsid polypeptide comprising an amino acid sequence comprising at least a first and second substitutions at amino acid positions selected from (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, and 416, wherein the first substitution is at a position selected from (a), and the second substitution is at a position selected from (a), (b) and (c), wherein the amino acid positions are relative to the reference sequence corresponding to residues 203-736 of SEQ ID NO: 2.

In some embodiments, the recombinant polynucleotide comprises a polynucleotide sequence encoding the engineered AAV VP3 capsid polypeptide comprising an amino acid sequence comprising at least a substitution set at amino acid positions 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to the reference sequence corresponding to residues 203-736 of SEQ ID NO: 2.

In some embodiments, the recombinant polynucleotide comprises a polynucleotide sequence encoding the engineered AAV VP3 capsid polypeptide comprising an amino acid sequence comprising at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 331/449, 211/509/712, 331/416/478, 507, 331/584/597, 478, 349/465/479/504/508, 349/640, 331/416/478/483/507/584/597, 416, 233/465/640, 508/510/589, 597, 331/597, 331/416/665, 465/504/508/510, 331/478/483/529/597, 384/479, 233/640, 419/504/508/530, 504/510/640, 419/504/508, 331/361, 416/665, 211/361/416/478/584, 331/416, 175/211, 211/478/483/712, 349/504/508, 508/510, 331/416, 331, 479/504/510/589, 331/478, 331/478/483/597, 479, 504/508, 479/589, or 479/510/589, wherein the amino acid positions are relative to the reference sequence corresponding to residues 203-736 of SEQ ID NO: 2.

In some embodiments, the recombinant polynucleotide comprises a polynucleotide sequence encoding the engineered AAV VP3 capsid polypeptide comprising an amino acid sequence comprising at least a substitution at an amino acid position between residues 203-736 of AAV capsid polypeptide, wherein the amino acid positions are set forth in Tables 5.1, 5.2, 6.1, 6.2, 7.1, and 7.2, and wherein the amino acid positions are relative to the reference sequence corresponding to residues 203-736 of SEQ ID NO: 2.

In some embodiments, the recombinant polynucleotide comprises a polynucleotide sequence encoding the engineered AAV VP3 capsid polypeptide comprising an amino acid sequence comprising at least one substitution between residues 203-736 of AAV capsid polypeptide, wherein the substitutions are set forth in Tables 5.1, 5.2, 6.1, 6.2, 7.1, and 7.2, and wherein the amino acid positions are relative to the reference sequence corresponding to residues 203-736 of SEQ ID NO: 2.

In some embodiments, the recombinant polynucleotide comprises a polynucleotide sequence encoding the engineered AAV VP3 capsid polypeptide comprising an amino acid sequence comprising at least a substitution or substitution set at amino acid position(s) between residues 203-736 of AAV capsid polypeptide, wherein the substitution or substitution set is set forth in Tables 5.1, 5.2, 6.1, 6.2, 7.1, and 7.2, and wherein the amino acid positions are relative to the reference sequence corresponding to residues 203-736 of SEQ ID NO: 2.

In some embodiments, the recombinant polynucleotide comprises a polynucleotide sequence encoding the engineered AAV VP3 capsid polypeptide comprising an amino acid sequence comprising at least a substitution or substitution set between residues 203-736 of an engineered AAV capsid polypeptide set forth in Tables 5.1, 5.2, 6.1, 6.2, 7.1, and 7.2, wherein the amino acid positions are relative to the reference sequence corresponding to residues 203-736 of SEQ ID NO: 2.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278 or 522.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO, of SEQ ID NOs: 4-1134.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO, of SEQ ID NOs: 4-1134, wherein the amino acid sequence comprises one or more substitutions relative to the reference sequence corresponding to residues 203-736 of SEQ ID NO: 278 or 522.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278 or 522, wherein the amino acid sequence comprises one or more substitutions relative to the reference sequence corresponding to residues 203-736 of SEQ ID NO: 278 or 522.

In some embodiments, the recombinant polynucleotide comprises a polynucleotide sequence encoding the engineered AAV VP3 capsid polypeptide comprising an amino acid sequence comprising at least a substitution at amino acid position 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 203-736 of SEQ ID NO: 278 or 522.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278, wherein the amino acid sequence comprises one or more substitutions relative to the reference sequence corresponding to residues 203-736 of SEQ ID NO: 278.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO, of SEQ ID NOs: 332-722, wherein the amino acid sequence comprises one or more substitutions relative to the reference sequence corresponding to residues 203-736 of SEQ ID NO: 278.

In some embodiments, the recombinant polynucleotide comprises a polynucleotide sequence encoding the engineered AAV VP3 capsid polypeptide comprising an amino acid sequence comprising at least a substitution or substitution set at amino acid position(s) 485, 421, 391, 469, 628, 461, 642, 595, 414, 512, 519, 598, 449, 736, 462, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 663, 401, 579, 444, 459/508/663/667, 412, 440, 387, 453/586, 419, 472, 450, 585, 437, 562, 169, 588, 565, 589, 495/497, or 142/552, wherein the amino acid positions are relative to the reference sequence corresponding to residues 203-736 of SEQ ID NO: 278.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of SEQ ID NO: 552, wherein the amino acid sequence comprises one or more substitutions relative to the reference sequence corresponding to residues 203-736 of SEQ ID NO: 552.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO, of SEQ ID NOs: 724-1134, wherein the amino acid sequence comprises one or more substitutions relative to the reference sequence corresponding to residues 203-736 of SEQ ID NO: 552.

In some embodiments, the recombinant polynucleotide comprises a polynucleotide sequence encoding the engineered AAV VP3 capsid polypeptide comprising an amino acid sequence comprising at least a substitution or substitution set at amino acid position(s) 725, 414/456/459/485/736, 454, 668, 485/595, 454/485, 346, 421/456/459, 218, 205, 348, 421, 551, 736, 529/552/585, 535, 529/585, 535/659, 594, 468, 726, 468/592, 451, 559, 575, 465, 560, 273, 389/510, 470, 724, 485/597, 510, 256, 334, 211, 264, 595, 391, 391/661, 391/454/471, 391/421, 401/414/485, 391/454/595, 529, 529/552/588/716, 493/585/667, 269, 552, 515, 565, 470/663, 414, 597, 564, 389, 720, 451/452, 496, 214, or 320, wherein the amino acid positions are relative to the reference sequence corresponding to residues 203-736 of SEQ ID NO: 552.

In some embodiments, the recombinant polynucleotide comprises a polynucleotide sequence encoding the engineered AAV VP3 capsid polypeptide comprising an amino acid sequence comprising at least a substitution or substitution set at amino acid position(s) 333, 254, 725, 258, 268, 214/451, 331, 328, 274, 576, 560, 272, 263, 270, 267, 210/266, 259, 259/466, 264, 266, 535, 329, or 470, wherein the amino acid positions are relative to the reference sequence corresponding to residues 203-736 of SEQ ID NO: 552.

In some embodiments, the recombinant polynucleotide comprises a polynucleotide sequence encoding the engineered AAV VP3 capsid polypeptide comprising at least a substitution at an amino acid position between residues 203-736 of AAV capsid polypeptide, wherein the amino acid positions are set forth in Tables 5.1, 5.2, 6.1, 6.2, 7.1, and 7.2, and wherein the amino acid positions are relative to the reference sequence corresponding to residues 203-736 of SEQ ID NO: 278 or 552.

In some embodiments, the recombinant polynucleotide comprises a polynucleotide sequence encoding the engineered AAV VP3 capsid polypeptide comprising at least one substitution between residues 203-736 of AAV capsid polypeptide, wherein the substitutions are forth in Tables 5.1, 5.2, 6.1, 6.2, 7.1, and 7.2, wherein the amino acid positions are relative to the reference sequence corresponding to residues 203-736 of SEQ ID NO: 278 or 552.

In some embodiments, the recombinant polynucleotide comprises a polynucleotide sequence encoding the engineered AAV VP3 capsid polypeptide comprising at least a substitution or substitution set at amino acid position(s) between residues 203-736 of AAV capsid polypeptide, wherein the substitution or substitution set are set forth in Tables 5.1, 5.2, 6.1, 6.2, 7.1, and 7.2, wherein the amino acid positions are relative to the reference sequence corresponding to residues 203-736 of SEQ ID NO: 278 or 552.

In some embodiments, the recombinant polynucleotide comprises a polynucleotide sequence encoding the engineered AAV VP3 capsid polypeptide comprising at least a substitution or substitution set between residues 203-736 of an engineered AAV capsid polypeptide set forth in Tables 5.1, 5.2, 6.1, 6.2, 7.1, and 7.2, wherein the amino acid positions are relative to the reference sequence corresponding to residues 203-736 of SEQ ID NO: 278 or 552.

In some embodiments, the recombinant polynucleotide comprises a polynucleotide sequence encoding the engineered AAV VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a sequence corresponding to residues 203-736 an engineered AAV capsid polypeptide set forth in Tables 5.1, 5.2, 6.1, 6.2, 7.1, and 7.2.

In some embodiments, the recombinant polynucleotide comprises a polynucleotide sequence encoding the engineered AAV VP3 capsid polypeptide comprising an amino acid sequence comprising residues 203-736 of an even-numbered SEQ ID NO, of SEQ ID NOs: 4-1134, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions.

In some embodiments, the recombinant polynucleotide comprises a polynucleotide sequence having at least 60%, 65%, 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 607-2208 of an odd-numbered SEQ ID NO, of SEQ ID NOs: 3-1133, wherein the recombinant polynucleotide encodes an AAV VP3 capsid polypeptide.

In some embodiments, the recombinant polynucleotide comprises a polynucleotide sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 607-2208 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 337, 339, 341, 343, 345, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 443, 445, 447, 449, 451, 453, 459, 461, 463, 467, 469, 471, 481, 483, 485, 487, 489, 495, 499, 501, 503, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 599, 601, 609, 611, 617, 621, 623, 625, 629, 631, 633, 637, 641, 643, 645, 649, 651, 655, 657, 661, 663, 665, 669, 671, 677, 681, 683, 685, 689, 697, 703, 707, 709, 711, 713, 715, 717, 719, 721, 723, 725, 729, 731, 733, 735, 737, 739, 741, 743, 745, 747, 749, 753, 755, 757, 759, 761, 763, 767, 769, 775, 781, 783, 785, 787, 795, 797, 799, 803, 807, 809, 811, 813, 815, 817, 819, 823, 825, 827, 829, 831, 833, 835, 839, 843, 847, 851, 853, 855, 857, 859, 861, 865, 867, 875, 877, 879, 883, 885, 887, 889, 891, 895, 897, 899, 901, 903, 905, 909, 915, 919, 921, 923, 925, 927, 929, 931, 933, 935, 937, 941, 945, 947, 949, 953, 955, 961, 963, 965, 967, 969, 973, 975, 979, 981, 985, 987, 993, 995, 999, 1003, 1005, 1007, 1011, 1015, 1017, 1019, 1021, 1025, 1027, 1033, 1035, 1037, 1043, 1049, 1051, 1053, 1055, 1061, 1063, 1069, 1073, 1075, 1077, 1079, 1081, 1083, 1079, 1093, 1095, 1097, 1099, 1101, 1103, 1105, 1107, 1109, 1111, 1113, 1115, 1117, 1119, 1121, 1123, 1125, 1127, 1131, or 1133, wherein the recombinant polynucleotide encodes an engineered AAV VP3 capsid polypeptide.

In some embodiments, the recombinant polynucleotide comprises a polynucleotide sequence comprising nucleotide residues 607-2208 of an odd-numbered SEQ ID NO, of SEQ ID NOs: 3-1133.

In some embodiments, the recombinant polynucleotide comprises a polynucleotide sequence comprises residues 607-2208 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 337, 339, 341, 343, 345, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 443, 445, 447, 449, 451, 453, 459, 461, 463, 467, 469, 471, 481, 483, 485, 487, 489, 495, 499, 501, 503, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 599, 601, 609, 611, 617, 621, 623, 625, 629, 631, 633, 637, 641, 643, 645, 649, 651, 655, 657, 661, 663, 665, 669, 671, 677, 681, 683, 685, 689, 697, 703, 707, 709, 711, 713, 715, 717, 719, 721, 723, 725, 729, 731, 733, 735, 737, 739, 741, 743, 745, 747, 749, 753, 755, 757, 759, 761, 763, 767, 769, 775, 781, 783, 785, 787, 795, 797, 799, 803, 807, 809, 811, 813, 815, 817, 819, 823, 825, 827, 829, 831, 833, 835, 839, 843, 847, 851, 853, 855, 857, 859, 861, 865, 867, 875, 877, 879, 883, 885, 887, 889, 891, 895, 897, 899, 901, 903, 905, 909, 915, 919, 921, 923, 925, 927, 929, 931, 933, 935, 937, 941, 945, 947, 949, 953, 955, 961, 963, 965, 967, 969, 973, 975, 979, 981, 985, 987, 993, 995, 999, 1003, 1005, 1007, 1011, 1015, 1017, 1019, 1021, 1025, 1027, 1033, 1035, 1037, 1043, 1049, 1051, 1053, 1055, 1061, 1063, 1069, 1073, 1075, 1077, 1079, 1081, 1083, 1079, 1093, 1095, 1097, 1099, 1101, 1103, 1105, 1107, 1109, 1111, 1113, 1115, 1117, 1119, 1121, 1123, 1125, 1127, 1131, or 1133.

In some embodiments, the recombinant polynucleotide encoding any of the engineered AAV capsid polypeptides (e.g., VP1, VP2 and/or VP3) is codon optimized for expression in a host cell. In some embodiments, the recombinant polynucleotide encoding an engineered AAV capsid polypeptide described herein is codon optimized for expression in an insect cell or mammalian cell. In some embodiments, the recombinant polynucleotide encoding an engineered AAV capsid polypeptide is codon optimized for expression in a mammalian cell, in particular a human cell.

### Polynucleotides Encoding Engineered MAAP Polypeptides

In some embodiments, the recombinant polynucleotide comprises a polynucleotide sequence encoding an engineered MAAP polypeptide described herein.

In some embodiments, the recombinant polynucleotide comprises a polynucleotide sequence encoding the engineered MAAP polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 1136, wherein the amino acid sequence comprises one or more substitutions relative to the reference sequence corresponding to SEQ ID NO: 1136.

In some embodiments, the recombinant polynucleotide comprises a polynucleotide sequence encoding the engineered MAAP polypeptide comprising an amino acid sequence comprising at least a substitution at amino acid position 5, 7, 17, 27, 30, 34, 40, 64, 73, 74, 75, 93, 99, 100, or 116, or combinations thereof, wherein the amino acid positions are relative to SEQ ID NO: 1136.

In some embodiments, the recombinant polynucleotide comprises a polynucleotide sequence encoding the engineered MAAP polypeptide comprising an amino acid sequence comprising at least a substitution or substitution set at amino acid position(s) 99, 30, 17, 93, 116, 73, 5, 75, 34, 74, 27, 7, 73/74, 64, 40, or 100, wherein the amino acid positions are relative to SEQ ID NO: 1136.

In some embodiments, the recombinant polynucleotide comprises a polynucleotide sequence encoding the engineered MAAP polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, or 1232.

In some embodiments, the recombinant polynucleotide comprises a polynucleotide sequence encoding the engineered MAAP polypeptide comprising an amino acid sequence comprising SEQ ID NO: 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, or 1232, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions.

In some embodiments, the recombinant polynucleotide comprises a polynucleotide sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to SEQ ID NO: 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, or 1231, wherein the recombinant polynucleotide encodes an engineered MAAP polypeptide.

In some embodiments, the recombinant polynucleotide comprises a polynucleotide sequence comprising SEQ ID NO: 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, or 1231.

In some embodiments, the recombinant polynucleotide encoding any of the engineered MAAP polypeptides is codon optimized for expression in a host cell. In some embodiments, the recombinant polynucleotide encoding an engineered MAAP polypeptide described herein is codon optimized for expression in an insect cell or mammalian cell. In some embodiments, the recombinant polynucleotide encoding an engineered MAAP polypeptide is codon optimized for expression in a mammalian cell, in particular a human cell.

### Polynucleotides Encoding Engineered AAP Polypeptides

In some embodiments, a recombinant polynucleotide comprises a polynucleotide sequence encoding an engineered AAP polypeptide described herein.

In some embodiments, the recombinant polynucleotide comprises a polynucleotide sequence encoding an engineered AAP polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 1234, wherein the amino acid sequence comprises one or more substitutions relative to the reference sequence corresponding to SEQ ID NO: 1234.

In some embodiments, the recombinant polynucleotide comprises a polynucleotide sequence encoding the engineered AAP polypeptide comprising an amino acid sequence comprising at least a substitution at amino acid position 14, 15, 31, 49, 58, 79, 83, 84, 85, 88, 89, 91, 92, 93, 94, 95, 97, 99, 100, 102, 104, 105, 120, 153, 154, 155, 156, 157, 158, 160, 173, 174, or 194, or combinations thereof, wherein the amino acid positions are relative to SEQ ID NO: 1234.

In some embodiments, the recombinant polynucleotide comprises a polynucleotide sequence encoding the AAP polypeptide comprising an amino acid sequence comprising at least a substitution or substitution set at amino acid position(s) 58, 156, 49/156, 174, 157/158, 160, 158, 15, 79, 104, 31/156, 83, 92/93, 102, 155/156, 14/15, 153, 194, 99, 85, 105, 97, 88, 94/95, 91/92, 100, 91, 92, 83/84, 89, 94, 94/95/173, 97/120, 95, or 154, wherein the amino acid positions are relative to SEQ ID NO: 1234.

In some embodiments, the recombinant polynucleotide comprises a polynucleotide sequence encoding the engineered AAP polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, or 1410.

In some embodiments, the recombinant polynucleotide comprises a polynucleotide sequence encoding the engineered AAP polypeptide comprising an amino acid sequence comprising SEQ ID NO: 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, or 1410, optionally wherein the amino acid sequence has 1, 2, 3, 4, 5, 6, 7, 8, 9, or up to 10 insertions, substitutions, and/or deletions. In some embodiments, optionally the amino acid sequence has 1, 2, 3, 4, 5, 6, 7, 8, 9, or up to 10 substitutions.

In some embodiments, the recombinant polynucleotide comprises a polynucleotide sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to SEQ ID NO: 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1367, 1371, 1373, 1375, 1377, 1379, 1381, 1383, 1385, 1387, 1389, 1391, 1393, 1395, 1397, 1399, 1401, 1403, 1405, 1407, or 1409, wherein the recombinant polynucleotide encodes an engineered AAP polypeptide.

In some embodiments, the recombinant polynucleotide comprises a polynucleotide sequence comprising SEQ ID NO: 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1367, 1371, 1373, 1375, 1377, 1379, 1381, 1383, 1385, 1387, 1389, 1391, 1393, 1395, 1397, 1399, 1401, 1403, 1405, 1407, or 1409.

In some embodiments, the recombinant polynucleotide encoding any of the engineered AAP polypeptides is codon optimized for expression in a host cell. In some embodiments, the recombinant polynucleotide encoding an engineered AAP polypeptide described herein is codon optimized for expression in an insect cell or mammalian cell. In some embodiments, the recombinant polynucleotide encoding an engineered AAP polypeptide is codon optimized for expression in a mammalian cell, in particular a human cell.

### Expression Vectors and Host Cells

In some embodiments, a recombinant polynucleotide encoding any of the engineered polypeptides provided herein is manipulated in a variety of ways to provide for expression of the engineered polypeptide. In some embodiments, the recombinant polynucleotides encoding the engineered polypeptides are provided as expression vectors where one or more control sequences is operably linked to regulate the expression of the polynucleotides and/or polypeptides, particularly in an appropriate host cell.

In some embodiments, the expression vector comprises a recombinant polynucleotide encoding an engineered AAV capsid polypeptide. In some embodiments, the expression vector comprises a recombinant polynucleotide encoding an engineered AAV VP1, VP2 and/or VP3 capsid polypeptide. In some embodiments, the expression vector further comprises a recombinant polynucleotide comprising the rep gene encoding the rep proteins (e.g., Rep78, Rep68, Rep52, and Rep40) required for viral genome replication and packaging. In some embodiments, the polynucleotide encoding the AAV capsid polypeptide is in an expression vector or plasmid separate from the expression vector used for expression of the rep proteins.

In some embodiments, the expression vector comprises a recombinant polynucleotide encoding an engineered MAAP polypeptide described herein. In some embodiments, the recombinant polynucleotide encoding the engineered MAAP polypeptide comprises a recombinant polynucleotide encoding an engineered AAV capsid polypeptide described herein.

In some embodiments, the expression vector comprises a recombinant polynucleotide encoding an engineered AAP polypeptide described herein. In some embodiments, the recombinant polynucleotide encoding the engineered AAP polypeptide comprises a recombinant polynucleotide encoding an engineered AAV capsid polypeptide herein.

In some embodiments, the control sequences in the expression vectors include among others, promoter sequences, leader sequences, polyadenylation sequences, pro-peptide sequences, signal peptide sequences, DNA based regulatory elements for gene therapy retention, and transcription terminators (see, e.g., Makrides, S.C., New Comprehensive Biochemistry, 2003; 38:9-26).

As known in the art, suitable promoters can be selected based on the host cells used, e.g., mammalian cells, Exemplary promoters for expression in mammalian cells include, among others, AAV promoter, chicken β-actin promoter fused with the CMV enhancer, and promoters from cytomegalovirus (CMV), rous sarcoma virus (RSV), Simian virus 40 (SV40), Homo sapiens phosphoglycerate kinase, beta actin, elongation factor-1a (EF1a), or glyceraldehyde-3-phosphate dehydrogenase, cytokeratin 14, and cytokeratin 18, In some embodiments, where the host cell in an insect cell, promoters functioning in insect cells can be used, including, among others, AcMNPV polyhedrine promoter and promoter of the immediate-early 1 gene (AIE1) from polyhedrosis virus (see, e.g., Ruffing et al., J Virol., 1992, 66:6922-6930), In some embodiments, the promoter is an inducible promoter that allows regulated transcription, Exemplary inducible promoters regulated by exogenous agents include the zinc-inducible sheep metallothionine (MT) promoter, dexamethasone (Dex)-inducible promoter, mouse mammary tumor virus (MMTV) promoter; ecdysone insect promoter, tetracycline-inducible promoter system, RU486-inducible promoter system, and the rapamycin-inducible promoter system.

In some embodiments, the control sequence is a suitable transcription terminator sequence, a sequence recognized by a host cell to terminate transcription. The terminator sequence is operably linked to the 3′ terminus of the nucleic acid sequence encoding the polypeptide, Any terminator which is functional in the host cell of choice finds use in the present invention, Exemplary terminators for insect and mammalian cells include, but are not limited to those from cytomegalovirus (CMV), Simian vacuolating virus 40 (SV40), from Homo sapiens growth hormone hGH, from bovine growth hormone BGH, and from human or rabbit beta globulin.

In some embodiments, the control sequence is a suitable leader sequence, 5′-cap modification, 5′ UTR, etc. In some embodiments, these regulatory sequence elements mediate binding to molecules involved in mRNA trafficking and translation, inhibit 5′-exonucleolytic degradation and confer resistance to de-capping. The leader sequence is operably linked to the 5′ terminus of the nucleic acid sequence encoding the polypeptide, Any leader sequence that is functional in the host cell of choice may be used. Suitable leaders for insect and mammalian host cells include but are not limited to the 5′-UTR element present in orthopoxvirus mRNA.

In some embodiments, the control sequence comprises a 3′ untranslated nucleic acid region and polyadenylation tail nucleic acid sequence, sequences operably linked to the 3′ terminus of the protein coding nucleic acid sequence which mediate binding to proteins involved in mRNA trafficking and translation and mRNA half-life, Any polyadenylation sequence and 3′ UTR which is functional in the host cell of choice may be used in the present invention, Useful polyadenylation and 3′ UTR sequences for insect and mammalian host cells include, but are not limited to, the 3′-UTRs of α- and β-globin mRNAs that harbor several sequence elements that increase the stability and translation of mRNA.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome), The vector may contain any means for assuring self-replication. In some embodiments, the origin of replication may include, among others, the SV40 origin of replication, the Epstein-Barr (EBV) origin of replication, as well as others known to those skilled in the art.

In some alternative embodiments, the vector may be one which, when introduced into the host cell, is integrated into the genome and replicated together with the chromosome(s) into which it has been integrated. In some embodiments, the vector comprising a polynucleotide encoding an engineered AAV capsid polypeptide are stably integrated into the genome of the host cell, Host cells for stable integration including, among others, HeLa cells, A549 lung cancer cells, BHK21 hamster kidney fibroblasts, Sf9 insect cells (see, e.g., Gao et al., Mol. Ther., 2002, 5, 644-649).

In some embodiment, recombinant polynucleotides may be provided on a non-replicating expression vector or plasmid, which does not contain a functional origin of replication. In some embodiments, the non-replicating expression vector or plasmid comprises a polynucleotide encoding an AAV capsid polynucleotide and a polynucleotide encoding the rep polypeptide, Because the rep polypeptide can be toxic to cells, such non-replicating expression vector or plasmid can improve the replication of recombinant AAV genomes and optimize production of the AAV virus or viral particles. In some embodiments, the non-replicating expression vector or plasmid can be based on recombinant HSV vectors defective in replication.

In another aspect, the present disclosure provides a host cell comprising a recombinant polynucleotide encoding at least one engineered polypeptide described herein (e.g., AAV capsid polypeptide, AAV VP2 capsid polypeptide, AAV VP3 capsid polypeptide, MAAP polypeptide, AAP polypeptide), the polynucleotide being operatively linked to one or more control sequences for expression of the engineered polypeptides(s) in the host cell, Host cells for use in expressing the polypeptides encoded by the expression vectors of the present disclosure include but are not limited to, insect cells (e.g., Drosophila S2 and Spodoptera Sf9 cells), mammalian cells (e.g., CHO, CHO-KI, COS, and BHK), and human cells (e.g., HEK293 based cells, human fibroblasts, A549 lung cancer cells, HeLa cells, etc.), In some embodiments, the host cell comprises a AAV helper virus or a helper plasmid that expresses helper function necessary for virus production.

Accordingly, in another aspect, the present disclosure provides a method for producing the engineered polypeptides, where the method comprises culturing a host cell capable of expressing a polynucleotide encoding the engineered polypeptide under conditions suitable for expression of the polypeptide such that the engineered polypeptide is produced. In some embodiments, the engineered polypeptide produced in the host cell comprises an engineered AAV capsid polypeptide, an engineered AAV VP2 capsid polypeptide, and/or an engineered AAV VP3 capsid polypeptide. In some embodiments, the engineered polypeptide produced in the host cell comprises an engineered MAAP polypeptide. In some embodiments, the engineered produced in the host cell comprises an engineered AAP polypeptide. In some embodiments, the engineered polypeptide produced in the host cell comprises an engineered AAV VP1 capsid polypeptide, an engineered AAV VP2 capsid polypeptide, an engineered AAV VP3 capsid polypeptide, an engineered MAAP polypeptide, and an engineered AAP polypeptide described herein.

In some embodiments, the method further comprises the step of isolating the engineered polypeptide produced, such as from the culture media or cells. In some embodiments, the method comprises the step of isolating the engineered AAV VP1 capsid polypeptide. In some embodiments, the method comprises the step of isolating the engineered AAV VP2 capsid polypeptide. In some embodiments, the method comprises the step of isolating the engineered AAV VP3 capsid polypeptide. In some embodiments, the method comprises the step of isolating the engineered MAAP polypeptide. In some embodiments, the method comprises the step of isolating the engineered AAP polypeptide.

In some embodiments, the method further comprises the step of purifying the expressed engineered polypeptide, as described herein. In some embodiments, the method comprises the step of purifying the engineered AAV VP1 capsid polypeptide. In some embodiments, the method comprises the step of purifying the engineered AAV VP2 capsid polypeptide. In some embodiments, the method comprises the step of purifying the engineered VP3 capsid polypeptide. In some embodiments, the method comprises the step of purifying the engineered MAAP polypeptide. In some embodiments, the method comprises the step of purifying the engineered AAP polypeptide.

Appropriate culture media and growth conditions for the above-described host cells are well known in the art. Polynucleotides for expression of the engineered polypeptides may be introduced into cells by various methods known in the art. Techniques include, among others, electroporation, biolistic particle bombardment, liposome mediated transfection, calcium chloride transfection, and protoplast fusion.

In some embodiments, the engineered polypeptides with the properties disclosed herein can be obtained by subjecting the polynucleotide encoding the naturally occurring or engineered polypeptide to mutagenesis and/or directed evolution methods known in the art, and as described herein. An exemplary directed evolution technique is mutagenesis and/or DNA shuffling (See e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 1998, 16:258-261), mutagenic PCR (See e.g., Caldwell et al., PCR Methods Appl., 1994, 3: S136-S140), and cassette mutagenesis (see e.g., Black et al., Proc. Natl. Acad. Sci. USA, 1996, 93:3525-3529).

For example, mutagenesis and directed evolution methods can be readily applied to polynucleotides to generate variant libraries that can be expressed, screened, and assayed. Mutagenesis and directed evolution methods are known in the art (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,489,146, 6,506,602, 6,506,603, 6,519,065, 6,521,453, 6,528,311, 6,537,746, 6,573,098, 6,576,467, 6,579,678, 6,586,182, 6,602,986, 6,613,514, 6,653,072, 6,716,631, 6,946,296, 6,961,664, 6,995,017, 7,024,312, 7,058,515, 7,105,297, 7,148,054, 7,288,375, 7,421,347, 7,430,477, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,873,499, 7,904,249, 7,957,912, 8,383,346, 8,504,498, 8,849,575, 8,876,066, 8,768,871, 9,593,326, and all related non-US counterparts; Ling et al., Anal. Biochem., 1997, 254 (2): 157-78; Dale et al., Meth. Mol. Biol., 1996, 57:369-74; Smith, Ann. Rev. Genet., 1985, 19:423-462; Botstein et al., Science, 1985, 229:1193-1201; Carter, Biochem. J., 1986, 237:1-7; Kramer et al., 1984, Cell, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. USA, 1997, 94:4504-4509; Crameri et al., Nat. Biotechnol., 1996, 14:315-319; Stemmer, Nature, 1994, 370:389-391; Stemmer, Proc. Nat. Acad. Sci. USA, 1994, 91:10747-10751; U.S. patent publication Nos. 2008/0220990, US 2009/0312196, US2014/0005057, US2014/0214391, US2014/0221216; US2015/0050658, US2015/0133307, US2015/0134315 and all related non-US counterparts; WO 95/22625, WO 97/0078, WO 97/35966, WO 98/27230, WO 00/42651, WO 01/75767, and WO 2009/152336; all of which are incorporated herein by reference).

In some embodiments, the engineered AAV capsid polypeptide obtained following mutagenesis treatment can be used for expression in appropriate host cells to assess expression efficiency, and in the presence of a helper virus or helper plasmid to produce rAAV virus or rAAV virions, which are then screened, for example, by measuring titer and packaging efficacy. In some embodiments, rAAV virus or rAAV virions can be used for infection of appropriate host cells or organisms, which allows determining of transduction and cell tropism. In some embodiments, cultured host cells are used to assess properties such as packaging efficiency, capsid expression levels, transduction efficiency, and transgene expression. In some embodiments, infection of an appropriate mammal, e.g., mouse, rat or primate, with AAVs with engineered capsids followed by isolation/detection of the virus in different organs are used to identify AAV capsid variants having differing tissue/cell tropism characteristics. DNA encoding the AAV capsid polypeptide is sequenced to identify the nucleotide sequence changes (if any), and used to generate AAV virions in a different or the same host cell. Host mammals can also be used to assess immunogenicity or presence of neutralizing antibodies (Nabs) to the engineered AAV.

For engineered AAV polypeptides of known sequence, the polynucleotides encoding the polypeptide can be prepared by standard solid-phase methods, according to known synthetic methods. In some embodiments, polynucleotide fragments can be individually synthesized, then joined (e.g., by enzymatic or chemical litigation methods, or polymerase mediated methods) to form any desired continuous sequence. For example, polynucleotides and oligonucleotides disclosed herein can be prepared by chemical synthesis, e.g., using the classical phosphoramidite method (see e.g., Beaucage et al., Tetra. Lett., 1981, 22:1859-69; and Matthes et al., EMBO J . . . 1984, 3:801-05).

rAAV Viruses, rAAV Vectors, Pharmaceutical Compositions Thereof, and Methods of Producing rAAV Viruses

In another aspect, the present disclosure provides a rAAV virus or rAAV virion or rAAV viral particle comprising the engineered AAV capsid polypeptides described herein. In some embodiments, the rAAV virus or viral particle comprises at least an engineered AAV capsid polypeptide, e.g., VP1 capsid, an engineered AAV VP2 capsid polypeptide, and/or an engineered AAV VP3 capsid polypeptide described herein. In some embodiments, the rAAV virus or virion or viral particle comprises at least an engineered AAV VP1 capsid polypeptide described herein. In some embodiments, the rAAV virus or virion or viral particle comprises at least an engineered AAV VP2 capsid polypeptide described herein. In some embodiments, the rAAV virus or virion or viral particle comprises at least an engineered AAV VP3 capsid polypeptide described herein. In some embodiments, the rAAV virus or virion or viral particle comprises at least an engineered AAV VP1, VP2, and VP3 capsid polypeptide described herein.

In some embodiments, the rAAV virus or rAAV virion or rAAV viral particle further comprises an rAAV vector. In some embodiments, the rAAV vector comprises at least ITRs flanking a transgene of interest, wherein the transgene is operably linked to one or more appropriate control sequences, e.g., a promoter, for expression of the transgene (also collectively referred to herein as a transgene expression cassette).

In some embodiments, the transgene is typically heterologous to the vector sequences. In some embodiments, the transgene comprises a polynucleotide encoding a reporter polypeptide, for example, fluorescent protein (green fluorescent proteins, blue fluorescent proteins, cyan fluorescent proteins, yellow fluorescent proteins, orange fluorescent proteins, and red fluorescent proteins; GFP, EGFP, ECFP, DsRed2FP, etc.), bacterial β-galactosidase, firefly luciferase, beta-lactamase, and alkaline phosphatase.

In some embodiments, the transgene comprises a polynucleotide encoding a selection protein that selectively kills (i.e., negative selection) or allows survival (i.e., positive selection) of the transduced cell, for example, chloramphenicol acetyl transferase (CAT), nourseothricin N-acetyl transferasc, blasticidin-S deaminase, blastcidin S acetyltransferase, Sh blc (Zcocin® resistance), aminoglycoside 3′-phosphotransferase (neomycin resistance), hph (hygromycin resistance), thymidine kinase, and puromycin N-acetyl-transferase. Other example of transgenes for selection include polynucleotide encoding diphtheria toxin.

In some embodiments, the transgene comprises a polynucleotide encoding an enzyme, a signal transduction polypeptide or protein, a ligand polypeptide or protein, or a structural polypeptide or protein. In some embodiments, the transgene comprises a polynucleotide encoding a growth factor, cytokine, or interferon. In some embodiments, the transgene comprises a polynucleotide encoding a functioning gene associated with a disease or condition, for example, glucose-6-phosphatase (glycogen storage deficiency type 1A); phosphoenolpyruvatecarboxykinase (Pepck deficiency); galactose-1 phosphate uridyl transferase (galactosemia); phenylalanine hydroxylase (phenylketonuria): branched chain alpha-ketoacid dehydrogenase (Maple syrup urine disease); fumarylacetoacetate hydrolase (tyrosinemia type 1); methylmalonyl-CoA mutase (methylmalonic academia); medium chain acyl CoA dehydrogenase. (medium chain acetyl CoA deficiency); omithine transcarbamylase (omithine transcarbamylase deficiency); argininosuccinic acid synthetase (citrullinemia); low density lipoprotein receptor protein (familial hypercholesterolemia); UDP-glucouronosyltransferase (Crigler-Najjar disease); adenosine deaminase (severe combined immunodeficiency disease); hypoxanthine guanine phosphoribosyl transferase (Gout and Lesch-Nyan syndrome); biotinidase (biotinidase deficiency); beta-glucocerebrosidase (Gaucher disease); beta-glucuronidase (Sly syndrome); peroxisome membrane protein 70 kDa (Zellweger syndrome); porphobilinogen deaminase (acute intermittent porphyria); alpha-1 antitrypsin (alpha-1 antitrypsin deficiency; emphysema); and erythropoietin (thalassemia).

In some embodiments, the transgene comprises a polynucleotide capable of transcribing a non-coding RNA molecule, such as shRNA, miRNA, iRNA, etc. In some embodiments, by way of example and not limitation, the non-coding RNA molecule is a miRNA which regulates gene expression by targeting RNA transcript cleavage/degradation or 5 translational repression of the target messenger RNA (mRNA).

In some embodiments, the transgene comprises one or more components of a gene editing system or recombination system. In some embodiments, the transgene comprises a polynucleotide encoding a CRISPR associated protein 9 (Cas9). In some embodiments, the transgene comprises a polynucleotide capable of transcribing a guide RNA. In some embodiments, the transgene comprises a polynucleotide encoding a zinc-finger nuclease (ZFN) or transcription factor-like effector nucleases (TALEN). In some embodiments, the transgene comprises a polynucleotide encoding a recombinase, such as Cre recombinase or flp recombinase.

In some embodiments, the rAAV vector further comprises a recombination sequence, such as Cre-lox or FLP. In some embodiments, the recombination sequence flanks a transgene. In some embodiments, the recombination sequences are present flanking a polynucleotide segment between a promoter and a transgene, allowing for recombinase mediated juxtaposition of the promoter and transgene upon recombination.

In some embodiments, a host cell comprises a rAAV virus or rAAV virion or rAAV virus particle described herein. In some embodiments, the host cell includes those used for expression of the engineered AAV capsid polypeptides, engineered MAAP polypeptides, and/or engineered AAP polypeptides. In some embodiments, the host cells are competent for expressing the engineered AAV capsid polypeptides, engineered MAAP polypeptides, and/or engineered AAP polypeptides and for producing rAAV viruses, rAAV virions, and rAVV viral particles when provided with appropriate helper functions, e.g., a helper virus or helper plasmid expressing helper functions. In some embodiments, the host cell is an insect cell or mammalian cell. In some embodiments, the mammalian cell is a human cell.

In some embodiments, the rAAV virus, rAAV virion or rAAV viral particle comprising the engineered AAV capsid polypeptides is provided as a pharmaceutical composition. In some embodiments, the pharmaceutical composition comprises a rAAV virus or rAAV virion or rAAV viral particle comprising an engineered AAV capsid polypeptide described herein, and a pharmaceutically acceptable excipient and/or carrier. In some embodiments, the pharmaceutically composition is suitable for administration by any suitable route, including, among others, intravenous, intracerebral, intrathecal, oral, subcutaneous, intramuscular, nasal, intravitreal, intraretinal, intraputamen, intrastriatal, intraventricular, intraparenchymal, and intraperitoneal.

In some embodiments, the pharmaceutically acceptable excipients and/or carriers, include, among others, diluents such as water, saline, glycerol, and ethanol. In some embodiments, the excipients or carriers comprise pharmaceutically acceptable salts, for example, mineral acid salts such as hydrochlorides, hydrobromides, phosphates, sulfates, and the like; and the salts of organic acids such as acetates, propionates, malonates, and benzoates. In some embodiments, the pharmaceutically acceptable excipients or carriers include, among others, wetting or emulsifying agents, and pH buffering agents. A description of pharmaceutically acceptable excipients is available in Remington's Pharmaceutical Sciences (Mack Pub. Co., N.J. 1991).

In some embodiments, the pharmaceutically acceptable excipients or carriers provide a protective effect on the rAAV virion such that loss of rAAV virions, as well as transduccability resulting from, among others, formulation procedures, packaging, storage, and transport, is minimized or reduced. In some embodiments, the excipients or carriers useful for protecting an rAAV virion from activity degradative conditions include, among others, detergents, proteins, e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, with molecular weights of 1500 to 6000 preferred, propylene glycols (PG), sugar alcohols, such as a carbohydrate (e.g., sorbitol).

In a further aspect, the present disclosure provides a method for producing an rAAV virus or rAAV virion or AAV viral particle, comprising (i) preparing or providing a host cell comprising at least a recombinant polynucleotide encoding at least an engineered AAV capsid polypeptide described here, a polynucleotide comprising the rep gene encoding the rep polypeptides, a polynucleotide comprising adenoviral helper sequences or functions, and optionally a polynucleotide comprising a transgene flanked by ITRs, and (ii) culturing the host cells under suitable conditions allowing for the production of the rAAV virus or rAAV virion or rAAV viral particle. In some embodiments, the host cell is permissive or competent for AAV replication.

In some embodiments, the polynucleotide comprising the rep gene encoding the rep polypeptides are provided in the expression vector that also includes the recombinant polynucleotide encoding the engineered AAV capsid polypeptide, i.e., as a single expression vector or plasmid. In some embodiments, the polynucleotide comprising the rep gene and the recombinant polynucleotide encoding the engineered AAV capsid polypeptide are independently driven by separate promoters. In some embodiments, the polynucleotide comprising the rep gene are provided in an expression vector or plasmid separate from the expression vector or plasmid comprising the recombinant polynucleotide encoding the engineered AAV capsid polypeptide. In some embodiments, the polynucleotide comprising the rep gene is present in the host cell, e.g., integrated into the host genome.

In some embodiments, the adenoviral helper sequences or functions include one or more of E1A, E1B, E2A, E4, and VARNA. In some embodiments the adenoviral helper functions are provided by infection of the cell with an appropriate helper virus. In some embodiments, the helper virus is an adenoviruses, herpesviruses, and poxviruses such as vaccinia. The adenoviruses encompass a number of different subgroups, although Adenovirus type 5 of subgroup C is commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

In some embodiments, the adenoviral helper sequences or functions are provided in an expression vector or plasmid that is introduced into the host cell. In some embodiments, the expression vector comprises a recombinant polynucleotide capable of expressing at least E2A, E4, and VARNA, particularly where the host cell expresses E1A and E1B, e.g., a producer cell line having a polynucleotide encoding E1A and E1B integrated into the host genome. In some embodiments, the expression vector comprises a recombinant polynucleotide capable of expressing E1A, E1B, E2A, E4, and VARNA. In some embodiments, the expression vector or plasmid expressing the helper functions are introduced into the host cell by appropriate methods.

In some embodiments, the method of producing an rAAV virus or rAAV viral particle further includes a polynucleotide comprising an ITR-flanked genome, particularly a transgene expression cassette flanked by ITRs. In some embodiments, the ITRs can be from any naturally occurring AAV virus serotype or a recombinant AAV. In some embodiments, the ITRs comprise ITRs from AAV1, AAV2, AAV3, AAV4, AAV5, AAV6, AAV7, AAV8, AAV9, AAV10, AAV11, AAV12 and AAV13, or hybrid ITRs comprising ITRs from at least two different AAV virus serotypes (see, e.g., Earley et al., Hum Gene Ther . . . 2020, 31 (3-4): 151-162). In some embodiments, the ITRs flanking the transgene comprise synthetic ITRs, including, among others, truncated or mutated ITR. In some embodiments, the ITR, whether naturally occurring, a hybrid, or synthetic, includes (i) an AAV rep binding element; (ii) an AAV terminal resolution sequence; and (ii) an AAV RBE′ element.

In some embodiments, as discussed above, the transgene comprises a polynucleotide, typically heterologous to the vector sequences, which encodes a polypeptide or protein, or transcriptionally competent to transcribe a non-coding RNA molecule, such as shRNA, miRNA, iRNA, etc., or any other gene product of interest, as described herein. In some embodiments, the transgene is operably linked to one or more control sequences which provides for transgene expression, and where appropriate, translation of an encoded polypeptide or protein, and/or expression in a target cell or tissue. In some embodiments, the transgene can include an intron operably located between the promoter/enhancer and the transgene. In some embodiments, the transgene of interest is operably linked to a promoter, and in certain embodiments, an enhancer, for expression of the transgene. In some embodiments, the promoter can be any of the promoters described herein. In particular, the promoter is functional in cells tropic to the engineered AAV capsid described herein, e.g., neuronal cells or CNS tissues. In some embodiments, the promoter comprises a tissue specific promoter. In some embodiments, a transgene flanked by ITRs is provided by the host cell, for example, as stably integrated into the host cell genome. In some embodiments, such host cells are infected with an helper virus, such as Adenovirus (e.g. Ad type 5) or AAV ITR flanked genome-containing Adenovirus/AAV hybrid virus, and transfected with an expression vector comprising a recombinant polynucleotide encoding an engineered AAV capsid polypeptide to generate the rAAV virus, virions or particles which contain the transgene packaged into the rAAV virus, virion or viral particle comprising an engineered AAV capsid polypeptide of the present disclosure.

In some embodiments, a method for producing an rAAV virus or AAV viral particle comprises (a) transfecting a host cell with a first expression vector comprising a polynucleotide encoding an engineered AAV capsid polypeptide, and a polynucleotide comprising a rep gene encoding the rep polypeptides, a second expression vector comprising a polynucleotide comprising adenoviral helper sequences or functions, and a third expression vector comprising a transgene of interest flanked by ITR sequences; and (b) culturing the host cell under conditions such that the rAAV virus or the rAAV viral particle comprising the engineered AAV capsid polypeptide is produced.

In some embodiments, the method further comprises isolating or purifying the rAAV virus or the AAV viral particle. Various methods for isolating and purifying the rAAV virus and rAAV viral particle are known in the art. In some embodiments, the rAAV virus or viral particles are isolated by ion exchange chromatography or chromatography mediums (e.g., membranes), density gradient centrifugation, affinity chromatography (e.g., with AAV capsid antibody, heparin, etc.) (see, e.g., Nass et al., Mol Ther Methods Clin Dev., 2018, 9:33-46), and/or extraction and partitioning in aqueous two-phase systems (see. e.g., Guo et al., J Virol Methods. 2012, 183 (2): 139-146: Negrini et al., Current Protocols in Neuroscience, 2020, 93:1 e103). In some embodiments, the method further comprises isolating or purifying rAAV virus or rAAV viral particle containing an rAAV vector from rAAV virus or rAAV viral particle that do not contain an rAAV vector (e.g., “empty viral capsid” or “empty viral particle”). Methods for isolating rAAV virus or rAAV viral particles containing rAAV vectors include, ion exchange chromatography (see, e.g., Urabe et al., Molecular Therapy, 2006, 13 (4): 823-828; see also U.S. Pat. No. 9,528,126), metal affinity chromatograph (see, e.g., Gagnon et al., J. Chromatography A, 2021, 1649:462210), density gradient centrifugation, membrane chromatography, and electrophoresis (see, e.g., De Pena et al., ACS Omega, 2022, 7 (27): 23457-23466). In some embodiments, purification of the rAAV virus or AAV viral particles can use commercial systems, such as POROS CaptureSelect AAV resins (Thermo Fisher).

### Uses and Methods

In another aspect, the rAAV virus or rAAV virion comprising an engineered AAV capsid polypeptide is used to deliver a rAAV vector to a cell. In some embodiments, a method of transducing a cell with a rAAV virus or virion or viral particle comprising an engineered AAV capsid polypeptide, comprises contacting a cell with a rAAV virus or virion or viral particle described herein under conditions suitable for infection of the cell by the rAAV virus or virion or viral particle. In some embodiments, the cell is a neuronal cell or glial cell. In some embodiments, the rAAV virus, virion, or viral particle further comprises a rAAV vector comprising a transgene, and the method is used to deliver the transgene to the cell.

In some embodiments, the rAAV virus or rAAV virion comprising an engineered AAV capsid polypeptide is used to deliver a rAAV vector to a subject, such as the CNS of a subject. In some embodiments, a method of delivering a rAAV virus or rAAV virion described herein comprises administering to a subject a rAAV virus or rAAV virion comprising an engineered AAV capsid polypeptide, wherein the rAAV virus or rAAV virion further comprises a rAAV vector. In some embodiments, the administering is by any suitable route, including, among others, intravenously, intracerebrally, intrathecally, oral, subcutaneously, intramuscularly, intranasally, intravitreally, intraretinally, intraputamenly intrastriatally, intraventricularly, intraparenchymally, and intraperitoneally.

In some embodiments, for administration to a subject, the dose of rAAV virus or virions administered is tailored to, among others, purpose of the administrations, nature of the transgene, nature of the subject (age, sex, body weight, animal type, etc.), route of administration, the cells or organ being targeted for transduction, the strength of the promoter in the cell type, and the effectiveness of the rAAV virus in transducing the cell type or organ(s).

In some embodiments, an “effective amount” of the rAAV virus or rAAV virion is from about 10⁵ to 10¹⁶ of the rAAV virions, preferably 10⁸ to 10¹⁴ rAAV virions. For in vitro transduction, an effective amount of rAAV virions is from about 10⁵ to 10¹³, preferably 10⁸ to 10¹³ of the rAAV virions. For in vivo administration, the dosage may be a single dose schedule or a multiple dose schedule to ultimately deliver the amount specified above.

In some embodiments, for treatment of a disease or condition as gene therapy, a therapeutically effective amount of the rAAV virus is administered, which in some embodiments, is about 10⁵ to 10¹⁶ of the rAAV virions, preferably 10⁸ to 10¹⁴ rAAV virions. For gene therapy, the rAAV virus can be administered as a single dose or as multiple doses, sufficient to treat the disease or condition.

### EXAMPLES

The following Examples, including experiments and results achieved, are provided for illustrative purposes only and are not to be construed as limiting the present invention.

### Example 1

### Gene Acquisition and Construction of Expression Vectors

A synthetic wild-type (WT) adeno-associated virus 9 (AAV9) cap gene (SEQ ID NO: 1) coding for the AAV9 capsid protein (SEQ ID NO: 2) and a synthetic WT adeno-associated virus 2 (AAV2) rep gene (Acc. No: Q89268) were synthesized and cloned into mammalian expression vectors containing a CMV promoter and SV40 pA sequence and a p5 promoter and SV40 pA sequence, respectively. Directed evolution techniques generally known by those skilled in the art were used to generate gene variants derived from SEQ ID NO: 1 within this plasmid construct (see e.g., U.S. Pat. No. 8,383,346 and WO2010/144103).

Expression of AAV capsids was achieved using the two plasmids described above as well as a pHelper plasmid purchased from the AAV Helper-Free System (Agilent Technologies, Inc.) and a dual reporter plasmid containing the firefly luciferase (FLuc2) and enhanced green fluorescent protein (eGFP) genes created by replacing the lacZ gene in pAAV-lacZ vector from the AAV Helper-Free System (Agilent Technologies, Inc.). All four plasmids were used to obtain expression of AAV capsids primarily from HEK293T cells and Viral Production Cells 2.0 (ThermoFisher Scientific). This approach provided lysates with measurable capsids, measurable DNA packaging, and measurable transduction in the neutralization assays and the potency assays primarily using HEK293T cells or human induced pluripotent stem cell (iPSC)-derived neurons.

In some embodiments, to identify mutational diversity with similar or improved properties compared to SEQ ID NO: 2, a combinatorial library was constructed, generating AAV capsid variants derived from SEQ ID NO: 1/2. Equivalent volumes of lysates were assessed for their titers and screened for their transduction capabilities in an unchallenged condition (no incubation, pH 7.4) or following a one-hour incubation (pH 7.4, 37° C.) in the presence of IVIG containing a pool of neutralizing antibodies (GAMMAGARD LIQUID, Takeda Pharmaceuticals). AAV capsid variants with improved titers due to increased capsid yield or increased packaging or improved transduction due to increased titer or increased transduction were identified based on their fold improvement over the parent AAV capsid. AAV capsid variants with reduced neutralization were identified by dividing the fold-improvement observed under challenged conditions by the fold-improvement observed under unchallenged conditions. This approach reduces the bias towards selecting variants based on increased titer or transduction but without changes in neutralization. The fold-improvements for all conditions and screens were used to rank mutations in improved variants for inclusion in subsequent AAV capsid libraries. In additional embodiments, the additional methods and sequences described in the Examples were used.

### Example 2

### High-Throughput Growth

The following four plasmids, as described in Example 1, were used to express the WT AAV9 capsid, or variants: (1) the pHelper plasmid (Agilent Technologies, Inc.), (2) a pAAV-derived plasmid containing the dual reporters FLuc2 and eGFP, (3) a p5 promoter and SV40 pA containing vector including the AAV2 rep gene, and (4) a CMV promoter and SV40 pA containing vector including the AAV9 cap gene (SEQ ID NO: 1), or variants. HEK293T cells or Viral Production Cells 2.0 (ThermoFisher Scientific) were transfected with these four plasmids, or PCR products derived from those plasmids, using the lipofection method with the AAV-MAX Transfection Kit (ThermoFisher Scientific) or the polymer transfection method with Polyethylenimine (PEI) Transporter 5® Transfection Reagent (Polysciences, Inc.). Viral Production Cells 2.0 (ThermoFisher Scientific) were cultured in growth medium (Viral Production Medium [ThermoFisher Scientific]). Immediately before transfection, cells were transferred to NUNC™ 96-Well DeepWell™ plates (ThermoFisher Scientific) at densities of 10E+6 cells/well/400 μL in growth medium, and incubated at 37° C., 8% CO₂ in an incubator. HEK293T cells were cultured in growth medium (DMEM with 10% fetal bovine serum [both from Corning]). 24 hours before transfection, cells were seeded to NUNC® Edge 2.0 96-well plate (ThermoFisher Scientific) at densities of 10E+4 cells/well/250 μL in growth medium, and incubated at 37° C., 5% CO₂ in an incubator. Cells were incubated for 24-96 hours at specific conditions to allow for expression of AAV capsid variants. Lysates (15-300 μL) from the transfections were transferred into Corning 96-well solid black plates (Corning) prior to titer, transduction, and neutralization analysis.

### Example 3

### Generation of Human iPSC-Derived Neurons

In this example, human iPSC-derived neurons are described. GM25256 and GM23279 human fibroblast-derived iPSCs were obtained from Coriell Institute and cultured and maintained under standard conditions: mTeSR plus medium from STEMCELL™ Technologies on growth factor reduced Matrigel (Corning). Passaging of iPSCs was performed using ReLeSR reagent (STEMCELL™ Technologies), according to the manufacturer protocol. Human iPSC-derived forebrain neurons were differentiated using kits from STEMCELL™ Technologies: 1) the STEMdiff™ SMADi Neural Induction Kit, 2) STEMdiff™ Forebrain Neuron Differentiation Kit, and 3) STEMdiff™ Forebrain Neuron Maturation Kit. These kits are designed to drive cells from iPSC stage to neural progenitor stage to differentiated neurons and finally mature neurons. For neural induction, we used the embryoid body-based protocol with SMADi reagent included. Experiments were performed on cultures matured for greater than eight days, also according to the manufacturer's guidelines. Cultures were either differentiated from iPSC to mature forebrain neuron cultures directly or were generated from previously cryopreserved neural progenitor cells (NPCs). Cryopreservation was performed using 1-2E+6 low passage NPCs in 1 mL STEMdiff™ Neural Progenitor Freezing Medium (STEMCELL™ Technologies), protocol directly from STEMCELL™ Technologies. All experiments were performed in the presence of antibiotic-antimycotic (Sigma-Aldrich).

### Example 4

### In Vitro Characterization of Variants in HTP

### HTP Analysis of Lysates (Titer)

Titer was determined through quantitative polymerase chain reaction (qPCR). In this assay, a 10 μL reaction mixture that comprised the TaqMan™ Fast Advanced Master Mix (ThermoFisher Scientific) at a 1× concentration, 900 nM primer 1 (SEQ ID NO: 1411), 900 nM primer 2 (SEQ ID NO: 1412), 250 nM FAM-labeled probe (SEQ ID NO: 1413), and 0.5-2 μL of lysates from HEK293T cells or Viral Production Cells 2.0 (ThermoFisher Scientific), containing AAV capsid variants, diluted with DPBS (Corning), was prepared for each variant tested. Then, reactions were incubated at 50° C. for 2 min and then at 95° C. for 20 sec before qPCR cycling (95° C. 1 sec, 60° C. 20 sec for 40 cycles) was conducted in a QuantStudio™ 6 Pro Real-Time PCR System (ThermoFisher Scientific).

### HTP Analysis of Lysates (Transduction)

AAV capsid variant transduction potency was determined by measuring the activity of FLuc2 or eGFP after transduction of HEK293T cells or human iPSC-derived neurons using the AAV capsid variants. Human iPSC-derived neurons were obtained as described in Example 3 and cultured at densities of 0.5-2E+4 cells/well with 25-200 μL of media (BrainPhys™ Neuronal Medium [STEMCELL™ Technologies]) in appropriate 96- or 384-well tissue culture plates. The cells were maintained with media (BrainPhys™ Neuronal Medium [STEMCELL™ Technologies]), changing the media every 2-3 days, prior to use in the assay. HEK293T cells were seeded in appropriate 96- or 384-well tissue culture plates at densities of 0.5-2E+4 cells/well with 25-200 μL of media (DMEM [Corning] alone, DMEM with 10% fetal bovine serum [Corning], or DMEM with 10% ultra-low IgG fetal bovine serum [ThermoFisher Scientific]) 1 day before use in the assay. For an unchallenged assay, 40 μL of lysates from HEK293T cells or Viral Production Cells 2.0 (ThermoFisher Scientific), containing AAV capsid variants, diluted with media (DMEM [Corning] alone, DMEM with 10% fetal bovine serum [Corning], or DMEM with 10% ultra-low IgG fetal bovine serum [ThermoFisher Scientific]) were added to HEK293T cells already containing 160 μL of the same media. Alternatively, for an unchallenged assay, 100 μL of lysates from HEK293T cells or Viral Production Cells 2.0 (ThermoFisher Scientific), containing AAV capsid variants, diluted with media (BrainPhys™ Neuronal Medium [STEMCELL™ Technologies]) were added to iPSC-derived neurons after removal of the media from the cultures. In some experiments, 0.5-10 μM Etoposide (Sigma-Aldrich) was added to the diluted lysate containing AAV capsid variants prior to adding to the iPSC-derived neurons to boost transduction. After 1-7 days, transduction potency was determined by measuring eGFP fluorescence (488 nm excitation and 510 nm emission) or FLuc2 luminescence after lysis using ONE-Glo™ Luciferase Assay System (Promega Corporation).

### HTP Analysis of Lysates (Neutralization)

AAV capsid variants were challenged with IVIG containing a pool of neutralizing antibodies (GAMMAGARD LIQUID, Takeda Pharmaceuticals). First, 20 μL of lysates from HEK293T cells or Viral Production Cells 2.0 (ThermoFisher Scientific), containing AAV capsid variants, diluted with media (DMEM [Corning] alone, DMEM with 10% fetal bovine serum [Corning], or DMEM with 10% ultra-low IgG fetal bovine serum [ThermoFisher Scientific]) and 20 μL of the IVIG (GAMMAGARD LIQUID, Takeda Pharmaceuticals), diluted with the same media to a final of concentration of 1-20 mg/mL, were added to the wells of a 96-well round bottom microtiter plate. The plates were sealed with a PlateLoc® thermal microplate sealer (Agilent) and incubated at 37° C. for 1 h. The samples were then analyzed by the transduction assay as described above.

### Example 5

### AAV Capsid Variants Derived from SEQ ID NO: 2

In this Example, experiments conducted to determine AAV capsid variant characteristics are described. These variants were assessed for their titer, transduction, and neutralization using the assays described in Example 4. Results are shown in Table 5.1 and Table 5.2. In Table 5.1, the sequences of MAAP (SEQ ID NO: 1135/1136) and AAP (SEQ ID NO: 1233/1234) are identical for all variants. In Table 5.2, the sequences of MAAP and AAP for some Capsid protein variants differ from the reference sequences of MAAP and AAP, as specified in the table.

TABLE 5-1
Performance of AAV variants relative to SEQ ID NO: 2

| SEQ | Amino Acid | | | | |
|---|---|---|---|---|---|
| ID NO | Differences in | | | | |
| Capsid | Capsid protein | FIOP | FIOP | FIOP | FIOP |
| protein: | (Relative to | CNS | HEK293T | HEK293T + | qPCR |
| (nt/aa) | SEQ ID NO: 2) | assay | assay | Nab assay | assay |
| 1/2 | | + | + | + | + |
| 3/4 | G547Q | | ++ | + | ++ |
| 5/6 | G453R | | +++ | ++ | ++ |
| 7/8 | G547E | | ++ | + | |
| 9/10 | A555W | | ++ | + | ++ |
| 11/12 | F535A | | ++ | ++ | ++ |
| 13/14 | F501M | | ++ | | ++ |
| 15/16 | A555R | | ++ | + | |
| 17/18 | Q456L | | + | + | ++ |
| 19/20 | R550A | | ++ | | |
| 21/22 | N497L | ++ | + | | + |
| 23/24 | E529Y | | + | | ++ |
| 25/26 | Y705W | ++ | + | | |
| 27/28 | D554V | | ++ | ++ | ++ |
| 29/30 | E500D | | ++ | + | |
| 31/32 | T548L | | ++ | + | ++ |
| 33/34 | T582G | | ++ | + | |
| 35/36 | S586W | | ++ | + | |
| 37/38 | G453L | | ++ | | |
| 39/40 | H584C | ++ | + | ++ | + |
| 41/42 | F535G | +++ | ++ | ++ | ++ |
| 43/44 | Q585M | ++ | ++ | ++ | ++ |
| 45/46 | Q592A | ++ | ++ | + | ++ |
| 47/48 | Q459A | | +++ | | ++ |
| 49/50 | T492Q | | ++ | + | |
| 51/52 | Q459R | +++ | ++ | + | ++ |
| 53/54 | A661W | ++ | ++ | | ++ |
| 55/56 | Q592L | | ++ | + | ++ |
| 57/58 | K666I | | ++ | + | +++ |
| 59/60 | F535L | | ++ | + | + |
| 61/62 | A587V | | ++ | + | + |
| 63/64 | G547R | ++ | ++ | + | ++ |
| 65/66 | E500A | | ++ | + | |
| 67/68 | N552S | +++ | + | | |
| 69/70 | Y706W | ++ | ++ | + | + |
| 71/72 | N552W | | ++ | ++ | ++ |
| 73/74 | A661V | | ++ | | |
| 75/76 | A661H | ++ | ++ | ++ | ++ |
| 77/78 | E500L | ++ | ++ | ++ | |
| 79/80 | S586A | ++ | ++ | + | ++ |
| 81/82 | S499G | | + | + | ++ |
| 83/84 | E529V | | ++ | | ++ |
| 85/86 | S586R | | +++ | +++ | + |
| 87/88 | D665M | | ++ | | |
| 89/90 | K666P | + | | | ++ |
| 91/92 | N497R | | ++ | + | |
| 93/94 | T548R | | ++ | | |
| 95/96 | N552R | ++ | ++ | ++ | + |
| 97/98 | D554R | | +++ | + | ++ |
| 99/100 | S538G | ++ | + | + | + |
| 101/102 | T548M | | ++ | | |
| 103/104 | N552V | | ++ | | |
| 105/106 | D556T | | ++ | | + |
| 107/108 | T492R | | ++ | | |
| 109/110 | D532V | ++ | ++ | | |
| 111/112 | Q495N | | ++ | ++ | + |
| 113/114 | Q588R | | +++ | + | |
| 115/116 | T460G | | ++ | +++ | ++ |
| 117/118 | Q456M | | ++ | ++ | + |
| 119/120 | Q456V | ++ | ++ | + | ++ |
| 121/122 | N663R | +++ | ++ | ++ | ++ |
| 123/124 | G453F | | ++ | | ++ |
| 125/126 | D665R | | ++ | | |
| 127/128 | D554I | | ++ | | |
| 129/130 | S454R | ++ | +++ | ++ | + |
| 131/132 | D554S | ++ | ++ | ++ | ++ |
| 133/134 | T660G | | ++ | ++ | + |
| 135/136 | F501N | ++ | | + | ++ |
| 137/138 | D554G | ++ | + | + | |
| 139/140 | V489C | ++ | ++ | + | ++ |
| 141/142 | T460C | | ++ | + | ++ |
| 143/144 | N457G | | + | ++ | ++ |
| 145/146 | E500G | | ++ | ++ | + |
| 147/148 | D554L | ++ | ++ | ++ | |
| 149/150 | G453W | | ++ | + | + |
| 151/152 | N552I | ++ | ++ | ++ | |
| 153/154 | E500R | | ++ | ++ | |
| 155/156 | N709G | | ++ | + | |
| 157/158 | T494L | ++ | ++ | + | ++ |
| 159/160 | Q495F | | ++ | + | |
| 161/162 | P659N | ++ | | | |
| 163/164 | H584V | | ++ | | ++ |
| 165/166 | V493T | ++ | ++ | + | ++ |
| 167/168 | G505Y | | ++ | + | + |
| 169/170 | N668C | | +++ | | ++ |
| 171/172 | G547H | | ++ | | |
| 173/174 | A589R | ++ | +++ | + | |
| 175/176 | H584L | | ++ | + | + |
| 177/178 | F662P | ++ | + | | |
| 179/180 | Q456K | +++ | ++ | + | |
| 181/182 | E500W | ++ | ++ | + | ++ |
| 183/184 | G547D | | ++ | | |
| 185/186 | D556R | | +++ | ++ | |
| 187/188 | Q456P | | ++ | ++ | |
| 189/190 | Q588P | ++ | ++ | + | |
| 191/192 | P659R | | ++ | | |
| 193/194 | Q456F | ++ | ++ | ++ | ++ |
| 195/196 | Q585T | ++ | ++ | + | ++ |
| 197/198 | D532Q | | ++ | + | |
| 199/200 | T548A | ++ | + | + | |
| 201/202 | L667N | | ++ | + | + |
| 203/204 | L537R | ++ | ++ | + | |
| 205/206 | S586V | ++ | ++ | + | + |
| 207/208 | N457L | | ++ | + | ++ |
| 209/210 | G547W | ++ | ++ | ++ | + |
| 211/212 | T492V | | ++ | | |
| 213/214 | N452G | + | ++ | + | ++ |
| 215/216 | G549M | ++ | ++ | ++ | ++ |
| 217/218 | N710V | | ++ | + | |
| 219/220 | T582R | | ++ | ++ | |
| 221/222 | D556G | | ++ | ++ | + |
| 223/224 | T548V | ++ | ++ | | + |
| 225/226 | Q459M | ++ | + | | |
| 227/228 | G453Q | ++ | + | | |
| 229/230 | T460A | | ++ | + | |
| 231/232 | S586G | | + | + | ++ |
| 233/234 | Q588L | | ++ | | ++ |
| 235/236 | D532T | ++ | ++ | + | + |
| 237/238 | Q592Y | | ++ | + | |
| 239/240 | N457T | ++ | ++ | | + |
| 241/242 | T582L | ++ | ++ | | ++ |
| 243/244 | N552K | | ++ | + | |
| 245/246 | D532C | | ++ | | |
| 247/248 | E500H | ++ | ++ | ++ | ++ |
| 249/250 | A555S | | ++ | + | + |
| 251/252 | M640L | + | + | ++ | |
| 253/254 | E529A | | ++ | + | ++ |
| 255/256 | L25S/T492W | | ++ | ++ | |
| 257/258 | D665W | ++ | ++ | | ++ |
| 259/260 | D554Q | + | ++ | ++ | ++ |
| 261/262 | Q585I | | ++ | + | ++ |
| 263/264 | N457R | ++ | ++ | + | ++ |
| 265/266 | K545P | ++ | | | + |
| 267/268 | V211M/E416T | ++ | + | ++ | ++ |
| 269/270 | G539A | ++ | ++ | + | + |
| 271/272 | E529D | ++ | ++ | + | |
| 273/274 | A589T/M640L | | ++ | + | + |
| 275/276 | Q592G | + | + | | + |
| 277/278 | Q588R/D665G | + | +++ | ++ | |
| 279/280 | D551F | ++ | ++ | + | |
| 281/282 | P504T/A510H | ++ | ++ | ++ | |
| 283/284 | S586K | +++ | +++ | ++ | ++ |
| 285/286 | N710G | | ++ | ++ | + |
| 287/288 | G455R | | ++ | ++ | ++ |
| 289/290 | D551M | ++ | | | |
| 291/292 | S508K | ++ | ++ | ++ | |
| 293/294 | N457F | ++ | ++ | + | + |
| 295/296 | V465Q/A589T/ | | ++ | + | ++ |
| | M640L | | | | |
| 297/298 | N497S | ++ | ++ | ++ | + |
| 299/300 | N419D/P504T/ | ++ | ++ | + | |
| | S508K/A510H | | | | |
| 301/302 | L667T | | + | + | |
| 303/304 | G549W | | | | ++ |
| 305/306 | K528I | | + | | ++ |
| 307/308 | N497C | | | | ++ |
| 309/310 | D532R | | + | + | ++ |
| 311/312 | A661S | | | | ++ |
| 313/314 | S490P | | | | ++ |
| 315/316 | K664G | | + | + | ++ |
| 317/318 | V553K | | | | ++ |
| 319/320 | G505T | | + | + | ++ |
| 321/322 | Q592H | | + | | ++ |
| 323/324 | S586I | | + | + | ++ |
| 325/326 | T660M | | + | ++ | |
| 327/328 | Q458S | | + | | ++ |
| 329/330 | T494I | | + | ++ | + |
| 403/404 | K449R/H584L | | + | + | ++ |
| 407/408 | D384N/I479L/ | | | | ++ |
| | P504T/S508K/ | | | | |
| | A510H | | | | |
| 411/412 | S507T/W509Y/ | | | | ++ |
| | E529D | | | | |
| 423/424 | S490V | | | | ++ |

Levels of increased performance in all conditions were determined relative to the Capsid protein reference polypeptide of SEQ ID NO: 2. Levels of increased performance for the CNS transduction assay are defined as follows: “+” > 0.9, “++” > 1.3, “+++” > 3. Levels of increased performance in the HEK293T transduction assay are defined as follows: “+” > 0.9, “++” > 1.3, “+++” > 3. Levels of increased performance in the HEK293T transduction assay in the presence of Nabs are defined as follows: “+” > 0.9, “++” > 1.5, “+++” > 3. Levels of increased activity in the qPCR assay are defined as follows: “+” > 0.9, “++” > 1.4, “+++” > 3.
Note:
the sequence of MAAP (SEQ ID NO: 1135/1136) and AAP (SEQ ID NO: 1233/1234) are identical for all variants

TABLE 5-2
Performance of AAV variants relative to SEQ ID NO: 278

| SEQ ID | Amino Acid | | Amino Acid | | Amino Acid | | | | |
|---|---|---|---|---|---|---|---|---|---|
| NO | Differences in | SEQ ID | Differences in | | Differences in | | | FIOP | |
| Capsid | Capsid protein | NO | MAAP (Relative | SEQ ID | AAP (Relative | FIOP | FIOP | HEK293T + | FIOP |
| protein: | (Relative to | MAAP | to SEQ ID | NO AAP | to SEQ ID | CNS | HEK293T | Nab | qPCR |
| (nt/aa) | SEQ ID NO: 2) | (nt/aa) | NO: 1136) | (nt/aa) | NO: 1234) | assay | assay | assay | assay |
| 331/332 | A510H/M640L | 1137/1138 | F99C | 1233/1234 | | | ++ | + | + |
| 333/334 | Q233T/S508K | 1135/1136 | | 1237/1238 | N58R | | | | ++ |
| 335/336 | G56F/E529D | 1139/1140 | A30L | 1233/1234 | | + | ++ | ++ | + |
| 337/338 | V331T/K449R | 1135/1136 | | 1239/1240 | S156R | ++ | ++ | + | ++ |
| 339/340 | V211M/W509Y/E712D | 1141/1142 | A30V | 1241/1242 | P49H/S156* | | | | ++ |
| 341/342 | V331T/E416T/Y478W | 1141/1142 | A30V | 1239/1240 | S156R | | + | | +++ |
| 343/344 | N668A | 1137/1138 | F99C | 1233/1234 | | | ++ | ++ | |
| 345/346 | G56F/S507T | 1139/1140 | A30L | 1243/1244 | S156* | | | | ++ |
| 347/348 | G56F/V331T/H584L/ | 1139/1140 | A30L | 1239/1240 | S156R | | ++ | + | |
| | Q597N | | | | | | | | |
| 349/350 | G56F/Y478W | 1139/1140 | A30L | 1233/1234 | | | + | + | ++ |
| 351/352 | D349E/V465Q/I479L/ | 1137/1138 | F99C | 1245/1246 | T174S | +++ | ++ | ++ | + |
| | P504T/S508K | | | | | | | | |
| 353/354 | D349E/M640L | 1137/1138 | F99C | 1245/1246 | T174S | ++ | + | + | + |
| 355/356 | V331T/H584L/Q597N | 1141/1142 | A30V | 1239/1240 | S156R | ++ | + | + | ++ |
| 357/358 | V331T/E416T/Y478W/ | 1141/1142 | A30V | 1239/1240 | S156R | + | ++ | + | ++ |
| | S483C/S507T/H584L/ | | | | | | | | |
| | Q597N | | | | | | | | |
| 359/360 | G56F/E416T | 1139/1140 | A30L | 1233/1234 | | ++ | +++ | ++ | ++ |
| 361/362 | Q233T/V465Q/M640L | 1137/1138 | F99C | 1237/1238 | N58R | +++ | + | + | ++ |
| 363/364 | L125V/S508K/A510H/ | 1143/1144 | F99C | 1233/1234 | | ++ | | | ++ |
| | A589T | | | | | | | | |
| 365/366 | Q597N | 1141/1142 | A30V | 1233/1234 | | | | + | ++ |
| 367/368 | V331T/Q597N | 1135/1136 | | 1239/1240 | S156R | | ++ | ++ | ++ |
| 369/370 | D665A | 1141/1142 | A30V | 1233/1234 | | ++ | | | |
| 371/372 | G56F/V331T/E416T/ | 1139/1140 | A30L | 1239/1240 | S156R | + | ++ | + | ++ |
| | D665A | | | | | | | | |
| 373/374 | L125V/V465Q/P504T/ | 1143/1144 | F99C | 1233/1234 | | +++ | ++ | | |
| | S508K/A510H | | | | | | | | |
| 375/376 | G56F/V331T/Y478W/ | 1139/1140 | A30L | 1239/1240 | S156R | | ++ | + | ++ |
| | S483C/E529D/Q597N | | | | | | | | |
| 377/378 | D384N/I479L | 1137/1138 | F99C | 1233/1234 | | | | + | ++ |
| 379/380 | Q233T/M640L | 1135/1136 | | 1237/1238 | N58R | ++ | ++ | ++ | + |
| 381/382 | L125V/N419D/P504T/ | 1143/1144 | F99C | 1233/1234 | | | ++ | + | ++ |
| | S508K/G530D | | | | | | | | |
| 383/384 | P504T/A510H/M640L | 1137/1138 | F99C | 1233/1234 | | ++ | ++ | ++ | |
| 385/386 | N419D/P504T/S508K | 1135/1136 | | 1247/1248 | T174I | ++ | ++ | + | |
| 387/388 | G56F/V331T/E361Q | 1139/1140 | A30L | 1249/1250 | S156R | + | ++ | ++ | |
| 389/390 | E416T/D665A | 1141/1142 | A30V | 1233/1234 | | | | ++ | |
| 391/392 | G56F/V211M/E361Q/ | 1139/1140 | A30L | 1251/1252 | | ++ | ++ | ++ | + |
| | E416T/Y478W/H584L | | | | | | | | |
| 393/394 | G56F | 1139/1140 | A30L | 1233/1234 | | | | + | ++ |
| 395/396 | E361Q/S483C | 1135/1136 | | 1253/1254 | | | | | ++ |
| 397/398 | G56F/V331T/E416T | 1139/1140 | A30L | 1239/1240 | S156R | ++ | ++ | ++ | |
| 399/400 | Q175E/V211M | 1141/1142 | A30V | 1255/1256 | S156* | | ++ | + | + |
| 401/402 | G56F/V211M/Y478W/ | 1139/1140 | A30L | 1235/1236 | | ++ | ++ | + | |
| | S483C/E712D | | | | | | | | |
| 405/406 | D349E/P504T/S508K | 1137/1138 | F99C | 1245/1246 | T174S | ++ | ++ | ++ | ++ |
| 409/410 | L125V/S508K/A510H | 1143/1144 | F99C | 1233/1234 | | | | + | ++ |
| 413/414 | V331T/E416T | 1141/1142 | A30V | 1239/1240 | S156R | | + | + | ++ |
| 415/416 | V331T | 1141/1142 | A30V | 1239/1240 | S156R | | + | | ++ |
| 417/418 | I479L/P504T/A510H/ | 1137/1138 | F99C | 1233/1234 | | ++ | ++ | ++ | + |
| | A589T | | | | | | | | |
| 419/420 | V331T/Y478W | 1141/1142 | A30V | 1239/1240 | S156R | ++ | ++ | + | ++ |
| 421/422 | G56F/V331T/Y478W/ | 1139/1140 | A30L | 1239/1240 | S156R | | ++ | + | ++ |
| | S483C/Q597N | | | | | | | | |
| 425/426 | I479L | 1135/1136 | | 1247/1248 | T174I | ++ | | | + |
| 427/428 | L125V/P504T/S508K | 1143/1144 | F99C | 1233/1234 | | ++ | ++ | ++ | + |
| 429/430 | I479L/A589T | 1135/1136 | | 1247/1248 | T174I | | ++ | | |
| 431/432 | I479L/A510H/A589T | 1137/1138 | F99C | 1233/1234 | | +++ | ++ | + | ++ |

Levels of increased performance in all conditions were determined relative to the Capsid protein reference polypeptide of SEQ ID NO: 2. Levels of increased performance for the CNS transduction assay are defined as follows: “+” >0.9, “++” >1.3, “+++” >3. Levels of increased performance in the HEK293T transduction assay are defined as follows: “+” > 0.9, “++” >1.3, “+++” >3. Levels of increased performance in the HEK293T transduction assay in the presence of Nabs are defined as follows: “+” >0.9. “++” >1.5, “+++” >3. Levels of increased activity in the qPCR assay are defined as follows: “+” >0.9, “++” >1.4, “+++” >3
Note:
the sequences of MAAP and AAP for some Capsid protein variants differ from the reference sequences of MAAP and AAP, as specified in the table

### Example 6

### AAV Capsid Variants Derived from SEQ ID NO: 278

In this Example, experiments conducted to determine AAV capsid variant characteristics are described. These variants were assessed for their titer, transduction, and neutralization using the assays described in Example 4. Results are shown in Table 6.1 and Table 6.2. For Table 6.1, the sequences of MAAP (SEQ ID NO: 1135/1136) and AAP (SEQ ID NO: 1233/1234) are identical for all variants. In Table 6.2, the sequence of AAP (SEQ ID NO: 1233/1234) is identical for all variants, and the sequences of MAAP for Capsid protein variants differ from the reference sequence of MAAP, as specified in the table.

TABLE 6-1
Performance of AAV variants relative to SEQ ID NO: 278

| SEQ | Amino Acid | | | | |
|---|---|---|---|---|---|
| ID NO | Differences in | | | | |
| Capsid | Capsid protein | FIOP | FIOP | FIOP | FIOP |
| protein: | (Relative to | CNS | HEK293T | HEK293T + | qPCR |
| (nt/aa) | SEQ ID NO: 278) | assay | assay | Nab assay | assay |
| 433/434 | N14R | | ++ | ++ | |
| 435/436 | F173S | + | | | + |
| 437/438 | I159R | | ++ | + | |
| 439/440 | A157T | ++ | + | ++ | |
| 441/442 | E152Q | | ++ | + | |
| 443/444 | R485G | | ++ | + | |
| 445/446 | P421G | ++ | + | | |
| 447/448 | R391Q | ++ | | | ++ |
| 449/450 | S469W | | ++ | ++ | |
| 451/452 | N628Q | ++ | + | | ++ |
| 453/454 | L461W | +++ | | | |
| 455/456 | Q165G | | ++ | ++ | |
| 457/458 | N14L | | ++ | +++ | |
| 459/460 | S469D | | ++ | | |
| 461/462 | H642E | | ++ | | |
| 463/464 | W595F | ++ | | + | |
| 465/466 | A24R | | ++ | ++ | |
| 467/468 | S414R | | ++ | | |
| 469/470 | N512G | | ++ | + | ++ |
| 471/472 | N519S | + | ++ | ++ | +++ |
| 473/474 | A157S | | ++ | | + |
| 475/476 | N14V | | ++ | ++ | |
| 477/478 | N172W | | ++ | ++ | |
| 479/480 | K168R | | ++ | | |
| 481/482 | N598W | | ++ | ++ | |
| 483/484 | K449E | ++ | | | + |
| 485/486 | K449R | | ++ | | |
| 487/488 | L736M | ++ | | | ++ |
| 489/490 | N598T | | ++ | | |
| 491/492 | S156G | | ++ | + | |
| 493/494 | A24E | | ++ | +++ | |
| 495/496 | K462A | | ++ | ++ | |
| 497/498 | A24T | | ++ | ++ | |
| 499/500 | W595G | | ++ | | |
| 501/502 | S414Q | | ++ | + | |
| 503/504 | R485S | ++ | ++ | ++ | + |
| 505/506 | S162V | | ++ | | ++ |
| 507/508 | R485L | | ++ | ++ | |
| 509/510 | H642W | | ++ | ++ | |
| 511/512 | E416Y | | ++ | + | |
| 513/514 | E416S | | ++ | +++ | |
| 515/516 | Q456I | + | ++ | + | + |
| 517/518 | A587I | | ++ | ++ | |
| 519/520 | S586N | | ++ | + | + |
| 521/522 | N668R | +++ | | + | + |
| 523/524 | V465K | ++ | | | + |
| 525/526 | S454P | | ++ | ++ | + |
| 527/528 | Q495F/G505Y/ | ++ | | | |
| | D532T | | | | |
| 529/530 | T494L/N497S | ++ | ++ | + | ++ |
| 531/532 | N497S/T548V/ | | ++ | + | + |
| | N552I/K664G | | | | |
| 533/534 | A661H | | ++ | ++ | + |
| 535/536 | N497S/G505Y/ | +++ | | | ++ |
| | S586I | | | | |
| 537/538 | Q495F | ++ | | | |
| 539/540 | A555W | | ++ | | |
| 541/542 | Q585M/Y706W | +++ | | | |
| 543/544 | Q456F | ++ | | | + |
| 545/546 | Y706W | | ++ | | ++ |
| 547/548 | E416T/M640L | | ++ | + | |
| 549/550 | Q456F/N457G/ | +++ | | | |
| | T494L/N497S/ | | | | |
| | G505Y/A661H/ | | | | |
| | Y706W | | | | |
| 551/552 | N457G/Q495F | +++ | + | | +++ |
| 553/554 | G505Y | | ++ | | |
| 555/556 | Q456F/Q495F/ | +++ | | | + |
| | G505Y | | | | |
| 557/558 | N457G/N497S/ | +++ | | | |
| | G549M/N552I/ | | | | |
| | K664G | | | | |
| 559/560 | T494L/G505Y/ | ++ | ++ | | |
| | Q592A | | | | |
| 561/562 | Q456F/N457G | ++ | | | + |
| 563/564 | T548V/N552I/ | ++ | | | |
| | Q592A/A661H/ | | | | |
| | Y706W | | | | |
| 565/566 | Q456F/N457G/ | ++ | | | |
| | T494L/N497S/ | | | | |
| | G505Y/A661H | | | | |
| 567/568 | T494L | | ++ | + | |
| 569/570 | T494L/Q495F/ | ++ | | | ++ |
| | G505Y/K664G | | | | |
| 571/572 | G505Y/K664G | ++ | + | | ++ |
| 573/574 | Q456F/T494L/ | ++ | ++ | | + |
| | N497S/A661H | | | | |
| 575/576 | V493T/M640L | | ++ | | |
| 577/578 | N457G/T548V/ | | ++ | + | |
| | A555W/Q592A/ | | | | |
| | A661H | | | | |
| 579/580 | A661H/K664G | | ++ | + | ++ |
| 581/582 | N457G/Q592A/ | | ++ | | |
| | A661H | | | | |
| 583/584 | E416T/A661W | + | ++ | ++ | + |
| 585/586 | K664G | | ++ | | |
| 587/588 | Q592A/K664G | +++ | ++ | ++ | + |
| 589/590 | N457G/T494L/ | +++ | ++ | + | |
| | N497S | | | | |
| 591/592 | N457G/A661H/ | | ++ | | +++ |
| | K664G | | | | |
| 593/594 | Q456F/N497S/ | +++ | ++ | | |
| | Q592A | | | | |
| 595/596 | N497S/G505Y | ++ | | | ++ |
| 597/598 | S155G | | | + | +++ |
| 599/600 | N663R | | | +++ | |
| 601/602 | P401A | | | ++ | |
| 603/604 | A157L | | | | ++ |
| 605/606 | W22R | | | | ++ |
| 607/608 | Q175G | | | | ++ |
| 609/610 | Q579L | | | | + |
| 613/614 | N172E | | | ++ | |
| 615/616 | A24W | | | + | |
| 617/618 | L444V | | | | ++ |
| 619/620 | W23R | | | ++ | |
| 621/622 | P421R | | | | ++ |
| 623/624 | Q459R/S508K/ | | | | ++ |
| | N663R/L667T | | | | |
| 625/626 | S414T | | ++ | | |
| 627/628 | E17T | | | | ++ |
| 629/630 | K462M | | ++ | | ++ |
| 631/632 | Q412R | | + | + | |
| 635/636 | W22I | | | | +++ |
| 637/638 | L461G | | | | ++ |
| 639/640 | A157Q | | | | ++ |
| 641/642 | R391L | | | | ++ |
| 643/644 | R485W | | + | + | ++ |
| 647/648 | F173T | | | + | ++ |
| 649/650 | I440A | | | | ++ |
| 651/652 | Q387G | | | | ++ |
| 653/654 | I159C | | + | | + |
| 655/656 | G453R/S586K | | | | ++ |
| 657/658 | N419G | | | | + |
| 659/660 | L25Q | | | | ++ |
| 661/662 | A472W | | | | +++ |
| 663/664 | K462E | | + | +++ | ++ |
| 665/666 | S162G/T450N | | | | ++ |
| 667/668 | W22M | | | | + |
| 669/670 | S414D | | | | ++ |
| 671/672 | Q585T | | + | | ++ |
| 673/674 | Q175D | | + | | |
| 675/676 | S156W | | | ++ | |
| 677/678 | N437V | | | | ++ |
| 679/680 | A157C | | ++ | + | ++ |
| 681/682 | N512L | | | ++ | |
| 683/684 | N598G | | + | ++ | |
| 685/686 | K449T | | | | ++ |
| 687/688 | F173W | | | ++ | |
| 689/690 | A472Q | | | | + |
| 691/692 | L25G | | | | ++ |
| 693/694 | W10I | | | | +++ |
| 695/696 | S162G | | | | ++ |
| 697/698 | N562L | | | | ++ |
| 699/700 | K169V | | | | ++ |
| 701/702 | S155R | | | | +++ |
| 703/704 | H642T | | | | ++ |
| 705/706 | A24V | | + | + | + |
| 707/708 | R588V | | | ++ | + |
| 709/710 | R588S | | | ++ | + |
| 711/712 | A587Q | | + | | |
| 713/714 | R588T | | | | + |
| 715/716 | N562R | | | ++ | |
| 717/718 | E565K | | | | ++ |
| 719/720 | A589Y | | + | + | |
| 721/722 | Q495F/N497S | | + | | +++ |

Levels of increased performance in all conditions were determined relative to the Capsid protein reference polypeptide of SEQ ID NO: 278. Levels of increased performance for the CNS transduction assay are defined as follows: “+” > 0.9, “++” > 1.4, “+++” > 3. Levels of increased performance in the HEK293T transduction assay are defined as follows: “+” > 0.9, “++” > 1.3, “+++” > 3. Levels of increased performance in the HEK293T transduction assay in the presence of Nabs are defined as follows: “+” > 0.9, “++” > 1.5, “+++” > 3. Levels of increased activity in the qPCR assay are defined as follows: “+” > 0.9, “++” > 1.3, “+++” > 3
Note:
the sequences of MAAP (SEQ ID NO: 1135/1136) and AAP (SEQ ID NO: 1233/1234) are identical for all variants

TABLE 6-2
Performance of AAV variants relative to SEQ ID NO: 278

| SEQ ID | Amino Acid | | Amino Acid | | | |
|---|---|---|---|---|---|---|
| NO | Differences in | SEQ ID | Differences in | | FIOP | |
| Capsid | Capsid protein | NO | MAAP | FIOP | HEK293T + | FIOP |
| protein: | (Relative to SEQ | MAAP | (Relative to SEQ | HEK293T | Nab | qPCR |
| (nt/aa) | ID NO: 278) | (nt/aa) | ID NO: 1136) | assay | assay | assay |
| 611/612 | R485P | 1145/1146 | E17G | + | + | ++ |
| 633/634 | F119S/T450G | 1147/1148 | S93P | | | ++ |
| 645/646 | K142N/N552S | 1149/1150 | R116T | | | ++ |

Levels of increased performance in all conditions were determined relative to the Capsid protein reference polypeptide of SEQ ID NO: 278. Levels of increased performance in the HEK293T transduction assay are defined as follows: “+” >1, “++” >1.3. Levels of increased performance in the HEK293T transduction assay in the presence of Nabs are defined as follows: “+” >1, “++” >1.5. Levels of increased activity in the qPCR assay are defined as follows: “+” >0.9, “++” >1.3
Note:
the sequence of AAP (SEQ ID NO: 1233/1234) is identical for all variants. The sequences of MAAP for Capsid protein variants differ from the reference sequence of MAAP, as specified in the table

### Example 7

### AAV Capsid Variants Derived from SEQ ID NO: 552

In this Example, experiments conducted to determine AAV capsid variant characteristics are described. These variants were assessed for their titer, transduction, and neutralization using the assays described in Example 4. Results are shown in Table 7.1 and Table 7.2. In Table 7.1, the sequences of MAAP (SEQ ID NO: 1135/1136) and AAP (SEQ ID NO: 1233/1234) are identical for all variants. In Table 7.2, the sequences of MAAP and AAP for some Capsid protein variants differ from the reference sequences of MAAP and AAP, as specified in the table.

TABLE 7-1
Performance of AAV variants relative to SEQ ID NO: 552

| SEQ | Amino Acid | | | | |
|---|---|---|---|---|---|
| ID NO | Differences in | | | | |
| Capsid | Capsid protein | FIOP | FIOP | FIOP | FIOP |
| protein: | (Relative to | CNS | HEK293T | HEK293T + | qPCR |
| (nt/aa) | SEQ ID NO: 552) | assay | assay | Nab assay | assay |
| 723/724 | R725G | | | | + |
| 725/726 | S414R/Q456N/ | | | | ++ |
| | Q459C/R485P/ | | | | |
| | L736M | | | | |
| 729/730 | A157W/Q165V/ | | ++ | | + |
| | S454Q | | | | |
| 731/732 | A157W/Q165V/ | | ++ | | |
| | N668R | | | | |
| 733/734 | R485S/W595F | ++ | ++ | ++ | ++ |
| 735/736 | Q165V/S454Q/ | ++ | | | ++ |
| | R485S | | | | |
| 739/740 | P421G/Q456N/ | + | | | |
| | Q459C | | | | |
| 747/748 | P421G | + | ++ | | |
| 749/750 | D551F | | ++ | | |
| 751/752 | Q165V | | ++ | | + |
| 753/754 | L736M | + | ++ | | |
| 755/756 | E529A/N552S/ | + | | | |
| | Q585T | | | | |
| 757/758 | F535G | ++ | | | |
| 759/760 | E529A/Q585T | + | | | |
| 761/762 | F535G/P659Q | ++ | | | |
| 767/768 | G594W | + | | ++ | + |
| 769/770 | P468V | + | | | |
| 775/776 | P726H | | | | + |
| 783/784 | G594V | | | | |
| 785/786 | P468W/Q592L | + | | | |
| 787/788 | I451P | + | | | |
| 795/796 | G594T | + | | | + |
| 797/798 | M559C | + | ++ | | ++ |
| 807/808 | E575D | + | ++ | ++ | ++ |
| 809/810 | V465P | + | | | |
| 811/812 | V465I | + | ++ | | |
| 819/820 | I560L | + | | | |
| 823/824 | P468A | + | + | + | |
| 827/828 | I451L | ++ | ++ | | + |
| 829/830 | G594N | + | ++ | ++ | |
| 833/834 | V389M/A510C | + | | | |
| 835/836 | I451Y | + | ++ | | |
| 843/844 | N470Q | + | + | + | |
| 853/854 | V465T | + | ++ | + | |
| 855/856 | P468Q | + | | | + |
| 857/858 | G594Q | + | + | + | |
| 859/860 | G594S | ++ | + | | |
| 875/876 | P724V | + | + | | |
| 877/878 | R485Q/Q597I | + | ++ | ++ | |
| 879/880 | A510T | ++ | | | + |
| 889/890 | V211M | ++ | | | ++ |
| 897/898 | G594M | | ++ | | |
| 899/900 | R725A | | | | + |
| 901/902 | W595F | | ++ | | |
| 903/904 | R391Q | | | | ++ |
| 905/906 | R391Q/A661K | | | | ++ |
| 909/910 | Q165V/R391Q/ | | | | + |
| | S454Q/M471I | | | | |
| 911/912 | A157W/Q165V/ | | | | + |
| | R391Q | | | | |
| 913/914 | Q165V/R391Q | | | | + |
| 915/916 | A157W/R391Q/ | | | | ++ |
| | P421T | | | | |
| 919/920 | P401G/S414R/ | + | | | |
| | R485P | | | | |
| 921/922 | R391Q/S454Q/ | | | | ++ |
| | W595F | | | | |
| 923/924 | E529A | | | | + |
| 925/926 | E529A/N552S/ | | | | ++ |
| | R588Q/N716D | | | | |
| 927/928 | V493T/Q585T/ | ++ | | | |
| | L667T | | | | |
| 931/932 | N552S | | ++ | | ++ |
| 933/934 | N515V | | | | + |
| 941/942 | P724I | + | | | ++ |
| 949/950 | V465N | ++ | + | | |
| 955/956 | I451G | + | + | ++ | ++ |
| 963/964 | I451H | ++ | ++ | | ++ |
| 965/966 | E565T | | | | ++ |
| 967/968 | A510G | ++ | | | + |
| 975/976 | I451S | ++ | | | |
| 979/980 | N470G/N663D | ++ | ++ | | + |
| 985/986 | S414R | ++ | + | | |
| 993/994 | Q597C | | | | + |
| 999/1000 | V465L | + | | | + |
| 1011/1012 | Q597I | + | | | |
| 1021/1022 | E565G | | | | ++ |
| 1025/1026 | E564D | | | | ++ |
| 1033/1034 | R725W | | | | ++ |
| 1037/1038 | V389P | + | | | + |
| 1053/1054 | I451T | + | ++ | | ++ |
| 1055/1056 | A510E | + | | | + |
| 1061/1062 | P468Y | | + | + | + |
| 1063/1064 | N515T | ++ | | | |
| 1069/1070 | E564A | | | | ++ |
| 1073/1074 | A510C | ++ | | | |
| 1077/1078 | N515L | | | | + |
| 1083/1084 | P468W | + | ++ | | |
| 1089/1090 | P726I | | | | + |
| 1093/1094 | V720F | ++ | | | |
| 1097/1098 | I451V/N452D | | | | ++ |
| 1099/1100 | I451N | + | | | + |
| 1101/1102 | A510I | ++ | ++ | | |
| 1103/1104 | N496D | ++ | | | |
| 1105/1106 | V389F | | | | ++ |
| 1121/1122 | A510Q | + | + | | + |
| 1127/1128 | R725M | | | | ++ |

Levels of increased performance in all conditions were determined relative to the Capsid protein reference polypeptide of SEQ ID NO: 552. Levels of increased performance for the CNS transduction assay are defined as follows: “+” > 0.9, “++” > 1.3, “+++” > 3. Levels of increased performance in the HEK293T transduction assay are defined as follows: “+” > 0.9, “++” > 1.3, “+++” > 3. Levels of increased performance in the HEK293T transduction assay in the presence of Nabs are defined as follows: “+” > 0.9, “++” > 1.6, “+++” > 3. Levels of increased activity in the qPCR assay are defined as follows: “+” > 0.9, “++” > 1.3, “+++” > 3
Note:
the sequences of MAAP (SEQ ID NO: 1135/1136) and AAP (SEQ ID NO: 1233/1234) are identical for all variants

TABLE 7-2
Performance of AAV variants relative to SEQ ID NO: 552

| | Amino Acid | | | | Amino Acid | | | | |
|---|---|---|---|---|---|---|---|---|---|
| SEQ ID | Differences in | | Amino Acid | | Differences | | | | |
| NO | Capsid | SEQ ID | Differences in | | in AAP | | | FIOP | |
| Capsid | protein | NO | MAAP (Relative | SEQ ID | (Relative to | FIOP | FIOP | HEK293T + | FIOP |
| protein: | (Relative to | MAAP | to SEQ ID NO: | NO AAP | SEQ ID NO: | CNS | HEK293T | Nab | qPCR |
| (nt/aa) | SEQ ID NO: 2) | (nt/aa) | 1136) | (nt/aa) | 1234) | assay | assay | assay | assay |
| 727/728 | A193S | 1135/1136 | | 1257/1258 | | + | ++ | +++ | |
| 737/738 | T346S | 1135/1136 | | 1259/1260 | | ++ | ++ | ++ | |
| 741/742 | A218S | 1135/1136 | | 1261/1262 | | + | + | | |
| 743/744 | S205P | 1135/1136 | | 1263/1264 | | + | ++ | ++ | |
| 745/746 | S348T | 1135/1136 | | 1265/1266 | | | ++ | +++ | |
| 763/764 | T333S | 1135/1136 | | 1267/1268 | R157S/P158L | | ++ | ++ | + |
| 765/766 | | 1135/1136 | | 1269/1270 | P160R | + | + | | |
| 771/772 | Q101E | 1151/1152 | | 1233/1234 | | ++ | | | ++ |
| 773/774 | | 1135/1136 | | 1271/1272 | P158L | ++ | ++ | | |
| 777/778 | E190A | 1135/1136 | | 1273/1274 | N15R | + | ++ | ++ | + |
| 779/780 | | 1153/1154 | L27* | 1233/1234 | | + | | | |
| 781/782 | N254C | 1135/1136 | | 1275/1276 | I79V | | | +++ | |
| 789/790 | | 1135/1136 | | 1277/1278 | P104H | ++ | | | ++ |
| 791/792 | F100T | 1155/1156 | S73R/S74L | 1233/1234 | | | | | ++ |
| 793/794 | | 1135/1136 | | 1279/1280 | V31A/S156L | ++ | | | ++ |
| 799/800 | E190D/R725H | 1135/1136 | | 1281/1282 | N15I | + | | | + |
| 801/802 | Q31L | 1157/1158 | N5Y | 1233/1234 | | + | ++ | | + |
| 803/804 | T333I | 1135/1136 | | 1283/1284 | P158S | ++ | + | | |
| 805/806 | Q101R | 1159/1160 | R75E | 1233/1234 | | + | | | |
| 813/814 | K258T | 1135/1136 | | 1285/1286 | S83L | | + | ++ | |
| 815/816 | S268I | 1135/1136 | | 1287/1288 | D92E/L93S | | | | +++ |
| 817/818 | N214D/I451L | 1135/1136 | | 1289/1290 | T102I | ++ | ++ | + | |
| 821/822 | Q101V | 1161/1162 | R75W | 1233/1234 | | + | | + | ++ |
| 825/826 | V331R | 1135/1136 | | 1291/1292 | E155D/S156V | + | | | |
| 831/832 | A273T | 1135/1136 | | 1293/1294 | | ++ | ++ | | |
| 837/838 | E190L | 1135/1136 | | 1295/1296 | E14D/N15Y | ++ | ++ | | |
| 839/840 | N328T | 1135/1136 | | 1297/1298 | T153R | + | ++ | ++ | |
| 841/842 | K33L | 1163/1164 | R7W | 1233/1234 | | ++ | ++ | + | ++ |
| 845/846 | | 1135/1136 | | 1299/1300 | R194P | + | | | |
| 847/848 | Y274F | 1135/1136 | | 1301/1302 | T99S | | + | + | |
| 849/850 | F100L | 1165/1166 | | 1233/1234 | | | | | ++ |
| 851/852 | K33R/S576Y | 1167/1168 | R7A | 1233/1234 | | ++ | | | |
| 861/862 | T333N | 1135/1136 | | 1303/1304 | P158I | ++ | ++ | | |
| 863/864 | | 1135/1136 | | 1305/1306 | S85Y | + | | | |
| 865/866 | V331T | 1135/1136 | | 1307/1308 | S156Q | + | | | |
| 867/868 | I560H | 1135/1136 | | 1309/1310 | P105L | + | | | + |
| 869/870 | K33G | 1169/1170 | R7G | 1233/1234 | | + | + | ++ | |
| 871/872 | E190C | 1135/1136 | | 1311/1312 | E14D/N15A | + | | | |
| 873/874 | K51E/E190V | 1171/1172 | | 1313/1314 | N15S | ++ | | | |
| 881/882 | L45F | 1173/1174 | | 1233/1234 | | ++ | | | ++ |
| 883/884 | L256I | 1135/1136 | | 1315/1316 | | ++ | | ++ | ++ |
| 885/886 | I334V | 1135/1136 | | 1317/1318 | | + | | + | |
| 887/888 | E63Q | 1175/1176 | | 1233/1234 | | ++ | | | ++ |
| 891/892 | T264S | 1135/1136 | | 1319/1320 | | | | | ++ |
| 893/894 | D60S | 1177/1178 | T34V | 1233/1234 | | + | | | |
| 895/896 | Y79H/N272G | 1179/1180 | | 1321/1322 | T97G | | | | + |
| 907/908 | S200P | 1135/1136 | | 1323/1324 | | + | ++ | +++ | |
| 917/918 | Q81E | 1181/1182 | | 1233/1234 | | + | | | |
| 929/930 | S269T | 1135/1136 | | 1325/1326 | | ++ | | | |
| 935/936 | S263A | 1135/1136 | | 1327/1328 | A88L | + | | | + |
| 937/938 | N270L | 1135/1136 | | 1329/1330 | Q94H/M95W | | | | ++ |
| 939/940 | | 1135/1136 | | 1331/1332 | A88L | ++ | + | | + |
| 943/944 | F100P | 1183/1184 | S74L | 1233/1234 | | | | | ++ |
| 945/946 | S263T | 1135/1136 | | 1333/1334 | A88L | | | | ++ |
| 947/948 | G267L | 1135/1136 | | 1335/1336 | E91D/D92C | | | | ++ |
| 951/952 | | 1135/1136 | | 1337/1338 | S100L | + | | | + |
| 953/954 | P210S/G266F | 1135/1136 | | 1339/1340 | E91S | | | | ++ |
| 957/958 | | 1135/1136 | | 1341/1342 | D92V | + | + | | + |
| 959/960 | D60K | 1185/1186 | T34R | 1233/1234 | | + | | | |
| 961/962 | Q259K | 1135/1136 | | 1343/1344 | S83R/K84R | | | | + |
| 969/970 | G267L | 1135/1136 | | 1345/1346 | E91D/D92S | | | | ++ |
| 971/972 | L53R | 1187/1188 | L27V | 1233/1234 | | | | | ++ |
| 973/974 | S263T | 1135/1136 | | 1347/1348 | A88R | | | | + |
| 977/978 | K33F | 1189/1190 | R7L | 1233/1234 | | + | | | |
| 981/982 | Q259S/A466T | 1135/1136 | | 1349/1350 | S83R/K84V | | | | ++ |
| 983/984 | F100A | 1191/1192 | S73R/S74R | 1233/1234 | | | | | + |
| 987/988 | V331T | 1135/1136 | | 1351/1352 | S156P | + | ++ | ++ | |
| 989/990 | Y90L | 1193/1194 | T64F | 1233/1234 | | | | | ++ |
| 991/992 | E190H | 1135/1136 | | 1353/1354 | E14D/N15T | ++ | | + | ++ |
| 995/996 | T333A | 1135/1136 | | 1355/1356 | P158R | + | + | | |
| 997/998 | Y90G | 1195/1196 | T64V | 1233/1234 | | | | | ++ |
| 1001/1002 | Q101S | 1197/1198 | | 1233/1234 | | ++ | ++ | +++ | |
| 1003/1004 | T264E | 1135/1136 | | 1357/1358 | H89S | | | | ++ |
| 1005/1006 | N270H | 1135/1136 | | 1359/1360 | Q94H | | | | ++ |
| 1007/1008 | N172D/G266V | 1135/1136 | | 1361/1362 | E91L | | | | + |
| 1009/1010 | Y90R | 1199/1200 | T64G | 1233/1234 | | | | | ++ |
| 1013/1014 | Y90A/F535G | 1201/1202 | T64P | 1233/1234 | | | | | ++ |
| 1015/1016 | G267V | 1135/1136 | | 1363/1364 | D92S | | | | +++ |
| 1017/1018 | N270P | 1135/1136 | | 1365/1366 | Q94H/M95R/ | | | | ++ |
| | | | | | Q173R | | | | |
| 1019/1020 | T264N | 1135/1136 | | 1367/1368 | H89T | | | | ++ |
| 1023/1024 | F100S | 1203/1204 | S74P | 1233/1234 | | | | | ++ |
| 1027/1028 | G266M | 1135/1136 | | 1369/1370 | E91W | | | | ++ |
| 1029/1030 | F100R | 1205/1206 | S74G | 1233/1234 | | | | | ++ |
| 1031/1032 | E190S | 1135/1136 | | 1371/1372 | N15V | + | | | |
| 1035/1036 | N270Q | 1135/1136 | | 1373/1374 | Q94H/M95R | | | | ++ |
| 1039/1040 | N66R | 1207/1208 | T40E | 1233/1234 | | | | | + |
| 1041/1042 | F100V | 1209/1210 | S73R/S74F | 1233/1234 | | | | | ++ |
| 1043/1044 | G266Q | 1135/1136 | | 1375/1376 | E91K | | | | ++ |
| 1045/1046 | K33E | 1211/1212 | R7K | 1233/1234 | | | | | ++ |
| 1047/1048 | L53W | 1213/1214 | L27G | 1233/1234 | | | | | ++ |
| 1049/1050 | G266L | 1135/1136 | | 1377/1378 | E91W | | | | ++ |
| 1051/1052 | T264G | 1135/1136 | | 1379/1380 | H89V | | | | ++ |
| 1057/1058 | L53C | 1215/1216 | L27V | 1233/1234 | | | | | ++ |
| 1059/1060 | L53P | 1217/1218 | L27Q | 1233/1234 | | | | | ++ |
| 1065/1066 | Y90D | 1219/1220 | T64I | 1381/1382 | T97M/H120R | | | | + |
| 1067/1068 | F100V | 1221/1222 | S73R/S74Y | 1233/1234 | | | | | ++ |
| 1071/1072 | Y90T | 1223/1224 | T64R | 1233/1234 | | | | | ++ |
| 1075/1076 | N270M | 1135/1136 | | 1383/1384 | M95W | | | | ++ |
| 1079/1080 | G267A | 1135/1136 | | 1385/1386 | D92H | | | | ++ |
| 1081/1082 | N270A | 1135/1136 | | 1387/1388 | M95R | | | | ++ |
| 1085/1086 | E190V | 1135/1136 | | 1313/1314 | N15S | + | | | |
| 1087/1088 | D60Y | 1225/1226 | | 1233/1234 | | | | | ++ |
| 1091/1092 | | 1135/1136 | | 1389/1390 | T153I | ++ | ++ | | ++ |
| 1095/1096 | N329G | 1135/1136 | | 1391/1392 | M154G | + | ++ | + | |
| 1107/1108 | L53S | 1227/1228 | L27R | 1233/1234 | | | | | ++ |
| 1109/1110 | G266S | 1135/1136 | | 1393/1394 | E91P | | | | ++ |
| 1111/1112 | N270V | 1135/1136 | | 1395/1396 | M95S | | | | ++ |
| 1113/1114 | S263W | 1135/1136 | | 1397/1398 | A88G | + | + | | |
| 1115/1116 | | 1135/1136 | | 1399/1400 | P158R | + | ++ | | |
| 1117/1118 | G266H | 1135/1136 | | 1401/1402 | E91T | | | | +++ |
| 1119/1120 | L126R/N470L | 1229/1230 | L100V | 1233/1234 | | | | | ++ |
| 1123/1124 | G266T | 1135/1136 | | 1403/1404 | E91R | | | | ++ |
| 1125/1126 | V331S | 1135/1136 | | 1405/1406 | E155D/S156R | + | ++ | | |
| 1129/1130 | F100S | 1231/1232 | S74R | 1233/1234 | | | | | ++ |
| 1131/1132 | N214D | 1135/1136 | | 1407/1408 | | ++ | | | ++ |
| 1133/1134 | I320V | 1135/1136 | | 1409/1410 | | + | | | ++ |

Levels of increased performance in all conditions were determined relative to the Capsid protein reference polypeptide of SEQ ID NO: 552. Levels of increased performance for the CNS transduction assay are defined as follows: “+” >0.9, “++” >1.3, “+++” >3. Levels of increased performance in the HEK293T transduction assay are defined as follows: “+” > 0.9, “++” >1.3, “+++” >3. Levels of increased performance in the HEK293T transduction assay in the presence of Nabs are defined as follows: “+” >0.9, “++” >1.6, “+++” >3. Levels of increased activity in the qPCR assay are defined as follows: “+” >0.9, “++” >1.3, “+++” >3
Note:
the sequences of MAAP and AAP for some Capsid protein variants differ from the reference sequences of MAAP and AAP, as specified in the table

### Example 8

### In Vitro Characterization of AAV Capsid Variants

In this Example, experiments conducted to characterize AAV capsid variants produced as indicated herein are described.

### Production of AAV Capsids Using HEK293T Cells or Viral Production Cells 2.0

Viral Production Cells 2.0 (ThermoFisher Scientific) were cultured in growth medium (Viral Production Medium [ThermoFisher Scientific]). HEK293T cells were cultured in growth medium (DMEM with 10% fetal bovine serum [both from Corning]). The following four plasmids, as described in Example 1, were used to express the WT AAV9 capsid, or variants: (1) the pHelper plasmid (Agilent Technologies, Inc.), (2) a pAAV-derived plasmid containing the dual reporters FLuc2 and eGFP, (3) a p5 promoter and SV40 pA containing vector including the AAV2 rep gene, and (4) a CMV promoter and SV40 pA containing vector including the AAV9 cap gene (SEQ ID NO: 1). HEK293T cells or Viral Production Cells 2.0 (ThermoFisher Scientific) were transfected with these four plasmids using the lipofection method with the AAV-MAX Transfection Kit (ThermoFisher Scientific) or the polymer transfection method with Polyethylenimine (PEI) Transporter 5® Transfection Reagent (Polysciences, Inc.) and cultured for 3-7 days before lysing with AAV-MAX Lysis Buffer (ThermoFisher Scientific) and benzonase (Sigma-Aldrich). Lysates were clarified by centrifugation and filtration and precipitated with PEG 8000 (Sigma-Aldrich) overnight at 4° C. Precipitate was pelleted by centrifugation and the supernatant was discarded. The pellet was resuspended in buffer and stored at 4° C. until purification.

### Purification of AAV Capsids Using Ultracentrifugation

AAV capsid variants were purified using cesium chloride ultracentrifugation. Briefly, the resuspended pellets containing AAV capsid variants were loaded into a cesium chloride gradient and ultracentrifuged for 12-24 hours. Following centrifugation, the band containing the capsid variants was visually identified and extracted and loaded into a second cesium chloride gradient and ultracentrifuged for 12-24 hours. The band containing the capsid variants packaging full-length transgenes was visually identified and extracted. This material was then buffer exchanged into phosphate-buffered saline with 0.001% pluronic F-68 (ThermoFisher Scientific).

In Vitro Characterization of AAV Capsids Using Human iPSC-Derived Neurons

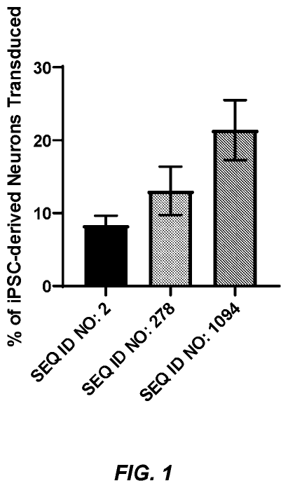

AAV capsid variant transduction potency was determined through high content imaging after transduction of human iPSC-derived neurons using the AAV capsid variants packaging eGFP transgenes. Human iPSC-derived neurons were obtained as described in Example 3 and cultured at densities of 0.5-2E+4 cells/well with 25-200 μL of media (BrainPhys™ Neuronal Medium [STEMCELL™ Technologies]) in appropriate 96- or 384-well tissue culture. The cells were maintained with media (BrainPhys™ Neuronal Medium [STEMCELL™ Technologies]), changing the media every 2-3 days, prior to use in the assay. AAV capsid variants, diluted with media (BrainPhys™ Neuronal Medium [STEMCELL™ Technologies]), were added to iPSC-derived neurons, after removal of the media from the cultures, at doses of 1E+2 to 1E+6 multiplicity of infection (MOI). In some experiments, 0.5-10 μM Etoposide (Sigma-Aldrich) was added to the diluted AAV capsid variants prior to adding to the iPSC-derived neurons to boost transduction. After 1-7 days, the cultures were fixed and immunocytochemistry (ICC) was performed with DAPI (ThermoFisher Scientific), anti-GFP antibodies (Abcam), and anti-NeuN antibodies (Abcam). Fixed cultures were imaged with the ImageXpress Micro Confocal High-Content Imaging System (Molecular Devices). Images were analyzed with the MetaXpress software to determine percent of cells transduced. The results from this analysis are presented in FIG. 1, plotting mean±standard deviation of triplicates.

### Example 9

### In Vivo Characterization of AAV Capsid Variants

In this Example, mouse studies conducted to characterize AAV capsid variants are described.

### Mouse Study to Characterize AAV Capsid Variants

AAV capsid variants, produced and purified as described in Example 8, were characterized in vivo for their transduction potency and biodistribution in the brain. Wildtype male C57BL6 mice (8 weeks old; Charles River Laboratories) were used. Mice were administered a bilateral intracerebroventricular (ICV) infusion with the AAV capsid variants (1E+11 vector genomes/mouse). Animals were anesthetized using isoflurane and sacrificed at four weeks post-dosing. Mice were perfused with phosphate-buffered saline and relevant tissues (i.e., brain, heart, liver, and others) were collected and dissected into two portions. One portion was flash frozen, and stored at −80° C. until analysis of DNA, RNA, and/or protein content. The other portion was fixed for microscopy analysis.

### Microscopy Analysis of Mouse Tissues to Characterize AAV Capsid Variants

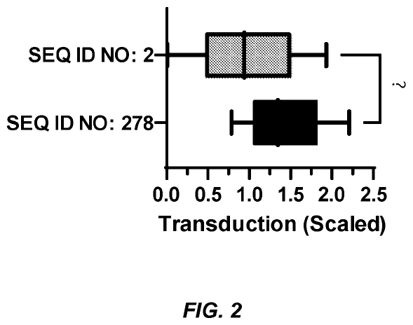

The fixed tissues were paraffin-embedded and sliced into 5 micron thick slices. Fluorescent immunohistochemistry (IHC) was performed with DAPI (ThermoFisher Scientific) and anti-GFP antibodies (Abcam) before mounting on standard microscopy slides. Slides were imaged using the LEICA DMi8 THUNDER Imaging System (Leica). Images were analyzed with the ImageJ software to determine the total number of cells transduced. The results from this analysis were scaled to the mean of SEQ ID NO: 2 and are presented in FIG. 2, showing a box-and-whisker plot of the data.

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

What is claimed is:

1. An engineered adeno-associated virus (AAV) capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to an even-numbered SEQ ID NO. of SEQ ID NOs: 2-1134, wherein the amino acid sequence comprises one or more substitutions relative to the reference sequence corresponding to SEQ ID NO: 2, 278, or 522.

2. The engineered AAV capsid polypeptide of claim 1, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 2, 278, or 522, wherein the amino acid sequence comprises one or more substitutions relative to the reference sequence corresponding to SEQ ID NO: 2.278, or 522.

3. The engineered AAV capsid polypeptide of claim 1, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 2, wherein the amino acid sequence comprises one or more substitutions relative to the reference sequence corresponding to SEQ ID NO: 2.

4. The engineered AAV capsid polypeptide of claim 1, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522, wherein the amino acid sequence comprises one or more substitutions relative to the reference sequence corresponding to SEQ ID NO: 2.

5. The engineered AAV capsid polypeptide of claim 1, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO. of SEQ ID NOs: 4-1134, wherein the amino acid sequence comprises one or more substitutions relative to the reference sequence corresponding to SEQ ID NO: 2.

6. The engineered AAV capsid polypeptide of any one of claims 1-5, wherein the amino acid sequence of the engineered AAV capsid polypeptide comprises at least a substitution at amino acid position 10, 14, 17, 22, 23, 24, 25, 31, 33, 45, 51, 53, 56, 60, 63, 66, 79, 81, 90, 100, 101, 119, 125, 126, 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to SEQ ID NO: 2.

7. The engineered AAV capsid polypeptide of any one of claims 1-6, wherein the amino acid sequence of the engineered AAV capsid polypeptide comprises substitutions in at least two of amino acid positions 588, 665, 457, 495, 456, 505, 494, 497, 592, 586, 585, 706, 549, 552, 664, 661, 461, 668, 548, 510, 640, 419, 349, 504, 508, 453, 589, 56, and 416, wherein the amino acid positions are relative to the reference sequence corresponding to SEQ ID NO: 2.

8. The engineered AAV capsid polypeptide of any one of claims 1-6, wherein the amino acid sequence of the engineered AAV capsid polypeptide comprises at least two substitutions comprising a first and second substitution, wherein the substitutions are at amino acid positions selected from (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, 56, and 416, wherein the first substitution is at a position selected from (a), and the second substitution is at a position selected from (a), (b) and (c), wherein the amino acid positions are relative to the reference sequence corresponding to SEQ ID NO: 2.

9. The engineered AAV capsid polypeptide of any one of claims 1-6, wherein the amino acid sequence of the engineered AAV capsid polypeptide comprises at least a substitution set at amino acid positions 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to the reference sequence corresponding to SEQ ID NO: 2.

10. The engineered AAV capsid polypeptide of any one of claims 1-6, wherein the amino acid sequence of the engineered AAV capsid polypeptide comprises at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 56/529, 331/449, 211/509/712, 331/416/478, 56/507, 56/331/584/597, 56/478, 349/465/479/504/508, 349/640, 331/584/597, 331/416/478/483/507/584/597, 56/416, 233/465/640, 125/508/510/589, 597, 331/597, 56/331/416/665, 125/465/504/508/510, 56/331/478/483/529/597, 384/479, 233/640, 125/419/504/508/530, 504/510/640, 419/504/508, 56/331/361, 416/665, 56/211/361/416/478/584, 56, 56/331/416, 175/211, 56/211/478/483/712, 349/504/508, 125/508/510, 331/416, 331, 479/504/510/589, 331/478, 56/331/478/483/597, 479, 125/504/508, 479/589, or 479/510/589, wherein the amino acid positions are relative to the reference sequence corresponding to SEQ ID NO: 2.

11. The engineered AAV capsid polypeptide of any one of claims 1-6, wherein the amino acid sequence of the engineered AAV capsid polypeptide comprises at least a substitution or substitution set at amino acid position(s) 14/588/665, 173/588/665, 159/588/665, 157/588/665, 152/588/665, 485/588/665, 421/588/665, 391/588/665, 469/588/665, 588/665/628, 461/588/665, 165/588/665, 588/642/665, 588/595/665, 24/588/665, 414/588/665, 512/588/665, 519/588/665, 168/588/665, 588/598/665, 449/588/665, 588/665/736, 156/588/665, 462/588/665, 162/588/665, 416/588/665, 456/588/665, 587/588/665, 586/588/665, 588/665/668, 465/588/665, 454/588/665, 495/505/532/588/665, 494/497/588/665, 497/548/552/588/664/665, 588/661/665, 497/505/586/588/665, 495/588/665, 585/588/665/706, 588/665/706, 416/588/640/665, 665/456/457/494/497/505/588/661/706, 457/495/588/665, 505/588/665, 456/495/505/588/665, 457/497/549/552/588/664/665, 494/505/588/592/665, 456/457/588/665, 548/552/592/588/661/665/706, 456/457/494/497/505/588/661/665, 494/588/665, 494/495/505/588/664/665, 505/588/664/665, 456/494/497/588/661/665, 493/588/640/665, 457/548/555/588/592/661/665, 588/661/664/665, 457/588/592/661/665, 416/588/661/665, 588/664/665, 588/592/664/665, 457/494/497/588/665, 457/588/661/664/665, 456/497/588/592/665, 497/505/588/665, 155/588/665, 588/663/665, 401/588/665, 22/588/665, 175/588/665, 579/588/665, 172/588/665, 444/588/665, 23/588/665, 459/508/588/663/665/667, 17/588/665, 412/588/665, 440/588/665, 387/588/665, 453/586/588/665, 419/588/665, 25/588/665, 472/588/665, 162/450/588/665, 588/665, 437/588/665, 10/588/665, 562/588/665, 169/588/665, 565/588/665, 588/589/665, 495/497/588/665, 119/450/588/665, or 142/552/588/665, wherein the amino acid positions arc relative to the reference sequence corresponding to SEQ ID NO: 2.

12. The engineered AAV capsid polypeptide of any one of claims 1-6, wherein the amino acid sequence of the engineered AAV capsid polypeptide comprises at least a substitution or substitution set at amino acid position(s) 457/495/588/665/725, 414/456/457/459/485/495/588/665/736, 193/457/495/588/665, 157/165/454/457/495/588/665, 157/165/457/495/588/665/668, 457/485/495/588/595/665, 165/454/457/485/495/588/665, 346/457/495/588/665, 421/456/457/459/495/588/665, 218/457/495/588/665, 205/457/495/588/665, 348/457/495/588/665, 421/457/495/588/665, 457/495/551/588/665, 165/457/495/588/665, 457/495/588/665/736, 457/495/529/552/585/588/665, 457/495/535/588/665, 457/495/529/585/588/665, 457/495/535/588/659/665, 457/495/588/594/665, 457/468/495/588/665, 101/457/495/588/665, 457/495/588/665/726, 457/468/495/588/592/665, 451/457/495/588/665, 457/495/559/588/665, 457/495/575/588/665, 457/465/495/588/665, 457/495/560/588/665, 273/457/495/588/665, 389/457/495/510/588/665, 457/470/495/588/665, 100/457/495/588/665, 457/495/588/665/724, 457/485/495/588/597/665, 457/495/510/588/665, 45/457/495/588/665, 256/457/495/588/665, 334/457/495/588/665, 63/457/495/588/665, 211/457/495/588/665, 264/457/495/588/665, 457/495/588/595/665, 391/457/495/588/665, 391/457/495/588/661/665, 200/457/495/588/665, 165/391/454/457/471/495/588/665, 157/165/391/457/495/588/665, 165/391/457/495/588/665, 157/391/421/457/495/588/665, 81/457/495/588/665, 401/414/457/485/495/588/665, 391/454/457/495/588/595/665, 457/495/529/588/665, 457/495/529/552/665/716, 457/493/495/585/588/665/667, 269/457/495/588/665, 457/495/552/588/665, 457/495/515/588/665, 457/495/565/588/665, 457/470/495/588/663/665, 414/457/495/588/665, 457/495/588/597/665, 457/495/597/588/665, 457/495/564/588/665, 389/457/495/588/665, 60/457/495/588/665, 457/495/588/665/720, 451/452/457/495/588/665, 457/495/496/588/665, 214/457/495/588/665, or 320/457/495/588/665, wherein the amino acid positions are relative to the reference sequence corresponding to SEQ ID NO: 2.

13. The engineered AAV capsid polypeptide of any one of claims 1-6, wherein the amino acid sequence of the engineered AAV capsid comprises at least a substitution or substitution set at amino acid position(s) 333/457/495/588/665, 190/457/495/588/665, 254/457/495/588/665, 100/457/495/588/665, 190/457/495/588/665/725, 31/457/495/588/665, 101/457/495/588/665, 258/457/495/588/665, 268/457/495/588/665, 214/451/457/495/588/665, 331/457/495/588/665, 328/457/495/588/665, 33/457/495/588/665, 274/457/495/588/665, 33/457/495/576/588/665, 457/495/560/588/665, 51/190/457/495/588/665, 60/457/495/588/665, 79/272/457/495/588/665, 263/457/495/588/665, 270/457/495/588/665, 267/457/495/588/665, 210/266/457/495/588/665, 259/457/495/588/665, 53/457/495/588/665, 259/457/466/495/588/665, 90/457/495/588/665, 264/457/495/588/665, 172/266/457/495/588/665, 90/457/495/535/588/665, 266/457/495/588/665, 66/457/495/588/665, 329/457/495/588/665, or 126/457/470/495/588/665, wherein the amino acid positions are relative to the reference sequence corresponding to SEQ ID NO: 2.

14. The engineered AAV capsid polypeptide of claim 1, wherein the amino acid sequence of the engineered AAV capsid polypeptide comprises at least one substitution set forth in Tables 5.1, 5.2, 6.1, 6.2, 7.1, and 7.2, wherein the amino acid positions are relative to the reference sequence corresponding to SEQ ID NO: 2.

15. The engineered AAV capsid polypeptide of claim 1, wherein the amino acid sequence of the engineered AAV capsid polypeptide comprises at least a substitution or substitution set of an engineered AAV capsid polypeptide set forth in Tables 5.1, 5.2, 6.1, 6.2, 7.1, and 7.2, wherein the amino acid positions are relative to the reference sequence corresponding to SEQ ID NO: 2.

16. The engineered AAV capsid polypeptide of claim 1, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522.

17. The engineered AAV capsid polypeptide of claim 1, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to an even-numbered SEQ ID NO. of SEQ ID NOs: 4-1134.

18. The engineered AAV capsid polypeptide of claim 1, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278 or 522, wherein the amino acid sequence comprises one or more substitutions relative to the reference sequence corresponding to SEQ ID NO: 278 or 522.

19. The engineered AAV capsid polypeptide of claim 1, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to an even-numbered SEQ ID NO. of SEQ ID NOs: 4-1134, wherein the amino acid sequence comprises one or more substitutions relative to the reference sequence corresponding to SEQ ID NO: 278 or 522.

20. The engineered AAV capsid polypeptide of claim 18 or 19, wherein the amino acid sequence of the engineered AAV capsid polypeptide comprises at least a substitution at amino acid position 10, 14, 17, 22, 23, 24, 25, 31, 33, 45, 51, 53, 56, 60, 63, 66, 79, 81, 90, 100, 101, 119, 125, 126, 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to SEQ ID NO: 278, or 522.

21. The engineered AAV capsid polypeptide of claim 18, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 278, wherein the amino acid sequence comprises one or more substitutions relative to the reference sequence corresponding to SEQ ID NO: 278.

22. The engineered AAV capsid polypeptide of claim 19, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO. of SEQ ID NOs: 332-722, wherein the amino acid sequence comprises one or more substitutions relative to the reference sequence corresponding to SEQ ID NO: 278.

23. The engineered AAV capsid polypeptide of claim 21 or 22, wherein the amino acid sequence of the engineered AAV capsid polypeptide comprises at least a substitution or substitution set at amino acid position(s) 14, 173, 159, 157, 152, 485, 421, 391, 469, 628, 461, 165, 642, 595, 24, 414, 512, 519, 172, 168, 598, 449, 736, 156, 462, 162, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 155, 663, 401, 22, 175, 579, 444, 23, 459/508/663/667, 17, 412, 440, 387, 453/586, 419, 25, 472, 162/450, 585, 437, 10, 562, 169, 588, 565, 589, 495/497, 119/450, or 142/552, wherein the amino acid positions are relative to the reference sequence corresponding to SEQ ID NO: 278.

24. The engineered AAV capsid polypeptide of claim 18, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 552, wherein the amino acid sequence comprises one or more substitutions relative to the reference sequence corresponding to SEQ ID NO: 552.

25. The engineered AAV capsid polypeptide of claim 19, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to an even-numbered SEQ ID NO. of SEQ ID NOs: 724-1134, wherein the amino acid sequence comprises one or more substitutions relative to the reference sequence corresponding to SEQ ID NO: 552.

26. The engineered AAV capsid polypeptide of claim 24 or 25, wherein the amino acid sequence of the engineered AAV capsid polypeptide comprises at least a substitution or substitution set at amino acid position(s) 725, 414/456/459/485/736, 193, 157/165/454, 157/165/668, 485/595, 165/454/485, 346, 421/456/459, 218, 205, 348, 421, 551, 165, 736, 529/552/585, 535, 529/585, 535/659, 594, 468, 101, 726, 468/592, 451, 559, 575, 465, 560, 273, 389/510, 470, 100, 724, 485/597, 510, 45, 256, 334, 63, 211, 264, 595, 391, 391/661, 200, 165/391/454/471, 157/165/391, 165/391, 157/391/421, 81, 401/414/485, 391/454/595, 529, 529/552/588/716, 493/585/667, 269, 552, 515, 565, 470/663, 414, 597, 564, 389, 60, 720, 451/452, 496, 214, or 320, wherein the amino acid positions are relative to the reference sequence corresponding to SEQ ID NO: 552.

27. The engineered AAV capsid polypeptide of claim 24 or 25, wherein the amino acid sequence of the engineered AAV capsid polypeptide comprises at least a substitution or substitution set at amino acid position(s) 333, 190, 254, 100, 190/725, 31, 101, 258, 268, 214/451, 331, 328, 33, 274, 33/576, 560, 51/190, 60, 79/272, 263, 270, 267, 210/266, 259, 53, 259/466, 90, 264, 172/266, 90/535, 266, 66, 329, or 126/470, wherein the amino acid positions are relative to the reference sequence corresponding to SEQ ID NO: 552.

28. The engineered AAV capsid polypeptide of claim 1, wherein the amino acid sequence of the engineered AAV capsid polypeptide comprises at least one substitution set forth in Tables 5.1, 5.2, 6.1, 6.2, 7.1, and 7.2, wherein the amino acid positions are relative to the reference sequence corresponding to SEQ ID NO: 2, 278, or 552.

29. The engineered AAV capsid polypeptide of claim 1, wherein the amino acid sequence of the engineered AAV capsid polypeptide comprises at least a substitution or substitution set of an engineered AAV capsid polypeptide set forth in Tables 5.1, 5.2, 6.1, 6.2, 7.1, and 7.2, wherein the amino acid positions are relative to the reference sequence corresponding to SEQ ID NO: 2, 278, or 552.

30. The engineered AAV capsid polypeptide of claim 1, comprising an amino acid sequence comprising an even-numbered SEQ ID NO. of SEQ ID NOs: 4-1134, optionally wherein the amino acid sequence has 1, 2, 3, 4, 5, 6, 7, 8, 9, or up to 10 substitutions.

31. An engineered adeno-associated virus (AAV) VP2 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 138-736 of an even-numbered SEQ ID NO. of SEQ ID NOs: 2-1134, wherein the amino acid sequence comprises one or more substitutions relative to the reference sequence corresponding to residues 138-736 of SEQ ID NO: 2, 278, or 522.

32. The engineered AAV VP2 capsid polypeptide of claim 31, wherein the amino acid sequence of the engineered AAV VP2 capsid polypeptide comprises residues 138-736 of an engineered AAV capsid of any one of claims 1-30.

33. The engineered AAV VP2 capsid polypeptide of claim 31, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 2, wherein the amino acid sequence comprises one or more substitutions relative to the reference sequence corresponding to residues 138-736 of SEQ ID NO: 2.

34. The engineered AAV VP2 capsid polypeptide of claim 31, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522, wherein the amino acid sequence comprises one or more substitutions relative to the reference sequence corresponding to residues 138-736 of SEQ ID NO: 2.

35. The engineered AAV VP2 capsid polypeptide of claim 31, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO. of SEQ ID NOs: 4-1134, wherein the amino acid sequence comprises one or more substitutions relative to the reference sequence corresponding to residues 138-736 of SEQ ID NO: 2.

36. The engineered AAV VP2 capsid polypeptide of any one of claims 31-35, wherein the amino acid sequence of the engineered AAV VP2 capsid polypeptide comprises at least a substitution at amino acid position 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 138-736 of SEQ ID NO: 2.

37. The engineered AAV VP2 capsid polypeptide of any one of claims 31-36, wherein the amino acid sequence of the engineered AAV VP2 capsid polypeptide comprises substitutions in at least two amino acid positions 588, 665, 457, 495, 456, 505, 494, 497, 592, 586, 585, 706, 549, 552, 664, 661, 461, 668, 548, 510, 640, 419, 349, 504, 508, 453, 589, and 416, wherein the amino acid positions are relative to the reference sequence corresponding to residues 138-736 of SEQ ID NO: 2.

38. The engineered AAV VP2 capsid polypeptide of any one of claims 31-36, wherein the amino acid sequence of the engineered AAV VP2 capsid polypeptide comprises at least two substitutions comprising a first and second substitution, wherein the substitutions are at amino acid positions (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, and 416, wherein the first substitution is at a position selected from (a), and the second substitution is at a position selected from (a), (b) and (c), wherein the amino acid positions are relative to the reference sequence corresponding to residues 138-736 of SEQ ID NO: 2.

39. The engineered AAV VP2 capsid polypeptide of any one of claims 31-36, wherein the amino acid sequence of the AAV VP2 capsid polypeptide comprises at least a substitution set at amino acid positions 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to residues 138-736 of SEQ ID NO: 2.

40. The engineered AAV VP2 capsid polypeptide of any one of any one of claims 31-36, wherein the amino acid sequence of the engineered AAV VP2 capsid polypeptide comprises at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 331/449, 211/509/712, 331/416/478, 507, 331/584/597, 478, 349/465/479/504/508, 349/640, 331/416/478/483/507/584/597, 416, 233/465/640, 508/510/589, 597, 331/597, 331/416/665, 465/504/508/510, 331/478/483/529/597, 384/479, 233/640, 419/504/508/530, 504/510/640, 419/504/508, 331/361, 416/665, 211/361/416/478/584, 331/416, 175/211, 211/478/483/712, 349/504/508, 508/510, 331/416, 331, 479/504/510/589, 331/478, 331/478/483/597, 479, 504/508, 479/589, or 479/510/589, wherein the amino acid positions are relative to the reference sequence corresponding to residues 138-736 of SEQ ID NO: 2.

41. The engineered AAV VP2 capsid polypeptide of any one of claims 31-36, wherein the amino acid sequence of the engineered AAV VP2 capsid polypeptide comprises at least a substitution or substitution set at amino acid position(s) 173/588/665, 159/588/665, 157/588/665, 152/588/665, 485/588/665, 421/588/665, 391/588/665, 469/588/665, 588/665/628, 461/588/665, 165/588/665, 588/642/665, 588/595/665, 414/588/665, 512/588/665, 519/588/665, 168/588/665, 588/598/665, 449/588/665, 588/665/736, 156/588/665, 462/588/665, 162/588/665, 416/588/665, 456/588/665, 587/588/665, 586/588/665, 588/665/668, 465/588/665, 454/588/665, 495/505/532/588/665, 494/497/588/665, 497/548/552/588/664/665, 588/661/665, 497/505/586/588/665, 495/588/665, 585/588/665/706, 588/665/706, 416/588/640/665, 665/456/457/494/497/505/588/661/706, 457/495/588/665, 505/588/665, 456/495/505/588/665, 457/497/549/552/588/664/665, 494/505/588/592/665, 456/457/588/665, 548/552/592/588/661/665/706, 456/457/494/497/505/588/661/665, 494/588/665, 494/495/505/588/664/665, 505/588/664/665, 456/494/497/588/661/665, 493/588/640/665, 457/548/555/588/592/661/665, 588/661/664/665, 457/588/592/661/665, 416/588/661/665, 588/664/665, 588/592/664/665, 457/494/497/588/665, 457/588/661/664/665, 456/497/588/592/665, 497/505/588/665, 155/588/665, 588/663/665, 401/588/665, 175/588/665, 579/588/665, 172/588/665, 444/588/665, 459/508/588/663/665/667, 412/588/665, 440/588/665, 387/588/665, 453/586/588/665, 419/588/665, 472/588/665, 162/450/588/665, 588/665, 437/588/665, 562/588/665, 169/588/665, 565/588/665, 588/589/665, 495/497/588/665, 450/588/665, or 142/552/588/665, wherein the amino acid positions are relative to the reference sequence corresponding to residues 138-736 of SEQ ID NO: 2.

42. The engineered AAV VP2 capsid polypeptide of any one of claims 31-36, wherein the amino acid sequence of the engineered AAV VP2 capsid polypeptide comprises at least a substitution or substitution set at amino acid position(s) 457/495/588/665/725, 414/456/457/459/485/495/588/665/736, 193/457/495/588/665, 157/165/454/457/495/588/665, 157/165/457/495/588/665/668, 457/485/495/588/595/665, 165/454/457/485/495/588/665, 346/457/495/588/665, 421/456/457/459/495/588/665, 218/457/495/588/665, 205/457/495/588/665, 348/457/495/588/665, 421/457/495/588/665, 457/495/551/588/665, 165/457/495/588/665, 457/495/588/665/736, 457/495/529/552/585/588/665, 457/495/535/588/665, 457/495/529/585/588/665, 457/495/535/588/659/665, 457/495/588/594/665, 457/468/495/588/665, 457/495/588/665/726, 457/468/495/588/592/665, 451/457/495/588/665, 457/495/559/588/665, 457/495/575/588/665, 457/465/495/588/665, 457/495/560/588/665, 273/457/495/588/665, 389/457/495/510/588/665, 457/470/495/588/665, 457/495/588/665/724, 457/485/495/588/597/665, 457/495/510/588/665, 256/457/495/588/665, 334/457/495/588/665, 211/457/495/588/665, 264/457/495/588/665, 457/495/588/595/665, 391/457/495/588/665, 391/457/495/588/661/665, 200/457/495/588/665, 165/391/454/457/471/495/588/665, 157/165/391/457/495/588/665, 165/391/457/495/588/665, 157/391/421/457/495/588/665, 401/414/457/485/495/588/665, 391/454/457/495/588/595/665, 457/495/529/588/665, 457/495/529/552/665/716, 457/493/495/585/588/665/667, 269/457/495/588/665, 457/495/552/588/665, 457/495/515/588/665, 457/495/565/588/665, 457/470/495/588/663/665, 414/457/495/588/665, 457/495/588/597/665, 457/495/597/588/665, 457/495/564/588/665, 389/457/495/588/665, 457/495/588/665/720, 451/452/457/495/588/665, 457/495/496/588/665, 214/457/495/588/665, or 320/457/495/588/665, wherein the amino acid positions are relative to the reference sequence corresponding to residues 138-736 of SEQ ID NO: 2.

43. The engineered AAV VP2 capsid polypeptide of any one of claims 31-36, wherein the amino acid sequence of the engineered AAV VP2 capsid polypeptide comprises at least a substitution or substitution set at amino acid position(s) 333/457/495/588/665, 190/457/495/588/665, 254/457/495/588/665, 190/457/495/588/665/725, 258/457/495/588/665, 268/457/495/588/665, 214/451/457/495/588/665, 331/457/495/588/665, 328/457/495/588/665, 274/457/495/588/665, 457/495/576/588/665, 457/495/560/588/665, 60/457/495/588/665, 79/272/457/495/588/665, 263/457/495/588/665, 270/457/495/588/665, 267/457/495/588/665, 210/266/457/495/588/665, 259/457/495/588/665, 259/457/466/495/588/665, 90/457/495/588/665, 264/457/495/588/665, 172/266/457/495/588/665, 266/457/495/588/665, 66/457/495/588/665, 329/457/495/588/665, or 457/470/495/588/665, wherein the amino acid positions are relative to the reference sequence corresponding to residues 138-736 of SEQ ID NO: 2.

44. The engineered AAV VP2 capsid polypeptide of claim 31, wherein the amino acid sequence of the engineered AAV VP2 capsid polypeptide comprises at least one substitution between residues 138-736 of AAV capsid polypeptide, wherein the substitution are set forth in Tables 5.1, 5.2, 6.1, 6.2, 7.1, and 7.2, and wherein the amino acid positions are relative to the reference sequence corresponding to residues 138-736 of SEQ ID NO: 2.

45. The engineered AAV VP2 capsid polypeptide of claim 31, wherein the amino acid sequence of the engineered AAV VP2 capsid polypeptide comprises at least a substitution or substitution set between residues 138-736 of an engineered AAV capsid polypeptide set forth in Tables 5.1, 5.2, 6.1, 6.2, 7.1, and 7.2, wherein the amino acid positions are relative to the reference sequence corresponding to residues 138-736 of SEQ ID NO: 2.

46. The engineered AAV VP2 capsid polypeptide of claim 31, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522.

47. The engineered AAV VP2 capsid polypeptide of claim 31, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO. of SEQ ID NOs: 4-1134.

48. The engineered AAV VP2 capsid polypeptide of claim 31, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278 or 522, wherein the amino acid sequence comprises one or more substitutions relative to the reference sequence corresponding to residues 138-736 of SEQ ID NO: 278 or 522.

49. The engineered AAV VP2 capsid polypeptide of claim 31, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO. of SEQ ID NOs: 4-1134, wherein the amino acid sequence comprises one or more substitutions relative to the reference sequence corresponding to residues 138-736 of SEQ ID NO: 278 or 522.

50. The engineered AAV VP2 capsid polypeptide of claim 48 or 49, wherein the amino acid sequence of the engineered AAV VP2 capsid polypeptide comprises at least a substitution at amino acid position 142, 152, 155, 156, 157, 159, 162, 165, 168, 169, 172, 173, 175, 190, 193, 200, 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 138-736 of SEQ ID NO: 278, or 522.

51. The engineered AAV VP2 capsid polypeptide of claim 48, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 278, wherein the amino acid sequence comprises one or more substitutions relative to the reference sequence corresponding to residues 138-736 of SEQ ID NO: 278.

52. The engineered AAV VP2 capsid polypeptide of claim 49, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO. of SEQ ID NOs: 332-722, wherein the amino acid sequence comprises one or more substitutions relative to the reference sequence corresponding to residues 138-736 of SEQ ID NO: 278.

53. The engineered AAV VP2 capsid polypeptide of claim 51 or 52, wherein the amino acid sequence of the engineered AAV VP2 capsid polypeptide comprises at least a substitution or substitution set at amino acid position(s) 173, 159, 157, 152, 485, 421, 391, 469, 628, 461, 165, 642, 595, 414, 512, 519, 172, 168, 598, 449, 736, 156, 462, 162, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 155, 663, 401, 175, 579, 444, 459/508/663/667, 412, 440, 387, 453/586, 419, 472, 162/450, 585, 437, 562, 169, 588, 565, 589, 495/497, 119/450, or 142/552, wherein the amino acid positions are relative to the reference sequence corresponding to residues 138-736 of SEQ ID NO: 278.

54. The engineered AAV VP2 capsid polypeptide of claim 48, wherein the amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of SEQ ID NO: 552, wherein the amino acid sequence comprises one or more substitutions relative to the reference sequence corresponding to residues 138-736 of SEQ ID NO: 552.

55. The engineered AAV VP2 capsid polypeptide of claim 49, wherein the amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 138-736 of an even-numbered SEQ ID NO. of SEQ ID NOs: 724-1134, wherein the amino acid sequence comprises one or more substitutions relative to the reference sequence corresponding to residues 138-736 of SEQ ID NO: 552.

56. The engineered AAV VP2 capsid polypeptide of claim 54 or 55, wherein the amino acid sequence of the engineered AAV VP2 capsid polypeptide comprises at least a substitution or substitution set at amino acid position(s) 725, 414/456/459/485/736, 193, 157/165/454, 157/165/668, 485/595, 165/454/485, 346, 421/456/459, 218, 205, 348, 421, 551, 165, 736, 529/552/585, 535, 529/585, 535/659, 594, 468, 726, 468/592, 451, 559, 575, 465, 560, 273, 389/510, 470, 724, 485/597, 510, 256, 334, 63, 211, 264, 595, 391, 391/661, 200, 165/391/454/471, 157/165/391, 165/391, 157/391/421, 401/414/485, 391/454/595, 529, 529/552/588/716, 493/585/667, 269, 552, 515, 565, 470/663, 414, 597, 564, 389, 720, 451/452, 496, 214, or 320, wherein the amino acid positions are relative to the reference sequence corresponding to residues 138-736 of SEQ ID NO: 552.

57. The engineered AAV VP2 capsid polypeptide of claim 54 or 55, wherein the amino acid sequence of the engineered AAV VP2 capsid polypeptide comprises at least a substitution or substitution set at amino acid position(s) 333, 190, 254, 190/725, 258, 268, 214/451, 331, 328, 274, 576, 560, 79/272, 263, 270, 267, 210/266, 259, 259/466, 264, 172/266, 535, 266, 329, or 470, wherein the amino acid positions are relative to the reference sequence corresponding to residues 138-736 of SEQ ID NO: 552.

58. The engineered AAV VP2 capsid polypeptide of claim 31, wherein the amino acid sequence of the engineered AAV VP2 capsid polypeptide comprises at least one substitution between residues 138-736 of AAV capsid polypeptide, wherein the substitutions are set forth in Tables 5.1, 5.2, 6.1, 6.2, 7.1, and 7.2, and wherein the amino acid positions are relative to the reference sequence corresponding to residues 138-736 of SEQ ID NO: 2, 278, or 552.

59. The engineered AAV VP2 capsid polypeptide of claim 31, wherein the amino acid sequence of the engineered AAV VP2 capsid polypeptide comprises at least a substitution or substitution set between residues 138-736 of an engineered AAV capsid polypeptide set forth in Tables 5.1, 5.2, 6.1, 6.2, 7.1, and 7.2, wherein the amino acid positions are relative to the reference sequence corresponding to residues 138-736 of SEQ ID NO: 2, 278, or 552.

60. The engineered AAV VP2 capsid polypeptide of claim 31, wherein the amino acid sequence of the engineered AAV VP2 capsid polypeptide comprising residues 138-736 of an even-numbered SEQ ID NO. of SEQ ID NOs: 4-1134, optionally wherein the amino acid sequence has 1, 2, 3, 4, 5, 6, 7, 8, 9, or up to 10 substitutions.

61. An engineered adeno-associated virus (AAV) VP3 capsid polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 203-736 of an even-numbered SEQ ID NO. of SEQ ID NOs: 2-1134, wherein the amino acid sequence comprises one or more substitutions relative to the reference sequence corresponding to residues 203-736 of SEQ ID NO: 2, 278, or 522.

62. The engineered AAV VP3 capsid polypeptide of claim 61, wherein the amino acid sequence of the engineered AAV VP3 capsid polypeptide comprises residues 203-736 of an engineered AAV capsid of any one of claims 1-30.

63. The engineered AAV VP3 capsid polypeptide of claim 61, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 2, 278, or 522, wherein the amino acid sequence comprises one or more substitutions relative to the reference sequence corresponding to residues 203-736 of SEQ ID NO: 2, 278, or 522.

64. The engineered AAV VP3 capsid polypeptide of claim 61, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 2, wherein the amino acid sequence comprises one or more substitutions relative to the reference sequence corresponding to residues 203-736 of SEQ ID NO: 2.

65. The engineered AAV VP3 capsid polypeptide of claim 61, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278 or 522, wherein the amino acid sequence comprises one or more substitutions relative to the reference sequence corresponding to residues 203-736 of SEQ ID NO: 2.

66. The engineered AAV VP3 capsid polypeptide of claim 61, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO. of SEQ ID NOs: 4-1134, wherein the amino acid sequence comprises one or more substitutions relative to the reference sequence corresponding to residues 203-736 of SEQ ID NO: 2.

67. The engineered AAV VP3 capsid polypeptide of any one of claims 61-66, wherein the amino acid sequence of the engineered AAV VP3 capsid polypeptide comprises at least a substitution at amino acid position 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 203-736 of SEQ ID NO: 2.

68. The engineered AAV VP3 capsid polypeptide of any one of claims 61-66, wherein the amino acid sequence of the engineered AAV VP3 capsid polypeptide comprises substitutions in at least two amino acid positions 588, 665, 457, 495, 456, 505, 494, 497, 592, 586, 585, 706, 549, 552, 664, 661, 461, 668, 548, 510, 640, 419, 349, 504, 508, 453, 589, and 416, wherein the amino acid positions are relative to the reference sequence corresponding to residues 203-736 of SEQ ID NO: 2.

69. The engineered AAV VP3 capsid polypeptide of any one of claims 61-66, wherein the amino acid sequence of the engineered AAV VP3 capsid polypeptide comprises at least two substitutions comprising a first and second substitution, wherein the substitutions are at amino acid positions selected from (a) 588, 665, 457, and 495; (b) 456, 505, 494, 497, 592, 586, 585, 706, 549, 552, 664, 661, 461, 668, and 548; and (c) 510, 640, 419, 349, 504, 508, 453, 589, and 416, wherein the first substitution is at a position selected from (a), and the second substitution is at a position selected from (a), (b) and (c), wherein the amino acid positions are relative to the reference sequence corresponding to residues 203-736 of SEQ ID NO: 2.

70. The engineered AAV VP3 capsid polypeptide of any one of claims 61-66, wherein the amino acid sequence of the AAV VP3 capsid polypeptide comprises at least a substitution set at amino acid positions 588/665, 457/495/588/665, 461/588/665, 588/665/668, 495/588/665, 494/495/505/588/664/665, 494/588/665, 588/592/664/665, 456/497/588/592/665, 494/497/588/665, 456/495/505/588/665, 456/457/588/665, 497/505/586/588/665, 456/457/494/497/505/588/661/665/706, 457/497/549/552/588/664/665, 585/588/665/706, 495/588/661/665, 497/548/552/588/664/665, 456/457/494/497/505/588/661/665, 456/494/497/588/661/665, or 457/494/497/588/665, wherein the amino acid positions are relative to residues 203-736 of SEQ ID NO: 2.

71. The engineered AAV VP3 capsid polypeptide of any one of claims 61-66, wherein the amino acid sequence of the engineered AAV VP3 capsid polypeptide comprises at least a substitution or substitution set at amino acid position(s) 547, 453, 555, 535, 501, 456, 550, 497, 529, 705, 554, 500, 548, 582, 586, 584, 585, 592, 459, 492, 661, 666, 587, 552, 706, 499, 665, 538, 556, 532, 495, 588, 460, 663, 454, 660, 489, 457, 709, 494, 659, 493, 505, 668, 589, 662, 667, 537, 452, 549, 710, 640, 25/492, 545, 211/416, 539, 589/640, 588/665, 551, 504/510, 455, 508, 465/589/640, 419/504/508/510, 528, 490, 664, 553, 458, 361/483, 449/584, 384/479/504/508/510, 507/509/529, 510/640, 233/508, 331/449, 211/509/712, 331/416/478, 507, 331/584/597, 478, 349/465/479/504/508, 349/640, 331/416/478/483/507/584/597, 416, 233/465/640, 508/510/589, 597, 331/597, 331/416/665, 465/504/508/510, 331/478/483/529/597, 384/479, 233/640, 419/504/508/530, 504/510/640, 419/504/508, 331/361, 416/665, 211/361/416/478/584, 331/416, 175/211, 211/478/483/712, 349/504/508, 508/510, 331/416, 331, 479/504/510/589, 331/478, 331/478/483/597, 479, 504/508, 479/589, or 479/510/589, wherein the amino acid positions are relative to the reference sequence corresponding to residues 203-736 of SEQ ID NO: 2.

72. The engineered AAV VP3 capsid polypeptide of any one of claims 61-66, wherein the amino acid sequence of the engineered AAV VP3 capsid polypeptide comprises at least a substitution or substitution set at amino acid position(s) 485/588/665, 421/588/665, 391/588/665, 469/588/665, 588/665/628, 461/588/665, 588/642/665, 588/595/665, 414/588/665, 512/588/665, 519/588/665, 588/598/665, 449/588/665, 588/665/736, 462/588/665, 416/588/665, 456/588/665, 587/588/665, 586/588/665, 588/665/668, 465/588/665, 454/588/665, 495/505/532/588/665, 494/497/588/665, 497/548/552/588/664/665, 588/661/665, 497/505/586/588/665, 495/588/665, 585/588/665/706, 588/665/706, 416/588/640/665, 665/456/457/494/497/505/588/661/706, 457/495/588/665, 505/588/665, 456/495/505/588/665, 457/497/549/552/588/664/665, 494/505/588/592/665, 456/457/588/665, 548/552/592/588/661/665/706, 456/457/494/497/505/588/661/665, 494/588/665, 494/495/505/588/664/665, 505/588/664/665, 456/494/497/588/661/665, 493/588/640/665, 457/548/555/588/592/661/665, 588/661/664/665, 457/588/592/661/665, 416/588/661/665, 588/664/665, 588/592/664/665, 457/494/497/588/665, 457/588/661/664/665, 456/497/588/592/665, 497/505/588/665, 155/588/665, 588/663/665, 401/588/665, 579/588/665, 444/588/665, 459/508/588/663/665/667, 412/588/665, 440/588/665, 387/588/665, 453/586/588/665, 419/588/665, 472/588/665, 450/588/665, 588/665, 437/588/665, 562/588/665, 565/588/665, 588/589/665, 495/497/588/665, or 552/588/665, wherein the amino acid positions are relative to the reference sequence corresponding to residues 203-736 SEQ ID NO: 2.

73. The engineered AAV VP3 capsid polypeptide of any one of claims 61-66, wherein the amino acid sequence of the engineered AAV VP3 capsid polypeptide comprises at least a substitution or substitution set at amino acid position(s) 457/495/588/665/725, 414/456/457/459/485/495/588/665/736, 457/485/495/588/595/665, 454/457/485/495/588/665, 346/457/495/588/665, 421/456/457/459/495/588/665, 218/457/495/588/665, 205/457/495/588/665, 348/457/495/588/665, 421/457/495/588/665, 457/495/551/588/665, 457/495/588/665/736, 457/495/529/552/585/588/665, 457/495/535/588/665, 457/495/529/585/588/665, 457/495/535/588/659/665, 457/495/588/594/665, 457/468/495/588/665, 457/495/588/665/726, 457/468/495/588/592/665, 451/457/495/588/665, 457/495/559/588/665, 457/495/575/588/665, 457/465/495/588/665, 457/495/560/588/665, 273/457/495/588/665, 389/457/495/510/588/665, 457/470/495/588/665, 457/495/588/665/724, 457/485/495/588/597/665, 457/495/510/588/665, 256/457/495/588/665, 334/457/495/588/665, 211/457/495/588/665, 264/457/495/588/665, 457/495/588/595/665, 391/457/495/588/665, 391/457/495/588/661/665, 391/454/457/471/495/588/665, 391/421/457/495/588/665, 401/414/457/485/495/588/665, 391/454/457/495/588/595/665, 457/495/529/588/665, 457/495/529/552/665/716, 457/493/495/585/588/665/667, 269/457/495/588/665, 457/495/552/588/665, 457/495/515/588/665, 457/495/565/588/665, 457/470/495/588/663/665, 414/457/495/588/665, 457/495/588/597/665, 457/495/597/588/665, 457/495/564/588/665, 389/457/495/588/665, 457/495/588/665/720, 451/452/457/495/588/665, 457/495/496/588/665, 214/457/495/588/665, or 320/457/495/588/665, wherein the amino acid positions are relative to the reference sequence corresponding to residues 203-736 SEQ ID NO: 2.

74. The engineered AAV VP3 capsid polypeptide of any one of claims 61-66, wherein the amino acid sequence of the engineered AAV VP3 capsid polypeptide comprises at least a substitution or substitution set at amino acid position(s) 333/457/495/588/665, 254/457/495/588/665, 258/457/495/588/665, 268/457/495/588/665, 214/451/457/495/588/665, 331/457/495/588/665, 328/457/495/588/665, 274/457/495/588/665, 457/495/576/588/665, 457/495/560/588/665, 272/457/495/588/665, 263/457/495/588/665, 270/457/495/588/665, 267/457/495/588/665, 210/266/457/495/588/665, 259/457/495/588/665, 259/457/466/495/588/665, 264/457/495/588/665, 266/457/495/588/665, 329/457/495/588/665, or 457/470/495/588/665, wherein the amino acid positions are relative to the reference sequence corresponding to residues 203-736 SEQ ID NO: 2.

75. The engineered AAV VP3 capsid polypeptide of claim 61, wherein the amino acid sequence of the engineered AAV VP3 capsid polypeptide comprises at least one substitution between residues 203-736 set forth in Tables 5.1, 5.2, 6.1, 6.2, 7.1, and 7.2, wherein the amino acid positions are relative to the reference sequence corresponding to residues 203-736 SEQ ID NO: 2.

76. The engineered AAV VP3 capsid polypeptide of claim 61, wherein the amino acid sequence of the engineered AAV VP3 capsid polypeptide comprises at least a substitution or substitution set between residues 203-736 of an engineered AAV capsid polypeptide set forth in Tables 5.1, 5.2, 6.1, 6.2, 7.1, and 7.2, wherein the amino acid positions are relative to the reference sequence corresponding to residues 203-736 SEQ ID NO: 2.

77. The engineered AAV VP3 capsid polypeptide of claim 61, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278 or 522.

78. The engineered AAV VP3 capsid polypeptide of claim 61, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO. of SEQ ID NOs: 4-1134.

79. The engineered AAV VP3 capsid polypeptide of claim 61, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278 or 522, wherein the amino acid sequence comprises one or more substitutions relative to the reference sequence corresponding to residues 203-736 of SEQ ID NO: 278 or 522.

80. The engineered AAV VP3 capsid polypeptide of claim 61, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO. of SEQ ID NOs: 4-1134, wherein the amino acid sequence comprises one or more substitutions relative to the reference sequence corresponding to residues 203-736 of SEQ ID NO: 278 or 522.

81. The engineered AAV VP3 capsid polypeptide of claim 79 or 80, wherein the amino acid sequence of the engineered AAV VP3 capsid polypeptide comprises at least a substitution at amino acid position 205, 210, 211, 214, 218, 233, 250, 254, 256, 258, 259, 263, 264, 266, 267, 268, 269, 270, 272, 273, 274, 320, 328, 329, 331, 333, 334, 346, 348, 349, 361, 384, 387, 389, 391, 401, 412, 414, 416, 419, 421, 437, 440, 444, 449, 450, 451, 452, 453, 454, 455, 456, 457, 458, 459, 460, 461, 462, 465, 466, 468, 469, 470, 471, 472, 478, 479, 483, 485, 489, 490, 492, 492, 493, 494, 495, 496, 497, 499, 500, 501, 504, 505, 507, 508, 509, 510, 512, 515, 519, 528, 529, 530, 532, 535, 537, 538, 539, 545, 547, 548, 549, 550, 551, 552, 553, 554, 555, 556, 559, 560, 562, 562, 564, 565, 575, 576, 579, 582, 584, 585, 586, 586, 587, 588, 589, 592, 594, 595, 597, 598, 628, 640, 642, 659, 660, 661, 662, 663, 664, 665, 666, 667, 668, 705, 706, 709, 710, 712, 716, 720, 724, 725, 726, or 736, or combinations thereof, wherein the amino acid positions are relative to the reference sequence corresponding to residues 203-736 SEQ ID NO: 278 or 522.

82. The engineered AAV VP3 capsid polypeptide of claim 79, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 278, wherein the amino acid sequence comprises one or more substitutions relative to the reference sequence corresponding to residues 203-736 of SEQ ID NO: 278.

83. The engineered AAV VP3 capsid polypeptide of claim 80, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO. of SEQ ID NOs: 332-722, wherein the amino acid sequence comprises one or more substitutions relative to the reference sequence corresponding to residues 203-736 of SEQ ID NO: 278.

84. The engineered AAV VP3 capsid polypeptide of claim 82 or 83, wherein the amino acid sequence of the engineered AAV VP3 capsid polypeptide comprises at least a substitution or substitution set at amino acid position(s) 485, 421, 391, 469, 628, 461, 642, 595, 414, 512, 519, 598, 449, 736, 462, 416, 456, 587, 586, 668, 465, 454, 495/505/532, 494/497, 497/548/552/664, 661, 497/505/586, 495, 555, 585/706, 706, 416/640, 456/457/494/497/505/661/706, 457/495, 505, 456/495/505, 457/497/549/552/664, 494/505/592, 456/457, 548/552/592/661/706, 456/457/494/497/505/661, 494, 494/495/505/664, 505/664, 456/494/497/661, 493/640, 457/548/555/592/661, 661/664, 457/592/661, 416/661, 664, 592/664, 457/494/497, 457/661/664, 456/497/592, 497/505, 663, 401, 579, 444, 459/508/663/667, 412, 440, 387, 453/586, 419, 472, 450, 585, 437, 562, 169, 588, 565, 589, 495/497, or 142/552, wherein the amino acid positions are relative to the reference sequence corresponding to residues 203-736 of SEQ ID NO: 278.

85. The engineered AAV VP3 capsid polypeptide of claim 79, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of SEQ ID NO: 552, wherein the amino acid sequence comprises one or more substitutions relative to the reference sequence corresponding to residues 203-736 of SEQ ID NO: 552.

86. The engineered AAV VP3 capsid polypeptide of claim 80, comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 203-736 of an even-numbered SEQ ID NO. of SEQ ID NOs: 724-1134, wherein the amino acid sequence comprises one or more substitutions relative to the reference sequence corresponding to residues 203-736 of SEQ ID NO: 552.

87. The engineered AAV VP3 capsid polypeptide of claim 85 or 86, wherein the amino acid sequence of the engineered AAV VP3 capsid polypeptide comprises at least a substitution or substitution set at amino acid position(s) 725, 414/456/459/485/736, 454, 668, 485/595, 454/485, 346, 421/456/459, 218, 205, 348, 421, 551, 736, 529/552/585, 535, 529/585, 535/659, 594, 468, 726, 468/592, 451, 559, 575, 465, 560, 273, 389/510, 470, 724, 485/597, 510, 256, 334, 211, 264, 595, 391, 391/661, 391/454/471, 391/421, 401/414/485, 391/454/595, 529, 529/552/588/716, 493/585/667, 269, 552, 515, 565, 470/663, 414, 597, 564, 389, 720, 451/452, 496, 214, or 320, wherein the amino acid positions are relative to the reference sequence corresponding to residues 203-736 of SEQ ID NO: 552.

88. The engineered AAV VP3 capsid polypeptide of claim 85 or 86, wherein the amino acid sequence of the engineered AAV VP3 capsid polypeptide comprises at least a substitution or substitution set at amino acid position(s) 333, 254, 725, 258, 268, 214/451, 331, 328, 274, 576, 560, 272, 263, 270, 267, 210/266, 259, 259/466, 264, 266, 535, 329, or 470, wherein the amino acid positions are relative to the reference sequence corresponding to residues 203-736 of SEQ ID NO: 552.

89. The engineered AAV VP3 capsid polypeptide of claim 61, wherein the amino acid sequence of the engineered AAV VP3 capsid polypeptide comprises at least a substitution between residues 203-736 set forth in Tables 5.1, 5.2, 6.1, 6.2, 7.1, and 7.2, wherein the amino acid positions are relative to the reference sequence corresponding to residues 203-736 of SEQ ID NO: 278 or 522.

90. The engineered AAV VP3 capsid polypeptide of claim 61, wherein the amino acid sequence of the engineered AAV VP3 capsid polypeptide comprises at least a substitution or substitution set between residues 203-736 of an engineered AAV capsid polypeptide set forth in Tables 5.1, 5.2, 6.1, 6.2, 7.1, and 7.2, wherein the amino acid positions are relative to the reference sequence corresponding to residues 203-736 of SEQ ID NO: 278 or 522.

91. The engineered AAV VP3 capsid polypeptide of claim 61, wherein the amino acid sequence of the engineered AAV VP3 capsid polypeptide comprises residues 203-736 of an even-numbered SEQ ID NO. of SEQ ID NOs: 4-1134, optionally wherein the amino acid sequence has 1, 2, 3, 4, 5, 6, 7, 8, 9, or up to 10 substitutions.

92. An engineered MAAP polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 1136, wherein the amino acid sequence comprises one or more substitutions relative to the reference sequence corresponding to SEQ ID NO: 1136.

93. The engineered MAAP polypeptide of claim 92, wherein the amino acid sequence of the engineered MAAP polypeptide comprises at least a substitution at amino acid position 5, 7, 17, 27, 30, 34, 40, 64, 73, 74, 75, 93, 99, 100, or 116, or combinations thereof, wherein the amino acid positions are relative to SEQ ID NO: 1136.

94. The engineered MAAP polypeptide of claim 92, wherein the amino sequence of the engineered MAAP polypeptide comprises at least a substitution or substitution set at amino acid position(s) 99, 30, 17, 93, 116, 73, 5, 75, 34, 74, 27, 7, 73/74, 64, 40, or 100, wherein the amino acid positions are relative to SEQ ID NO: 1136.

95. The engineered MAAP polypeptide of claim 92, wherein the amino acid sequence of the engineered MAAP polypeptide comprises SEQ ID NO: 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, or 1232, optionally wherein the amino acid sequence has 1, 2, 3, 4, 5, 6, 7, 8, 9, or up to 10 substitutions.

96. An engineered AAP polypeptide comprising an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 1234, wherein the amino acid sequence comprises one or more substitutions relative to the reference sequence corresponding to SEQ ID NO: 1234.

97. The engineered AAP polypeptide of claim 96, wherein the amino acid sequence of the engineered AAP polypeptide comprises at least a substitution at amino acid position 14, 15, 31, 49, 58, 79, 83, 84, 85, 88, 89, 91, 92, 93, 94, 95, 97, 99, 100, 102, 104, 105, 120, 153, 154, 155, 156, 157, 158, 160, 173, 174, or 194, or combinations thereof, wherein the amino acid positions are relative to SEQ ID NO: 1234.

98. The engineered AAP polypeptide of claim 96, wherein the amino acid sequence of the engineered AAP polypeptide comprises at least a substitution or substitution set at amino acid position(s) 58, 156, 49/156, 174, 157/158, 160, 158, 15, 79, 104, 31/156, 83, 92/93, 102, 155/156, 14/15, 153, 194, 99, 85, 105, 97, 88, 94/95, 91/92, 100, 91, 92, 83/84, 89, 94, 94/95/173, 97/120, 95, or 154, or combinations thereof, wherein the amino acid positions are relative to SEQ ID NO: 1234.

99. The engineered AAP polypeptide of claim 96, wherein the amino acid sequence of the engineered AAP polypeptide comprises SEQ ID NO: 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, or 1410, optionally wherein the amino acid sequence has 1, 2, 3, 4, 5, 6, 7, 8, 9, or up to 10 substitutions.

100. A recombinant polynucleotide comprising a polynucleotide sequence encoding an engineered AAV capsid polypeptide of any one of claims 1-30.

101. The recombinant polynucleotide of claim 100, comprising a polynucleotide sequence having at least 60%, 65%, 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to an SEQ ID NO: 277 or 521, wherein the recombinant polynucleotide encodes an AAV capsid polypeptide.

102. The recombinant polynucleotide of claim 100, comprising a polynucleotide sequence having at least 60%, 65%, 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to an odd-numbered SEQ ID NO. of SEQ ID NOs: 3-1133, wherein the recombinant polynucleotide encodes an AAV capsid polypeptide.

103. The recombinant polynucleotide of claim 100, comprising a polynucleotide sequence comprising an odd-numbered SEQ ID NO. of SEQ ID NOs: 3-1133.

104. A recombinant polynucleotide comprising a polynucleotide sequence encoding an engineered AAV VP2 capsid of any one of claims 31-60.

105. The recombinant polynucleotide of claim 104, comprising a polynucleotide sequence having at least 60%, 65%, 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 412-2208 of SEQ ID NO: 277 or 521, wherein the recombinant polynucleotide encodes an AAV VP2 capsid polypeptide.

106. The recombinant polynucleotide of claim 104, wherein the polynucleotide sequence comprises at least 60%, 65%, 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 412-2208 of an odd-numbered SEQ ID NO. of SEQ ID NOs: 33-1133, wherein the recombinant polynucleotide encodes an AAV VP2 capsid polypeptide.

107. The recombinant polynucleotide of claim 104, wherein the polynucleotide sequence comprises nucleotide residues 412-2208 of an odd-numbered SEQ ID NO. of SEQ ID NOs: 33-1133.

108. A recombinant polynucleotide comprising a polynucleotide sequence encoding an engineered AAV VP3 capsid of any one of claims 61-91.

109. The recombinant polynucleotide of claim 108, comprising a polynucleotide sequence having at least 60%, 65%, 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 607-2208 of SEQ ID NO: 277 or 521, wherein the recombinant polynucleotide encodes an AAV VP3 capsid polypeptide.

110. The recombinant polynucleotide of claim 108, wherein the polynucleotide sequence comprises at least 60%, 65%, 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 607-2208 of an odd-numbered SEQ ID NO. of SEQ ID NOs: 33-1133, wherein the recombinant polynucleotide encodes an AAV VP3 capsid polypeptide.

111. The recombinant polynucleotide of claim 108, wherein the polynucleotide sequence comprises nucleotide residues 607-2208 of an odd-numbered SEQ ID NO. of SEQ ID NOs: 33-1133.

112. An expression vector comprising at least one recombinant polynucleotide of any one of claims 100-111.

113. The expression vector of claim 112, wherein the recombinant polynucleotide is operably linked to a control sequence.

114. The expression vector of claim 113, wherein the control sequence comprises at least a promoter.

115. A host cell comprising an expression vector of any one of claims 112-114.

116. The host cell of claim 115, comprising an insect cell or mammalian cell.

117. A method of producing an engineered AAV VP1, VP2, and/or VP3 capsid polypeptide in a host cell comprising culturing a host cell of claim 115 or 116, under suitable culture conditions, such that the engineered AAV VP1, VP2, and/or VP3 capsid is produced.

118. The method of claim 117, further comprising recovering at least one engineered AAV capsid polypeptide from the culture and/or host cells.

119. The method of claim 117 or 118, further comprising a step of purifying the engineered AAV VP1, VP2, and/or VP3 capsid polypeptide.

120. A recombinant polynucleotide comprising a polynucleotide sequence encoding an engineered MAAP polypeptide of any one of claims 92-95.

121. The recombinant polynucleotide of claim 120, wherein the polynucleotide sequence comprises at least 60%, 65%, 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to SEQ ID NO: 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, or 1231, wherein the polynucleotide sequence encodes an engineered MAAP polypeptide.

122. The recombinant polynucleotide of claim 120, wherein the polynucleotide sequence comprises SEQ ID NO: 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, or 1231.

123. An expression vector comprising at least one recombinant polynucleotide of any one of claims 120-122.

124. The expression vector of claim 123, wherein the polynucleotide is operably linked to a control sequence.

125. The expression vector of claim 124, wherein the control sequence comprises at least a promoter.

126. A host cell comprising an expression vector of any one of claims 123-125.

127. The host cell of claim 126, comprising an insect cell or mammalian cell.

128. A method of producing an engineered MAAP polypeptide in a host cell comprising culturing a host cell of claim 126 or 127, under suitable culture conditions such that at least one engineered MAAP polypeptide is produced.

129. The method of claim 128, further comprising recovering at least one engineered MAAP polypeptide from the culture and/or host cells.

130. The method of claim 128 or 129, further comprising a step of purifying the at least one engineered MAAP polypeptide.

131. A recombinant polynucleotide comprising a polynucleotide sequence encoding an engineered AAP polypeptide of any one of claims 96-99.

132. The recombinant polynucleotide of claim 131, wherein the polynucleotide sequence comprises at least 60%, 65%, 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to SEQ ID NO: 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1367, 1371, 1373, 1375, 1377, 1379, 1381, 1383, 1385, 1387, 1389, 1391, 1393, 1395, 1397, 1399, 1401, 1403, 1405, 1407, or 1409, wherein the polynucleotide sequence encodes an engineered AAP polypeptide.

133. The recombinant polynucleotide of claim 131, wherein the polynucleotide sequence encoding the engineered AAP polypeptide comprises SEQ ID NO: 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1367, 1371, 1373, 1375, 1377, 1379, 1381, 1383, 1385, 1387, 1389, 1391, 1393, 1395, 1397, 1399, 1401, 1403, 1405, 1407, or 1409.

134. An expression vector comprising at least one recombinant polynucleotide of any one of claims 131-133.

135. The expression vector of claim 134, wherein the polynucleotide is operably linked to a control sequence.

136. The expression vector of claim 135, wherein the control sequence comprises at least a promoter.

137. A host cell comprising an expression vector of any one of claims 134-136.

138. The host cell of claim 137, comprising an insect cell or mammalian cell.

139. A method of producing an engineered AAP polypeptide in a host cell comprising culturing a host cell of claim 137 or 138, under suitable culture conditions such that at least one engineered AAP polypeptide is produced.

140. The method of claim 139, further comprising recovering at least one engineered AAP polypeptide from the culture and/or host cells.

141. The method of claim 139 or 140, further comprising purifying the at least one engineered AAP polypeptide.

142. An rAAV virus or virion comprising an engineered AAV capsid polypeptide of any one of claims 1-91.

143. The rAAV virus or virion of claim 142, wherein the engineered AAV capsid polypeptide comprises VP1, VP2, and/or VP3 capsid polypeptide.

144. The rAAV virus or virion of claim 142 or 143, wherein the rAAV virus or virion comprises at least the engineered AAV VP1 capsid polypeptide.

145. The rAAV virus or virion of claim 142 or 143, wherein the rAAV virus or virion comprises at least the engineered AAV VP2 capsid polypeptide.

146. The rAAV virus or virion of claim 142 or 143, wherein the rAAV virus or virion comprises at least the engineered AAV VP3 capsid polypeptide.

147. The rAAV virus or virion of any one of claims 142-146, further comprising a rAAV vector.

148. The rAAV virus or virion of claim 147, wherein the rAAV vector comprises an inverted terminal repeat and a transgene.

149. The rAAV virus or virion of claim 148, wherein the transgene encodes a polypeptide.

150. The rAAV virus or virion of claim 149, wherein the polypeptide encoded by the transgene is a reporter polypeptide, enzyme, ligand polypeptide, a signal transduction polypeptide, or structural polypeptide.

151. The rAAV virus or virion of claim 148, wherein the transgene is capable of transcribing a non-coding RNA molecule.

152. The rAAV virus or virion of claim 151, wherein the non-coding RNA effector molecule comprises a shRNA, miRNA, or antisense-RNA.

153. A host cell comprising a rAAV virus or virion of any one of claims 142-152.

154. The host cell of claim 153, comprising an insect or mammalian cell.

155. A method of preparing a rAAV virus or virion comprising an AAV capsid polypeptide of any one of claims 1-91, the method comprising providing a competent cell comprising an expression vector of any one of claims 112-114 and a suitable AAV helper virus or AAV helper plasmid, and culturing the cell under suitable conditions such that the AAV virus or virion is produced.

156. The method of claim 155, comprising further providing an rAAV vector in the cell.

157. A method of transducing a cell with an rAAV virus or virion of any one of claims 142-152, comprising contacting a cell with a rAAV virus or virion of any one of claims 142-152 under conditions suitable for transduction of the cell by the rAAV virus or virion.

158. The method of claim 157, wherein the cell is a neuronal cell or glial cell.

159. A method of transducing a rAAV virus or virion of any one of claims 142-152 to the CNS, comprising administering a rAAV virus or virion of any one of claims 142-152 to a subject.

160. The method of claim 159, wherein the rAAV virus or virion is by administered intravenously, intranasally, intrathecally, or intracerebrally.
